SEQUENCE LISTING (20 sequences)

SEQ ID NO: 1 - DNA, 2544 bp, Homo sapines
  CDS (1)..(2499)
atgaatagttacatcgtcccaccagaggaagcacttttttttatcacaacaatttatcatgaaagaggtggtgatttaagagaaaacattgaaacgaaaggtgtcactctgggagctgcgcctggggataaactgattcctcagtggggctgtaacattgctcagggtcagtggtcactaggcagggaatggagcccaggaggcgtggggaaaggggatggaggccggacgtggccagcggattcagggcttgactcacacactcacacaaaagaggccggaaagtgtccggaggaagccggcgcgtcacggaggacggatccgggaccctgccgcgcccgcccagcggcgcccggtcccgggtccacagccgcactcactccgccgcgctctccgccaccgccaccactgcggccaccgccaatgaaacgcctcccgctcctagtggttttttccactttgttgaattgttcctatactcaaaattgcaccaagacaccttgtctcccaaatgcaaaatgtgaaatacgcaatggaattgaagcctgctattgcaacatgggattttcaggaaatggtgtcacaatttgtgaagatgataatgaatgtggaaatttaactcagtcctgtggcgaaaatgctaattgcactaacacagaaggaagttattattgtatgtgtgtacctggcttcagatccagcagtaaccaagacaggtttatcactaatgatggaaccgtctgtatagaaaatgtgaatgcaaactgccatttagataatgtctgtatagctgcaaatattaataaaactttaacaaaaatcagatccataaaagaacctgtggctttgctacaagaagtctatagaaattctgtgacagatctttcaccaacagatataattacatatatagaaatattagctgaatcatcttcattactaggttacaagaacaacactatctcagccaaggacaccctttctaactcaactcttactgaatttgtaaaaaccgtgaataattttgttcaaagggatacatttgtagtttgggacaagttatctgtgaatcataggagaacacatcttacaaaactcatgcacactgttgaacaagctactttaaggatatcccagagcttccaaaagaccacagagtttgatacaaattcaacggatatagctctcaaagttttcttttttgattcatataacatgaaacatattcatcctcatatgaatatggatggagactacataaatatatttccaaagagaaaagctgcatatgattcaaatggcaatgttgcagttgcatttgtatattataagagtattggtcctttgctttcatcatctgacaacttcttattgaaacctcaaaattatgataattctgaagaggaggaaagagtcatatcttcagtaatttcagtctcaatgagctcaaacccacccacattatatgaacttgaaaaaataacatttacattaagtcatcgaaaggtcacagataggtataggagtctatgtgcattttggaattactcacctgataccatgaatggcagctggtcttcagagggctgtgagctgacatactcaaatgagacccacacctcatgccgctgtaatcacctgacacattttgcaattttgatgtcctctggtccttccattggtattaaagattataatattcttacaaggatcactcaactaggaataattatttcactgatttgtcttgccatatgcatttttaccttctggttcttcagtgaaattcaaagcaccaggacaacaattcacaaaaatctttgctgtagcctatttcttgctgaacttgtttttcttgttgggatcaatacaaatactaataagctcttctgttcaatcattgccggactgctacactacttctttttagctgcttttgcatggatgtgcattgaaggcatacatctctatctcattgttgtgggtgtcatctacaacaagggatttttgcacaagaatttttatatctttggctatctaagcccagccgtggtagttggattttcggcagcactaggatacagatattatggcacaaccaaagtatgttggcttagcaccgaaaacaactttatttggagttttataggaccagcatgcctaatcattcttgttaatctcttggcttttggagtcatcatatacaaagtttttcgtcacactgcagggttgaaaccagaagttagttgctttgagaacataaggtcttgtgcaagaggagccctcgctcttctgttccttctcggcaccacctggatctttggggttctccatgttgtgcacgcatcagtggttacagcttacctcttcacagtcagcaatgctttccaggggatgttcatttttttattcctgtgtgttttatctagaaagattcaagaagaatattacagattgttcaaaaatgtcccctgttgttttggatgtttaagctgttgaaatgaagtctgccaaatcttgctctaacaaataaaatgttatcta

SEQ ID NO: 2 - protein, 832 aa, Homo sapines [sequence omitted]

SEQ ID NO: 3 - DNA, 4274 bp, Rattus norvegicus
  CDS (236)..(2452)
ccacaggctgagactagagtccaggctgtttgggtgaaggggcctggcggccggacgtggcctgcagagtctgggctgtgcacacattcacacaaaagaggccgggaagtgacaggaggaagctgtgcgtcacaagggactgagcgggaccctgccgcgcctgcccagctccaggacagaccccaactcttgccttcagcgctctgcggagccagccagctccacccggcttccaatgagactcctcctgcttctagtgggtctctccactttgctgaatcactcctacacacaaaactgcaagacaccgtgtctcccaaatgccaagtgtgaggtgttggacgaagtggcagcctgcttctgcagtacaggctacactgggaatggcatcacgatttgtgaagatgtagacgagtgcaacgagacctccgtctgcggtgatcacgctgtgtgtgaaaacacgaatggaggatttagctgcttctgcgtggaaggttatcagacctccaccgggaagacgcagttcacgcctaatgatggctcttactgccaagatgtagacgagtgcaacgagacctccgtctgcggtgatcacgctgtgtgtgaaaacacgaacggaggatttagctgcttctgcgtggaaggttatcagacctccaccgggaagacgcagttcacgcctaatgatggctcttactgccaagaaattgtgaattcaaattgccacttagagcatgactgcattgctgcaaacattaataaaactctaaaaagaattggacccataacagaacagctgactttactccatgaaatctacaagaattctgaggctgagctttctctggtggatatagtcacatacatagagatactaacagaatcatcctcactacaaggctacataaagaacaccacttcgcccaaggatgcctacttcggttcagctcttactgaatttggaaaaaccgtcaataattttgttgaaaagaacacacatgaaatgtgggaccagttacctacaaatcgtagaagactccatctcacaaaactgatgcacgctgctgagcacgtcaccttacagatctctcagaacatccagaagaatactcagtttgacatgaattctaccgacttggctctcaaggttttcgtttttgattcagttcacatgaagcatactcatccccatatgaatgtggacggaggctatgtaaaaatatccccgaggagaaaatctgcatatgacccaaatggcaacgtcattgttgcattcctgtgctataggagcattggccccttgctttcctcatctgacgacttcttactgggcgctcagagtgacaattccaaaggaaaggagaaggtcatttcttcagtgatttctgcctcaattagctcaaacccacccacactgtatgaacttgaaaaaattacatttacactgagtcatgtaaagctctcagataagcaccagacacagtgcgccttttggaactactcagtcgatgacatgaacaatggcagctggtcatctgagggctgtgagctgacatactccaacgacacccatacttcctgccgatgtagtcatctgacacactttgcgattttgatgtcccccagtacctccattgaagttaaagattacaatatcctgacgaggatcactcagctgggaataatcatctccctgatctgcctcgccatatgcattttcaccttctggttcttcagtgagattcaaagcaccaggaccacaatccacaagaatctctgctgcagcctctttcttgcacaactagtttttcttgtcggcatcaacataaacacaaacaagctggtctgctctatcatcgctggcctgctccattacttcttcttagctgcctttgcctggatgtgcattgaaggcatctacctatacctcatcgttgttgggctcatctataacaaggggtttttacacaagaacttctatatctttggctatcttagcccggctgtagttgttggattctcggcctctttgggatacagatattatggtaccaccaaagtatgttggttgagcactgaaaacaactttatctggagcttcatagggccagcgtgtctaatcattcttgttaatctcttggcttttggagttatcatatacaaagtgttccgccacactgctggactgaagccagaagttagttgctacgagaacataaggtcttgcgccagaggagccctggccctcctcttccttctgggtaccacctggacctttggggttctccacgtagtgcatgcatctgttgtgacagcctacctcttcacagtcagcaacgctttccaagggatgtttattttcttattcctatgtgttttatctagaaagattcaagaagaatattacagattgttcaaaaatgtcccctgctgttttgaatgtttaagataaacaacgagaagacacaataattatagctgaaatgaaatggaaattccaagatttcggatagcctgtgtgacaaaaatgagcctgccttcattgttagtaattaatttcaaattcgcttttctgttcgcagtataaaagatgtagttaatgtgagataaaattatgggccagagagctcctgtgtgttttcctacatgacatagttagatatgtcaaaaatagtactgcagatatttggaaagtaattggtttctctggagtgatatcactgtgcccaaggaaagatttctttctaacacaagaaatagatgaatgtcctcaaggaagcgactggcttgatatctttgtgactcatgttgcctttcaaacgagtcccctaccaccatagtaatgagttcctttgcagaaaggagagtataagaaacttggaggggcagaatatgaagcaatggagaagccttctctgacaaggaattgtcattccaataaaattggctttctccaaaattgaagaggaaaaaattttcaggctaaaataacgaaaaaggaaatgcatcctagcactttgggaattggtctgaacttaaaaggcccagacctaaatttactacatccatgttcttccttactgttctaaaccaaagaaaaaccttaaaatttacagatacatggatgagtgttctcacataacatcatatttgaatgtaaatttttttcattcctcacagattaagacttcagcaacatatttggtaaaacataaatttgtcaaactataagactgttcatatctttagtgaaaaaatagaatgtgaagtattttgtctataatattttactgttatgaaaataatcttttcatattagagcagtatacttgaatactttactgtttttaatcttacaaatagtgtgattcatgttgcaaccagcccttttaattgactgtatttaaaagggcattataaatttaaactattgatgaagtaaattataatggttttctgatcagaaaatacatacttaaagcattatttataacaaataaaaagtcactgagcactgcaggggtttcacagtggatctgatatttttagaccgtttcctatcacctatcagtctatttacttaaatgtacagctctaccaattctcttactcaaaggaagaggcagtatttttctcagaagtgagtcattgttctgtaccttcctggagacatgattcgatccattgaacattgtggttttaattcttgtgctgttgaatgaagcctgacaagacacctcctaaaaaatgaaatgtcagctggatgaagcagccctgctactgcctgactgagttgttctctcaggaaagaccactcacctgccaagaagcacgttgcatctctacagatctcagggtttctcccatgccaagtctgtagcccacgagcatcattgtcattctaagatgggactgtagaaataggatatcaaaacataatccgttcaatcaatggataagaaactatcacatgtagtagacagaataacccttctcaaatattcatacactcctcttcacaagctgtggccgtggtggatagtgaggagcaggaggtcctgtcaggaggaagagtagctgaggtccactcagttggagaaggctctcactgtgctgggggaagtcagcatgctgacgatgttactttagtttgggtctcttgttttggacatctcatttctagagctgtaaagacaataaaattctattatcaaagccaaaaaaaaaaaaaaaaaaaaaaaaaaaaaaaaaaaaaaaaaaaaaaaaaaaaaaaa

SEQ ID NO: 4 - protein, 738 aa, Rattus norvegicus
MRLLLLLVGLSTLLNHSYTQNCKTPCLPNAKCEVLDEVAACFCSTGYTGNGITICEDVDECNETSVCGDHAVCENTNGGFSCFCVEGYQTSTGKTQFTPNDGSYCQDVDECNETSVCGDHAVCENTNGGFSCFCVEGYQTSTGKTQFTPNDGSYCQEIVNSNCHLEHDCIAANINKTLKRIGPITEQLTLLHEIYKNSEAELSLVDIVTYIEILTESSSLQGYIKNTTSPKDAYFGSALTEFGKTVNNFVEKNTHEMWDQLPTNRRRLHLTKLMHAAEHVTLQISQNIQKNTQFDMNSTDLALKVFVFDSVHMKHTHPHMNVDGGYVKISPRRKSAYDPNGNVIVAFLCYRSIGPLLSSSDDFLLGAQSDNSKGKEKVISSVISASISSNPPTLYELEKITFTLSHVKLSDKHQTQCAFWNYSVDDMNNGSWSSEGCELTYSNDTHTSCRCSHLTHFAILMSPSTSIEVKDYNILTRITQLGIIISLICLAICIFTFWFFSEIQSTRTTIHKNLCCSLFLAQLVFLVGININTNKLVCSIIAGLLHYFFLAAFAWMCIEGIYLYLIVVGLIYNKGFLHKNFYIFGYLSPAVVVGFSASLGYRYYGTTKVCWLSTENNFIWSFIGPACLIILVNLLAFGVIIYKVFRHTAGLKPEVSCYENIRSCARGALALLFLLGTTWTFGVLHVVHASVVTAYLFTVSNAFQGMFIFLFLCVLSRKIQEEYYRLFKNVPCCFECLR

SEQ ID NO: 5 - DNA, 4139 bp, Mus musculus
  CDS (105)..(2324)
acgcggggacaggggacagagcgagaccctgcggtgcctgcctagctccaggacagacccgcactctagcctcgagcgctctgccgccagctccgcggcttccaatgagactcctcccgcttctagtgggtttctccactttgctgaattgttcctacacacaaaactgcagcaagacaacgtgtctccccaatgccaagtgcgaagtgcacaatggtgtggaagcctgcttctgcagccaggggtactctgggaatggtgtcacgatttgtgaagatatagatgagtgcagcgagtcttctgtctgcggcgatcatgctgtgtgtgaaaacgtgaacgggggcttcagctgcttctgcagggaaggttatcagaccgccacggggaagtcacagttcacacctaatgatggctcttactgccaagatatagatgagtgcagcgagtcttctgtctgcggtgatcatgctgtgtgtgaaaacgtgaacgggggcttcagctgcttctgcagggaaggttatcagaccgccacggggaagtcacagttcacacctaatgatggctcttactgccaagaaagcatgaattcaaattgccacttagagcatgcctgcatcgctgcaaacattaataaaactttaaaaagaattggacccataacagaacagacaactttactccaagaaatctacagaaattctgaggctgagctctctctgatggatatagtcacatacatagagatcctaactgaatcatcctcactactaggccacccgaacagcaccacttcatacaaggatgcccacttcaactcaactcttactgaatttggggaaaccatcaataattttgttgaaaggagtacacataaaatgtgggaccagttaccgacaaatcacagaagacttcatctcacaaaactgatgcacactgctgagctagtcaccttacagatcgctcagaacatccagaagaattctcagtttgatatgaattctactgacttggctctcaaggtttttgcttttgattcaactcacatgaagcatgctcacccccacatgaatgtggatggaggctatgtgaaaatatccccaaggagaaaggctgcacatggcacaactggcaatgtagtagttgcattcctctgctataagagcattggtcccttgctatcctcatctgacaacttcttactggacactcaaaatgataattctgaaggaaaggaaaaagtcatttcttcagtgatttctgcctcaattagctcaaatccacccacattatatgaacttgaaaaaattacatttacactaagtcatgtaaagctctcagataagcaccggacccagtgtgccttttggaactactcagttgatgccatgaacaatggcarctggtcaacggagggctgtgagctgacacactcaaacgacacccacacctcctgccgctgtagtcacctgacacactttgcgattttgatgtcctctacttcttccattgggattaaggattataatatcctgacgaggatcactcaactcgggataatcatctccctgatctgcctcgccatctgcatcttcaccttctggttcttcagtgaaatccaaagcaccaggaccacgattcacaagaacctgtgctgcagcctctttcttgcagaacttgtttttcttattgggatcaacataaatacgaataagttggtctgctctatcattgctggcctgctccattacttcttcttagctgcctttgcctggatgtgcatcgaaggcattcacctatacctcatcgttgtcggcgtcatctacaacaaggggtttttacacaagaacttttatatctttggctatctcagcccagctgtagttgttggattctcagcatctttaggatacagatattatggaaccacgaaagtatgttggctgagcactgaaaacaacttcatttggagctttataggaccagcgtgtctaatcattcttgtgaatctcttggcttttggagttatcatatacaaagttttccgccacactgctggactgaaaccagaagttagttgctatgagaacataaggtcttgtgccagaggtgccctagccctcctcttccttctgggtaccacctggatctttggggttctccatgtagtgcatgcatctgttgtgacagcctacctcttcacagtcagcaatgctttccaagggatgttcattttcttattcctatgcgttttgtctagaaagattcaagaggaatattatagattgttcaaaaatgtcccctgctgctttggatgtttaagataaacaatgagaagtcatgataattacagctgcaatgagatgaaaattccaagattcagataacctgtgtggcaaaaaatgaagcctgtttttattgttagtaattaatttcaaatccatttttctgttcacagtataagagatgtagttaatgtgagataaaattatggaccagagagctacagtgtgttttcttacatgacatagttagagatatgtcaaaaatagtactgcagatatttggaaagtaattggtttctctggagtgatatcactgtgcccaaggaaagatttctttctaacacgagaaatatatgaatgtcctcaaggaaaccactggctcgatatctttgtgactcatgttgcctttcaaacgagttccctaccaccttagtaatgagttcctttgcaggaaggagagcataagagacgtggaggggcagagtatgaagcagtgacgaaggcttctctgacaaggaattgtcattccaataaactcagcttctctaaacttgatgagaaaatctcaagataaaataacgagaaaggaaatatatcctagcagtttgggaattggtctgaagtaaaaagccccagatctaaatttgctacatccatgttcttccttactcttctaaaaccagagaaaagccttacaactgacattatcagagatggatgctcttacactaacattagatttgagtgtaaaatgttttcattccacacagattaagacttcaaatatgtagtcagtaaaacatagatttgtcaaagtataatactgtttatgtctttagtgaaaagaatgtgtgcagtattttgtctataatattttactgttatgaaaattaccttttaatattaaatcagtatacttgaatactttgctgtttttaatcttacaaatagtgtaattcatgttgcaacaggcccttttaattgactgtacataaaaggtcattataaatttaaagtattgatgaagtgaattataattcttttctgatcagaaaatacacaattaaagcattatttataacaaataagaagtcactgagtgctgtaggggtttcacagtgggtctagttttagactgtttctactatctctcaragtctattggctcaaatgtatggctctatctattctcttgttcaaatgaagaggcagattttttttcagaagtgagtcattgttctgaaccttcstgaacagcataattcaatctactggacattgcaattttaattcttgtgctgttgaatgaagcctgtcgagacctctcctgaaaaatgaacagtcagctggatgaagcagccctatcgctgcctgaccgagttgttctctcaggagagaccactcacctgtcaagaagggctttgcatttctagagcctgtgagatggtacactttgactaaatctctggatttcttctgtgctaagtctgtggcccatgactgccattgtcattctgggttgggactgtagaaataggatatcaaaacctagtctgctcaatcagtggatatgaaactattgcacgtggtagacagagtgaccctccacaaatatctgtacgctcctcttcagagcctgtgbctgtggtggatggcgaggagcaggagggccctgtgkggagggagggtagctgacctccactcagttggagcactctcactaccctgagggaagtcagcatgctgctgatgtatttcagtgtgggtctcctggtttggaactctcatttctagagctacaagacaataaaattctattatcaaagccaaaaaaaaaaaaaaaaaaaaaaaaaaaaa

SEQ ID NO: 6 - protein, 739 aa, Mus musculus
  misc_feature (432)..(432): The ′Xaa′ at location 432 stands for Ser, or Asn.
MRLLPLLVGFSTLLNCSYTQNCSKTTCLPNAKCEVHNGVEACFCSQGYSGNGVTICEDIDECSESSVCGDHAVCENVNGGFSCFCREGYQTATGKSQFTPNDGSYCQDIDECSESSVCGDHAVCENVNGGFSCFCREGYQTATGKSQFTPNDGSYCQESMNSNCHLEHACIAANINKTLKRIGPITEQTTLLQEIYRNSEAELSLMDIVTYIEILTESSSLLGHPNSTTSYKDAHFNSTLTEFGETINNFVERSTHKMWDQLPTNHRRLHLTKLMHTAELVTLQIAQNIQKNSQFDMNSTDLALKVFAFDSTHMKHAHPHMNVDGGYVKISPRRKAAHGTTGNVVVAFLCYKSIGPLLSSSDNFLLDTQNDNSEGKEKVISSVISASISSNPPTLYELEKITFTLSHVKLSDKHRTQCAFWNYSVDAMNNGXWSTEGCELTHSNDTHTSCRCSHLTHFAILMSSTSSIGIKDYNILTRITQLGIIISLICLAICIFTFWFFSEIQSTRTTIHKNLCCSLFLAELVFLIGININTNKLVCSIIAGLLHYFFLAAFAWMCIEGIHLYLIVVGVIYNKGFLHKNFYIFGYLSPAVVVGFSASLGYRYYGTTKVCWLSTENNFIWSFIGPACLIILVNLLAFGVIIYKVFRHTAGLKPEVSCYENIRSCARGALALLFLLGTTWIFGVLHVVHASVVTAYLFTVSNAFQGMFIFLFLCVLSRKIQEEYYRLFKNVPCCFGCLR

SEQ ID NO: 7 - DNA, 2504 bp, Danio rerio
  CDS (51)..(2231)
aaagacattccagcttcacctctaaaatggagtttttccacagttctccaatgaaactcctgcttttcgctgcttggttttcaagtttgctggatccatgcagatttcttgacatttgccagtcttgtcacccaaatgctgactgcgatgacatctgtaaatgcagaacagggtacactggtaatggaatcagcgattgtagagatgacaatgaatgtgagactgtacctgaaatttgtggcttacatgccaactgcaccaactatgttggcggttactactgtaactgtttatctggattcatttcgaatggaacagagcaatttcaaactaatgatgggacctcctgcaacgatataaatgagtgtgaggaggacagaaaatgtggacccaattcaaaatgccacaacaatattggctcattcatctgctcatgtttgagaggttatacatcgccagcagggccctggttcatgcccaaccatgggacagactgcatagaaaacaagatacactgccatcaggaccacaaatgcactaaagagacagttaacagcacactggaaagaatgaccaatctgtccatttcagagcgtctgaaagagataagatatcaaacatcagctgcactctctcctgttcttctcatatcatacattgaggccatggtcagcagtaaattaaattttggagatgtgtctaaagattcaaaggaacacgtcaatgaaaccatcacgaatttggtttttagtgtaaataacttagtcgagaaggatgaaaaggtggaatggaagaagatcaatgaagatttgaggaagtattatgtcaccaaactccttcacacagcagagaaggaaacactggccctgtctgctggatacacacatgctactcagatgcaggtgcatgccggtgatgtagaaatgaaattatacacatttgaaccccgccaagcacagaagcatccactgtcagccaacatccaaggaaactccatttcactaagcaccaagaaagcaagacatgcaaacaacaatggcagtacatccgtcgtattcctcatctatcacagcatcggggatcttttaaagcctgccgatgaccctggagtggccgattactctcgctatgcagcagcgggtgaaattacagttaattcacctgtaatagcagcagccatcagtaaccagaagacacttccactgaacgatgtgaccttcactctcaaacacacacaggagatagatccagcccgtgatgagactaagtgtgcatcctgggaatattcaccaagcatgatgggacactggagtttagatggctgtatacgcactcgtgtgaacaccacacacacttcatgttcttgtaatcacctgacacacttcgctattctcatgtcctccgctcgcgctaatctgcttgcacactataatgttctgacgaggataacacagctgggaatggtgatctctctcatctgcctgtccatgtgtattttcaccttctggttcttccgagacatccagaacaccagaaccacgatccacaagaacctgtgctgcagcctcttcatggcccagttcatctttctcattgggattaacaaaagtgcacacaagtggttctgttctctgatcgctggcctgctgcactacttcttcttggctgcatttgcttggatgtgcatagaagggatccatttatatctcattgttgttggcgtcatctataacaaaggtttcctgcaccggaacttctacgcatttggttatgggagccctgctgtggttgtcgcaatatctgcaactcttggttataaatattatgggaccagttctgtgtgttggctaagcactgaaaataacttcatctggagtttcataggcccacccattctgatcatcctggttaatctgctggcatttgctgtgatcatctataaggtttatcgtcacactgcagtaaagaaacctgaaatcagtcactatgaaaatatcaggtcttgtgcaagaggagccattgctctcctgtttgttctgggagtaacctgggcatttggtgtgatgtacatcttatatgagaccaccctgaccgcgtatctcttcaccttcgccaatgtgttccaagggatgttcattttcatattcctttgcgttctgtccagaaggattcaagaggagtactacagattattcaagaacatgccatgttgttttgagtgtctgcgatgaacaggtgaactacaatgcaatgcttttagatcaaggcattaaaaagacgtacttggcatgaatgttacaatatatttatttttgaaagaaatgtattaaaaaactaagtgatactgcatgctatgagtttcataacctgctggagtcagtagcttcaagacaataacaatattttctatctgattactggttaaaatgattgtatatatactgtattgcattctaataaaatcattgtctacatttgaagtattaaaaaaaaaaaaaaaaaaa

SEQ ID NO: 8 - protein, 726 aa, Danio rerio
MKLLLFAAWFSSLLDPCRFLDICQSCHPNADCDDICKCRTGYTGNGISDCRDDNECETVPEICGLHANCTNYVGGYYCNCLSGFISNGTEQFQTNDGTSCNDINECEEDRKCGPNSKCHNNIGSFICSCLRGYTSPAGPWFMPNHGTDCIENKIHCHQDHKCTKETVNSTLERMTNLSISERLKEIRYQTSAALSPVLLISYIEAMVSSKLNFGDVSKDSKEHVNETITNLVFSVNNLVEKDEKVEWKKINEDLRKYYVTKLLHTAEKETLALSAGYTHATQMQVHAGDVEMKLYTFEPRQAQKHPLSANIQGNSISLSTKKARHANNNGSTSVVFLIYHSIGDLLKPADDPGVADYSRYAAAGEITVNSPVIAAAISNQKTLPLNDVTFTLKHTQEIDPARDETKCASWEYSPSMMGHWSLDGCIRTRVNTTHTSCSCNHLTHFAILMSSARANLLAHYNVLTRITQLGMVISLICLSMCIFTFWFFRDIQNTRTTIHKNLCCSLFMAQFIFLIGINKSAHKWFCSLIAGLLHYFFLAAFAWMCIEGIHLYLIVVGVIYNKGFLHRNFYAFGYGSPAVVVAISATLGYKYYGTSSVCWLSTENNFIWSFIGPPILIILVNLLAFAVIIYKVYRHTAVKKPEISHYENIRSCARGAIALLFVLGVTWAFGVMYILYETTLTAYLFTFANVFQGMFIFIFLCVLSRRIQEEYYRLFKNMPCCFECLR

SEQ ID NO: 9 - DNA, 2816 bp, Homo sapines
  CDS (867)..(2414)
tgggggcgtcctccttcgtccccgcccggctgtcaagctgtgttctagcggccgagggaccgaggggggctaagaaagggggcgcccagccatgcagaggcaaaaaggcgctgcggaacggggtccccgtcgccagtgctgaggcaggaggtcggagccacaagtgaggggctgggaagcaggacccagcacgggcgtcttggcaggcggccgggcgcagggccaggctgctggggacgctcagggctttccacccaagccatgggcgctgtcgggcactcgggggtcccctcgtggctccggccactcggcgtgggcattacgttggcttcacatcgccatccagcctcgaagccaacaggactgaaaaatagcttcggccaaacgttctcctcccgctaaggagaggggtcgagtgcgtcagcccgaggggactggagagggatgccctagccctcgaggggcggaggacccgcggttgaaggaggcagcgggagcggagagcgccctccttgaccatcgaatgcctccttctgtgtttccattcctgtcgagtgggctgggccacgctgaccaccctggaggagggacggacgacgctcggcgggctctgaccgtgccgccttcttgtggctgctgactgggatccaggagggagtgggcatggggcgcagccgcgcctccctccctccccgcctcccgggcgccggggttggcgatgtggagacgtgaggggacccgtcggctgctccggcttctccaggactccgccaggcgcccgcgcgtccctcctcacccggaggaggagaggctccgcgcggggctccgaggcgggcggcgcgcggagccggagtcccagcctcgccatgggacataacgggagctggatctctccaaatgccagcgagccgcacaacgcgtccggcgccgaggctgcgggtgtgaaccgcagcgcgctcggggagttcggcgaggcgcagctgtaccgccagttcaccaccaccgtgcaggtcgtcatcttcataggctcgctgctcggaaacttcatggtgttatggtcaacttgccgcacaaccgtgttcaaatctgtcaccaacaggttcattaaaaacctggcctgctcggggatttgtgccagcctggtctgtgtgcccttcgacatcatcctcagcaccagtcctcactgttgctggtggatctacaccatgctcttctgcaaggtcgtcaaatttttgcacaaagtattctgctctgtgaccatcctcagcttccctgctattgctttggacaggtactactcagtcctctatccactggagaggaaaatatctgatgccaagtcccgtgaactggtgatgtacatctgggcccatgcagtggtggccagtgtccctgtgtttgcagtaaccaatgtggctgacatctatgccacgtccacctgcacggaagtctggagcaactccttgggccacctggtgtacgttctggtgtataacatcaccacggtcattgtgcctgtggtggtggtgttcctcttcttgatactgatccgacgggccctgagtgccagccagaagaagaaggtcatcatagcagcgctccggaccccacagaacaccatctctattccctatgcctcccagcgggaggccgagctgcacgccaccctgctctccatggtgatggtcttcatcttgtgtagcgtgccctatgccaccctggtcgtctaccagactgtgctcaatgtccctgacacttccgtcttcttgctgctcactgctgtttggctgcccaaagtctccctgctggcaaaccctgttctctttcttactgtgaacaaatctgtccgcaagtgcttgatagggaccctggtgcaactacaccaccggtacagtcgccgtaatgtggtcagtacagggagtggcatggctgaggccagcctggaacccagcatacgctcgggtagccagctcctggagatgttccacattgggcagcagcagatctttaagcccacagaggatgaggaagagagtgaggccaagtacattggctcagctgacttccaggccaaggagatatttagcacctgcctggagggagagcaggggccacagtttgcgccctctgccccacccctgagcacagtggactctgtatcccaggtggcaccggcagcccctgtggaacctgaaacattccctgataagtattccctgcagtttggctttgggccttttgagttgcctcctcagtggctctcagagacccgaaacagcaagaagcggctgcttccccccttgggcaacaccccagaagagctgatccagacaaaggtgcccaaggtaggcagggtggagcggaagatgagcagaaacaataaagtgagcatttttccaaaggtggattcctagcaaggattgtaaattcttggaagcaacggggggcttccatattcccaccagagtgtgggaatgctgtggccatgtgattgtatgatctccttgcaactcagtgtgagttgattcctccaatatgggccagatgcttttgaatgatagggaaatctacataaaatccagtgtcctctttattgagggagtatatgtatccatctcagtgatccatgtccttagtgaagtccacattattctctgtggggacaagagctgggcagttttgaatgggtcttgaggtgggtaccccatgtgcactttctgaggatgcctcacttccctgggctctgcagagaacacacagagagaagactttcagagctcacaggagcagggagcaggagcactctaagggaattc

SEQ ID NO: 10 - protein, 515 aa, Homo sapines
MGHNGSWISPNASEPHNASGAEAAGVNRSALGEFGEAQLYRQFTTTVQVVIFIGSLLGNFMVLWSTCRTTVFKSVTNRFIKNLACSGICASLVCVPFDIILSTSPHCCWWIYTMLFCKVVKFLHKVFCSVTILSFPAIALDRYYSVLYPLERKISDAKSRELVMYIWAHAVVASVPVFAVTNVADIYATSTCTEVWSNSLGHLVYVLVYNITTVIVPVVVVFLFLILIRRALSASQKKKVIIAALRTPQNTISIPYASQREAELHATLLSMVMVFILCSVPYATLVVYQTVLNVPDTSVFLLLTAVWLPKVSLLANPVLFLTVNKSVRKCLIGTLVQLHHRYSRRNVVSTGSGMAEASLEPSIRSGSQLLEMFHIGQQQIFKPTEDEEESEAKYIGSADFQAKEIFSTCLEGEQGPQFAPSAPPLSTVDSVSQVAPAAPVEPETFPDKYSLQFGFGPFELPPQWLSETRNSKKRLLPPLGNTPEELIQTKVPKVGRVERKMSRNNKVSIFPKVDS

SEQ ID NO: 11 - DNA, 3654 bp, Rattus norvegicus
  CDS (1)..(1557)
atgaaccgaggcaggacaggctgtgaggtacatctgaagatgggccctgggcagcttgacgggctgcaggctccgtgtctaggagtccgagggttgaattcagagcctttaaaatatacaagctgtggggccagtgcagttgtaggtcctcggtcccaaacaggcctccacccagtatgtcactggaacttcacggtgttatggtcaacctgccgcacgaccgtgttcaaatctgtcaccaacaggttcattaaaaacctggcctgctccgggatttgtgccagcgtggtctgcgtgcccttcgacatcatcctgagcaccagccctcactgctgctggtggatctacacgatgctcttctgcaaggtgcttaaattcctacacaaggtgttctgctcggtgactgtcctcagctttcctgccattgccctggacaggtactattcagtcctctaccctctggagagaaaaatctctgacgccaagtctcgggaactagtgatgtacatctgggcccatgcagtggtggccagcgtccctgtgtttgctgtgaccaatgtagctgacatctatgctacatccacctgcacggaagtctggagcaactccttgggccacctggtgtatgtcctgatctacaacgtcaccacggtcattgtgcctgtggctgtggtattcctctttcttatcctgatccggagggccctgagtgccagtcagaagaagaaagtcatcatcgctgcactgaggaccccacagaacaccatctctatcccctatgcatcccagcgggaggcagagctacatgccaccctgctctccatggtgacagttttcatcctctgcagcgtaccctatgccaccctggttgtctaccagactgtgctcaatgtacctaacacttctgtgttcttgcttctcactgctatttggctgcccaaagtctcactgttggccaaccctgtgctcttcctaactgtgaacagatcagtacgcaaatgcttggtggggaccctggtgcagttgcaccaccggtacagtcgccgcaatgtggttagtacagggagtggggtggttgagcctagccttgagcccagcatgcgctctggtagccagctcttggagatgttccacattggacagcaacagatctttaagccctcggaggatgaggaagagagtgaggccaagtatcttggctcaactgatttccaggccaaggaggtactcacctcctccccagagggcgaggagtcacagcttgcaccctctgtgccgcctccaggcacagtagactctgtatctcgggtgtccccagtagcccccgtggaacctggcatattccctgataagtattccctgcagtttggctttgggcctttcgagttgccccctcagtggctctctgagactcgaaatagcaagaagaggctgcttcccccactgggcaataccccagaagagctgatccagacaaaggtgcccagagtgagcagggtggagcggaagatgagcagaaacaataaagtgagcatttttccaaaggtagactcctagcgaggactatgagtgcctgagagtgtaagggagcttgtatattcctgttctcccttccctctggctgccactgtggggcgataactcctttacatggcaaatgcttctgaatgaaagggaagtctacagaaaacccaatgtcctctttattgagggagtatatatctatcttagtgatctgtgtccttcatcacattcagtgtaagctgggcagaatggaacaggtctgagggctctctgacacattttctgggagattcctcctttttttgtgtgcagcaaagaactcaaatggagcaggcactccaaggaggcccaggataattctgggcttcaatggccacagagaagaagctaatttgtttttaaaatgtttgttttgttttgttttgtttttttttttttgagactggatttctctatgtagccccaccctacagattgggctggccttgaactcagagagccacctgtctctgccttctgagtgctgggcttaaaggtctgtgcccacatgcccacttaagaagcctatttgggtagctaaataagaataacaaataggaaaatatctgagaagacagagccatggtgtcgaatggtttttgctagatttcacaatcacccaactcctggttgctaatacccctcgtctggatttctgggtcataaagggggggatgactggatttatcttaggatagacagggcagtgtctgagggagcctttttgtccttgattatctctccaccctgttggtatcaccaaggcctccaacaaatgtacctgcttccaccagtcagccatgctgcctggaaaccactgccaaccattcactgtgtgtccattttatcagcaacaagaagggaagatttgtggtggcgaagacaggctgagaaatgcccaccatgaataaccttttttctctctcacgtgattaagagcaaaagactagactcagtataggggggtataggcacaaagtaaaacgtcctaactggcaaatgtttggaattaaaaggtgcttaagaaatgtaggtggcatgcactgggtcgtttggcaccattaaggtaaaaatccttgattagcacaagcgactctgccctaagacttattagtttgacatacaggacacgcccacccgccagtctcattttcagtgtgttctgattctggatcccacagaaggtgagcagagcaggagaaataatgattgcaaatgctaaagcaattcccaccctgatttagcactccatcctctacttccaatgaagttcaggtatgagccatggtggatgggagaatgtgctctctcagcagctcagtgccctggctctcatgtgtgagccactctgggactgatgtaccagacctaattcaaacccaactctcccctcatacatcaagttgggagctctgctcttcttagggtgttttgttcttgtttgtggtccttggtggagccagtcgggcaccttgaatggataggtattcctacgagcaaacttctacatctcaaaggcagtggtcctagcagcacgtttagccatgctggcccatcgcccctaagaggaagctcagcccgtgtatagggagactccttttcttttcttctattgtgttatgtcttgtgaaccccaacatctcctccttggagagggggcgccttcctttctgtgcactgcctcacagacttgttttctcacgtcagagacagggtactgtgcccctcttgtggctctgtggccctctgggccctgtgtacctgccatctgtagctctttgtcctgatgctgtgttaatgctgtggccctccaagctcatctttcaaagccttcttaattgttacagcttttactaaggggaaaaataatccctcagagaatcctatgtaccagagaagaacctgtgaaatgtacgtgtgttgctgtcgtcctgacctgtatgtgtgacttaataaattctcaacagcggagtttggttcaccaaattctcatgtatgttggaaagagaaggctgacgtctagcaggctgtcgccgagtgtatttctatactctttgaagaataacattttaataaaaattataaagctgt

SEQ ID NO: 12 - protein, 518 aa, Rattus norvegicus
MNRGRTGCEVHLKMGPGQLDGLQAPCLGVRGLNSEPLKYTSCGASAVVGPRSQTGLHPVCHWNFTVLWSTCRTTVFKSVTNRFIKNLACSGICASVVCVPFDIILSTSPHCCWWIYTMLFCKVLKFLHKVFCSVTVLSFPAIALDRYYSVLYPLERKISDAKSRELVMYIWAHAVVASVPVFAVTNVADIYATSTCTEVWSNSLGHLVYVLIYNVTTVIVPVAVVFLFLILIRRALSASQKKKVIIAALRTPQNTISIPYASQREAELHATLLSMVTVFILCSVPYATLVVYQTVLNVPNTSVFLLLTAIWLPKVSLLANPVLFLTVNRSVRKCLVGTLVQLHHRYSRRNVVSTGSGVVEPSLEPSMRSGSQLLEMFHIGQQQIFKPSEDEEESEAKYLGSTDFQAKEVLTSSPEGEESQLAPSVPPPGTVDSVSRVSPVAPVEPGIFPDKYSLQFGFGPFELPPQWLSETRNSKKRLLPPLGNTPEELIQTKVPRVSRVERKMSRNNKVSIFPKVDS

SEQ ID NO: 13 - DNA, 3813 bp, Mus musculus
  CDS (125)..(1672)
cggctggccggctctgcaggacgctgcctggctcccgagcgtcggtccctccacgcccggggcaggagcggctgtgcgctgggctcagaggccaggcggacgagcgcaaaccccgggcttcgctatggggcacaacagcagctgggtgtcaccgaacaccagccacccgcgcaacacgtcgggcgccgaggctggcgccaacctcagcgcgttcggggagctgagcgaggctcagctgtaccgccagttcaccaccaccgtgcaggtcgtcatcttcattggctcgctactgggaaacttcatggtgttatggtcaacttgccgcacaacagtgttcaaatctgtcaccaacaggttcattaaaaacctggcctgctccgggatttgtgccagtgtggtctgtgtgcccttcgacatcatcctgagcagcagccctcactgttgctggtggatctatacgatgctcttctgcaaggtgctcaaattcctacacaaggtgttctgctctgtgactgtcctcagttttcctgctattgccctggacaggtactattcagtcctttatcctctggagagaaaaatatctgatgccaagtctcgggagctagtgatgtacatctgggctcatgcagtggtggccagtgtccctgtgtttgctgtgaccaatgtcgctgacatctatgctatgtccacctgcacggaagtctggagcaactccttgggccacctggtgtatgttctgatctacaatgtcaccacagtcattgtgcccgtggctgtggtatttctctttctcatcctgatccggagggccctgagtgccagccagaagaagaaagtcataattgctgcactgaggaccccacagaacaccatctctatcccctatgcttcccagcgagaggcagagctacatgccaccctgctctccatggtgacagttttcattctctgcagtgtaccctatgccaccctggttgtctaccagactgtgctcaatgtacctaacacctctgtcttcttgcttctcacggccatttggctgcccaaagtctctctgttggccaaccctgtgctcttcttaactgtgaacaaatcggtacgcaagtgcttggtggggaccctggtgcagttgcaccaccggtacagtcgccgcaatgtggttagtacagggagtggggtggctgagcccagccttgagcccagcatgcgctctggtagccagctcttggagatgttccacattgggcagcaacagatctttaagccctcggaggatgaggaagaaagtgaggccaagtatctcggctcagccgatttccaggcaaaggaggtgctcacttcctgcccagagggcgagcaggagccaccacagcttgcaccgtctgtgccacctccaggcacagtggactctgaacctcgggtgtccccagtagctcccatggaatctgggatatttcctgataagtattccctgcagtttggctttgggccttttgagttaccccctcagtggctctctgagacccgaaatagcaagaagaggctgcttcccccactgggcaataccccagaagagctgatccagacaaaggtgcccagagtgaacagggtggaacggaagatgagcagaaacaataaagtgagcatttttccaaaggtagactcctagcaaggcctgtgagggcccggaagccacagggagcttgtgtagtcctgttcccccctcactctggctgcaagccaccgtggggcgacaactcctctatggggcagatgcttctgagtgaaagggaagtctacataaatccaatgtcctctttattgagggagtatatattatctatcttagtggtctgtgtccttcgtcacattcagtgtaaactgggcagatcggaacatttttttctgggagattcctcctttccctgtgtacaacagagaactcaaagggagtaggcactctgagaggcccaggataattatgggcatcattggccacaaagaggaagctgatttggttttatttattttttattttattttattttttttgagactggatctctctatatagccccaccatatagaccgggctggctttgaactcagagatccacttgcctctgccttctgagagctgggattaaaggccttgcccgcatgcccacttaagaaacatatttggatagctaaataagaataacaaacagaaagatatctgtactgagaagacagagccatggtgtccaatgctgttcgctagattttgcaaccacctggatcctgtttgccgatacccctcatccggatttctgggtcataaaagggggatgactggtcctaccttgtggcagacagggcagtatcggagggagcctttttgtccttgattatctttccatcctgttggtatcaccaaggcctcccacaaatgtcccttctgtcaccagtcaccactgctgccaggaaaccactgccaacccttccttcgctgtatgtccgtttaaactgcacttgatcagcaacaaggagagaagctttgtggcagtgaaggcaggttgagaaatgcccacgatgcatagcttttctttctttgacgtgatctagaacaaaagactagagtcagtatagtgggatatattgagtgacgtataggcacaaagtaaaacgtcatagttggcaaatgtttggagttaaaaggtgctgaagaaatgtaggtgacatgcactgggtcctttgacacctttaaggtaaacttcctttattagcacaagtgattctgccccaggacttgttagtctgacctatagaatgggcccgcctgacagtctcattttcaatatgttcaatgattctggtccccagagaaggtgagcagagcaaggagaaataatgattgcaaaatgctaaagcaatacccaccctgatttagcacctcaccccctacttcccagtgaaggttcagtctgagccatggtggatgggagaacgtgttctttcagcagcccaatgtcctggttctcacgtgtgagccactctgggactgatgtaccaggcctaattccaaccaaaccctcccctcatacgtcaagtctgcagctctgctcttctttagggtgctttgttcttgttcgtggtacttagtggagccagccggctggcttgaatggatgggtgttccttactggcaaacctctacatctcaaaggcagtggtcctagcagcctgtttagtcatgctggcccatctcccctaggcagaagttcagtccatgtgtggggagactccttttctttccttctgttgtgttatgtcttgtgaacctcaaaatcttctccttgaagagggagcaccttcctttctgtgcaccacctcacagacttgtttgcttgccatagagacagggcactgcacccctcttgaggctctgtggccctccgggccccgtgtacatgccatctgtagcccttggtcctgactctgtgttaatgctgtggcccttcaagctcatctttcaaagccttcttaattgatacagcttttactaaggggaaaaaacaatctgtcagaatattgtatgtacctgagaagaacctgtgaaataaacctgtgttgctgtcaccgtcctgacatgtgtatgtatgactttagtacattctcaacaacagagtttcgttcaccaaattctcatgtaagcttgaaagagaaggctgacgcctaacaggctgtcaccgagtgtatttctatactctttgaagaataacattttaataaaaattacaaagctgaaaaaaaaaaaaaaaaaaaaaa

SEQ ID NO: 14 - protein, 515 aa, Mus musculus
MGHNSSWVSPNTSHPRNTSGAEAGANLSAFGELSEAQLYRQFTTTVQVVIFIGSLLGNFMVLWSTCRTTVFKSVTNRFIKNLACSGICASVVCVPFDIILSSSPHCCWWIYTMLFCKVLKFLHKVFCSVTVLSFPAIALDRYYSVLYPLERKISDAKSRELVMYIWAHAVVASVPVFAVTNVADIYAMSTCTEVWSNSLGHLVYVLIYNVTTVIVPVAVVFLFLILIRRALSASQKKKVIIAALRTPQNTISIPYASQREAELHATLLSMVTVFILCSVPYATLVVYQTVLNVPNTSVFLLLTAIWLPKVSLLANPVLFLTVNKSVRKCLVGTLVQLHHRYSRRNVVSTGSGVAEPSLEPSMRSGSQLLEMFHIGQQQIFKPSEDEEESEAKYLGSADFQAKEVLTSCPEGEQEPPQLAPSVPPPGTVDSEPRVSPVAPMESGIFPDKYSLQFGFGPFELPPQWLSETRNSKKRLLPPLGNTPEELIQTKVPRVNRVERKMSRNNKVSIFPKVDS

SEQ ID NO: 15 - DNA, 1632 bp, Danio rerio
  CDS (1)..(1632)
atggaggcggataacgcggagagctggttttctcagtcagaaaacggaacaatactaaactcctcgaagctgggtttcctaaacgcgtcgtctcctgtgggtctgtggggtgacgctcaggataaagaggtgacacggctggaggagattgtgcacgcgcaggaaagagcctacaaagatttcaccaccaccattcaagttttcatcctcatcggatcccttttgggcaatactgcagtgctgtggtgcacgatctacacaaatgtcttcaagtccgtcacaaatctcttcatcaagaatctggcgtgttctggcatttgtgctggtcttgtgtgtgtgccgtttgacatagcgctgagtgccagtcctctatgctgcctgtgggttcacacactcctcctgtgcaaagccattaagttcctgcacaggcttttctgctctgccactgtgctcagtttcgccgtcattgccctggacaggtactactcagtgttgtatcctcttgagagaaagatatcagatgccagatccagagacctggtaatctacatttgggttcatgcagtgttggccagtgtccctgttttcgctgtaagcaatgtgacagatgtctacgccacctcatcttgctctgacggacaatcgcttggacatcttgtttacatgattacatacagcatcaccactcttattctccctctagcagtggtgtttctcgccatggtattaatccgccgcgcattaagcaccagccaaaagaagaaagtgatcatcgcggcgctgcgcacccctcagaacagcatttccatcccgtatgtgtctcaacgtgaggctgaacttcatgcaactctgttatccattgtgctcgtcttcacactctgcagcacaccatatgctgctctgctaatataccacgccatactgggggctgcaaaagcctcatcttggttacgtctgactgccatatggctcccgaaggtctcgctcctcaccaacccactgtttctcctcacagttaaccgatctgtgcgacggtgccttcttgatatgctagcacgcctccatcgacgctacagcagaaggaacatggtcagcattggaggtctggctgaggtaggaccactaggtggtgagactgtggttcgctctggtagtcaactcctggagatgttcaatatcggccaacagcagatcttcagacccaacgaggatgaagaagatgagaatgagatttcctcagtggcgtctgccaactttcaacccaaagatgaaggacaaggtggaaatgcaggggcgacagataacacacatcagccgcagccaggggaaggggttaaagaagaggtcttagttttaccaaaacattcccctattcagtcatgtgcctactcctcttcgcaagttgcccccgcaactcctacggaccctgaggatgcgacacaatttggttttgggccgtttgagctgccccctcaatggctgcctgaaaccaggaacagtaagaaaagactcttgcctccattaggtaacacaccagaggagctcatccagaccaaacagcccagagtccgacctgagagacgcatcagtagaaacaataaagttagtacctttcctaatgtggacccctga

SEQ ID NO: 16 - protein, 543 aa, Danio rerio
MEADNAESWFSQSENGTILNSSKLGFLNASSPVGLWGDAQDKEVTRLEEIVHAQERAYKDFTTTIQVFILIGSLLGNTAVLWCTIYTNVFKSVTNLFIKNLACSGICAGLVCVPFDIALSASPLCCLWVHTLLLCKAIKFLHRLFCSATVLSFAVIALDRYYSVLYPLERKISDARSRDLVIYIWVHAVLASVPVFAVSNVTDVYATSSCSDGQSLGHLVYMITYSITTLILPLAVVFLAMVLIRRALSTSQKKKVIIAALRTPQNSISIPYVSQREAELHATLLSIVLVFTLCSTPYAALLIYHAILGAAKASSWLRLTAIWLPKVSLLTNPLFLLTVNRSVRRCLLDMLARLHRRYSRRNMVSIGGLAEVGPLGGETVVRSGSQLLEMFNIGQQQIFRPNEDEEDENEISSVASANFQPKDEGQGGNAGATDNTHQPQPGEGVKEEVLVLPKHSPIQSCAYSSSQVAPATPTDPEDATQFGFGPFELPPQWLPETRNSKKRLLPPLGNTPEELIQTKQPRVRPERRISRNNKVSTFPNVDP

SEQ ID NO: 17 - DNA, 25 bp, Artificial
  ETL morpholino (MO) sequence
gcaggagtttcattggagaactgtg

SEQ ID NO: 18 - DNA, 25 bp, Artificial
  mismatch ETL MO
gcacgactttgattggacaactctg

SEQ ID NO: 19 - DNA, 25 bp, Artificial
  GPR MO
agctctccgcgttatccgcctccat

SEQ ID NO: 20 - DNA, 25 bp, Artificial
  GPR mismatch MO
aggtctgcgccttatccccctcgat

(19) United States
(12) Patent Application Publication
(10) Pub. No.: US 2009/0178145 A1
(43) Pub. Date: Jul. 9, 2009

(54) Methods and targets for identifying compounds for regulating angiogenesis

(75) Inventors: Andrew Nicholas Carr, Maineville, OH (US); Kevin Gene Peters, Loveland, OH (US); Claus-Jens Walter Doersen, Mason, OH (US); Xiaoyan Qu, Mason, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(73) Assignee: The Procter & Gamble Company, Cincinnati, OH (US)

(21) Appl. No.: 11/431,846

(22) Filed: May 10, 2006

Related U.S. Application Data
(60) Provisional application No. 60/679,881, filed on May 11, 2005.

(51) Int. Cl. A61K 48/00; C12Q 1/68

(52) U.S. Cl. 800/3; 435/6; 514/44

(57) ABSTRACT

The present invention provides methods for identifying genes and proteins that may regulate angiogenesis. The genes identified may be used as markers for the disease onset and progression and to measure efficacy of a therapeutic. The present invention also provides methods to screen agents that are capable of regulating angiogenesis. The present invention also provides methods of identifying therapeutic compounds that may treat various disorders by regulating the expression and activity of genes and proteins identified.

### CROSS REFERENCE TO RELATED APPLICATION

This application claims the benefit of U.S. Provisional Application Ser. No. 60/679,881, filed 11 May 2005, which is herein incorporated by reference in its entirety.

### REFERENCE TO ELECTRONIC SUBMISSION OF A TABLE

This Application contains a data table (designated as Table 4 in the specification) as an appendix on a compact disc as required under 37 CFR §1.52(e)(1)(iii) and 37 CFR § 1.58, and is herein incorporated by reference in its entirety in accordance with 37 CFR § 1.77(b)(4). A duplicate disc is also provided as required under 37 CFR § 1.52(e)(4). Both the compact discs are identical in their contents. The compact discs each contain a single ASCII (.txt) file for the Table 4, entitled “9986M_Table_{—}4.txt”, which were created on 4 May 2006, using an IBM-PC machine format, are 71 kb in size, and are Windows XP compatible. A statement that both the files on the discs are identical is also submitted separately as required under 37 CFR § 1.52(e)(4).

### REFERENCE TO A SEQUENCE LISTING

This Application contains a Sequence Listing appendix on a computer readable form as required by 37 CFR 1.821(e), and is incorporated by reference in its entirety in accordance with 37 CFR 1.77(b)(4). A paper copy of the Sequence Listing is also provided as required by 37 CFR 1.821(c). The Sequence Listing on the computer readable form is identical to the one on the paper copy.

### FIELD OF THE INVENTION

The present invention relates to methods of identifying target genes and compounds for regulating angiogenesis. The invention also relates to methods for the treatment of angiogenesis regulated disorders using the genes or proteins of the invention as targets for intervention.

### BACKGROUND OF THE INVENTION

Angiogenesis, the sprouting of new blood vessels from the pre-existing vasculature, plays a crucial role in a wide range of physiological and pathological processes (Nguyen, L. L. et al, Int. Rev. Cytol., 204, 1-48, (2001)). Angiogenesis is a complex process, mediated by communication between the endothelial cells that line blood vessels and their surrounding environment. In the early stages of angiogenesis, tissue or tumor cells produce and secrete pro-angiogenic growth factors in response to environmental stimuli such as hypoxia. These factors diffuse to nearby endothelial cells and stimulate receptors that lead to the production and secretion of proteases that degrade the surrounding extracellular matrix. These activated endothelial cells begin to migrate and proliferate into the surrounding tissue toward the source of these growth factors (Bussolino, F., Trends Biochem. Sci., 22, 251-256, (1997)). Endothelial cells then stop proliferating and differentiate into tubular structures, which is the first step in the formation of stable, mature blood vessels. Subsequently, periendothelial cells, such as pericytes and smooth muscle cells, are recruited to the newly formed vessel in a further step toward vessel maturation.

Angiogenesis is regulated by a balance of naturally occurring pro- and anti-angiogenic factors. Vascular endothelial growth factor, fibroblast growth factor, and angiopoeitin represent a few of the many potential pro-angiogenic growth factors. These ligands bind to their respective receptor tyrosine kinases on the endothelial cell surface and transduce signals that promote cell migration and proliferation. Whereas many regulatory factors have been identified, the molecular mechanisms of this process are still not fully understood.

There are many disease states driven by persistent unregulated or improperly regulated angiogenesis. In such disease states, unregulated or improperly regulated angiogenesis may either cause a particular disease or exacerbate an existing pathological condition. For example, ocular neovascularization has been implicated as the most common cause of blindness and underlies the pathology of approximately 20 eye diseases. In certain previously existing conditions such as arthritis, newly formed capillary blood vessels invade the joints and destroy cartilage. In diabetes, new capillaries formed in the retina invade the vitreous humor, causing bleeding and blindness.

Both the growth and metastasis of solid tumors are also angiogenesis-dependent, Folkman et al., “Tumor Angiogenesis,” Chapter 10, 206-32, in The Molecular Basis of Cancer, Mendelsohn et al., eds., W. B. Saunders, (1995). It has been shown that tumors that enlarge to greater than 2 mm in diameter must obtain their own blood supply and do so by inducing the growth of new capillary blood vessels. After these new blood vessels become embedded in the tumor, they provide nutrients and growth factors essential for tumor growth as well as a means for tumor cells to enter the circulation and metastasize to distant sites, such as liver, lung or bone (Weidner, New Eng. J. Med., 324, 1, 1-8 (1991)). When used as drugs in tumor-bearing animals, natural inhibitors of angiogenesis may prevent the growth of small tumors (O'Reilly et al., Cell, 79, 315-28 (1994)). In some protocols, the application of such inhibitors leads to tumor regression and dormancy even after cessation of treatment (O'Reilly et al., Cell, 88, 277-85 (1997)). Moreover, supplying inhibitors of angiogenesis to certain tumors may potentiate their response to other therapeutic regimens (see, e.g., Teischer et al., Int. J. Cancer, 57, 920-25 (1994)).

Although many disease states are driven by persistent unregulated or improperly regulated angiogenesis, many disease states could be treated by increased angiogenesis. Tissue growth and repair are biologic events wherein cellular proliferation and angiogenesis occur. Thus an important aspect of wound repair is the revascularization of damaged tissue by angiogenesis.

Chronic, non-healing wounds are a major cause of prolonged morbidity in the aged human population. This is especially the case in bedridden or diabetic patients who develop severe, non-healing skin ulcers. In many of these cases, the delay in healing is a result of inadequate blood supply either as a result of continuous pressure or of vascular blockage. Poor capillary circulation due to small artery atherosclerosis or venous stasis contributes to the failure to repair damaged tissue. Such tissues are often infected with microorganisms that proliferate unchallenged by the innate defense systems of the body which require well vascularized tissue to effectively eliminate pathogenic organisms. As a result, most therapeutic intervention centers on restoring blood flow to ischemic tissues thereby allowing nutrients and immunological factors access to the site of the wound.

Atherosclerotic lesions in large vessels may cause tissue ischemia that could be ameliorated by modulating blood vessel growth to the affected tissue. For example, atherosclerotic lesions in the coronary arteries cause angina and myocardial infarction that could be prevented if one could restore blood flow by stimulating the growth of collateral arteries. Similarly, atherosclerotic lesions in the large arteries that supply the legs cause ischemia in the skeletal muscle that limits mobility and in some cases necessitates amputation, which may also be prevented by improving blood flow with angiogenic therapy.

Other diseases such as diabetes and hypertension are characterized by a decrease in the number and density of small blood vessels such as arterioles and capillaries. These small blood vessels are critical for the delivery of oxygen and nutrients. A decrease in the number and density of these vessels contributes to the adverse consequences of hypertension and diabetes including claudication, ischemic ulcers, accelerated hypertension, and renal failure. These common disorders and many other less common ailments, such as Burgers disease, could be ameliorated by increasing the number and density of small blood vessels using angiogenic therapy.

Thus, there is a continuing need to identify regulators of angiogenesis. However, one problem associated with identification of compounds for use in the treatment of angiogenesis has been the lack of good screening targets and of screening methods for the identification of such compounds.

### SUMMARY OF THE INVENTION

The present invention relates to screening for compounds that modulate expression or activity of a gene involved in regulating angiogenesis. The present invention identifies such genes as targets to screen for compounds that modulate their expression or activity and thereby regulate angiogenesis.

In one embodiment, the invention provides for a method of screening compounds useful for regulating angiogenesis, comprising the steps of: (a) exposing a protein of the invention to a compound; and (b) measuring binding or activity of the protein; wherein binding of the compound to the protein or a modulation in the activity of the protein indicates that the compound is useful for regulating angiogenesis.

In another embodiment, the invention provides for a method of screening compounds useful for regulating angiogenesis, comprising the steps of: (a) expressing a protein of the invention in a suitable cell; (b) exposing the cells to a compound; and (c) measuring activity of the protein; wherein a modulation in the activity of the protein indicates that the compound is useful for regulating angiogenesis.

In another embodiment, the invention provides for a method of screening compounds useful for modulating expression of a gene or a family of genes involved in regulating angiogenesis comprising the steps of: (a) exposing a gene of the invention to a compound; and (b) measuring expression of the gene; wherein a modulation in the expression of the gene indicates the compound is useful for regulating angiogenesis.

In another embodiment, the invention provides for a method of screening compounds useful for regulating angiogenesis, comprising (a) selecting a compound that binds or regulates the activity or the expression of a protein of the invention; (b) further determining whether the compound regulates angiogenesis in an in vitro or in vivo angiogenesis model system; and (c) identifying those compounds that modulate angiogenesis in the angiogenesis model system as compounds for regulating angiogenesis.

In another embodiment, the invention provides a method for diagnosing a condition characterized by unregulated or improperly regulated angiogenesis, comprising detecting the level of expression of, or assaying for activity of a protein encoded by a gene of Table 4 in a tissue, wherein difference in expression and/or activity compared to expression and/or activity in a healthy/control tissue is indicative of unregulated or improperly regulated angiogenesis.

In another embodiment, the present invention also provides methods of monitoring the effectiveness of treatment, or monitoring the progression/regression of a disorder that is characterized by unregulated or improperly regulated angiogenesis, comprising administering a pharmaceutical composition to the subject, preparing a gene or gene family expression profile and/or assaying for an activity of a protein encoded by a gene or a member of a gene family of Table 4 from a tissue sample from the patient at various time intervals during the treatment and comparing the patient expression profile and/or activity to the expression profile and/or activity to each other and to control datasets. The profiles of gene expression or protein activity and their comparison to datasets from control samples would be indicative of effectiveness of the treatment or progression/regression of a dysregulated angiogenesis.

In another embodiment, the invention provides a pharmaceutical composition, comprising: a safe and effective amount of an agonist or an antagonist of a protein involved in regulating angiogenesis identified in Table 4; and a pharmaceutically acceptable carrier.

In another embodiment, the invention provides a method for regulating angiogenesis in a subject in which such a regulation is desirable, comprising: identifying a subject in which regulation of angiogenesis is desirable; and administering to the subject a safe and effective amount of compound that is an agonist or an antagonist of a protein identified in Table 4. In one embodiment, the desired regulation of angiogenesis is an increase in angiogenesis in the subject. In another embodiment, the desired regulation of angiogenesis is a decrease in angiogenesis in the subject.
28. A method for identifying compounds for regulating angiogenesis, comprising:
   a. contacting a compound with ETL protein;
   b. determining whether the compound binds the ETL protein; and
   c. identifying those compounds that bind the ETL protein as compounds for regulating angiogenesis.
29. A method for identifying compounds for regulating angiogenesis, comprising:
   a. contacting a compound with ETL protein;
   b. determining whether the compound binds the ETL protein;
   c. selecting those compounds that bind the ETL protein, and further
25. determining whether the compound regulates angiogenesis in an angiogenesis model system; and
   d. identifying those compounds that regulates angiogenesis in an angiogenesis model system as compounds for regulating angiogenesis.
30. The method according to claim 28, further comprising: administering the compound identified in step (c) of claim 28 to a non-human animal, and determining whether the compound regulates angiogenesis in the animal, wherein compounds that regulate angiogenesis in the animal are identified as compounds for regulating angiogenesis in vivo.
31. The method according to claim 29, further comprising: administering the compound identified in step (d) of claim 29 to a non-human animal, and determining whether the compound regulates angiogenesis in the animal, wherein compounds that regulate angiogenesis in the animal are identified as compounds for regulating angiogenesis in vivo.
32. A method for identifying compounds for regulating angiogenesis, comprising:
   a. contacting a compound with a cell population expressing ETL protein;
   b. determining and comparing the level of activity of the ETL protein in the cell population that is contacted with the compound to the level of activity of the ETL protein in the cell population that is not contacted with the compound; and
   c. identifying those compounds that modulate the activity of the ETL protein in the cell population that is contacted with the compound compared to the activity in the cell population that is not contacted with the compound as compounds for regulating angiogenesis.
33. The method according to claim 32, further comprising:
   a. further determining whether the compound identified in step (c) of claim 32 regulates angiogenesis in an angiogenesis model system; and
   b. identifying those compounds that regulate angiogenesis in an angiogenesis model system as compounds for regulating angiogenesis.
34. The method according to claim 32, further comprising: administering the compound identified in step (c) of claim 32 to a non-human animal, and determining whether the compound regulates angiogenesis in the animal, wherein compounds that regulate angiogenesis in the animal are identified as compounds for regulating angiogenesis.
35. The method according to claim 33, further comprising: administering the compound identified in step (b) of claim 33 to a non-human animal, and determining whether the compound regulates angiogenesis in the animal, wherein compounds that regulate angiogenesis in the animal are identified as compounds for regulating angiogenesis.
36. A method for identifying compounds for regulating angiogenesis, comprising:
   a. contacting a compound with a cell population expressing ETL protein;
   b. determining and comparing the level of expression of the ETL protein in the cell population that is contacted with the compound to the level of expression of the ETL protein in the cell population that is not contacted with the compound; and
   c. identifying those compounds that modulate the expression of the ETL protein in the cell population that is contacted with the compound compared to the expression of the ETL protein in the cell population that is not contacted with the compound as compounds for regulating angiogenesis.
37. A method according to claim 36, further comprising:
   a. determining whether the compound identified in step (c) of claim 36 regulates angiogenesis in an angiogenesis model system; and
   b. identifying those compounds that regulate angiogenesis in an angiogenesis model system as compounds for regulating angiogenesis.
38. The method according to claim 36, further comprising: administering the compound identified in step (c) of claim 36 to a non-human animal, and determining whether the compound regulates angiogenesis in the animal, wherein compounds that regulate angiogenesis in the animal are identified as compounds for regulating angiogenesis.
39. The method according to claim 37, further comprising: administering the compound identified in step (b) of claim 37 to a non-human animal, and determining whether the compound regulates angiogenesis in the animal, wherein compounds that regulate angiogenesis in the animal are identified as compounds for regulating angiogenesis.
40. A method for identifying compounds for regulating angiogenesis, comprising:
   a. contacting a compound with a cell population expressing ETL gene;
   b. determining and comparing the level of expression of the ETL gene in the cell population that is contacted with the compound to the level of expression of the ETL gene in the cell population that is not contacted with the compound; and
   c. identifying those compounds that modulate the expression of the ETL gene in the cell population that is contacted with the compound compared to the expression of the ETL gene in the cell population that is not contacted with the compound as compounds for regulating angiogenesis.
41. A method according to claim 40, further comprising:
   a. determining whether the compound identified in step (c) of claim 40 regulates angiogenesis in an angiogenesis model system; and
   b. identifying those compounds that regulate angiogenesis in an angiogenesis model system as compounds for regulating angiogenesis.
42. The method according to claim 40, further comprising: administering the compound identified in step (c) of claim 40 to a non-human animal, and determining whether the compound regulates angiogenesis in the animal, wherein compounds that regulate angiogenesis in the animal are identified as compounds for regulating angiogenesis.
43. The method according to claim 41, further comprising: administering the compound identified in step (b) of claim 41 to a non-human animal, and determining whether the compound regulates angiogenesis in the animal, wherein compounds that regulate angiogenesis in the animal are identified as compounds for regulating angiogenesis.
44. A method of diagnosing unregulated or improperly regulated angiogenesis, comprising:
   a. preparing gene expression profile for ETL gene; or measuring the level of expression or activity of ETL protein in a biological sample;
   b. comparing levels of expression of the ETL gene from the sample to level of expression of the ETL gene from a control sample or database, or comparing levels of expression or activity of the ETL protein from the sample to levels of expression or activity of the ETL protein from a control sample or from a database, wherein significant deviation from control levels is indicative of unregulated or improperly regulated angiogenesis.
45. A method of monitoring progression of angiogenesis, comprising:
   a. preparing gene expression profile for ETL gene; or preparing ETL protein expression profile, or ETL protein activity profile from a suitable angiogenesis model system;
   b. administering a therapeutic regimen to the subject;
   c. preparing a similar expression or activity profile as in step (a) after a suitable time after the therapeutic regimen;
   d. comparing the profiles prior to the intervention with profiles after the intervention; and
   e. repeating the steps (b), (c) and (d) during the course of the therapy and evaluating the data to monitor progression of angiogenesis.
46. A method of monitoring the treatment or progression of a disorder in a patient with unregulated or improperly regulated angiogenesis, comprising:
   a. preparing a gene expression profile for one or more gene involved in regulating angiogenesis identified in Table 4; or preparing a protein expression profile, or protein activity profile of one or more proteins involved in regulating angiogenesis identified in Table 4 from a subject;
   b. administering a therapeutic regimen to the subject;
   c. preparing a similar expression or activity profile as in step (a) from a cell or tissue sample from the subject after a suitable time after the therapeutic regimen;
   d. comparing the profiles prior to the therapy with profiles after the therapy; and
   e. repeating the steps (b), (c) and (d) during the course of the treatment or disorder and evaluating the data to monitor efficacy of the treatment or progression of the disorder.
47. A pharmaceutical composition, comprising:
   a. a safe and effective amount of an agonist or an antagonist ETL protein; and
   b. a pharmaceutically acceptable carrier.
48. A pharmaceutical composition comprising a safe and effective amount of a compound identified by the method according to claim 28.
49. A pharmaceutical composition comprising a safe and effective amount of a compound identified by the method according to claim 32.
50. A pharmaceutical composition comprising a safe and effective amount of a compound identified by the method according to claim 36.
51. A pharmaceutical composition comprising a safe and effective amount of a compound identified by the method according to claim 40.
52. A method for regulating angiogenesis in a subject in which such a regulation is desirable, comprising:
   a. identifying a subject in which regulation of angiogenesis is desirable; and
   b. administering to the subject a safe and effective amount of compound that is an agonist or an antagonist of ETL protein.
53. The method of claim 52, wherein the desired regulation of angiogenesis is an increase in angiogenesis in the subject.
54. The method of claim 52, wherein the desired regulation of angiogenesis is a decrease in angiogenesis in the subject.
55. A method according to claim 28, wherein the ETL protein has the amino acid sequence selected from SEQ ID NOs: 2, 4, 6, and 8.
56. A method for identifying compounds for regulating angiogenesis, comprising:
   a. contacting a compound with GPR176 protein;
   b. determining whether the compound binds the GPR176 protein; and
   c. identifying those compounds that bind the GPR176 protein as compounds for regulating angiogenesis.
57. A method for identifying compounds for regulating angiogenesis, comprising:
   a. contacting a compound with GPR176 protein;
   b. determining whether the compound binds the GPR176 protein;
   c. selecting those compounds that bind the GPR176 protein, and further determining whether the compound regulates angiogenesis in an angiogenesis model system; and
   d. identifying those compounds that regulates angiogenesis in an angiogenesis model system as compounds for regulating angiogenesis.
58. The method according to claim 56, further comprising: administering the compound identified in step (c) of claim 56 to a non-human animal, and determining whether the compound regulates angiogenesis in the animal, wherein compounds that regulate angiogenesis in the animal are identified as compounds for regulating angiogenesis in vivo.
59. The method according to claim 57, further comprising: administering the compound identified in step (d) of claim 57 to a non-human animal, and determining whether the compound regulates angiogenesis in the animal, wherein compounds that regulate angiogenesis in the animal are identified as compounds for regulating angiogenesis in vivo.
60. A method for identifying compounds for regulating angiogenesis, comprising:
   a. contacting a compound with a cell population expressing GPR176 protein;
   b. determining and comparing the level of activity of the GPR176 protein in the cell population that is contacted with the compound to the level of activity of the GPR176 protein in the cell population that is not contacted with the compound; and
   c. identifying those compounds that modulate the activity of the GPR176 protein in the cell population that is contacted with the compound compared to the activity in the cell population that is not contacted with the compound as compounds for regulating angiogenesis.
61. The method according to claim 60, further comprising:
   a. further determining whether the compound identified in step (c) of claim 60 regulates angiogenesis in an angiogenesis model system; and
   b. identifying those compounds that regulate angiogenesis in an angiogenesis model system as compounds for regulating angiogenesis.
62. The method according to claim 60, further comprising: administering the compound identified in step (c) of claim 60 to a non-human animal, and determining whether the compound regulates angiogenesis in the animal, wherein compounds that regulate angiogenesis in the animal are identified as compounds for regulating angiogenesis.
63. The method according to claim 61, further comprising: administering the compound identified in step (b) of claim 61 to a non-human animal, and determining whether the compound regulates angiogenesis in the animal, wherein compounds that regulate angiogenesis in the animal are identified as compounds for regulating angiogenesis.
64. A method for identifying compounds for regulating angiogenesis, comprising:
   a. contacting a compound with a cell population expressing GPR176 protein;
   b. determining and comparing the level of expression of the GPR176 protein in the cell population that is contacted with the compound to the level of expression of the GPR176 protein in the cell population that is not contacted with the compound; and
   c. identifying those compounds that modulate the expression of the GPR176 protein in the cell population that is contacted with the compound compared to the expression of the GPR176 protein in the cell population that is not contacted with the compound as compounds for regulating angiogenesis.
65. A method according to claim 64, further comprising:
   a. determining whether the compound identified in step (c) of claim 64 regulates angiogenesis in an angiogenesis model system; and
   b. identifying those compounds that regulate angiogenesis in an angiogenesis model system as compounds for regulating angiogenesis.
66. The method according to claim 64, further comprising: administering the compound identified in step (c) of claim 64 to a non-human animal, and determining whether the compound regulates angiogenesis in the animal, wherein compounds that regulate angiogenesis in the animal are identified as compounds for regulating angiogenesis.
67. The method according to claim 65, further comprising: administering the compound identified in step (b) of claim 65 to a non-human animal, and determining whether the compound regulates angiogenesis in the animal, wherein compounds that regulate angiogenesis in the animal are identified as compounds for regulating angiogenesis.
68. A method for identifying compounds for regulating angiogenesis, comprising:
   a. contacting a compound with a cell population expressing GPR176 gene;
   b. determining and comparing the level of expression of the GPR176 gene in the cell population that is contacted with the compound to the level of expression of the GPR176 gene in the cell population that is not contacted with the compound; and
   c. identifying those compounds that modulate the expression of the GPR176 gene in the cell population that is contacted with the compound compared to the expression of the GPR176 gene in the cell population that is not contacted with the compound as compounds for regulating angiogenesis.
69. A method according to claim 68, further comprising:
   a. determining whether the compound identified in step (c) of claim 68 regulates angiogenesis in an angiogenesis model system; and
   b. identifying those compounds that regulate angiogenesis in an angiogenesis model system as compounds for regulating angiogenesis.
70. The method according to claim 68, further comprising: administering the compound identified in step (c) of claim 68 to a non-human animal, and determining whether the compound regulates angiogenesis in the animal, wherein compounds that regulate angiogenesis in the animal are identified as compounds for regulating angiogenesis.
71. The method according to claim 69, further comprising: administering the compound identified in step (b) of claim 69 to a non-human animal, and determining whether the compound regulates angiogenesis in the animal, wherein compounds that regulate angiogenesis in the animal are identified as compounds for regulating angiogenesis.
72. A method of diagnosing unregulated or improperly regulated angiogenesis, comprising:
   a. preparing gene expression profile for GPR176 gene; or measuring the level of expression or activity of GPR176 protein in a biological sample;
   b. comparing levels of expression of the GPR176 gene from the sample to level of expression of the GPR176 gene from a control sample or database, or comparing levels of expression or activity of the GPR176 protein from the sample to levels of expression or activity of the GPR176 protein from a control sample or from a database, wherein significant deviation from control levels is indicative of unregulated or improperly regulated angiogenesis.
73. A method of monitoring progression of angiogenesis, comprising:
   a. preparing gene expression profile for GPR176 gene; or preparing GPR176 protein expression profile, or GPR176 protein activity profile from a suitable angiogenesis model system;
   b. administering a therapeutic regimen to the subject;
   c. preparing a similar expression or activity profile as in step (a) after a suitable time after the therapeutic regimen;
   d. comparing the profiles prior to the intervention with profiles after the intervention; and
   e. repeating the steps (b), (c) and (d) during the course of the therapy and evaluating the data to monitor progression of angiogenesis.
74. A method of monitoring the treatment or progression of a disorder in a patient with unregulated or improperly regulated angiogenesis, comprising:
   a. preparing a gene expression profile for one or more gene involved in regulating angiogenesis identified in Table 4; or preparing a protein expression profile, or protein activity profile of one or more proteins involved in regulating angiogenesis identified in Table 4 from a subject;
   b. administering a therapeutic regimen to the subject;
   c. preparing a similar expression or activity profile as in step (a) from a cell or tissue sample from the subject after a suitable time after the therapeutic regimen;
   d. comparing the profiles prior to the therapy with profiles after the therapy; and
   e. repeating the steps (b), (c) and (d) during the course of the treatment or disorder and evaluating the data to monitor efficacy of the treatment or progression of the disorder.
75. A pharmaceutical composition, comprising:
   a. a safe and effective amount of an agonist or an antagonist GPR176 protein; and
   b. a pharmaceutically acceptable carrier.
76. A pharmaceutical composition comprising a safe and effective amount of a compound identified by the method according to claim 56.
77. A pharmaceutical composition comprising a safe and effective amount of a compound identified by the method according to claim 60.
78. A pharmaceutical composition comprising a safe and effective amount of a compound identified by the method according to claim 64.
79. A pharmaceutical composition comprising a safe and effective amount of a compound identified by the method according to claim 68.
80. A method for regulating angiogenesis in a subject in which such a regulation is desirable, comprising:
   a. identifying a subject in which regulation of angiogenesis is desirable; and
   b. administering to the subject a safe and effective amount of compound that is an agonist or an antagonist of GPR176 protein.
81. The method of claim 80, wherein the desired regulation of angiogenesis is an increase in angiogenesis in the subject.
82. The method of claim 80, wherein the desired regulation of angiogenesis is a decrease in angiogenesis in the subject.
83. A method according to claim 56, wherein the GPR176 protein has the amino acid sequence selected from SEQ ID NOs: 10, 12, 14, 16.

### SEQUENCE LISTING DESCRIPTION

Each of the nucleotide and protein sequences in the sequence listing, along with the corresponding Genbank or Derwent accession number(s), where applicable, and the species from which it is derived, is shown in Table I.

TABLE I

| | SEQ ID | | |
|---|---|---|---|
| | NOs: | | |
| Sequence | Nucleotide, | | Equivalent Genbank |
| Description | Protein | Species | Acc. No. |
| ETL | 1, 2 | Homo sapiens | XM_371262 |
| ETL | 3, 4 | Rattus | NM_022294 |
| | | norvagicus | |
| ETL | 5, 6 | Mus musculus | NM_133222 |
| ETL | 7, 8 | Danio rerio | NM_213367 |
| GPR176 | 9, 10 | Homo sapiens | NM_07223 |
| GPR176 | 11, 12 | Rattus | XM_243493 |
| | | norvagicus | |
| GPR176 | 13, 14 | Mus musculus | NM_201367 |
| GPR176 | 15, 16 | Danio rerio | NM_213367 |
| GPR176 | | | GENSCAN00000007429 |
| Morpholino | 17 | Artificial | |
| oligo for ETL | | | |
| Mismatched | 18 | Artificial | |
| Morpholino | | | |
| oligo | | | |
| for ETL | | | |
| Morpholino | 19 | Artificial | |
| oligo | | | |
| for GPR176 | | | |
| Mismatched | 20 | Artificial | |
| Morpholino | | | |
| oligo | | | |
| for GPR176 | | | |

### BRIEF DESCRIPTION OF THE FIGURES

FIG. 1. ETL expression in human aortic (AoSMC), coronary artery (CASMC) and pulmonary artery (PASMC) smooth muscle cells, and coronary artery (HCAEC), aortic (HAEC), pulmonary (HPAEC), umbilical vein (HUVEC), dermal (HMVEC-D) and lung (HMVEC-L) microvascular endothelial cells in culture.

FIG. 2. ETL expression in the cornea cauterization model.

FIG. 3. ETL expression in the rat aortic ring model.

FIG. 4. GPR expression in human aortic (AoSMC), coronary artery (CASMC) and pulmonary artery (PASMC) smooth muscle cells, and coronary artery (HCAEC), aortic (HAEC), pulmonary (HPAEC), umbilical vein (HUVEC), dermal (HMVEC-D) and lung (HMVEC-L) microvascular endothelial cells in culture.

FIG. 5. GPR expression in the cornea cauterization model.

FIG. 6. GPR expression in the rat aortic ring model.

FIG. 7. Images of morpholino oligo (MO) knockdown using mismatch control (A), ETL MO (B) or GPR MO (C) in zebrafish 48 hours post fertilization. ETL knockdown results in retarded eye formation, edema in the brain and disrupted intersegmental vessel development and disrupted blood circulation.

FIG. 8. Morpholino oligo knockdown of ETL (right) versus control (left) in intersomitic vessels of the zebrafish. MO oligos are injected at the 1-2 cell stage and zebrafish are grown at 28° C. for 48 hours post-fertilization. Fluorescent images are taken 48 hours post-fertilization to assess defects in vasculogenesis or angiogenesis.

FIG. 9. Morpholino oligo knockdown of GPR (right) versus control (left) in intersomitic vessels of the zebrafish. MO oligos are injected at the 1-2 cell stage and zebrafish are grown at 28° C. for 48 hours post-fertilization. Fluorescent images are taken 48 hours post-fertilization to assess defects in vasculogenesis or angiogenesis.

FIG. 10. Corneal Micropocket Study in wildtype (WT; n=6) and ETL knockout (ETL KO; n=6) mice. A sucralfate pellet containing 60 ng of VEGF₁₆₅ is inserted into the cornea and blood vessel growth is quantitated.

### DETAILED DESCRIPTION OF THE INVENTION

### Molecules of the Invention

The invention comprises of various molecules:

genes that are DNA;
transcripts that are RNA;
nucleic acids that regulate their expression such as antisense molecules, siRNAs, micro RNAs;
molecules that may be used to detect them, such as DNA or RNA probes;
primers that may be used to identify and isolate related genes; and
proteins and polypeptides, and compounds that inhibit or activate them.

Thus, the term molecule is used herein to describe all or some of the entities of the invention. It is to be construed in the context it is used in.

Many biological functions are accomplished by altering the expression of various genes through transcriptional (e.g. through control of initiation, provision of RNA precursors, RNA processing) or translational control. For example, fundamental biological processes such as cell cycle, cell differentiation and cell death, are often characterized by the variations in the expression levels of groups of genes and their translational products.

Changes in gene expression may also be associated with pathogenesis. For example, the lack of sufficient expression of functional tumor suppressor genes or the over expression of oncogene/proto-oncogenes could lead to tumorigenesis or hyperplastic growth of cells. Thus, changes in the expression levels of particular genes or gene families may serve as signposts for the presence and progression of various diseases.

Monitoring changes in gene expression may also provide certain advantages during drug screening. Often drugs are screened for the ability to interact with a major target without regard to other effects the drugs have on cells. Often such other effects cause toxicity in the whole animal, which prevent the use of the potential drug.

The present inventors have examined various models of angiogenesis to identify the global changes in gene expression during angiogenesis. These global changes in gene expression, also referred to as expression profiles, may provide novel targets for the treatment of angiogenesis. They may also provide useful markers for diagnostic uses as well as markers that may be used to monitor disease states, disease progression, toxicity, drug efficacy, and drug metabolism.

The expression profiles may be used to identify genes that are differentially expressed under different conditions. In addition, the present invention may be used to identify families of genes that are differentially expressed. As used herein, “gene families” includes, but is not limited to; the specific genes identified by accession numbers herein, as well as related sequences. Related sequences may be, for example, sequences having a high degree of sequence homology with an identified sequence either at the nucleotide level or at the amino acid level. A high degree of sequence identity is seen to be at least about 65% sequence identity at the nucleotide level to the genes; preferably at least about 80%, or more preferably at least about 85%, or more preferably at least about 90%, or more preferably at least about 95%, or more preferably at least about 98% or more sequence identity to the genes. With regard to amino acid identity, a high degree of identity is seen to be at least about 50% identity, more preferably at least about 75% identity, more preferably at least about 85% identity, more preferably at least about 95% identity, or more preferably at least about 98% or more sequence identity. Methods are known in the art for determining homologies and identities between various sequences some of which are described later. In particular, related sequences include homologs and orthologs from different organisms. For example, if an identified gene were from a non-human mammal, the gene family would encompass homologous genes from other vertebrates or mammals including humans. If the identified gene were a human gene, the gene family would encompass the homologous gene from different organisms. Those skilled in the art will appreciate that a homologous gene may be of different length and may comprise regions with differing amounts of sequence identity to a specifically identified sequence.

One of skill in the art would also recognize that genes and proteins from species other than those listed in the sequence listing, particularly vertebrate species, could be useful in the present invention. Such species include, but are not limited to, rats, guinea pigs, rabbits, dogs, pigs, goats, cows, monkeys, chimpanzees, sheep, hamsters and zebrafish. One of skill in the art would further recognize that by using probes from the known species' sequences, cDNA or genomic sequences homologous to the known sequence could be obtained from the same or alternate species by known cloning methods. Such homologs and orthologs are contemplated to be useful as gene and proteins of the invention.

By “variants” are intended similar sequences. For example, conservative variants may include those sequences that, because of the degeneracy of the genetic code, encode the amino acid sequence of one of the polypeptides of the invention. Naturally occurring allelic variants, and splice variants may be identified with the use of known techniques, e.g., with polymerase chain reaction (PCR), single nucleotide polymorphism (SNP) analysis, and hybridization techniques. In order to isolate orthologs and homologs, generally stringent hybridization conditions are utilized dictated by specific sequence, sequence length, guanine+cytosine (GC) content and other parameters. Variant nucleotide sequences also include synthetically derived nucleotide sequences, e.g., derived by using site-directed mutagenesis. Variants may contain additional sequences from the genomic locus alone or in combination with other sequences.

The molecules of the invention also include truncated and/or mutated proteins wherein regions of the protein not required for ligand binding or signaling have been deleted or modified. Similarly, they may be mutated to modify their ligand binding or signaling activities. Such mutations may involve non-conservative mutations, deletions, or additions of amino acids or protein domains. Variant proteins may or may not retain biological activity. Such variants may result from, e.g., genetic polymorphism or from human manipulation.

Fragments and variants of genes and proteins of the invention are also encompassed by the present invention. By “fragment” is intended a portion of the nucleotide or protein sequence. Fragments may retain the biological activity of the native protein. Fragments of a nucleotide sequence are also useful as hybridization probes and primers or to regulate expression of a gene, e.g., antisense, siRNA, or micro RNA. A biologically active portion may be prepared by isolating a portion of a nucleotide sequence, expressing the isolated portion (e.g., by recombinant expression), and assessing the activity of the encoded protein.

Fusions of a protein or a protein fragment to a different polypeptide are also contemplated. Using known methods, one of skill in the art would be able to make fusion proteins that, while different from native form, would be useful. For example, the fusion partner may be a signal (or leader) polypeptide sequence that co-translationally or post-translationally directs transfer of the protein from its site of synthesis to another site (e.g., the yeast α-factor leader). Alternatively, it may be added to facilitate purification or identification of the protein of the invention (e.g., poly-His, Flag peptide, or fluorescent proteins).

The molecules of the invention may be prepared by various methods, including, but not limited to, cloning, PCR-based cloning, site-directed mutagenesis, mutagenesis, DNA shuffling, and nucleotide sequence alterations known in the art. See, for example, Molecular Cloning: A Laboratory Manual, 2^{nd} Edition, Sambrook, Fristch, and Maniatis (1989), Cold Spring Harbor Laboratory Press; Current Protocols in Molecular Biology, Ausubel et al., (1996) and updates, John Wiley and Sons; Methods in Molecular Biology (series), volumes 158, and 182. Humana Press; PCR Protocols: A guide to Methods and Applications, Innis, Gelfand, Sninsky, and White, 1990, Academic Press.

Libraries of recombinant polynucleotides may also be generated from a population of related sequences comprising regions that have substantial sequence identity and may be recombined in vitro or in vivo. For example, using this approach, sequence motifs encoding a domain of interest may be shuffled between a gene of the invention and other known genes to obtain a new gene coding for a protein with an altered property of interest e.g. a dominant negative mutation (Ohba et al. (1998) Mol. Cell. Biol. 18:51199-51207, Matsumoto et al. (2001) J. Biol. Chem. 276:14400-14406).

The “percent identity” or “sequence identity” may be determined by aligning two sequences or subsequences over a comparison window, wherein the portion of the sequence in the comparison window may optionally comprise additions or deletions (i.e., gaps) as compared to the reference sequence (which may comprise additions or deletions) for optimal alignment of the two sequences. The percentage is calculated by determining the number of positions at which an identical residue (e.g., nucleic acid base or amino acid) occurs in both sequences, dividing the number of matched positions by the total number of positions in the window of comparison, and multiplying the result by 100 to yield the percentage of sequence identity.

Percentage sequence identity may be calculated by the local homology algorithm of Smith & Waterman, Adv. Appl. Math. 2:482-485 (1981); or by the homology alignment algorithm of Needleman & Wunsch, J. Mol. Biol. 48:443-445 (1970); either manually or by computerized implementations of these algorithms (GAP & BESTFIT in the GCG Wisconsin Software Package, Genetics Computer Group; various BLASTs from National Center for Biotechnology Information (NCBI), NIH).

A preferred method for determining homology or sequence identity is by BLAST (Basic Local Alignment Search Tool) analysis using the algorithm employed by the programs blastp, blastn, blastx, tblastn and tblastx (Karlin et al. (1990) Proc. Natl. Acad. Sci. USA 87, 2264-2268 and Altschul, (1993) J. Mol. Evol. 36, 290-300), which are tailored for sequence similarity searching.

As described herein, these various genes and proteins, their allelic and other variants (e.g. splice variants), their homologs and orthologs from other species and various fragments and mutants may exhibit sequence variations. The length of the sequence to be compared may be less than the full-length sequence.

Cell lines, Vectors, Cloning, and Expression of Recombinant Molecules

Molecules of the invention may be prepared for various uses, including, but not limited to: to purify the protein or nucleic acid product, to generate antibodies, for use as reagents in the screening assays, and for use as pharmaceutical compositions. Some embodiments may be carried out using an isolated gene or a protein, while other embodiments may require use of cells that express them.

Where the source of molecule is a cell line, the cells may endogenously express it; may have been stimulated to increase endogenous expression; or have been genetically engineered to express the molecule. Expression of a protein of interest may be determined by, for example, detection of the polypeptide with an appropriate antibody (e.g. Western blot), use of a DNA probe to detect mRNA encoding the protein (e.g., northern blot or various PCR-based techniques), or measuring binding of an agent selective for the polypeptide of interest (e.g., a suitably-labeled selective ligand).

The present invention further provides recombinant molecules that contain a coding sequence of, or a variant form of, a molecule of invention. In a recombinant DNA molecule, a coding DNA sequence is operably linked to other DNA sequences of interest including, but not limited to, various control sequences for integration, replication, transcription, expression, and modification.

The choice of vector and control sequences to which a gene sequence of the present invention is operably linked depends upon the functional properties desired (e.g., protein expression, the host cell to be transformed). A vector of the present invention may be capable of directing the replication or insertion into the host chromosome, and preferably expression of the gene.

Control elements that are used for regulating the expression of a gene are known in the art and include, but are not limited to, inducible or constitutive promoters, secretion signals, enhancers, termination signals, ribosome-binding sites, and other regulatory elements. Optimally, the inducible promoter is readily controlled, such as being responsive to a nutrient, or an antibiotic.

In one embodiment, the vector harboring a nucleic acid molecule may include a prokaryotic replicon, i.e., a DNA sequence having the ability to direct autonomous replication and maintenance of the recombinant DNA molecule extra-chromosomally in a prokaryotic host cell, such as a bacterial host cell. In addition, vectors that include a prokaryotic replicon may also include a gene whose expression confers a detectable characteristic (e.g., resistance to ampicillin).

Vectors may further include a prokaryotic or bacteriophage promoter capable of directing the expression (transcription and translation) of the coding gene sequences in a bacterial host cell, such as E. coli. Promoter sequences compatible with bacterial hosts may be provided in plasmid vectors containing convenient restriction sites for insertion of a DNA sequence of the present invention, e.g., pcDNA1, pcDNA3.

Expression vectors compatible with eukaryotic cells may also be used to form a recombinant molecule that contains a sequence of interest. Commercially available vectors often contain both prokaryotic and eukaryotic replicons and control sequences, for an easy switch from prokaryotic to eukaryotic cell to ES cells for generating transgenic cells or animals (e.g., pcDNA series from Invitrogen™).

Eukaryotic cell expression vectors used to construct the recombinant molecules of the present invention may further include a selectable marker that is effective in a eukaryotic cell (e.g., neomycin resistance). Alternatively, the selectable marker may be present on a separate plasmid, the two vectors introduced by co-transfection of the host cell, and transfectants selected by culturing in the appropriate drug for the selectable marker. Vectors may also contain fusion protein, or tag sequences that facilitate purification or detection of the expressed protein.

The present invention further provides host cells transformed with a recombinant molecules of the invention. The host cell may be a prokaryote, e.g., a bacterium, or a eukaryote, e.g., yeast, insect or vertebrate cells, including, but not limited to, cells from a mouse, monkey, frog, human, rat, guinea pig, rabbit, dog, pig, goat, cow, chimpanzee, sheep, hamster or zebrafish. Commonly used eukaryotic host cell lines include, but are not limited to, CHO cells, ATCC CCL61, NIH-3T3, and BHK cells. In many instances, primary cell cultures from animals may be preferred.

Transformation of appropriate host cells with a molecule of the present invention may be accomplished by known methods that depend on the host system employed. For transforming prokaryotic host cells, electroporation and salt treatment methods may be employed, while for transformation of eukaryotic cells, electroporation, cationic lipids, or salt treatment methods may be employed (See Sambrook et al. (1989) supra). Viral vectors, including, but not limited to, retroviral and adenoviral vectors have also been developed that facilitate transfection of primary or terminally differentiated cells. Other techniques may also be used that introduce DNA into cells e.g., liposome, gold particles, or direct injection of the DNA expression vector (as a projectile), containing the gene of interest, into human tissue.

Successfully transformed cells may be cloned to produce stable clones. Cells from these clones may be harvested, lysed and their content examined for the presence of the recombinant molecules using known methods.

### Biological Samples

As is apparent to one of ordinary skill in the art, nucleic acid samples, which may be DNA and/or RNA, used in the methods and assays of the invention may be prepared by available methods. Methods of isolating total mRNA are known. For example, methods of isolation and purification of nucleic acids are described in detail in Chapter 3 of Tijssen, (1993) Laboratory Techniques in Biochemistry and Molecular Biology: Hybridization with Nucleic Acid Probes, Elsevier Press. Such samples include RNA samples, but may also include cDNA synthesized from an mRNA sample isolated from a cell or tissue of interest. Such samples also include DNA amplified from the cDNA, and RNA transcribed from the amplified DNA.

Biological samples containing nucleic acids, or proteins may be of any biological tissue or fluid or cells from any organism as well as cells grown in vitro, such as cell lines and tissue culture cells. The sample may be a “clinical sample” which is a sample derived from a patient. Typical clinical samples include, but are not limited to, sputum, blood, blood-cells (e.g., white cells), various tissues or organs or parts thereof, or fine needle biopsy samples, urine, peritoneal fluid, and pleural fluid, or cells there from.

Biological samples may also include sections of tissues, such as frozen sections or formaldehyde-fixed sections taken for histological purposes.

### Isolation of Other Related Nucleic Acid Molecules

As described above, the identification of the human nucleic acid molecules of Table 4 allows a skilled artisan to isolate nucleic acid molecules that encode other members of the gene family in addition to the sequences herein described. Further, the presently disclosed nucleic acid molecules allow a skilled artisan to isolate nucleic acid molecules that encode other members of the gene families.

A skilled artisan may use the proteins of Table 4 or fragments thereof to generate antibody probes to screen expression libraries prepared from appropriate cells. In one embodiment, the fragments may contain amino acid insertion and substitution.

Polyclonal antiserum from mammals such as rabbits immunized with the purified protein, or monoclonal antibodies may be used to probe a mammalian cDNA or genomic expression library, such as lambda gt11 library, to obtain the appropriate coding sequence for other members of the protein family. The cloned cDNA sequence may be expressed as a fusion protein, expressed using its own control sequences, or expressed by constructs using control sequences appropriate to the particular host used for expression of a protein.

Alternatively, a portion of coding sequences herein described may be synthesized and used as a probe to retrieve DNA encoding a member of the protein family from any organism. Oligomers, e.g., containing 18-20 nucleotides, may be prepared and used to screen genomic DNA or cDNA libraries to obtain hybridization under stringent conditions or conditions of sufficient stringency to eliminate an undue level of false positives.

Additionally, pairs of oligonucleotide primers may be prepared for use in a polymerase chain reaction (PCR) to clone a nucleic acid molecule. Various PCR formats are known in the art and may be adapted for use in isolating other nucleic acid molecules.

### Selection of Test Compounds

Compounds that may be screened in accordance with the assays of the invention include, but are not limited to, libraries of known compounds, including natural products, such as plant or animal extracts. Also included are synthetic chemicals, biologically active materials, e.g., proteins, nucleic acids, and peptides, including, but not limited to, members of random peptide libraries and combinatorial chemistry derived molecular libraries made of D- or L-configuration amino acids, and phosphopeptides, antibodies (including, but not limited to, polyclonal, monoclonal, chimeric, human, anti-idiotypic or single chain antibodies, and Fab, F(ab′)₂ and Fab expression library fragments, and epitope-binding fragments thereof); and other organic and inorganic molecules.

In addition to the more traditional sources of test compounds, computer modeling and searching technologies permit the rational selection of test compounds by utilizing structural information from the ligand binding sites of proteins of the present invention.

Such rational selection of test compounds may decrease the number of test compounds that must be screened in order to identify a therapeutic compound. Knowledge of the protein sequences of the present invention may allow for generation of models of their binding sites that may be used to screen for potential ligands. This process may be accomplished in manners known in the art. A preferred approach involves generating a sequence alignment of the protein sequence to a template (derived from the crystal structures or NMR-based model of a similar protein(s), conversion of the amino acid structures and refining the model by molecular mechanics and visual examination. If a strong sequence alignment cannot be obtained then a model may also be generated by building models of the hydrophobic helices. Mutational data that point towards contact residues may also be used to position the helices relative to each other so that these contacts are achieved. During this process, docking of the known ligands into the binding site cavity within the helices may also be used to help position the helices by developing interactions that would stabilize the binding of the ligand. The model may be completed by refinement using molecular mechanics and loop building using standard homology modeling techniques. General information regarding modeling may be found in Schoneberg, T. et. al., Molecular and Cellular Endocrinology, 151:181-193 (1999), Flower, D., Biochim Biophys Acta, 1422, 207-234 (1999), and Sexton, P. M., Curr. Opin. Drug Discovery and Development, 2, 440-448 (1999).

Once the model is completed, it may be used in conjunction with one of several computer programs to narrow the number of compounds to be screened, e.g., the DOCK program (UCSF Molecular Design Institute, 533 Parnassus Ave, U-64, Box 0446, San Francisco, Calif. 94143-0446) or FLEXX (Tripos Inc., 1699 South Hanley Rd., St. Louis, Mo.). One may also screen databases of commercial and/or proprietary compounds for steric fit and rough electrostatic complementarity to the binding site.

### Screening Assays to Identify Compounds

The finding that the genes of the present invention may play a role in regulating angiogenesis enables various methods of screening one or more compounds to identify compounds that may be used for prophylactic or therapeutic treatment of angiogenesis.

When selecting compounds useful for prevention or treatment, it may be preferable that the compounds be selective for proteins of invention. For initial screening, it may be preferred that the in vitro screen be carried out using a protein of the invention with an amino acid sequence that is, e.g., at least about 80% identical, preferably at least about 90% identical, and more preferably identical to a protein sequence described in Table 4. Preferably, the test compounds may be screened against a vertebrate protein, more preferably a human protein. For screening compounds it may be preferable to use the protein from the species in which treatment is contemplated.

The methods of the present invention may be amenable to high throughput applications; however, use of as few as one compound in the method is encompassed by the term “screening”. This in vitro screen provides a means by which to select a range of compounds, i.e., the compounds, which merit further investigation. For example, compounds that activate a protein of the invention at concentrations of less than 200 nM might be further tested in an animal model, whereas those above that threshold may not be further tested.

The assay systems described below may be formulated into kits comprising a protein of the invention or cells expressing a protein of the invention, which may be packaged in a variety of containers, e.g., vials, tubes microtitre plates, bottles and the like. Other reagents may be included with the kit, e.g., positive and negative control samples, and buffers.

In one embodiment, the invention provides a method to identify compounds that bind to a protein of the invention. Methods to determine binding of a compound to a protein are known in the art. The assays include incubating a protein of the invention with a labeled compound, known to bind to the protein, in the presence or absence of a test compound and determining the amount of bound labeled compound. The source of a protein of the invention may either be cells expressing the protein or some form of isolated protein. The labeled compound may be a known ligand or a ligand analog labeled such that it may be measured, preferably quantitatively (e.g., labeled with ¹²⁵I, ³⁵S-methionine, or a fluorescent tag, or peptide or a fluorescent protein fusions). Such methods of labeling are known in the art. Test compounds that bind to a protein of the invention may reduce ligand bound to the protein, thereby reducing the signal level compared to control samples. Variations of this technique have been described Keen, M., Radioligand Binding Methods for Membrane Preparations and Intact cells in Receptor Signal Transduction Protocols, R. A. J. Challis, (ed), Humana Press Inc., Totoway N.J. (1997).

In another embodiment, the invention provides methods for screening test compounds to identify compounds that activate a protein of the invention. The assays are cell-based; however, cell-free assays are known which are able to differentiate agonist and antagonist binding. Cell-based assays include contacting cells that express a protein of the invention with a test compound or a control substance and measuring activation of the protein by measuring the expression or activity of components of the affected signal transduction pathways. For example, after suitable incubation with a test compound, lysates of the cells may be prepared and assayed for transcription, translation, or modification of a protein, e.g., phosphorylation, or glycosylation, or induction of second messengers like cAMP. Many high-throughput assays are available that measure the response without the need of lysing the cells, e.g. calcium imaging.

In one embodiment, cAMP induction may be measured with the use of recombinant constructs containing the cAMP responsive element linked to any of a variety of reporter genes. Such reporter genes include, but are not limited to, chloramphenicol acetyltransferase (CAT), luciferase, glucuronide synthetase, growth hormone, fluorescent proteins, or alkaline phosphatase. Following exposure of the cells to a test compound, the level of reporter gene expression may be quantified to determine the test compound's ability to increase cAMP levels and thus determine a test compound's ability to activate a protein of the invention.

In another embodiment, specific phospho-tyrosine or phospho-serine antibodies may be utilized to measure the level of phosphorylation of a signaling protein after the exposure to a test compound, whereby a significant deviation in phosphorylation levels compared to control samples would indicate activation of a protein of the invention. In some instances, a protein's (for example receptor) responses subside, or become desensitized, after prolonged exposure to an agonist. In many cases, the protein of interest may be an enzyme and thus the effect of the binding of the test compounds could be measured in terms of changes in the enzymatic activity. Similarly, changes in intracellular calcium concentration [Ca²⁺] are generally indicative of activation of many signaling cascades.

### Screening for Compounds Using Cell Culture, Tissue, and Animal Models of Angiogenesis

Compounds selected from one or more test compounds by an in vitro assay, as described above, may be further tested for their ability to regulate angiogenesis in various models of angiogenesis. Such models include both in vitro cell culture models and in vivo animal models. Such additional levels of screening are useful to further narrow the range of candidate compounds that merit additional investigation, e.g., clinical trials. Such model systems include, endothelial cell proliferation/survival assays, endothelial cell migration assays, tube-forming assays, microbead sprouting assay, rat aortic ring assay, chicken aortic arch assay, chicken (or other species) chorioallantoic membrane (CAM) assay, direct in vivo angiogenesis assay (DIVAA), examination of blood flow in the hind limb, heart or other organ in the presence and absence of vessel occlusion, examination of blood vessel growth and development in zebrafish, corneal angiogenesis assay or various modifications of these assays.

### Transgenic Animals and Gene Therapy

Animals of many species, preferably vertebrates, including, but not limited to, mice, rats, rabbits, guinea pigs, pigs, goats, dogs, frogs, and non-human primates may be used to generate transgenic animals expressing the proteins of the invention. Several techniques are known in the art and may be used to introduce transgenes into animals to produce the founder lines of transgenic animals. Such techniques include, but are not limited to, pronuclear microinjection, retrovirus-mediated gene transfer into germ lines, gene targeting in embryonic stem cells, electroporation of embryos and sperm-mediated gene transfer.

The overall activity of a protein of the invention may be increased by overexpressing the gene for that protein. Overexpression will increase the total cellular protein activity, and thereby the function. The gene or genes of interest are inserted into a vector suitable for expression in the subject. These vectors include, but are not limited to, adenoviruses, adenovirus associated viruses, retroviruses and herpes virus vectors. Other techniques may also be used that introduce DNA into cells e.g., liposome, gold particles, or direct injection of the DNA expression vector (as a projectile), containing the gene of interest, into human tissue.

### Treatment of Angiogenesis Regulated Disorders

The genes and proteins of the present invention (targets), and compounds that activate or inhibit them may be used in a method for the treatment of an angiogenesis regulated disorder. The term “regulate” is defined as in its accepted dictionary meanings. Thus, meaning of the term “regulate” includes, but is not limited to, up-regulate or down-regulate, to fix, to bring order or uniformity, to govern, or to direct by various means. In one aspect, a compound may be used in a method for the treatment of an “angiogenesis elevated disorder” or “angiogenesis reduced disorder”. As used herein, an “angiogenesis elevated disorder” is one that involves unwanted or elevated angiogenesis in the biological manifestation of the disease, disorder, and/or condition; in the biological cascade leading to the disorder; or as a symptom of the disorder. Similarly, the “angiogenesis reduced disorder” is one that involves wanted or reduced angiogenesis in the biological manifestations. This “involvement” of angiogenesis in an angiogenesis elevated/reduced disorder includes, but is not limited to, the following:

(1) The angiogenesis as a “cause” of the disorder or biological manifestation, whether the level of angiogenesis is elevated or reduced genetically, by infection, by autoimmunity, trauma, biomechanical causes, lifestyle, or by some other causes.

(2) The angiogenesis as part of the observable manifestation of the disease or disorder. That is, the disease or disorder is measurable in terms of the increased or reduced angiogenesis. From a clinical standpoint, angiogenesis indicates the disease; however, angiogenesis need not be the “hallmark” of the disease or disorder.

(3) The angiogenesis is part of the biochemical or cellular cascade that results in the disease or disorder. In this respect, regulation of angiogenesis may interrupt the cascade, and may control the disease. Non-limiting examples of angiogenesis regulated disorders that may be treated by the present invention are herein described below.

Targets and compounds of present invention may be used to treat diseases associated with retinal/choroidal neovascularization that include, but are not limited to, diabetic retinopathy, macular degeneration, sickle cell anemia, sarcoid, syphilis, pseudoxanthoma elasticum, Paget's disease, vein occlusion, artery occlusion, carotid obstructive disease, chronic uveitis/vitritis, mycobacterial infections, Lyme's disease, systemic lupus erythematosis, retinopathy of prematurity, Eales' disease, Behcet's disease, infections causing a retinitis or choroiditis, presumed ocular histoplasmosis, Best's disease, myopia, optic pits, Stargardt's disease, pars planitis, chronic retinal detachment, hyperviscosity syndromes, toxoplasmosis, trauma and post-laser complications. Other diseases include, but are not limited to, diseases associated with rubeosis (neovasculariation of the iris) and diseases caused by the abnormal proliferation of fibrovascular or fibrous tissue including all forms of proliferative vitreoretinopathy, whether or not associated with diabetes.

Targets and compounds of the present invention may be used to treat diseases associated with chronic inflammation. Diseases with symptoms of chronic inflammation include inflammatory bowel diseases such as Crohn's disease and ulcerative colitis, psoriasis, sarcoidosis and rheumatoid arthritis. Angiogenesis is a key element that these chronic inflammatory diseases have in common. The chronic inflammation depends on continuous formation of capillary sprouts to maintain an influx of inflammatory cells. The influx and presence of the inflammatory cells produce granulomas and thus, maintain the chronic inflammatory state. Inhibition of angiogenesis by the compositions and methods of the present invention would prevent the formation of the granulomas and alleviate the disease.

Crohn's disease and ulcerative colitis are characterized by chronic inflammation and angiogenesis at various sites in the gastrointestinal tract. Crohn's disease is characterized by chronic granulomatous inflammation throughout the gastrointestinal tract consisting of new capillary sprouts surrounded by a cylinder of inflammatory cells. Prevention of angiogenesis inhibits the formation of the sprouts and prevents the formation of granulomas. Crohn's disease occurs as a chronic transmural inflammatory disease that most commonly affects the distal ileum and colon but may also occur in any part of the gastrointestinal tract from the mouth to the anus and perianal area. Patients with Crohn's disease generally have chronic diarrhea associated with abdominal pain, fever, anorexia, weight loss and abdominal swelling. Ulcerative colitis is also a chronic, nonspecific, inflammatory and ulcerative disease arising in the colonic mucosa and is characterized by the presence of bloody diarrhea.

The inflammatory bowel diseases also show extraintestinal manifestations such as skin lesions. Such lesions are characterized by inflammation and angiogenesis and may occur at many sites other than the gastrointestinal tract. Targets and compounds of the present invention may be capable of treating these lesions by preventing the angiogenesis, thus reducing the influx of inflammatory cells and the lesion formation.

Sarcoidosis is another chronic inflammatory disease that is characterized as a multisystem granulomatous disorder. The granulomas of this disease may form anywhere in the body and thus the symptoms depend on the site of the granulomas and whether the disease active. The granulomas are created by the angiogenic capillary sprouts providing a constant supply of inflammatory cells.

Targets and compounds of the present invention may also treat the chronic inflammatory conditions associated with psoriasis. Psoriasis, a skin disease, is another chronic and recurrent disease that is characterized by papules and plaques of various sizes. Prevention of the formation of the new blood vessels necessary to maintain the characteristic lesions leads to relief from the symptoms.

Rheumatoid arthritis is a chronic inflammatory disease characterized by nonspecific inflammation of the peripheral joints. It is believed that the blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis. Other diseases that may be treated according to the present invention are hemangiomas, Osler-Weber-Rendu disease, or hereditary hemorrhagic telangiectasia, solid or blood borne tumors and acquired immune deficiency syndrome.

The compounds of the present invention may also be used to treat an “angiogenesis reduced disorder”. As used herein, an “angiogenesis reduced disorder” is one that involves wanted or stimulated angiogenesis to treat a disease, disorder, and/or condition. The disorder is one characterized by tissue that is suffering from or be at risk of suffering from ischemic damage, infection, and/or poor healing, which results when the tissue is deprived of an adequate supply of oxygenated blood due to inadequate circulation. As used herein, “tissue” is used in the broadest sense, to include, but not limited to, the following: cardiac tissue, such as myocardium and cardiac ventricles; erectile tissue; skeletal muscle; neurological tissue, such as from the cerebellum; internal organs, such as the brain, heart, pancreas, liver, spleen, and lung; or generalized area of the body such as entire limbs, a foot, or distal appendages such as fingers or toes.

Methods of Vascularizing Ischemic Tissue

In one aspect, targets or compounds may be used in a method of vascularizing ischemic tissue. As used herein, “ischemic tissue,” means tissue that is deprived of adequate blood flow. Examples of ischemic tissue include, but are not limited to, tissue that lack adequate blood supply resulting from mycocardial and cerebral infarctions, mesenteric or limb ischemia, or the result of a vascular occlusion or stenosis. In one example, the interruption of the supply of oxygenated blood may be caused by a vascular occlusion. Such vascular occlusion may be caused by arteriosclerosis, trauma, surgical procedures, disease, and/or other etiologies. Standard routine techniques are available to determine if a tissue is at risk of suffering ischemic damage from undesirable vascular occlusion. For example, in myocardial disease these methods include a variety of imaging techniques (e.g., radiotracer methodologies, x-ray, and MRI) and physiological tests. Therefore, induction of angiogenesis is an effective means of preventing or attenuating ischemia in tissues affected by or at risk of being affected by a vascular occlusion. Further, the treatment of skeletal muscle and myocardial ischemia, stroke, coronary artery disease, peripheral vascular disease, coronary artery disease is fully contemplated.

A person skilled in the art of using standard techniques may measure the vascularization of tissue. Non-limiting examples of measuring vascularization in a subject include SPECT (single photon emission computed tomography); PET (positron emission tomography); MRI (magnetic resonance imaging); and combination thereof, by measuring blood flow to tissue before and after treatment. Angiography may be used as an assessment of macroscopic vascularity. Histologic evaluation may be used to quantify vascularity at the small vessel level. These and other techniques are discussed in Simons, et al., “Clinical trials in coronary angiogenesis,” Circulation, 102, 73-86 (2000).

Methods of Repairing Tissue

In one aspect, targets or compounds may be used in a method of repairing tissue. As used herein, “repairing tissue” means promoting tissue repair, regeneration, growth, and/or maintenance including, but not limited to, wound repair or tissue engineering. One skilled in the art appreciates that new blood vessel formation is required for tissue repair. In turn, tissue may be damaged by, including, but not limited to, traumatic injuries or conditions including arthritis, osteoporosis and other skeletal disorders, and burns. Tissue may also be damaged by injuries due to surgical procedures, irradiation, laceration, toxic chemicals, viral infection or bacterial infections, or burns. Tissue in need of repair also includes non-healing wounds. Examples of non-healing wounds include non-healing skin ulcers resulting from diabetic pathology; or fractures that do not heal readily.

Targets or compounds may also be used in tissue repair in the context of guided tissue regeneration (GTR) procedures. Such procedures are currently used by those skilled in the arts to accelerate wound healing following invasive surgical procedures.

Targets or compounds may be used in a method of promoting tissue repair characterized by enhanced tissue growth during the process of tissue engineering. As used herein, “tissue engineering” is defined as the creation, design, and fabrication of biological prosthetic devices, in combination with synthetic or natural materials, for the augmentation or replacement of body tissues and organs. Thus, the present methods may be used to augment the design and growth of human tissues outside the body for later implantation in the repair or replacement of diseased tissues. For example, compounds may be useful in promoting the growth of skin graft replacements that are used as a therapy in the treatment of burns.

In another aspect of tissue engineering, targets or compounds of the present invention may be included in cell-containing or cell-free devices that induce the regeneration of functional human tissues when implanted at a site that requires regeneration. As previously discussed, biomaterial-guided tissue regeneration may be used to promote bone regrowth in, for example, periodontal disease. Thus, targets or compounds may be used to promote the growth of reconstituted tissues assembled into three-dimensional configurations at the site of a wound or other tissue in need of such repair.

In another aspect of tissue engineering, targets or compounds may be included in external or internal devices containing human tissues designed to replace the function of diseased internal tissues. This approach involves isolating cells from the body, placing them with structural matrices, and implanting the new system inside the body or using the system outside the body. For example, targets or compounds may be included in a cell-lined vascular graft to promote the growth of the cells contained in the graft. It is envisioned that the methods of the invention may be used to augment tissue repair, regeneration and engineering in products such as cartilage and bone, central nervous system tissues, muscle, liver, and pancreatic islet (insulin-producing) cells.

Pharmaceutical Formulations and Methods for Use

Compounds identified by screening methods described herein may be administered to individuals to treat or to prevent diseases or disorders that are regulated by genes and proteins of the invention. The term “treatment” is used herein to mean that administration of a compound of the present invention mitigates a disease or a disorder in a host. Thus, the term “treatment” includes, preventing a disorder from occurring in a host, particularly when the host is predisposed to acquiring the disease, but has not yet been diagnosed with the disease; inhibiting the disorder; and/or alleviating or reversing the disorder. Insofar as the methods of the present invention are directed to preventing disorders, it is understood that the term “prevent” does not require that the disease state be completely thwarted. (See Webster's Ninth Collegiate Dictionary.) Rather, as used herein, the term preventing refers to the ability of the skilled artisan to identify a population that is susceptible to disorders, such that administration of the compounds of the present invention may occur prior to onset of a disease. The term does not imply that the disease state be completely avoided. The compounds identified by the screening methods of the present invention may be administered in conjunction with other compounds.

Safety and therapeutic efficacy of compounds identified may be determined by standard procedures using in vitro or in vivo technologies. Compounds that exhibit large therapeutic indices may be preferred, although compounds with lower therapeutic indices may be useful if the level of side effects is acceptable. The data obtained from the in vitro and in vivo toxicological and pharmacological techniques may be used to formulate the range of doses.

Effectiveness of a compound may further be assessed either in animal models or in clinical trials of patients with unregulated or improperly regulated angiogenesis.

As used herein, “pharmaceutically acceptable carrier” is intended to include all solvents, dispersion media, coatings, antibacterial and antifungal agents, isotonic and absorption delaying agents, and the like, compatible with pharmaceutical administration. The use of such media and agents for pharmaceutically active substances is known in the art. Except insofar as any conventional media or agent is incompatible with the active compound, such media may be used in the compositions of the invention. Supplementary active compounds may also be incorporated into the compositions. A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application may include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH may be adjusted with acids or bases, such as hydrochloric acid or sodium hydroxide. The parenteral preparation may be enclosed in ampoules, disposable syringes or multiple dose vials made of glass or plastic.

Pharmaceutical compositions suitable for injectable use include sterile aqueous solutions (where water-soluble), or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier may be a solvent or dispersion medium containing, for example, water, ethanol, polyol (for example, glycerol, propylene glycol, and liquid polyethylene glycol, and the like), and suitable mixtures thereof. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions may be brought about by including in the composition an agent that delays absorption, for example, aluminum monostearate and gelatin.

Sterile injectable solutions may be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They may be enclosed in gelatin capsules or compressed into tablets. For oral administration, the agent may be contained in enteric forms to survive the stomach or further coated or mixed to be released in a particular region of the GI tract by known methods. For the purpose of oral therapeutic administration, the active compound may be incorporated with excipients and used in the form of tablets, troches, or capsules. Oral compositions may also be prepared using a fluid carrier for use as a mouthwash, wherein the compound in the fluid carrier is applied orally and swished and expectorated or swallowed. Pharmaceutically compatible binding agents, and/or adjuvant materials may be included as part of the composition. The tablets, pills, capsules, troches and the like may contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration may also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration may be accomplished using nasal sprays or suppositories. For transdermal administration, the active compounds are formulated into ointments, salves, gels, or creams as generally known in the art.

The compounds may also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, and polylactic acid. Methods for preparation of such formulations will be apparent to those skilled in the art. The materials may also be obtained commercially from Alza Corporation and Nova Pharmaceuticals, Inc. Liposomal suspensions (including liposomes targeted to infected cells with monoclonal antibodies to viral antigens) may also be used as pharmaceutically acceptable carriers. These may be prepared according to methods known to those skilled in the art, for example, as described in U.S. Pat. No. 4,522,811.

It is especially advantageous to formulate oral or parenteral compositions in dosage unit form for ease of administration and uniformity of dosage. “Dosage unit form” as used herein refers to physically discrete units suited as unitary dosages for the subject to be treated, each unit containing a predetermined quantity of active compound calculated to produce the desired therapeutic effect in association with the required pharmaceutical carrier. The specification for the dosage unit forms are dictated by and are directly dependent on the unique characteristics of the active compound and the particular therapeutic effect to be achieved, and the limitations inherent in the art of compounding such an active compound for the treatment of individuals.

### Diagnostic Uses

As described above, the genes and gene expression information provided in Table 1 and 2 may be used as diagnostic markers for the prediction or identification of the disease state of a sample tissue. For instance, a tissue sample may be assayed by any of the methods described above, and the expression levels for a gene or member of a gene family from Table 1 may be compared to the expression levels found in normal subject. The expression level may also be compared to the expression levels observed in sample tissues exhibiting a similar disease state, which may aid in its diagnosis. The comparison of expression data, as well as available sequences or other information may be done by researcher or diagnostician or may be done with the aid of a computer and databases as described above. Such methods may be used to diagnose or identify conditions characterized by abnormal expression of the genes that are described in Table 1.

The methods of the present invention may be particularly useful in diagnosing or monitoring effectiveness of treatment regimen. Compounds that modulate the expression of one or more genes or gene families or proteins identified in Table 4 and/or modulate the activity of one or more of the proteins encoded by one or more of the genes or members of a gene family identified in Table 4 will be useful in diagnosis, monitoring, and evaluation of patient responses to treatment regimen.

### EXAMPLES

### Example 1

### Identification of Genes that are Differentially Regulated in the Rat Cornea Model of Angiogenesis

The cornea has been widely used in the study of experimental angiogenesis because of the ease with which new blood vessels may be induced and studied in this normally transparent and avascular tissue. Corneal cauterization of anesthetized rats using silver nitrate stimulates a reproducible angiogenic response (Burger. P. C., et al., Lab Invest 48:169-180 (1983)). New blood vessels arise predominantly from limbal blood vessels beginning at day 1 post-cautery and invade the corneal stroma. By day 7 numerous vessels have reached the site of cauterization. The initial angiogenic response is followed by vessel pruning and remodeling. After euthanasia, cornea-scleral tissue samples, including the limbic vessels, are collected and flash frozen in liquid nitrogen on days 0, 1, 2, 4, 7, 15, and 38. After euthanasia, cornea samples dissected to exclude the limbal vessels are collected and flash frozen in liquid nitrogen on days 0, 4, 7, and 15. Six replicate samples are used for each experimental condition at the indicated time points.

### GeneChip Probe Preparation

Frozen corneal tissues from rats are homogenized in Trizol (Life Technologies, Rockville, Md.) using Tungsten Carbide Beads (Qiagen, Chatsworth, Calif.) with shaking in Mixer Mill (Qiagen). RNA samples are prepared according to Affymetrix (Santa Clara, Calif.) recommendations. Briefly, total RNA is prepared with the use of Trizol reagent (Life Technologies). The RNA is purified with an RNeasy Mini Kit (Qiagen). Reverse transcription is performed on 10 μg of total RNA with the use of SuperScript II Reverse Transcriptase (Life Technolohies) and a T7-(dT)₂₄ primer. Second strand DNA is synthesized with T4 DNA polymerase. The double stranded cDNA is extracted, and recovered by ethanol precipitation. The RNA Transcript Labeling Kit (Enzo Diagnostics, Farmingdale, N.Y.) is used for production of hybridizble biotin-labeled cRNA (complementary RNA) targets by in vitro transcription from T7 RNA polymerase promoters. The cDNA prepared from total RNA is used as a template in the presence of a mixture of unlabeled NTPs, and biotinylated CTP and UTP. In vitro transcription products are purified with an RNeasy Mini Kit to remove unincorporated NTPs and are fragmented to approximately 35 to 200 bases by incubation at 94° C. for 35 min in fragmentation buffer containing Tris-acetate, potassium acetate, and magnesium acetate. Fragmented cRNA is stored at −20° C. until the hybridization is performed.

### Array Hybridization, Stain, and Scan

Biotinylated and fragmented cRNA is hybridized for 16 h at 45° C. to a set of rat RAE230A and RAE230B arrays (Affymetrix) in a GeneChip Hybridization Oven 640 (Affymetrix). A series of stringency washes and staining with streptavidin-conjugated phycoerythrin is then performed in a GeneChip Fluidic Station 400 (Affymetrix) according to the protocol recommended by Affymetrix. Probe arrays are then scanned with an Agilent GeneArray Scanner. The images are analyzed with the GeneChip Analysis software (Affymetrix).

### Statistical Analysis of Affymetrix Microarray Experiments

The statistical analysis of the rat cornea samples is based on the Affy signal (MAS 5.0 algorithm). The data from chip RAE230A and RAE230B are rescaled (normalized) based on the 100 common genes from both sets. The first step in the analysis is to check data quality using exploratory statistical tools—summary statistics, pair plots, Principal Component Analysis (PCA). The results show that treatment and time effect are the major sources of data variability.

At this stage of data preprocessing, gene filtering is performed based on the minimum number of Affy Absent Calls per experimental condition. The Affy algorithm gives Absent Call for a gene on a chip if the gene expression level is very low compared to the background noise (i.e. the gene is not detected as expressed). To remove genes with no expression across all conditions, the minimum number of Absent Calls is determined across all experimental conditions. A gene is removed from further analysis if the Absent Call minimum is at least 4 (out of 6 replicates). Hence, the eliminated genes have at least 4 Absent Calls for each condition.

The next step in the analysis is to run ANalysis Of VAriance (ANOVA) statistical model to estimate Log Fold Change (LFC) and corresponding uncertainty measure, Standard Error (SE), for paired conditions of interest. The ratio of LFC to SE is investigated to determine the statistical significance of the differential gene expression between two compared experimental conditions. The statistical significance is summarized by a quantity called NLOGP (=−log₁₀[P-value]). The length of a gene list depends on desired Average False Positive Rate. An NLOGP threshold equal to −log₁₀ (Average False Positive Rate) is used to detect genes with statistically significant differential expression (corresponding NLOGP measure is greater then the NLOGP threshold). Using an NLOP=4 as a threshold, 7162 genes, or probe sets, are differentially regulated in at least one experimental condition and time point in the rat cornea model. These genes are further analyzed in Example 3.

### Example 2

### Identification of Genes that are Differentially Regulated in the Rat Aortic Ring Model of Angiogenesis

Various aspects of the angiogenesis process may be studied in vitro by culturing explants of rat aorta in gels of biological matrices (Nicosia, R. F. and Ottinetti, A., Lab Invest 63:115-122 (1990)). Angiogenesis in the serum-free rat aorta model is a self-limited process triggered by the injury of the dissection procedure. Endothelial cells sprout from the cut edges of the explant at days 3 to 4. During the second week of culture, microvessels elongate, branch, anastomose and eventually stop growing. Subsequent remodeling of the vascular outgrowth results in regression of the small branches which retract into the main stems of the larger vessels. Aortas are dissected from euthanized rats and cultured in a matrix of rat-tail collagen as previously described. In cases of denuded aortas, the endothelium is removed by gently rubbing the vessel between the thumb and forefinger, rinsing in medium, and subsequently placing the rings in the collagen matrix (Carr, A. N., et al., J. Physiology 534:357-366 (2001)). Intact and denuded aortas were cultured for a period of 0, 1, 2, 4, 7, 10, and 14 days. In total, 14 experimental conditions with 6 replicates were used in the study.

The steps of GeneChip probe preparation; array hybridization, stain, and scan; and statistical analysis of Affymetrix Microarray experiments are performed as described for Example 1 above.

Using an NLOP=4 as a threshold, 10171 genes, or probe sets, are differentially regulated in at least one experimental condition and time point in the rat aortic ring model. In comparing the normal aorta samples with the denuded aorta samples, 3475 genes, or probe sets, are found to be differentially regulated. These genes are further analyzed in Example 3.

### Example 3

### Identification of Genes Associated with Angiogenesis

Comparison of the genes that are differentially regulated in both the rat cornea and aortic ring models identifies 2274 common genes, or probe sets. A cluster analysis, based on the average Log Fold Change relative to cornea samples at Day 0 or the normal aortic ring samples at Day 0 for each experimental condition, are performed to group genes with similar expression profiles. Model-Based cluster analysis algorithm (“VII” model) gives 87 clusters. A total of 829 genes, or probe sets, in clusters with expression patterns consistent with the angiogenic process in both models are chosen for further analysis. The fold change of the expression values for the affected Affymetrix probe sets are detailed in Tables 1-3.

TABLE 1
Time course of gene expression in cornea only tissue

| | Affymetrix | Fold Change | NLogP | Fold Change | NLogP | Fold Change | NLogP |
|---|---|---|---|---|---|---|---|
| No. | Probe Set ID | Day 4 | Day 4 | Day 7 | Day 7 | Day 15 | Day 15 |
| 1 | 1367453_at | 1.32 | 6.2 | 1.26 | 4.5 | 1.09 | 0.9 |
| 2 | 1367463_at | 1.26 | 5.5 | 1.22 | 4.6 | −1.00 | 0 |
| 3 | 1367538_at | −1.16 | 1.8 | 1.15 | 1.5 | 1.25 | 3.2 |
| 4 | 1367574_at | 10.57 | 30.8 | 13.43 | 33.2 | 4.88 | 21.6 |
| 5 | 1367575_at | 1.79 | 9 | 1.44 | 4.5 | −1.08 | 0.4 |
| 6 | 1367579_s_at | 1.31 | 5 | 1.19 | 2.6 | −1.05 | 0.4 |
| 7 | 1367581_s_at | 61.77 | 18.9 | 48.38 | 18.7 | 12.52 | 11.2 |
| 8 | 1367590_at | 1.54 | 10 | 1.33 | 5.4 | −1.05 | 0.4 |
| 9 | 1367594_at | 1.98 | 8.8 | 3.56 | 19 | 2.87 | 15.4 |
| 10 | 1367619_at | 1.07 | 0.4 | 1.44 | 4.9 | 1.26 | 2.3 |
| 11 | 1367627_at | 16.14 | 14.3 | 20.94 | 15.9 | 6.66 | 8.6 |
| 12 | 1367646_at | 1.46 | 10.1 | 1.53 | 11.8 | 1.21 | 3.6 |
| 13 | 1367652_at | 11.29 | 15.9 | 19.83 | 20.1 | 6.00 | 10.9 |
| 14 | 1367658_at | 1.02 | 0.1 | 1.25 | 1.4 | −1.11 | 0.5 |
| 15 | 1367661_at | 10.70 | 24 | 6.93 | 19.6 | 1.84 | 4.2 |
| 16 | 1367663_at | 1.23 | 3.2 | 1.10 | 0.9 | 1.09 | 0.8 |
| 17 | 1367671_at | 1.09 | 0.5 | 1.06 | 0.3 | −1.02 | 0.1 |
| 18 | 1367682_at | 2.21 | 9.4 | 2.98 | 14.3 | 2.74 | 13 |
| 19 | 1367693_at | 1.95 | 13.4 | 1.93 | 13.1 | 1.26 | 2.8 |
| 20 | 1367701_at | 4.15 | 16 | 6.87 | 22.1 | 2.16 | 7.1 |
| 21 | 1367716_at | −1.52 | 4.6 | −1.19 | 1.2 | 1.02 | 0.1 |
| 22 | 1367721_at | 1.41 | 6.3 | 1.07 | 0.6 | −1.02 | 0.2 |
| 23 | 1367722_at | 1.39 | 3.1 | 2.39 | 13 | 2.91 | 16.6 |
| 24 | 1367732_at | 1.26 | 4.2 | 1.14 | 1.7 | −1.13 | 1.5 |
| 25 | 1367733_at | 3.67 | 9.4 | 3.25 | 8.2 | 1.02 | 0 |
| 26 | 1367749_at | 1.41 | 3.4 | 1.61 | 5.6 | 1.76 | 7.2 |
| 27 | 1367760_at | 1.37 | 7 | 1.16 | 2.3 | 1.03 | 0.2 |
| 28 | 1367765_at | 4.23 | 21.7 | 6.25 | 27.1 | 3.37 | 18.1 |
| 29 | 1367774_at | 1.04 | 0.1 | 1.63 | 2.2 | 1.41 | 1.3 |
| 30 | 1367784_a_at | 13.50 | 26.1 | 14.21 | 26.5 | 3.78 | 12.6 |
| 31 | 1367786_at | 2.73 | 7.8 | 2.92 | 8.5 | 2.00 | 4.4 |
| 32 | 1367800_at | 1.55 | 4.2 | 1.91 | 7.5 | −1.08 | 0.3 |
| 33 | 1367801_at | 1.09 | 0.8 | 1.35 | 5.4 | 1.13 | 1.3 |
| 34 | 1367807_at | 1.01 | 0.1 | 1.21 | 2.3 | 1.43 | 6.2 |
| 35 | 1367814_at | 1.14 | 0.4 | 1.19 | 0.5 | 1.24 | 0.8 |
| 36 | 1367823_at | 1.65 | 6.6 | 2.29 | 13.2 | 2.49 | 14.8 |
| 37 | 1367846_at | 8.46 | 18 | 11.65 | 20.9 | 4.02 | 10.7 |
| 38 | 1367850_at | 36.87 | 28.5 | 30.53 | 27.2 | 8.63 | 17.2 |
| 39 | 1367859_at | 2.00 | 5.8 | 3.41 | 13 | 3.29 | 12.5 |
| 40 | 1367881_at | 1.42 | 6.2 | 1.48 | 7.2 | 1.23 | 2.7 |
| 41 | 1367902_at | 2.04 | 5.9 | 3.93 | 14.2 | 2.24 | 6.5 |
| 42 | 1367905_at | 6.90 | 19.3 | 7.24 | 19.8 | 2.17 | 5.9 |
| 43 | 1367914_at | 2.92 | 18.1 | 3.83 | 22.8 | 2.11 | 11.7 |
| 44 | 1367930_at | −2.31 | 6.2 | −3.00 | 9.1 | −2.03 | 4.8 |
| 45 | 1367940_at | −1.62 | 5.9 | −1.85 | 8.4 | −1.19 | 1.3 |
| 46 | 1367942_at | 3.18 | 13.5 | 2.82 | 11.7 | 1.74 | 4.8 |
| 47 | 1367948_a_at | 1.25 | 1 | 1.81 | 4.5 | 1.14 | 0.5 |
| 48 | 1367959_a_at | 2.38 | 1.9 | 12.03 | 9.1 | 5.24 | 5 |
| 49 | 1367973_at | 134.66 | 21.9 | 99.03 | 20.5 | 15.74 | 11.2 |
| 50 | 1367974_at | 1.80 | 9.6 | 1.49 | 5.4 | 1.32 | 3.2 |
| 51 | 1367998_at | 4.50 | 13.6 | 2.70 | 7.8 | −1.65 | 2.7 |
| 52 | 1368000_at | 4.49 | 10.8 | 2.65 | 5.9 | −1.05 | 0.1 |
| 53 | 1368049_at | 1.14 | 0.9 | 1.23 | 1.6 | 1.18 | 1.2 |
| 54 | 1368052_at | 1.75 | 4.9 | 1.25 | 1.2 | 1.13 | 0.5 |
| 55 | 1368057_at | −1.74 | 7.3 | −1.45 | 4 | −1.11 | 0.6 |
| 56 | 1368078_at | 46.82 | 21.5 | 54.49 | 22.4 | 2.02 | 2.1 |
| 57 | 1368079_at | 1.60 | 3.2 | −1.02 | 0.1 | −1.20 | 0.8 |
| 58 | 1368089_at | 3.86 | 15 | 4.59 | 17.2 | 2.31 | 8 |
| 59 | 1368097_a_at | 2.99 | 7.2 | 3.21 | 7.9 | 1.46 | 1.4 |
| 60 | 1368103_at | −1.07 | 0.4 | −1.04 | 0.2 | 1.51 | 4.9 |
| 61 | 1368106_at | −1.11 | 0.5 | −1.27 | 1.9 | −1.42 | 3.4 |
| 62 | 1368145_at | 1.77 | 1.1 | 1.04 | 0 | 1.22 | 0.3 |
| 63 | 1368173_at | 1.25 | 2.6 | 1.26 | 2.7 | 1.07 | 0.4 |
| 64 | 1368183_at | 1.34 | 2.9 | 1.45 | 4.2 | 1.42 | 3.8 |
| 65 | 1368200_at | −1.18 | 0.6 | 1.01 | 0 | 1.27 | 1.1 |
| 66 | 1368221_at | −1.52 | 5.5 | −1.35 | 3.4 | 1.13 | 0.9 |
| 67 | 1368223_at | −2.20 | 5.8 | −1.36 | 1.4 | −1.24 | 0.8 |
| 68 | 1368259_at | 3.82 | 17.3 | 2.37 | 10 | 1.59 | 4 |
| 69 | 1368280_at | 3.64 | 13.7 | 4.46 | 16.3 | 1.82 | 4.6 |
| 70 | 1368281_at | 1.41 | 2 | 1.51 | 2.7 | 1.95 | 5.7 |
| 71 | 1368322_at | 1.55 | 2.8 | 1.78 | 4.2 | 1.80 | 4.4 |
| 72 | 1368332_at | 13.64 | 24 | 9.46 | 20.7 | 2.44 | 6.4 |
| 73 | 1368347_at | 24.06 | 11.7 | 97.46 | 18.1 | 17.87 | 10.2 |
| 74 | 1368370_at | 3.29 | 12.6 | 4.20 | 15.8 | 2.14 | 6.8 |
| 75 | 1368393_at | 38.80 | 27.3 | 69.83 | 30.8 | 10.58 | 17.8 |
| 76 | 1368395_at | −2.66 | 10.1 | −1.70 | 4.1 | 1.07 | 0.2 |
| 77 | 1368404_at | 2.42 | 9.4 | 4.38 | 17.8 | 1.87 | 5.7 |
| 78 | 1368419_at | 2.89 | 6.2 | 5.16 | 11.5 | 4.04 | 9.3 |
| 79 | 1368420_at | 2.56 | 5.1 | 3.62 | 8 | 2.40 | 4.5 |
| 80 | 1368430_at | 5.97 | 23.2 | 7.03 | 25.2 | 2.34 | 9.7 |
| 81 | 1368448_at | 2.33 | 2.6 | 2.53 | 3 | −1.07 | 0.1 |
| 82 | 1368464_at | 38.35 | 26.8 | 61.16 | 29.6 | 17.09 | 21.1 |
| 83 | 1368474_at | 9.83 | 25.5 | 11.19 | 26.8 | 8.97 | 24.5 |
| 84 | 1368519_at | 31.62 | 15.6 | 35.15 | 16.1 | 6.91 | 7.2 |
| 85 | 1368612_at | 1.09 | 0.6 | −1.53 | 7.3 | −1.58 | 8.1 |
| 86 | 1368655_at | 18.35 | 23.4 | 10.64 | 19 | 1.88 | 3 |
| 87 | 1368657_at | 33.14 | 26.6 | 20.80 | 23.3 | 4.21 | 9.7 |
| 88 | 1368731_at | −1.16 | 0.4 | −1.13 | 0.3 | −1.19 | 0.5 |
| 89 | 1368771_at | 1.83 | 3.6 | 2.73 | 7.8 | 2.01 | 4.5 |
| 90 | 1368813_at | 8.57 | 17.1 | 11.29 | 19.5 | 3.27 | 7.9 |
| 91 | 1368851_at | 14.82 | 27.5 | 18.69 | 29.5 | 7.44 | 20.8 |
| 92 | 1368885_at | 2.06 | 8.2 | 2.60 | 12 | 1.37 | 2.3 |
| 93 | 1368989_at | −2.14 | 2.4 | −1.14 | 0.2 | 1.20 | 0.3 |
| 94 | 1369006_at | 1.31 | 1.7 | 1.06 | 0.2 | −1.04 | 0.1 |
| 95 | 1369044_a_at | 3.84 | 10 | 3.52 | 9.2 | 1.76 | 2.8 |
| 96 | 1369065_a_at | 1.51 | 6.2 | 1.46 | 5.9 | 1.60 | 8.1 |
| 97 | 1369087_at | 1.74 | 5 | 1.90 | 6.2 | 1.33 | 1.8 |
| 98 | 1369166_at | 26.44 | 11.2 | 12.49 | 7.8 | 2.27 | 1.4 |
| 99 | 1369269_at | 1.18 | 0.9 | 1.62 | 4.5 | 1.24 | 1.3 |
| 100 | 1369294_at | 1.17 | 0.2 | 1.78 | 1.1 | −2.67 | 2.5 |
| 101 | 1369313_at | 27.47 | 15.9 | 34.77 | 18.1 | 9.96 | 10.7 |
| 102 | 1369422_at | 1.36 | 3.2 | 2.33 | 13.7 | 1.79 | 8.3 |
| 103 | 1369425_at | 2.55 | 6.6 | 2.81 | 7.6 | 1.54 | 2 |
| 104 | 1369484_at | 2.17 | 4.1 | 3.52 | 8.5 | 2.63 | 5.8 |
| 105 | 1369621_s_at | 3.57 | 16 | 4.64 | 19.7 | 1.81 | 5.7 |
| 106 | 1369633_at | 1.16 | 0.7 | 1.49 | 3.3 | 1.36 | 2.2 |
| 107 | 1369651_at | 3.43 | 16.5 | 7.35 | 26.8 | 2.36 | 10.5 |
| 108 | 1369652_at | 6.87 | 14.7 | 14.84 | 21.3 | 4.11 | 9.9 |
| 109 | 1369703_at | 1.43 | 3.1 | 1.76 | 6.2 | 1.40 | 2.8 |
| 110 | 1369735_at | −1.25 | 1.7 | 1.21 | 1.4 | 1.43 | 3.6 |
| 111 | 1369815_at | 20.70 | 13.2 | 4.87 | 5.3 | 1.56 | 0.8 |
| 112 | 1369895_s_at | 3.86 | 8.5 | 5.47 | 11.6 | 2.48 | 4.7 |
| 113 | 1369897_s_at | 1.60 | 14.8 | 1.55 | 13.5 | 1.24 | 5.1 |
| 114 | 1369926_at | 2.90 | 10.7 | 5.42 | 18.8 | 3.77 | 14.2 |
| 115 | 1369931_at | 1.76 | 10.9 | 1.19 | 1.8 | −1.17 | 1.6 |
| 116 | 1369947_at | 3.22 | 13.8 | 4.64 | 18.9 | 4.38 | 18.1 |
| 117 | 1369950_at | 1.39 | 5.3 | 1.52 | 7.7 | 1.15 | 1.5 |
| 118 | 1369953_a_at | 1.10 | 0.9 | −1.26 | 3.5 | −1.19 | 2.2 |
| 119 | 1369955_at | −2.03 | 11.5 | 1.26 | 2.2 | 1.92 | 10.3 |
| 120 | 1369956_at | 3.94 | 27.3 | 3.32 | 24.2 | 2.11 | 14.6 |
| 121 | 1369958_at | 1.43 | 4 | 1.86 | 9.2 | 1.10 | 0.6 |
| 122 | 1369960_at | 1.16 | 0.5 | 1.09 | 0.2 | 1.27 | 1 |
| 123 | 1369961_at | 2.97 | 18.3 | 4.04 | 23.6 | 2.23 | 12.8 |
| 124 | 1369973_at | 9.63 | 24.5 | 9.37 | 24.3 | 3.17 | 11.6 |
| 125 | 1370007_at | 1.46 | 3.8 | 2.16 | 10.8 | 2.12 | 10.5 |
| 126 | 1370023_at | 2.75 | 15.7 | 4.70 | 24.5 | 1.45 | 3.9 |
| 127 | 1370048_at | −2.36 | 11.2 | −1.98 | 8.2 | −1.00 | 0 |
| 128 | 1370051_at | 2.04 | 5.4 | 1.15 | 0.5 | −1.08 | 0.2 |
| 129 | 1370057_at | 2.59 | 17 | 3.27 | 21.6 | 1.71 | 8.2 |
| 130 | 1370062_at | 1.18 | 1.2 | −1.29 | 2.2 | −1.27 | 2 |
| 131 | 1370073_at | 1.15 | 0.9 | 1.27 | 2.1 | 1.37 | 3.2 |
| 132 | 1370097_a_at | 6.47 | 17.5 | 8.85 | 20.7 | 2.77 | 8.1 |
| 133 | 1370172_at | 3.52 | 11 | 2.60 | 7.5 | 1.36 | 1.3 |
| 134 | 1370184_at | 6.16 | 26.8 | 7.42 | 29.1 | 2.10 | 9.8 |
| 135 | 1370221_at | 1.48 | 2.8 | 2.75 | 11.3 | 1.93 | 6.1 |
| 136 | 1370244_at | 1.48 | 8.3 | 1.23 | 3.2 | 1.14 | 1.6 |
| 137 | 1370248_at | 1.34 | 1.8 | 1.58 | 3.7 | 1.41 | 2.4 |
| 138 | 1370256_at | 1.83 | 8.8 | 2.44 | 14.7 | 1.51 | 5 |
| 139 | 1370290_at | 1.35 | 5.6 | 1.59 | 10.8 | 1.18 | 2.2 |
| 140 | 1370301_at | 2.29 | 11.8 | 3.63 | 19.8 | 3.46 | 19 |
| 141 | 1370309_a_at | 1.10 | 0.7 | 1.04 | 0.2 | −1.02 | 0.1 |
| 142 | 1370312_at | 1.51 | 4.1 | 2.08 | 9.6 | 1.79 | 6.9 |
| 143 | 1370328_at | −3.26 | 10.1 | −2.06 | 4.9 | −1.14 | 0.4 |
| 144 | 1370408_at | 1.57 | 5 | 1.58 | 5.1 | −1.08 | 0.4 |
| 145 | 1370442_at | 1.53 | 1 | 4.06 | 5.9 | 1.42 | 0.7 |
| 146 | 1370570_at | 2.29 | 10.8 | 3.02 | 15.5 | 1.51 | 3.8 |
| 147 | 1370613_s_at | 14.09 | 15.7 | 10.54 | 13.7 | 3.29 | 5.3 |
| 148 | 1370624_at | 2.07 | 3.9 | 1.72 | 2.5 | 1.13 | 0.3 |
| 149 | 1370633_at | 64.91 | 11 | 14.23 | 5.8 | 1.17 | 0.1 |
| 150 | 1370634_x_at | 18.47 | 9.6 | 4.81 | 3.8 | 1.46 | 0.5 |
| 151 | 1370642_s_at | 2.10 | 7.6 | 5.84 | 22 | 4.85 | 19.6 |
| 152 | 1370693_a_at | 1.04 | 0.1 | 1.44 | 2.3 | 1.05 | 0.2 |
| 153 | 1370802_at | −1.70 | 4.6 | −1.47 | 2.7 | 1.14 | 0.6 |
| 154 | 1370809_at | 1.18 | 1.1 | 1.00 | 0 | 1.05 | 0.2 |
| 155 | 1370828_at | 2.15 | 3.5 | 2.27 | 3.9 | −1.25 | 0.6 |
| 156 | 1370838_s_at | −1.35 | 5.3 | −1.30 | 4.3 | 1.01 | 0.1 |
| 157 | 1370845_at | 1.13 | 0.5 | −1.40 | 2.3 | −1.31 | 1.6 |
| 158 | 1370854_at | 1.91 | 3 | 2.23 | 4.3 | 1.81 | 2.6 |
| 159 | 1370855_at | 1.79 | 5.7 | 2.59 | 11.5 | 2.93 | 13.4 |
| 160 | 1370862_at | −1.30 | 3 | 1.07 | 0.4 | 1.39 | 4.2 |
| 161 | 1370892_at | 11.63 | 12.9 | 22.46 | 17.1 | 11.53 | 12.8 |
| 162 | 1370905_at | −1.27 | 2.9 | −1.26 | 2.7 | −1.16 | 1.4 |
| 163 | 1370927_at | 1.30 | 2.1 | 1.89 | 8.2 | 2.22 | 11.2 |
| 164 | 1370950_at | 2.84 | 17 | 2.94 | 17.6 | 2.09 | 11.1 |
| 165 | 1370954_at | 1.75 | 3.8 | 2.15 | 6.2 | 1.91 | 4.8 |
| 166 | 1370956_at | 1.18 | 2.8 | 1.14 | 2 | 1.38 | 7.8 |
| 167 | 1370963_at | 5.88 | 15.2 | 6.45 | 16.1 | 4.41 | 12.2 |
| 168 | 1371016_at | 3.63 | 8.7 | 3.68 | 8.8 | 2.25 | 4.4 |
| 169 | 1371037_at | 4.25 | 16.6 | 6.25 | 21.4 | 3.02 | 11.9 |
| 170 | 1371079_at | 21.26 | 16.6 | 17.05 | 15.2 | 5.65 | 7.9 |
| 171 | 1371166_at | 3.61 | 6.3 | 6.32 | 10.5 | 1.53 | 1.2 |
| 172 | 1371232_a_at | 17.14 | 9.5 | 48.31 | 14.2 | 5.60 | 4.5 |
| 173 | 1371246_at | 1.49 | 5 | 1.50 | 5.2 | 1.04 | 0.2 |
| 174 | 1371250_at | 21.96 | 27.5 | 20.00 | 26.8 | 6.26 | 16.3 |
| 175 | 1371320_at | −1.13 | 2 | −1.17 | 3.1 | 1.07 | 0.8 |
| 176 | 1371329_at | 1.51 | 5.6 | 1.22 | 1.8 | 1.00 | 0 |
| 177 | 1371341_at | 1.20 | 1.5 | 1.15 | 1 | 1.09 | 0.5 |
| 178 | 1371349_at | −2.14 | 12.1 | 1.12 | 0.7 | 1.36 | 3.2 |
| 179 | 1371356_at | 1.02 | 0.1 | 1.08 | 0.3 | 1.20 | 1.1 |
| 180 | 1371360_at | 2.98 | 14 | 1.31 | 1.8 | 1.05 | 0.2 |
| 181 | 1371369_at | −2.05 | 10.3 | 1.30 | 2.2 | 1.54 | 4.9 |
| 182 | 1371392_at | 1.47 | 6.2 | 1.38 | 4.7 | 1.17 | 1.6 |
| 183 | 1371440_at | 3.93 | 17.4 | 4.43 | 19.1 | 2.95 | 13.2 |
| 184 | 1371447_at | −2.80 | 9.4 | −2.30 | 6.9 | −1.75 | 3.8 |
| 185 | 1371487_at | 1.31 | 4.4 | 1.29 | 4.1 | 1.07 | 0.6 |
| 186 | 1371498_at | 1.29 | 4.4 | 1.15 | 1.7 | 1.11 | 1.1 |
| 187 | 1371507_at | −1.29 | 3.6 | −1.12 | 1 | 1.18 | 1.9 |
| 188 | 1371525_at | 2.30 | 15.8 | 2.60 | 18.5 | 1.61 | 7.5 |
| 189 | 1371541_at | 1.31 | 1.1 | 1.99 | 4.5 | 2.00 | 4.5 |
| 190 | 1371545_at | 5.98 | 11.4 | 9.58 | 15.2 | 2.47 | 4.2 |
| 191 | 1371572_at | −1.13 | 1.5 | −1.04 | 0.3 | 1.11 | 1.1 |
| 192 | 1371575_at | 19.60 | 36.7 | 23.61 | 38.3 | 6.64 | 24.6 |
| 193 | 1371596_at | −1.03 | 0.2 | −1.09 | 0.8 | −1.13 | 1.4 |
| 194 | 1371600_at | 1.34 | 2.5 | 2.03 | 9.4 | 1.35 | 2.6 |
| 195 | 1371602_at | 1.22 | 2 | 1.08 | 0.5 | 1.02 | 0.1 |
| 196 | 1371618_s_at | 4.09 | 6.3 | 5.10 | 8.4 | 3.42 | 5.5 |
| 197 | 1371625_at | −1.97 | 8.4 | −1.85 | 7.3 | −1.12 | 0.6 |
| 198 | 1371632_at | −1.14 | 2 | −1.35 | 6.8 | −1.28 | 5 |
| 199 | 1371641_at | 1.09 | 0.8 | 1.11 | 0.9 | −1.07 | 0.5 |
| 200 | 1371691_at | 24.31 | 30.8 | 25.08 | 31.1 | 8.47 | 21.5 |
| 201 | 1371694_at | 1.35 | 3.2 | 2.03 | 11.5 | 1.28 | 2.4 |
| 202 | 1371727_at | 1.25 | 2.7 | 1.10 | 0.8 | −1.01 | 0.1 |
| 203 | 1371790_at | 1.11 | 0.8 | 1.13 | 0.9 | 1.02 | 0.1 |
| 204 | 1371824_at | 1.23 | 0.9 | 1.06 | 0.2 | 1.14 | 0.5 |
| 205 | 1371840_at | 11.38 | 14.3 | 20.20 | 18.3 | 4.88 | 8.1 |
| 206 | 1371849_at | 1.05 | 0.2 | 1.37 | 2 | 1.40 | 2.3 |
| 207 | 1371894_at | 1.15 | 1.8 | 1.47 | 8.3 | 1.22 | 3.1 |
| 208 | 1371924_at | −1.05 | 0.2 | 2.03 | 10.6 | 2.07 | 11 |
| 209 | 1371926_at | 1.27 | 4.2 | 1.35 | 6 | 1.15 | 1.8 |
| 210 | 1371951_at | 9.24 | 14.2 | 11.62 | 16.9 | 4.57 | 9.3 |
| 211 | 1371986_at | −2.16 | 14.6 | −1.63 | 8 | −1.23 | 2.2 |
| 212 | 1371988_at | 2.49 | 9.7 | 2.99 | 12.3 | 1.62 | 3.8 |
| 213 | 1371999_at | −1.22 | 2.8 | −1.34 | 5.1 | 1.02 | 0.1 |
| 214 | 1372005_at | 1.53 | 2.6 | 1.84 | 4.6 | 1.39 | 1.8 |
| 215 | 1372006_at | 2.66 | 12.4 | 6.33 | 24.8 | 3.89 | 17.4 |
| 216 | 1372028_at | 1.00 | 0 | 1.11 | 1 | 1.17 | 1.8 |
| 217 | 1372031_at | 3.61 | 14.5 | 5.62 | 20.2 | 2.47 | 9.2 |
| 218 | 1372050_at | 1.57 | 4.5 | 2.09 | 9.2 | 1.50 | 3.8 |
| 219 | 1372068_at | −1.52 | 7 | −1.25 | 2.6 | 1.06 | 0.4 |
| 220 | 1372084_at | 15.41 | 24.5 | 28.13 | 29.2 | 11.66 | 22.1 |
| 221 | 1372084_at | 15.41 | 24.5 | 28.13 | 29.2 | 11.66 | 22.1 |
| 222 | 1372088_at | −1.13 | 0.8 | −1.01 | 0.1 | 1.32 | 3 |
| 223 | 1372101_at | 2.34 | 11.9 | 2.71 | 14.5 | 2.09 | 9.9 |
| 224 | 1372104_at | 1.47 | 3.8 | 2.34 | 11.9 | 1.68 | 6 |
| 225 | 1372111_at | 2.77 | 18.2 | 3.13 | 20.5 | 1.45 | 4.7 |
| 226 | 1372127_at | 1.10 | 1.3 | 1.22 | 3.8 | −1.01 | 0.1 |
| 227 | 1372136_at | 1.52 | 16.3 | 1.44 | 13.7 | −1.17 | 4 |
| 228 | 1372164_at | 1.40 | 5.5 | 1.33 | 4.4 | 1.12 | 1.1 |
| 229 | 1372213_at | 1.83 | 5.8 | 2.56 | 10.9 | 1.17 | 0.8 |
| 230 | 1372223_at | −1.20 | 1.6 | −1.57 | 6.4 | −1.14 | 0.9 |
| 231 | 1372234_at | 1.68 | 4.9 | 2.93 | 13.7 | 2.21 | 9.1 |
| 232 | 1372251_at | −1.17 | 3.6 | −1.03 | 0.4 | 1.04 | 0.4 |
| 233 | 1372254_at | 7.71 | 26.2 | 9.47 | 28.4 | 6.70 | 24.5 |
| 234 | 1372256_at | 16.09 | 24.5 | 24.73 | 27.9 | 7.05 | 17.1 |
| 235 | 1372266_at | −1.74 | 4.6 | −1.59 | 3.5 | −1.03 | 0.1 |
| 236 | 1372294_at | 6.44 | 26.1 | 14.32 | 34.7 | 7.97 | 27.5 |
| 237 | 1372311_at | 1.26 | 3.4 | 1.28 | 3.9 | 1.12 | 1.2 |
| 238 | 1372326_at | 1.61 | 4.1 | 1.90 | 6.5 | 1.04 | 0.1 |
| 239 | 1372410_at | 1.45 | 2.9 | 1.98 | 7.5 | 1.73 | 5.4 |
| 240 | 1372439_at | 3.13 | 13.3 | 5.56 | 21 | 1.77 | 5 |
| 241 | 1372466_at | 1.88 | 13.4 | 2.40 | 19.5 | 1.97 | 14.6 |
| 242 | 1372518_at | 2.42 | 9.2 | 2.89 | 11.8 | 1.50 | 2.9 |
| 243 | 1372549_at | 1.48 | 4.9 | 1.35 | 3.3 | −1.11 | 0.7 |
| 244 | 1372569_at | 3.56 | 22.2 | 3.22 | 20.4 | 1.49 | 5 |
| 245 | 1372579_at | 12.69 | 20.8 | 16.14 | 22.8 | 6.32 | 14.6 |
| 246 | 1372585_at | 3.85 | 11.4 | 4.74 | 13.7 | 2.53 | 6.8 |
| 247 | 1372587_at | 6.85 | 22 | 10.99 | 27 | 3.61 | 14 |
| 248 | 1372593_at | −1.50 | 5.8 | −1.12 | 0.9 | 1.18 | 1.4 |
| 249 | 1372610_at | 1.08 | 0.5 | 1.25 | 2.1 | 1.55 | 5.9 |
| 250 | 1372613_at | −1.21 | 0.6 | 1.27 | 0.9 | 1.69 | 2.9 |
| 251 | 1372615_at | 1.07 | 0.2 | 2.35 | 7.6 | 3.24 | 11.9 |
| 252 | 1372638_at | 1.24 | 1.9 | 1.50 | 4.8 | 1.18 | 1.2 |
| 253 | 1372727_at | 2.30 | 8.7 | 2.70 | 11.2 | 1.71 | 4.5 |
| 254 | 1372729_at | 2.77 | 10.5 | 3.33 | 13 | 1.44 | 2.2 |
| 255 | 1372761_at | 10.31 | 19.5 | 12.27 | 21.1 | 5.36 | 13.4 |
| 256 | 1372769_at | 1.41 | 5.2 | 1.41 | 5.1 | 1.06 | 0.4 |
| 257 | 1372776_at | 1.18 | 1.7 | 1.09 | 0.6 | −1.03 | 0.1 |
| 258 | 1372818_at | 1.96 | 8.6 | 2.91 | 15.6 | 2.87 | 15.4 |
| 259 | 1372835_at | 2.19 | 6.9 | 3.27 | 12.3 | 1.47 | 2.3 |
| 260 | 1372836_at | 1.38 | 4.5 | 1.79 | 10.5 | 1.51 | 6.5 |
| 261 | 1372838_at | −1.26 | 3.3 | −1.20 | 2.2 | 1.06 | 0.5 |
| 262 | 1372844_at | 2.14 | 7.6 | 1.71 | 4.4 | 1.18 | 0.8 |
| 263 | 1372861_at | 1.25 | 2 | 1.50 | 5 | 1.21 | 1.6 |
| 264 | 1372919_at | 1.28 | 2.8 | 1.40 | 4.4 | 1.06 | 0.4 |
| 265 | 1372947_at | −1.41 | 6.3 | −1.34 | 5 | −1.11 | 1 |
| 266 | 1373000_at | 1.32 | 1.5 | 3.43 | 13.1 | 3.36 | 12.9 |
| 267 | 1373062_at | 1.43 | 1.7 | 2.56 | 7.7 | 1.73 | 3.4 |
| 268 | 1373079_at | −1.62 | 3.6 | 1.02 | 0.1 | 1.15 | 0.6 |
| 269 | 1373102_at | 2.34 | 8.4 | 2.50 | 9.4 | 1.25 | 1.1 |
| 270 | 1373140_at | 2.48 | 15.2 | 3.81 | 23 | 3.25 | 20.2 |
| 271 | 1373151_at | 1.24 | 1.3 | 2.19 | 9 | 1.52 | 3.5 |
| 272 | 1373180_at | 2.19 | 11.7 | 2.58 | 14.8 | 1.47 | 4.2 |
| 273 | 1373181_at | 2.08 | 8.1 | 2.41 | 10.6 | 1.12 | 0.5 |
| 274 | 1373217_at | −1.23 | 2.1 | −1.07 | 0.4 | 1.11 | 0.8 |
| 275 | 1373222_at | −1.02 | 0.1 | 1.41 | 3.9 | 1.65 | 6.9 |
| 276 | 1373245_at | 2.91 | 10.8 | 5.48 | 19 | 1.88 | 5.1 |
| 277 | 1373258_at | −1.37 | 2.4 | 1.12 | 0.5 | 1.23 | 1.3 |
| 278 | 1373286_at | 7.59 | 22.2 | 7.54 | 22.1 | 2.65 | 9.3 |
| 279 | 1373301_at | −1.39 | 4.8 | −1.27 | 2.9 | −1.14 | 1.2 |
| 280 | 1373310_at | −1.18 | 1 | −1.55 | 4.6 | 1.23 | 1.4 |
| 281 | 1373346_at | −1.11 | 1 | 1.07 | 0.5 | 1.12 | 1.1 |
| 282 | 1373363_at | 1.72 | 4.9 | 1.97 | 6.9 | 1.70 | 4.7 |
| 283 | 1373374_at | −1.75 | 10.3 | −1.77 | 10.6 | −1.13 | 1 |
| 284 | 1373379_at | −1.02 | 0.1 | 1.02 | 0.1 | −1.04 | 0.3 |
| 285 | 1373401_at | 37.43 | 20.8 | 110.59 | 26.6 | 15.46 | 15.3 |
| 286 | 1373415_at | 1.42 | 2.7 | 2.08 | 8.4 | 1.75 | 5.7 |
| 287 | 1373463_at | −1.44 | 3.9 | 1.62 | 6 | 1.74 | 7.3 |
| 288 | 1373466_at | −1.98 | 12.1 | −1.91 | 11.3 | −1.23 | 2.1 |
| 289 | 1373483_at | 2.81 | 9.1 | 3.87 | 13.1 | 1.81 | 4 |
| 290 | 1373487_at | 1.05 | 0.1 | 1.50 | 1.9 | 1.93 | 4 |
| 291 | 1373488_at | 1.43 | 6.4 | 1.28 | 3.8 | −1.02 | 0.1 |
| 292 | 1373515_at | −1.73 | 7.5 | −1.66 | 6.7 | −1.18 | 1.2 |
| 293 | 1373533_at | 2.07 | 7.3 | 3.00 | 12.9 | 1.86 | 5.8 |
| 294 | 1373554_at | 4.69 | 10.2 | 9.46 | 16.3 | 10.91 | 17.5 |
| 295 | 1373557_at | 1.23 | 1.5 | 1.18 | 1.1 | 1.06 | 0.3 |
| 296 | 1373590_at | 1.72 | 4.9 | 1.45 | 2.7 | 1.27 | 1.4 |
| 297 | 1373592_at | 2.89 | 12.2 | 3.19 | 13.6 | 1.12 | 0.4 |
| 298 | 1373599_at | 1.13 | 0.6 | 1.75 | 6.1 | 1.41 | 2.9 |
| 299 | 1373615_at | 2.00 | 5.1 | 2.20 | 6.2 | 1.25 | 0.9 |
| 300 | 1373628_at | −1.34 | 1.3 | 1.75 | 3.5 | 1.80 | 3.8 |
| 301 | 1373661_s_at | 27.10 | 25.1 | 35.85 | 27 | 10.31 | 17.7 |
| 302 | 1373666_at | 1.05 | 0.3 | 1.48 | 5.7 | 1.08 | 0.5 |
| 303 | 1373683_at | 2.70 | 11.6 | 3.74 | 16.4 | 1.75 | 5.1 |
| 304 | 1373695_at | −1.19 | 2.2 | −1.18 | 1.9 | −1.08 | 0.6 |
| 305 | 1373696_at | −1.16 | 0.3 | 1.34 | 0.8 | 1.56 | 1.4 |
| 306 | 1373740_at | −6.47 | 9.3 | −3.58 | 5.4 | −1.55 | 1 |
| 307 | 1373751_at | 3.22 | 16.1 | 3.28 | 16.4 | 1.71 | 5.6 |
| 308 | 1373829_at | −1.68 | 8.6 | −2.04 | 13.2 | −1.31 | 3.2 |
| 309 | 1373847_at | 3.31 | 14.3 | 5.23 | 20.5 | 2.64 | 10.9 |
| 310 | 1373874_at | −1.39 | 4.7 | −1.38 | 4.5 | −1.08 | 0.6 |
| 311 | 1373881_at | 1.65 | 13.8 | 1.92 | 18.7 | 1.68 | 14.5 |
| 312 | 1373911_at | 2.91 | 6.6 | 8.62 | 16.6 | 3.21 | 7.5 |
| 313 | 1373957_at | −2.08 | 4.3 | 1.32 | 1 | 1.68 | 2.5 |
| 314 | 1373970_at | 2.22 | 7.8 | 2.81 | 11.2 | 1.28 | 1.3 |
| 315 | 1374013_at | 1.64 | 5.7 | 3.62 | 19.9 | 3.88 | 21.1 |
| 316 | 1374034_at | 1.72 | 7 | 1.21 | 1.4 | −1.00 | 0 |
| 317 | 1374057_at | 2.24 | 7.6 | 2.62 | 9.8 | 1.57 | 3.1 |
| 318 | 1374061_at | 2.91 | 12.5 | 3.50 | 15.2 | 2.27 | 8.7 |
| 319 | 1374104_at | 2.62 | 6.4 | 6.98 | 16.4 | 2.78 | 7 |
| 320 | 1374118_at | 1.04 | 0.3 | −1.05 | 0.4 | −1.01 | 0 |
| 321 | 1374129_at | 1.51 | 4.5 | 2.05 | 10 | −1.09 | 0.5 |
| 322 | 1374134_at | 1.67 | 6.8 | 2.13 | 11.6 | 1.59 | 5.8 |
| 323 | 1374171_at | 5.17 | 6.8 | 8.53 | 10 | 4.66 | 6.2 |
| 324 | 1374172_at | −1.90 | 6.4 | 1.14 | 0.6 | 1.99 | 7.1 |
| 325 | 1374176_at | 2.71 | 11 | 3.88 | 16.1 | 2.49 | 9.7 |
| 326 | 1374207_at | 3.83 | 12.2 | 7.25 | 19.2 | 2.51 | 7.2 |
| 327 | 1374247_at | 20.43 | 25.9 | 29.01 | 28.5 | 11.65 | 21.4 |
| 328 | 1374266_at | −1.64 | 5.5 | −2.28 | 11.5 | −1.18 | 1.1 |
| 329 | 1374273_at | −1.74 | 8.9 | −1.80 | 9.5 | −1.13 | 0.9 |
| 330 | 1374276_at | 1.20 | 0.7 | 1.37 | 1.7 | 1.06 | 0.2 |
| 331 | 1374284_at | 2.06 | 6 | 1.70 | 3.8 | 1.60 | 3.1 |
| 332 | 1374285_at | −1.78 | 11 | −1.54 | 7.4 | −1.01 | 0.1 |
| 333 | 1374306_at | 1.20 | 1.8 | 1.12 | 1 | −1.06 | 0.4 |
| 334 | 1374320_at | 2.90 | 8.1 | 1.89 | 3.7 | 1.14 | 0.4 |
| 335 | 1374399_at | 1.72 | 5.1 | 1.85 | 6.1 | 1.09 | 0.4 |
| 336 | 1374474_at | 1.02 | 0.1 | 1.02 | 0.1 | −1.04 | 0.3 |
| 337 | 1374477_at | 2.32 | 9.1 | 3.59 | 15.6 | 2.02 | 7 |
| 338 | 1374479_at | −1.63 | 7.5 | −1.64 | 7.6 | −1.22 | 1.8 |
| 339 | 1374529_at | 2.67 | 11.4 | 4.15 | 17.9 | 3.87 | 16.9 |
| 340 | 1374531_at | 1.31 | 1.5 | 1.33 | 1.6 | 1.53 | 2.9 |
| 341 | 1374575_at | 1.09 | 0.3 | 1.14 | 0.5 | 1.10 | 0.3 |
| 342 | 1374586_at | −2.09 | 9.7 | −1.67 | 5.8 | −1.34 | 2.4 |
| 343 | 1374626_at | 20.39 | 20.2 | 15.39 | 18.2 | 4.12 | 7.9 |
| 344 | 1374635_at | 3.72 | 3.6 | 3.37 | 3.2 | 3.95 | 3.8 |
| 345 | 1374678_at | −1.52 | 3.9 | −2.35 | 11.2 | −1.51 | 3.9 |
| 346 | 1374742_at | −1.28 | 2.1 | −1.11 | 0.6 | −1.18 | 1.2 |
| 347 | 1374771_at | 1.66 | 5.3 | 2.02 | 8.7 | 1.33 | 2.2 |
| 348 | 1374779_at | 1.50 | 1.3 | 1.54 | 1.4 | −1.00 | 0 |
| 349 | 1374816_at | 1.29 | 2.6 | 1.28 | 2.6 | 1.03 | 0.2 |
| 350 | 1374849_at | 2.85 | 8 | 4.10 | 12 | 2.90 | 8.2 |
| 351 | 1374868_at | −2.09 | 7.4 | −1.53 | 3.2 | 1.33 | 1.8 |
| 352 | 1374897_at | 1.19 | 1.5 | 1.02 | 0.1 | −1.04 | 0.2 |
| 353 | 1374953_at | 9.52 | 7.1 | 12.91 | 8.6 | 3.09 | 2.5 |
| 354 | 1375028_at | 1.67 | 7.8 | −1.05 | 0.3 | 1.12 | 0.8 |
| 355 | 1375051_at | 1.75 | 5.4 | 1.59 | 4.1 | 1.10 | 0.4 |
| 356 | 1375074_at | 1.95 | 8.3 | 2.16 | 10 | 1.89 | 7.7 |
| 357 | 1375138_at | −1.29 | 1 | 1.27 | 0.9 | 1.70 | 3.2 |
| 358 | 1375144_at | 1.73 | 6.1 | 2.86 | 14.8 | 3.31 | 17.2 |
| 359 | 1375267_at | 2.57 | 10.8 | 4.81 | 19.7 | 2.80 | 12.1 |
| 360 | 1375270_at | −1.62 | 7.7 | −1.60 | 7.5 | −1.11 | 0.8 |
| 361 | 1375277_at | 1.49 | 4.4 | 1.13 | 0.7 | 1.17 | 1.1 |
| 362 | 1375337_at | 2.48 | 11.3 | 3.76 | 17.9 | 1.79 | 6 |
| 363 | 1375368_at | 1.07 | 0.5 | 1.02 | 0.1 | 1.09 | 0.6 |
| 364 | 1375369_at | 1.53 | 7.2 | 1.35 | 4.3 | 1.25 | 2.7 |
| 365 | 1375377_at | 1.12 | 0.6 | 1.06 | 0.3 | −1.05 | 0.2 |
| 366 | 1375420_at | 26.19 | 30.7 | 26.42 | 30.8 | 6.20 | 17.7 |
| 367 | 1375424_at | 1.37 | 5.1 | 1.57 | 8.6 | 1.07 | 0.5 |
| 368 | 1375463_at | 1.02 | 0.1 | 1.23 | 2.7 | 1.27 | 3.4 |
| 369 | 1375473_at | 2.04 | 8 | 2.81 | 13.3 | 1.51 | 3.6 |
| 370 | 1375657_at | 1.74 | 7 | 1.62 | 5.7 | 1.44 | 3.7 |
| 371 | 1375719_s_at | 9.03 | 9.1 | 12.98 | 11.2 | 4.57 | 5.3 |
| 372 | 1375721_at | 1.02 | 0.1 | 1.01 | 0 | 1.31 | 2.4 |
| 373 | 1375739_at | 1.92 | 14.1 | 2.01 | 15.1 | 1.14 | 1.3 |
| 374 | 1375862_at | 4.80 | 15.9 | 10.26 | 24 | 4.82 | 15.9 |
| 375 | 1375898_at | 2.39 | 12.8 | 2.40 | 12.9 | 1.46 | 3.9 |
| 376 | 1375951_at | −1.35 | 1.4 | 1.42 | 1.8 | −1.32 | 1.3 |
| 377 | 1376004_at | −1.22 | 2.3 | −1.16 | 1.6 | 1.22 | 2.4 |
| 378 | 1376045_at | 2.85 | 14.1 | 4.88 | 22.2 | 2.50 | 11.9 |
| 379 | 1376055_at | 1.13 | 0.8 | 1.17 | 1.1 | −1.04 | 0.2 |
| 380 | 1376099_at | −2.25 | 9.8 | 1.02 | 0.1 | 1.79 | 6.1 |
| 381 | 1376106_at | −1.40 | 1.5 | 1.13 | 0.4 | 1.04 | 0.1 |
| 382 | 1376128_at | −1.45 | 4.4 | −1.45 | 4.4 | 1.18 | 1.3 |
| 383 | 1376182_at | −1.34 | 3.3 | −1.06 | 0.3 | 1.19 | 1.4 |
| 384 | 1376265_at | 2.39 | 10.5 | 2.80 | 13.1 | 1.62 | 4.4 |
| 385 | 1376570_at | 1.36 | 5.5 | 1.29 | 4.2 | 1.18 | 2.2 |
| 386 | 1376574_at | 14.29 | 27.8 | 17.91 | 29.7 | 4.77 | 15.5 |
| 387 | 1376583_at | 1.33 | 5.3 | 1.21 | 2.9 | 1.11 | 1.1 |
| 388 | 1376624_at | 3.31 | 13.2 | 3.31 | 13.2 | 1.54 | 3 |
| 389 | 1376631_at | 1.79 | 8.2 | 1.56 | 5.5 | 1.35 | 2.9 |
| 390 | 1376640_at | 2.47 | 10.3 | 3.57 | 15.9 | 1.29 | 1.6 |
| 391 | 1376655_at | −1.15 | 1.2 | −1.17 | 1.3 | 1.01 | 0.1 |
| 392 | 1376678_at | 2.14 | 3.7 | 3.20 | 7 | 1.63 | 1.8 |
| 393 | 1376754_at | 1.43 | 5.9 | 1.11 | 0.9 | 1.02 | 0.1 |
| 394 | 1376775_at | 2.81 | 9.4 | 2.95 | 10 | 1.72 | 3.6 |
| 395 | 1376786_a_at | −1.56 | 4.1 | −1.39 | 2.5 | 1.08 | 0.3 |
| 396 | 1376788_at | −2.07 | 10.9 | −1.85 | 8.7 | −1.32 | 2.5 |
| 397 | 1376848_at | 1.41 | 5.8 | 1.86 | 13.1 | 1.15 | 1.4 |
| 398 | 1376858_at | −1.32 | 4 | −1.09 | 0.7 | 1.26 | 3 |
| 399 | 1376919_at | −1.50 | 7.4 | −1.64 | 9.7 | −1.26 | 3.1 |
| 400 | 1377023_at | 5.56 | 5.3 | 4.20 | 4 | 1.69 | 0.9 |
| 401 | 1377369_at | 1.06 | 0.2 | 1.33 | 2 | 1.25 | 1.4 |
| 402 | 1377390_at | −2.13 | 10.1 | −2.64 | 13.9 | −1.51 | 4.1 |
| 403 | 1377595_at | −1.83 | 4.9 | −2.11 | 6.8 | −1.69 | 4 |
| 404 | 1377625_at | 1.34 | 1.8 | 1.06 | 0.2 | 1.12 | 0.5 |
| 405 | 1377630_at | 3.58 | 18.1 | 4.01 | 19.8 | 2.37 | 11.3 |
| 406 | 1377671_at | 6.43 | 16.5 | 5.12 | 14.1 | 1.55 | 2 |
| 407 | 1377697_at | 2.23 | 8.9 | 3.81 | 17.1 | 1.67 | 4.6 |
| 408 | 1377702_at | −1.46 | 5.6 | −1.58 | 7.4 | −1.62 | 8.1 |
| 409 | 1377723_at | −1.26 | 4 | −1.02 | 0.1 | 1.10 | 1.1 |
| 410 | 1377862_at | 2.54 | 4.5 | 3.78 | 7.7 | 2.17 | 3.4 |
| 411 | 1377869_at | 1.53 | 5.1 | 1.38 | 3.3 | 1.60 | 6 |
| 412 | 1377950_at | 2.29 | 2.5 | 5.00 | 6.9 | 2.33 | 2.6 |
| 413 | 1377994_at | 34.03 | 17.2 | 31.90 | 16.9 | 4.00 | 4.8 |
| 414 | 1378134_at | −1.83 | 9.8 | −1.57 | 6.5 | −1.18 | 1.4 |
| 415 | 1378282_at | 1.15 | 1.1 | 1.11 | 0.8 | −1.11 | 0.8 |
| 416 | 1378305_at | 2.16 | 3.4 | 3.40 | 6.8 | 2.26 | 3.7 |
| 417 | 1378321_at | 1.97 | 4.9 | 1.55 | 2.5 | 1.56 | 2.5 |
| 418 | 1378342_at | 3.12 | 5.7 | 5.43 | 10.1 | 2.28 | 3.5 |
| 419 | 1378440_at | 2.18 | 8 | 2.09 | 7.4 | 1.36 | 1.9 |
| 420 | 1378507_at | 1.17 | 0.9 | 1.20 | 1.1 | 1.17 | 0.9 |
| 421 | 1378526_at | 1.53 | 2.9 | 1.69 | 4 | 1.39 | 1.9 |
| 422 | 1378925_at | 4.01 | 6.8 | 3.30 | 5.5 | 1.98 | 2.3 |
| 423 | 1379294_at | 1.16 | 1.3 | 1.50 | 5.9 | 1.34 | 3.7 |
| 424 | 1379322_at | 1.33 | 4.7 | 1.33 | 4.7 | 1.02 | 0.1 |
| 425 | 1379331_at | −1.36 | 0.3 | 8.19 | 5.4 | 5.95 | 4.2 |
| 426 | 1379335_at | −1.39 | 7.7 | −1.37 | 7.3 | −1.11 | 1.3 |
| 427 | 1379345_at | 41.88 | 25.4 | 55.09 | 27.1 | 9.81 | 15.2 |
| 428 | 1379461_at | −1.35 | 2.7 | −1.22 | 1.4 | −1.11 | 0.6 |
| 429 | 1379497_at | 4.42 | 5.2 | 1.79 | 1.2 | −1.11 | 0.1 |
| 430 | 1379604_at | 2.08 | 9.4 | 1.94 | 8.1 | 1.29 | 1.9 |
| 431 | 1379772_at | 4.77 | 13.8 | 6.31 | 16.8 | 2.75 | 7.7 |
| 432 | 1379790_at | 2.12 | 7.2 | 2.46 | 9.2 | 1.90 | 5.7 |
| 433 | 1379799_at | 1.41 | 2.1 | 1.60 | 3.4 | 1.99 | 6.1 |
| 434 | 1379847_at | 5.26 | 12.6 | 7.21 | 15.6 | 3.93 | 9.8 |
| 435 | 1379882_a_at | 1.32 | 0.7 | 3.22 | 5.9 | 2.77 | 4.8 |
| 436 | 1379935_at | 18.94 | 10.9 | 19.23 | 10.9 | 2.75 | 2.2 |
| 437 | 1380089_at | −1.24 | 0.6 | 1.53 | 1.5 | 3.18 | 7.1 |
| 438 | 1380110_at | 3.37 | 12.6 | 3.86 | 14.3 | 1.55 | 2.8 |
| 439 | 1380318_at | 1.72 | 4.2 | 1.54 | 3 | 1.43 | 2.2 |
| 440 | 1380474_at | 3.44 | 5.9 | 6.29 | 10.5 | 3.26 | 5.6 |
| 441 | 1380617_at | 10.47 | 19.2 | 13.36 | 21.2 | 6.04 | 14.1 |
| 442 | 1380688_at | −2.53 | 10.1 | −2.30 | 8.7 | −1.33 | 1.7 |
| 443 | 1380728_at | 1.62 | 2.9 | 2.27 | 6.6 | 1.34 | 1.4 |
| 444 | 1380908_at | 1.87 | 4.3 | 1.96 | 4.8 | 1.73 | 3.5 |
| 445 | 1381190_at | −1.12 | 0.8 | −1.37 | 3.8 | −1.27 | 2.4 |
| 446 | 1381305_at | 1.32 | 3.2 | 2.25 | 15.1 | 2.13 | 13.9 |
| 447 | 1381311_at | 5.30 | 10.4 | 7.72 | 13.6 | 4.32 | 8.7 |
| 448 | 1381335_at | 1.43 | 1 | −1.33 | 0.8 | 1.06 | 0.1 |
| 449 | 1381410_a_at | 4.12 | 9.3 | 5.32 | 11.6 | 2.32 | 4.3 |
| 450 | 1381461_at | 1.29 | 1.9 | 1.37 | 2.7 | 1.09 | 0.4 |
| 451 | 1381470_at | −2.19 | 9.2 | −2.30 | 10.1 | −1.89 | 6.9 |
| 452 | 1381678_at | 4.88 | 16.7 | 3.93 | 14 | 1.62 | 3.1 |
| 453 | 1381915_at | −2.35 | 6.4 | −1.69 | 3 | −1.26 | 0.9 |
| 454 | 1381971_at | 22.26 | 23.6 | 31.94 | 26.1 | 7.97 | 15.4 |
| 455 | 1381996_at | 1.86 | 4.6 | 2.34 | 7.4 | 2.32 | 7.3 |
| 456 | 1382008_at | 1.41 | 8.9 | 1.17 | 2.7 | −1.12 | 1.6 |
| 457 | 1382017_at | 11.65 | 8 | 11.15 | 7.8 | 2.69 | 2 |
| 458 | 1382108_at | 1.58 | 6.6 | 1.37 | 3.7 | 1.16 | 1.2 |
| 459 | 1382130_at | 2.80 | 8.4 | 3.91 | 12.2 | 2.07 | 5 |
| 460 | 1382138_at | 1.10 | 0.5 | −1.26 | 2 | 1.05 | 0.2 |
| 461 | 1382181_at | 17.89 | 26.2 | 18.42 | 26.5 | 8.72 | 19.9 |
| 462 | 1382212_at | 1.71 | 3.2 | 3.33 | 10.5 | 2.03 | 4.9 |
| 463 | 1382296_at | 1.74 | 5 | 2.17 | 8.3 | 1.28 | 1.4 |
| 464 | 1382375_at | 1.03 | 0.2 | 1.09 | 0.6 | 1.06 | 0.4 |
| 465 | 1382404_at | 2.04 | 3.9 | 1.73 | 2.6 | 1.23 | 0.6 |
| 466 | 1382431_at | 1.19 | 0.4 | 1.30 | 0.7 | 2.54 | 5 |
| 467 | 1382482_at | 2.23 | 7.5 | 3.42 | 13.4 | 1.52 | 2.8 |
| 468 | 1382536_at | 1.47 | 4.9 | 1.74 | 8.6 | 1.54 | 5.9 |
| 469 | 1382571_at | 2.53 | 7.6 | 3.14 | 10.2 | 1.74 | 3.5 |
| 470 | 1382628_at | 1.15 | 1.1 | 1.29 | 2.6 | 1.36 | 3.5 |
| 471 | 1382680_at | 18.12 | 10.2 | 15.79 | 9.6 | 3.40 | 2.8 |
| 472 | 1382692_at | 10.24 | 20.6 | 7.21 | 17.3 | 2.51 | 6.3 |
| 473 | 1382818_at | −1.56 | 6.3 | −1.41 | 4.3 | 1.11 | 0.7 |
| 474 | 1382995_at | 2.59 | 15.7 | 3.30 | 20.2 | 2.12 | 11.7 |
| 475 | 1383019_at | 5.12 | 11.9 | 8.31 | 16.4 | 4.48 | 10.6 |
| 476 | 1383073_at | 1.27 | 5.1 | 1.41 | 8.5 | 1.15 | 2.2 |
| 477 | 1383080_at | 1.08 | 0.9 | 1.20 | 3.4 | 1.07 | 0.8 |
| 478 | 1383131_at | 5.61 | 15.9 | 4.73 | 14.1 | 2.05 | 4.7 |
| 479 | 1383147_at | −1.42 | 2.8 | 1.02 | 0.1 | 1.16 | 0.8 |
| 480 | 1383193_at | 1.49 | 4.1 | 2.05 | 9.8 | 1.83 | 7.7 |
| 481 | 1383201_at | −1.26 | 3.3 | −1.05 | 0.3 | 1.18 | 2 |
| 482 | 1383240_at | 1.50 | 6.2 | −1.00 | 0 | −1.40 | 4.7 |
| 483 | 1383247_a_at | −1.13 | 0.7 | −1.34 | 2.5 | −1.33 | 2.4 |
| 484 | 1383261_at | −1.56 | 4.4 | −1.35 | 2.4 | −1.11 | 0.5 |
| 485 | 1383328_x_at | −2.42 | 10.5 | −1.80 | 5.8 | −1.37 | 2.3 |
| 486 | 1383355_at | 1.63 | 5.6 | 2.06 | 9.9 | 1.94 | 8.8 |
| 487 | 1383382_at | −1.91 | 13.7 | −1.91 | 13.6 | −1.41 | 5.6 |
| 488 | 1383469_at | −1.18 | 1.1 | −1.78 | 7.4 | −1.49 | 4.2 |
| 489 | 1383484_at | 1.02 | 0.3 | 1.09 | 2 | 1.24 | 8.4 |
| 490 | 1383489_at | 2.00 | 8.6 | 2.66 | 13.7 | 2.40 | 11.8 |
| 491 | 1383498_at | 1.39 | 3.1 | 2.06 | 9.9 | 1.44 | 3.6 |
| 492 | 1383516_at | 18.56 | 23.4 | 18.80 | 23.5 | 5.98 | 13.7 |
| 493 | 1383533_at | −1.08 | 0.4 | 1.15 | 1 | 1.52 | 5.7 |
| 494 | 1383574_at | 2.55 | 12.8 | 2.88 | 14.9 | 1.58 | 4.6 |
| 495 | 1383641_at | 2.99 | 14.8 | 4.34 | 20.4 | 2.99 | 14 |
| 496 | 1383644_at | −8.65 | 5.1 | −7.06 | 4.4 | −1.70 | 0.6 |
| 497 | 1383662_at | 1.69 | 4.7 | 1.69 | 4.7 | 1.60 | 3.9 |
| 498 | 1383776_at | −1.60 | 8.3 | −1.49 | 6.5 | −1.13 | 1.1 |
| 499 | 1383785_at | 1.95 | 5.3 | 1.62 | 3.2 | 1.15 | 0.5 |
| 500 | 1383824_at | 2.76 | 6 | 4.14 | 9.8 | 3.04 | 6.9 |
| 501 | 1383844_at | 3.38 | 7.9 | 5.27 | 12.1 | 2.50 | 5.2 |
| 502 | 1383863_at | 1.76 | 6.4 | 2.09 | 9.4 | 1.22 | 1.3 |
| 503 | 1383914_at | 1.50 | 3.4 | 2.34 | 10.2 | 1.43 | 2.8 |
| 504 | 1383935_at | 3.35 | 6.1 | 3.30 | 6 | 2.89 | 5 |
| 505 | 1383949_at | 1.28 | 1.9 | 1.69 | 6 | 1.51 | 4.2 |
| 506 | 1384051_at | 1.34 | 1.4 | 3.07 | 10.8 | 1.85 | 4.5 |
| 507 | 1384165_at | 9.00 | 6 | 7.67 | 5.4 | 2.48 | 1.6 |
| 508 | 1384183_at | −2.56 | 7.5 | −2.62 | 7.7 | −1.03 | 0.1 |
| 509 | 1384188_at | −1.25 | 2.1 | 1.09 | 0.6 | 1.55 | 5.8 |
| 510 | 1384192_at | 2.42 | 4.6 | 3.35 | 7.5 | 1.76 | 2.3 |
| 511 | 1384227_at | 1.73 | 5.7 | 1.63 | 4.8 | 1.01 | 0 |
| 512 | 1384232_at | 1.76 | 4.3 | 2.19 | 7.1 | 1.09 | 0.3 |
| 513 | 1384302_at | −1.49 | 1.6 | −1.15 | 0.4 | 1.14 | 0.4 |
| 514 | 1384335_at | −1.19 | 2.6 | −1.10 | 1 | 1.20 | 2.9 |
| 515 | 1384381_at | 1.91 | 2.9 | 2.34 | 4.4 | 2.48 | 4.9 |
| 516 | 1384392_at | 2.47 | 8.2 | 1.54 | 2.7 | −1.32 | 1.3 |
| 517 | 1384415_at | 5.80 | 27.5 | 4.47 | 23.7 | 3.34 | 19.1 |
| 518 | 1384724_at | 2.26 | 10.4 | 2.28 | 10.5 | 1.66 | 5.2 |
| 519 | 1384899_at | −2.57 | 9.5 | −1.27 | 1.2 | −1.10 | 0.3 |
| 520 | 1384900_at | 2.06 | 7.4 | 1.66 | 4.4 | 1.11 | 0.4 |
| 521 | 1384907_at | 2.69 | 5.8 | 3.18 | 7.3 | 2.16 | 3.9 |
| 522 | 1385057_at | 1.42 | 4 | −1.23 | 1.8 | −1.07 | 0.4 |
| 523 | 1385359_at | 10.30 | 13.7 | 7.59 | 11.5 | 1.92 | 2.1 |
| 524 | 1385397_at | 1.57 | 4 | 1.00 | 0 | −1.18 | 0.9 |
| 525 | 1385444_at | −1.48 | 4.9 | −1.23 | 2 | 1.26 | 2.4 |
| 526 | 1385506_at | −2.30 | 10.7 | −1.86 | 7.1 | −1.18 | 1 |
| 527 | 1385925_at | 21.92 | 20.6 | 26.25 | 21.8 | 2.91 | 5.1 |
| 528 | 1385926_at | 3.44 | 13.2 | 4.30 | 16.2 | 1.45 | 2.3 |
| 529 | 1386041_a_at | 6.25 | 4.6 | 12.89 | 7.5 | 2.49 | 1.6 |
| 530 | 1386080_at | 1.19 | 0.7 | 1.86 | 4.9 | 1.33 | 1.5 |
| 531 | 1386540_at | −1.33 | 3.2 | −1.50 | 5.5 | −1.20 | 1.7 |
| 532 | 1386721_at | 1.20 | 0.9 | 2.03 | 6.8 | 2.40 | 9.3 |
| 533 | 1386833_at | 2.04 | 3.7 | 3.18 | 7.6 | 1.43 | 1.3 |
| 534 | 1386864_at | 1.79 | 12.3 | 1.24 | 2.9 | 1.06 | 0.4 |
| 535 | 1386866_at | 1.50 | 8.4 | 1.56 | 9.7 | 1.15 | 1.8 |
| 536 | 1386881_at | 9.89 | 14.6 | 16.59 | 18.5 | 5.06 | 9.3 |
| 537 | 1386899_at | 1.95 | 17.4 | 2.13 | 19.7 | 1.88 | 16.2 |
| 538 | 1386912_at | 1.13 | 0.6 | 2.04 | 9.3 | 2.17 | 10.4 |
| 539 | 1386913_at | 7.99 | 30.7 | 5.57 | 26.1 | 2.31 | 12 |
| 540 | 1386922_at | 4.65 | 14.8 | 4.18 | 13.5 | −1.04 | 0.1 |
| 541 | 1386937_at | 1.00 | 0 | 1.05 | 0.1 | 1.13 | 0.3 |
| 542 | 1386938_at | 3.84 | 25.1 | 4.03 | 25.9 | 2.54 | 17.3 |
| 543 | 1386940_at | 2.19 | 13.1 | 2.70 | 17.3 | 2.22 | 13.3 |
| 544 | 1386948_at | 1.97 | 6.4 | 2.06 | 7 | 1.40 | 2.2 |
| 545 | 1387001_at | 1.84 | 11.3 | 1.81 | 10.8 | 1.19 | 1.8 |
| 546 | 1387005_at | 4.00 | 14.9 | 6.96 | 21.6 | 3.63 | 13.6 |
| 547 | 1387018_at | 2.46 | 5.4 | 3.69 | 9.3 | 1.65 | 2.2 |
| 548 | 1387076_at | 1.20 | 1.4 | 1.19 | 1.3 | 1.14 | 0.9 |
| 549 | 1387125_at | 106.55 | 21.7 | 39.51 | 16.8 | 1.89 | 1.3 |
| 550 | 1387137_at | −1.07 | 0.1 | −1.25 | 0.5 | −1.13 | 0.2 |
| 551 | 1387153_at | 1.96 | 7.6 | 3.02 | 14.6 | 2.12 | 8.3 |
| 552 | 1387154_at | −21.24 | 20.5 | −31.34 | 23.1 | −2.64 | 4.5 |
| 553 | 1387160_at | 4.03 | 4.8 | 6.95 | 7.9 | 1.40 | 0.6 |
| 554 | 1387197_at | 1.11 | 0.3 | 1.49 | 2.4 | 1.78 | 4.3 |
| 555 | 1387202_at | 6.88 | 21.7 | 5.76 | 19.7 | 2.93 | 11.1 |
| 556 | 1387219_at | 1.91 | 3 | 1.88 | 2.9 | 1.23 | 0.6 |
| 557 | 1387259_at | 1.35 | 1.4 | 1.80 | 4 | 1.14 | 0.5 |
| 558 | 1387270_at | 3.25 | 9 | 3.39 | 9.5 | 1.77 | 3 |
| 559 | 1387273_at | 7.43 | 10.4 | 12.82 | 14.2 | 2.47 | 3.2 |
| 560 | 1387279_at | 1.02 | 0.1 | −1.20 | 1.8 | −1.31 | 3.2 |
| 561 | 1387282_at | 3.32 | 10.7 | 2.84 | 8.9 | 1.50 | 2.1 |
| 562 | 1387294_at | 5.41 | 22.1 | 7.88 | 26.6 | 3.77 | 17.1 |
| 563 | 1387306_a_at | 3.17 | 9.9 | 2.95 | 9 | 1.82 | 3.7 |
| 564 | 1387343_at | 5.07 | 11.6 | 5.38 | 12.2 | 2.14 | 3.8 |
| 565 | 1387344_at | −1.46 | 2.7 | −1.62 | 3.9 | −1.10 | 0.4 |
| 566 | 1387348_at | 1.53 | 2.2 | 1.74 | 3.3 | 1.64 | 2.7 |
| 567 | 1387389_at | 6.06 | 6.2 | 12.25 | 9.9 | 1.30 | 0.4 |
| 568 | 1387395_at | 1.59 | 5.1 | 1.58 | 5 | 1.28 | 1.9 |
| 569 | 1387402_at | 2.56 | 10.3 | 2.78 | 11.5 | 1.43 | 2.4 |
| 570 | 1387530_a_at | 1.29 | 1.9 | −1.03 | 0.1 | −1.05 | 0.2 |
| 571 | 1387547_a_at | 11.47 | 20.4 | 16.29 | 23.3 | 5.57 | 13.7 |
| 572 | 1387548_at | 1.88 | 4.5 | 2.49 | 7.8 | 1.02 | 0.1 |
| 573 | 1387630_at | 1.89 | 9.7 | 1.50 | 5.1 | 1.14 | 0.9 |
| 574 | 1387648_at | 54.71 | 19.6 | 45.08 | 18.6 | 3.48 | 4.1 |
| 575 | 1387675_at | 4.90 | 20.6 | 3.35 | 15.2 | 1.66 | 4.5 |
| 576 | 1387759_s_at | 5.53 | 22.3 | 4.27 | 18.8 | 1.64 | 4.4 |
| 577 | 1387789_at | 1.25 | 1.3 | 1.69 | 5 | 1.87 | 6.5 |
| 578 | 1387805_at | 2.19 | 6.9 | 1.70 | 3.8 | 1.23 | 0.9 |
| 579 | 1387808_at | 1.67 | 6.5 | 2.54 | 14.5 | 2.13 | 11.1 |
| 580 | 1387817_at | 2.07 | 3.9 | 2.90 | 7 | 1.90 | 3.2 |
| 581 | 1387843_at | −1.66 | 2.2 | −1.55 | 1.8 | −1.89 | 3.1 |
| 582 | 1387856_at | 1.87 | 14.4 | 2.23 | 19.1 | 1.39 | 5.9 |
| 583 | 1387870_at | 1.16 | 1 | −1.09 | 0.4 | −1.04 | 0.2 |
| 584 | 1387871_at | 2.96 | 16.6 | 3.48 | 19.3 | 1.32 | 2.4 |
| 585 | 1387885_at | 1.63 | 5.9 | 2.71 | 15.5 | 2.15 | 11.1 |
| 586 | 1387892_at | 1.79 | 11.7 | 1.93 | 13.6 | 1.15 | 1.4 |
| 587 | 1387893_at | 5.21 | 26.3 | 7.06 | 30.3 | 5.80 | 27.7 |
| 588 | 1387896_at | −1.58 | 6 | −1.43 | 4.1 | −1.07 | 0.4 |
| 589 | 1387897_at | −1.36 | 2.6 | 1.06 | 0.3 | −1.05 | 0.2 |
| 590 | 1387975_at | 3.55 | 18.3 | 3.68 | 18.8 | 2.26 | 10.7 |
| 591 | 1387976_at | 2.12 | 6 | 3.74 | 13.1 | 2.09 | 5.8 |
| 592 | 1388032_a_at | 9.42 | 6.1 | 3.14 | 2.2 | 1.26 | 0.2 |
| 593 | 1388054_a_at | 10.09 | 10.5 | 23.66 | 15.6 | 3.01 | 3.5 |
| 594 | 1388089_a_at | 1.74 | 10 | 1.51 | 6.6 | 1.22 | 2.2 |
| 595 | 1388119_at | 1.07 | 0.7 | 1.11 | 1.4 | −1.10 | 1.3 |
| 596 | 1388120_at | −1.00 | 0 | 1.02 | 0.1 | 1.12 | 1.1 |
| 597 | 1388131_at | 6.36 | 18.2 | 8.65 | 21.4 | 2.00 | 4.9 |
| 598 | 1388142_at | 12.19 | 10.4 | 22.60 | 13.8 | 3.30 | 3.5 |
| 599 | 1388143_at | 5.14 | 22.5 | 5.97 | 24.5 | 1.85 | 6.7 |
| 600 | 1388151_at | 1.31 | 5.1 | 1.52 | 9.4 | 1.05 | 0.4 |
| 601 | 1388155_at | −1.13 | 0.2 | −1.33 | 0.6 | 1.09 | 0.1 |
| 602 | 1388170_at | 1.46 | 4.3 | 1.57 | 5.6 | 1.26 | 2 |
| 603 | 1388201_at | −1.21 | 0.6 | 1.18 | 0.5 | 1.56 | 2 |
| 604 | 1388204_at | 14.09 | 10.7 | 3.32 | 3.3 | 1.27 | 0.3 |
| 605 | 1388218_at | 1.25 | 1 | −2.43 | 7.7 | −1.36 | 1.6 |
| 606 | 1388243_at | 2.56 | 10.1 | 5.22 | 19.9 | 3.89 | 16 |
| 607 | 1388271_at | 6.33 | 23.1 | 5.12 | 20.5 | 2.79 | 11.9 |
| 608 | 1388312_at | 1.54 | 3.4 | 2.68 | 11.3 | 3.55 | 15.5 |
| 609 | 1388318_at | 2.06 | 14.8 | 1.64 | 9.1 | 1.10 | 0.8 |
| 610 | 1388335_at | 2.90 | 24.5 | 2.86 | 24.2 | 1.25 | 3.1 |
| 611 | 1388348_at | 1.95 | 12.8 | 1.62 | 8.3 | 1.08 | 0.5 |
| 612 | 1388356_at | −1.06 | 0.4 | −1.47 | 6.8 | −1.12 | 1.1 |
| 613 | 1388392_at | 2.33 | 19.5 | 2.13 | 17.3 | 1.22 | 2.5 |
| 614 | 1388397_at | 1.34 | 2.5 | 1.33 | 2.4 | 1.06 | 0.3 |
| 615 | 1388401_at | 1.81 | 8.4 | 2.00 | 10.5 | 1.07 | 0.3 |
| 616 | 1388449_at | 1.22 | 5 | 1.13 | 2.5 | 1.05 | 0.6 |
| 617 | 1388459_at | 17.57 | 29.4 | 21.80 | 31.2 | 6.44 | 18.8 |
| 618 | 1388486_at | −1.37 | 5.3 | −1.29 | 3.8 | 1.04 | 0.3 |
| 619 | 1388492_at | 1.54 | 10.1 | 1.10 | 1.1 | 1.13 | 1.5 |
| 620 | 1388493_at | 2.03 | 6.2 | 2.98 | 11.4 | 2.55 | 9.3 |
| 621 | 1388494_at | 2.49 | 9.1 | 5.09 | 18.7 | 1.94 | 5.7 |
| 622 | 1388495_at | −1.14 | 1.1 | −1.08 | 0.5 | 1.00 | 0 |
| 623 | 1388521_at | 1.39 | 3.1 | 1.82 | 7.6 | 1.46 | 3.8 |
| 624 | 1388533_at | −1.39 | 7.5 | −1.29 | 5.2 | 1.04 | 0.3 |
| 625 | 1388534_at | 1.36 | 5.8 | 1.54 | 9.5 | 1.31 | 4.9 |
| 626 | 1388568_at | 1.10 | 1.6 | −1.06 | 0.8 | −1.10 | 1.6 |
| 627 | 1388583_at | 4.14 | 13.2 | 6.43 | 18.1 | 2.78 | 8.5 |
| 628 | 1388602_at | 7.90 | 19.6 | 7.89 | 19.6 | 3.39 | 10.4 |
| 629 | 1388618_at | 2.43 | 13.6 | 3.38 | 19.6 | 1.99 | 9.8 |
| 630 | 1388666_at | −1.11 | 0.5 | 1.20 | 1.2 | −1.16 | 0.9 |
| 631 | 1388695_at | 1.43 | 6.5 | 1.30 | 4 | −1.04 | 0.3 |
| 632 | 1388699_at | 1.07 | 0.5 | 1.38 | 4.4 | 1.57 | 7.5 |
| 633 | 1388700_at | −1.41 | 5.8 | −1.09 | 0.7 | 1.21 | 2.3 |
| 634 | 1388703_at | 10.96 | 21.3 | 17.80 | 25.4 | 5.16 | 14 |
| 635 | 1388705_at | 2.67 | 15.9 | 4.51 | 24.8 | 2.66 | 15.8 |
| 636 | 1388711_at | 2.80 | 19.6 | 2.73 | 19.1 | 1.77 | 9.5 |
| 637 | 1388715_at | 1.61 | 12.8 | 1.52 | 10.7 | 1.23 | 3.9 |
| 638 | 1388728_at | −1.13 | 1.4 | −1.20 | 2.5 | −1.05 | 0.4 |
| 639 | 1388742_at | 1.09 | 0.6 | 1.43 | 5.3 | 1.50 | 6.4 |
| 640 | 1388750_at | 2.11 | 7.1 | 2.08 | 6.9 | 1.16 | 0.7 |
| 641 | 1388773_at | 1.45 | 3.9 | 1.01 | 0 | −1.01 | 0 |
| 642 | 1388792_at | 2.49 | 5.6 | 4.58 | 11.5 | −1.01 | 0 |
| 643 | 1388836_at | −1.33 | 4.8 | −1.66 | 11 | −1.40 | 6.2 |
| 644 | 1388870_at | −1.44 | 8 | −1.34 | 5.9 | −1.01 | 0 |
| 645 | 1388878_at | 1.40 | 2.5 | 1.32 | 1.9 | 1.33 | 1.9 |
| 646 | 1388889_at | −1.62 | 9.3 | −1.66 | 10 | −1.06 | 0.4 |
| 647 | 1388926_at | 1.50 | 3.4 | 1.55 | 3.9 | 1.62 | 4.5 |
| 648 | 1388945_at | 3.90 | 18.5 | 3.39 | 16.4 | 1.93 | 7.2 |
| 649 | 1388955_at | 1.94 | 17.8 | 2.28 | 22.3 | 1.58 | 11.3 |
| 650 | 1388970_at | 9.50 | 16.9 | 12.43 | 19.1 | 3.59 | 8.2 |
| 651 | 1389018_at | 1.99 | 4.4 | 1.39 | 1.4 | −1.04 | 0.1 |
| 652 | 1389039_at | 2.53 | 14.6 | 3.23 | 19.1 | 2.34 | 13.1 |
| 653 | 1389040_at | −1.69 | 10.5 | −1.32 | 4.1 | −1.05 | 0.4 |
| 654 | 1389059_at | −1.18 | 0.8 | −1.07 | 0.2 | 1.46 | 2.8 |
| 655 | 1389103_at | 1.14 | 1 | 1.08 | 0.5 | −1.13 | 0.9 |
| 656 | 1389123_at | 3.39 | 12.6 | 2.87 | 10.4 | 1.38 | 1.7 |
| 657 | 1389151_at | 3.40 | 12.9 | 6.30 | 20.5 | 2.67 | 9.6 |
| 658 | 1389153_at | −1.52 | 8 | −1.64 | 10.2 | −1.03 | 0.2 |
| 659 | 1389157_at | 1.53 | 7.1 | 1.35 | 4.1 | 1.27 | 3 |
| 660 | 1389186_at | 1.91 | 8.8 | 2.55 | 14.4 | 2.38 | 13.1 |
| 661 | 1389207_at | 1.29 | 3.4 | 1.03 | 0.1 | 1.05 | 0.3 |
| 662 | 1389213_at | 1.52 | 4.5 | 1.69 | 6.4 | 1.23 | 1.5 |
| 663 | 1389214_at | 12.17 | 22.7 | 24.95 | 28.5 | 5.90 | 15.7 |
| 664 | 1389227_at | 1.23 | 2.6 | 1.14 | 1.3 | −1.09 | 0.7 |
| 665 | 1389244_x_at | 29.30 | 21.2 | 41.03 | 23.3 | 12.76 | 15.6 |
| 666 | 1389255_at | 7.22 | 16.2 | 9.60 | 18.8 | 3.49 | 9 |
| 667 | 1389297_at | 1.64 | 4.2 | 1.31 | 1.6 | 1.16 | 0.7 |
| 668 | 1389324_at | 3.58 | 14.8 | 4.61 | 18.2 | 1.86 | 5.5 |
| 669 | 1389341_at | 8.09 | 17.5 | 12.18 | 21.2 | 3.44 | 9 |
| 670 | 1389373_at | 1.11 | 1.2 | 1.20 | 2.6 | 1.06 | 0.5 |
| 671 | 1389464_at | −6.15 | 3.7 | −8.09 | 4.6 | −1.85 | 0.7 |
| 672 | 1389470_at | 6.27 | 8.3 | 4.34 | 6 | 3.46 | 4.6 |
| 673 | 1389476_at | −1.40 | 5.5 | −1.16 | 1.5 | −1.12 | 1 |
| 674 | 1389478_at | 1.55 | 5.5 | 1.88 | 9.3 | 1.48 | 4.6 |
| 675 | 1389483_at | 3.02 | 16.4 | 5.63 | 25.8 | 2.79 | 14.2 |
| 676 | 1389533_at | −1.08 | 0.2 | 3.74 | 13 | 3.09 | 10.6 |
| 677 | 1389546_at | −1.06 | 0.4 | 1.10 | 0.7 | 1.20 | 1.9 |
| 678 | 1389573_at | 1.81 | 5.9 | 1.44 | 2.8 | 1.82 | 6 |
| 679 | 1389590_at | −2.12 | 9.2 | −1.70 | 5.5 | −1.55 | 4.2 |
| 680 | 1389611_at | −1.47 | 3 | −1.64 | 4.4 | 1.06 | 0.2 |
| 681 | 1389617_at | 1.79 | 8.9 | 1.59 | 6.3 | −1.09 | 0.5 |
| 682 | 1389651_at | 4.28 | 11.3 | 4.71 | 12.2 | 1.28 | 0.8 |
| 683 | 1389787_at | 1.46 | 4.5 | 2.08 | 11.7 | 1.39 | 3.6 |
| 684 | 1389789_at | −1.42 | 4.4 | −1.70 | 8.3 | −1.23 | 2 |
| 685 | 1389836_a_at | 1.13 | 0.5 | 1.56 | 3.3 | 1.93 | 6.1 |
| 686 | 1389883_at | −1.37 | 4.8 | −1.20 | 2.1 | −1.05 | 0.3 |
| 687 | 1389966_at | −1.69 | 8.7 | 1.02 | 0.1 | 1.60 | 7.4 |
| 688 | 1390031_at | 1.54 | 5.6 | 1.38 | 3.6 | 1.14 | 0.9 |
| 689 | 1390112_at | −2.17 | 4.8 | −2.17 | 4.8 | 1.10 | 0.3 |
| 690 | 1390141_at | 1.45 | 1.6 | 1.23 | 0.7 | 1.71 | 2.9 |
| 691 | 1390159_at | 2.20 | 4.7 | 4.10 | 11 | 1.69 | 2.5 |
| 692 | 1390173_at | 1.44 | 6 | −1.04 | 0.2 | 1.00 | 0 |
| 693 | 1390300_at | 1.95 | 4 | 2.44 | 6.3 | 1.42 | 1.5 |
| 694 | 1390306_at | 2.97 | 12 | 3.34 | 13.7 | 1.94 | 6 |
| 695 | 1390311_at | −1.10 | 0.8 | −1.10 | 0.7 | 1.03 | 0.2 |
| 696 | 1390380_at | −1.08 | 0.6 | 1.01 | 0 | 1.15 | 1.6 |
| 697 | 1390383_at | 4.26 | 12.5 | 4.00 | 11.8 | 1.46 | 1.7 |
| 698 | 1390388_at | 1.49 | 2.2 | 1.72 | 3.5 | 1.21 | 0.7 |
| 699 | 1390403_at | 3.31 | 12.2 | 2.97 | 10.8 | 2.30 | 7.4 |
| 700 | 1390406_at | 2.14 | 13.3 | 2.11 | 13 | 1.19 | 1.5 |
| 701 | 1390415_at | 1.77 | 5.1 | 1.48 | 2.9 | 1.00 | 0 |
| 702 | 1390420_at | 4.71 | 11.8 | 4.27 | 10.8 | 3.78 | 9.6 |
| 703 | 1390536_at | −2.33 | 13.4 | −2.01 | 10.4 | −1.33 | 2.7 |
| 704 | 1390555_at | 1.29 | 2.4 | 1.43 | 4 | 1.04 | 0.2 |
| 705 | 1390647_at | 1.23 | 1.7 | 1.18 | 1.2 | 1.23 | 1.7 |
| 706 | 1390706_at | 1.10 | 0.6 | 1.30 | 2.4 | 1.15 | 0.9 |
| 707 | 1390738_at | 3.37 | 8 | 3.69 | 8.8 | 2.10 | 3.8 |
| 708 | 1390782_at | 2.28 | 5.2 | 2.95 | 7.8 | 1.60 | 2.2 |
| 709 | 1390828_at | −1.20 | 0.2 | 1.53 | 0.6 | −1.78 | 1 |
| 710 | 1390832_at | −1.50 | 4.4 | −2.18 | 11.5 | −1.29 | 2.2 |
| 711 | 1390835_at | 1.92 | 3.9 | 1.40 | 1.4 | 1.09 | 0.2 |
| 712 | 1390846_at | −1.46 | 5.3 | 1.01 | 0 | 1.68 | 8.4 |
| 713 | 1390914_at | 2.98 | 15.3 | 4.12 | 20.4 | 1.78 | 6.5 |
| 714 | 1391167_at | −1.06 | 0.4 | −1.27 | 2.9 | −1.04 | 0.3 |
| 715 | 1391421_at | −1.09 | 0.5 | 1.14 | 1 | 1.12 | 0.7 |
| 716 | 1391435_at | 12.23 | 24.5 | 18.75 | 28.1 | 13.30 | 25.2 |
| 717 | 1391442_at | 1.30 | 1.4 | 1.97 | 6.1 | 1.43 | 2.3 |
| 718 | 1391450_at | 2.56 | 8.4 | 5.77 | 18.3 | 2.80 | 9.5 |
| 719 | 1391458_at | 5.00 | 15.4 | 1.87 | 4.1 | 1.23 | 0.8 |
| 720 | 1391503_at | 2.16 | 6.5 | 2.71 | 9.4 | 1.44 | 2 |
| 721 | 1391505_x_at | 3.46 | 5.6 | 1.95 | 2.2 | 1.04 | 0.1 |
| 722 | 1391607_at | 1.78 | 7.7 | −1.01 | 0 | 1.03 | 0.1 |
| 723 | 1391827_at | 1.80 | 8.7 | 1.65 | 7 | 1.13 | 0.8 |
| 724 | 1391856_at | 2.41 | 8.5 | 3.38 | 13.1 | 1.46 | 2.3 |
| 725 | 1391871_at | 1.52 | 2.1 | 3.08 | 9.5 | 1.60 | 2.5 |
| 726 | 1391946_at | 3.74 | 7.3 | 4.30 | 8.4 | 2.75 | 4.9 |
| 727 | 1392044_at | 1.19 | 1.1 | 1.45 | 3.5 | 1.86 | 7.7 |
| 728 | 1392171_at | 11.42 | 13.1 | 3.78 | 5.6 | 1.28 | 0.5 |
| 729 | 1392220_at | 1.12 | 0.4 | 1.55 | 3.1 | 1.22 | 1 |
| 730 | 1392264_s_at | 5.80 | 6.1 | 6.69 | 6.8 | 1.61 | 0.8 |
| 731 | 1392280_at | 1.67 | 3.5 | 1.49 | 2.4 | 1.06 | 0.2 |
| 732 | 1392471_at | −1.42 | 6.9 | −1.41 | 6.6 | −1.21 | 2.7 |
| 733 | 1392489_at | 3.21 | 18.1 | 5.06 | 25.2 | 2.70 | 15 |
| 734 | 1392490_at | −1.38 | 7.3 | −1.34 | 6.2 | 1.04 | 0.3 |
| 735 | 1392497_at | −1.55 | 6.2 | −1.13 | 0.9 | −1.06 | 0.3 |
| 736 | 1392578_at | 3.43 | 12.4 | 6.19 | 19.5 | 4.60 | 16.1 |
| 737 | 1392597_at | −1.10 | 0.6 | −1.15 | 1 | 1.31 | 2.6 |
| 738 | 1392648_at | 17.00 | 28.5 | 23.88 | 31.3 | 6.47 | 19.2 |
| 739 | 1392736_at | 4.41 | 5.7 | 1.39 | 0.6 | −1.05 | 0.1 |
| 740 | 1392784_at | 1.16 | 0.9 | 1.26 | 1.8 | 1.58 | 5.1 |
| 741 | 1392786_at | 5.95 | 16.2 | 8.42 | 19.7 | 2.89 | 8.3 |
| 742 | 1392905_at | 6.05 | 11.5 | 9.93 | 15.5 | 3.76 | 7.5 |
| 743 | 1392917_at | −1.29 | 5.2 | −1.27 | 4.9 | −1.06 | 0.6 |
| 744 | 1392934_at | −1.05 | 0.5 | 1.14 | 2.4 | 1.20 | 3.9 |
| 745 | 1392948_at | 1.07 | 0.1 | −1.07 | 0.1 | 1.30 | 0.8 |
| 746 | 1392953_at | 1.54 | 1.7 | 2.24 | 4.5 | 1.13 | 0.3 |
| 747 | 1392953_at | 1.54 | 1.7 | 2.24 | 4.5 | 1.13 | 0.3 |
| 748 | 1392990_at | 10.57 | 13.4 | 21.64 | 18.3 | 4.13 | 6.7 |
| 749 | 1393003_at | 1.45 | 3 | 1.81 | 6 | 1.64 | 4.7 |
| 750 | 1393067_at | 2.93 | 15.1 | 4.15 | 20.7 | 2.14 | 9.7 |
| 751 | 1393210_at | −1.39 | 1 | 1.13 | 0.3 | 1.68 | 2.1 |
| 752 | 1393235_at | 4.79 | 12 | 8.07 | 16.9 | 2.26 | 4.7 |
| 753 | 1393240_at | 3.34 | 19.5 | 4.47 | 24.2 | 2.56 | 14.8 |
| 754 | 1393249_at | 4.95 | 15.5 | 7.92 | 20.6 | 2.51 | 7.5 |
| 755 | 1393252_at | 5.29 | 17.4 | 7.73 | 21.6 | 3.31 | 11.6 |
| 756 | 1393281_at | 3.00 | 17.2 | 3.65 | 20.5 | 1.51 | 4.5 |
| 757 | 1393282_at | −1.08 | 0.4 | −1.31 | 2.4 | −1.12 | 0.7 |
| 758 | 1393316_at | 8.41 | 17.2 | 10.12 | 18.9 | 3.79 | 9.6 |
| 759 | 1393401_at | 5.79 | 6.5 | 14.09 | 11.5 | 3.78 | 4.2 |
| 760 | 1393427_s_at | 3.62 | 18.7 | 4.29 | 21.3 | 1.78 | 6.6 |
| 761 | 1393460_at | 3.25 | 16.8 | 3.90 | 19.6 | 1.90 | 7.6 |
| 762 | 1393706_at | 2.73 | 9.5 | 2.67 | 9.2 | 1.63 | 3.2 |
| 763 | 1393730_at | 6.74 | 11.5 | 11.77 | 15.8 | 2.41 | 3.7 |
| 764 | 1393799_at | 1.36 | 1.8 | 2.36 | 8.5 | 1.39 | 2 |
| 765 | 1393883_at | 1.81 | 3.4 | 3.09 | 8.9 | 1.50 | 1.9 |
| 766 | 1393917_at | 41.04 | 24.6 | 47.54 | 25.5 | 7.50 | 12.6 |
| 767 | 1393944_at | 1.62 | 2.9 | 2.83 | 9.2 | 2.90 | 9.5 |
| 768 | 1393987_s_at | 1.27 | 4.3 | 1.17 | 2.2 | 1.05 | 0.4 |
| 769 | 1394109_at | 3.24 | 12.2 | 5.66 | 19.3 | 4.27 | 15.8 |
| 770 | 1394315_at | 3.53 | 10.1 | 2.45 | 6.2 | 1.31 | 1 |
| 771 | 1394375_x_at | 2.47 | 6.7 | 3.37 | 10.2 | 1.73 | 3.1 |
| 772 | 1394459_at | −2.06 | 7.7 | −1.99 | 7.2 | 1.13 | 0.5 |
| 773 | 1394473_at | 1.86 | 5.5 | 1.98 | 6.3 | 1.36 | 1.9 |
| 774 | 1394483_at | 3.58 | 6.9 | 4.59 | 8.9 | 2.37 | 3.8 |
| 775 | 1394490_at | 1.82 | 2.9 | 1.90 | 3.2 | 3.16 | 7.8 |
| 776 | 1394597_at | 1.08 | 0.6 | 1.10 | 0.8 | 1.37 | 4.7 |
| 777 | 1394612_at | −1.73 | 5.6 | −1.42 | 2.8 | 1.18 | 0.9 |
| 778 | 1395079_at | 1.70 | 3.3 | 2.17 | 5.8 | 1.58 | 2.6 |
| 779 | 1395197_at | 1.48 | 2.5 | 1.84 | 4.9 | −1.07 | 0.2 |
| 780 | 1395265_at | 1.29 | 1.9 | 1.53 | 4.3 | 1.48 | 3.8 |
| 781 | 1395357_at | 1.77 | 3.5 | 2.10 | 5.1 | 1.70 | 3 |
| 782 | 1395508_at | 1.46 | 3.2 | 1.33 | 2.1 | 1.11 | 0.5 |
| 783 | 1395635_at | 2.09 | 4.5 | 2.50 | 6.3 | 1.37 | 1.2 |
| 784 | 1395663_at | −1.28 | 4.1 | −1.62 | 10.8 | −1.18 | 2.2 |
| 785 | 1395730_at | 1.76 | 5.5 | 1.87 | 6.4 | 1.31 | 1.8 |
| 786 | 1396055_at | −1.39 | 1.9 | 1.35 | 1.6 | 2.01 | 6 |
| 787 | 1396208_at | 2.72 | 8.3 | 2.27 | 6.2 | 1.43 | 1.8 |
| 788 | 1397272_at | −1.51 | 3.9 | −1.58 | 4.6 | −1.06 | 0.2 |
| 789 | 1397317_at | 7.02 | 13.2 | 12.73 | 18 | 3.21 | 6.5 |
| 790 | 1397644_at | 1.48 | 2.7 | 2.55 | 10.1 | −1.10 | 0.3 |
| 791 | 1397674_at | 1.56 | 4.2 | 1.35 | 2.2 | 1.09 | 0.4 |
| 792 | 1397808_at | 5.53 | 14.7 | 10.22 | 20.7 | 2.42 | 6 |
| 793 | 1398131_at | 1.36 | 2.2 | 1.40 | 2.4 | 1.76 | 5.5 |
| 794 | 1398246_s_at | 28.97 | 22.6 | 22.59 | 20.9 | 7.50 | 12.7 |
| 795 | 1398256_at | 44.01 | 17.7 | 15.34 | 11.9 | 2.24 | 1.9 |
| 796 | 1398275_at | 191.82 | 15.3 | 91.66 | 12.7 | 3.94 | 2.2 |
| 797 | 1398286_at | −1.58 | 4.9 | −1.44 | 3.4 | −1.10 | 0.5 |
| 798 | 1398287_at | 4.44 | 17.3 | 2.69 | 10.4 | 1.43 | 2.3 |
| 799 | 1398303_s_at | 3.76 | 20.8 | 3.91 | 21.4 | 1.72 | 6.8 |
| 800 | 1398332_at | 1.74 | 8.5 | 1.89 | 10.3 | 1.23 | 1.9 |
| 801 | 1398333_at | 1.11 | 1.1 | 1.49 | 9 | 1.32 | 5.4 |
| 802 | 1398335_at | 1.17 | 1.6 | 1.40 | 5.3 | 1.42 | 5.6 |
| 803 | 1398348_at | 3.79 | 6.8 | 5.16 | 9.2 | 1.31 | 0.6 |
| 804 | 1398350_at | 3.46 | 11.4 | 10.29 | 23.3 | 3.03 | 9.8 |
| 805 | 1398356_at | 1.52 | 12.8 | 1.52 | 12.9 | −1.01 | 0.1 |
| 806 | 1398380_at | 3.61 | 18.3 | 3.64 | 18.4 | 1.72 | 5.9 |
| 807 | 1398390_at | 1.54 | 1.1 | 2.32 | 3 | 2.63 | 3.7 |
| 808 | 1398397_at | 2.67 | 10.9 | 4.06 | 16.9 | 1.98 | 6.5 |
| 809 | 1398597_at | 1.75 | 4.9 | 2.27 | 8.5 | 2.06 | 7.1 |
| 810 | 1398626_s_at | 1.31 | 9 | 1.36 | 10.8 | −1.01 | 0.1 |
| 811 | 1398656_at | −34.18 | 15.7 | −25.57 | 14.2 | −17.45 | 12.1 |
| 812 | 1398664_at | 1.18 | 0.8 | 1.01 | 0 | −1.00 | 0 |
| 813 | 1398727_at | 2.09 | 3.6 | 3.06 | 6.8 | 1.98 | 3.2 |
| 814 | 1398757_at | 2.92 | 21.2 | 3.27 | 23.4 | 1.69 | 9 |
| 815 | 1398765_at | 1.36 | 9.8 | 1.53 | 15 | 1.08 | 1.2 |
| 816 | 1398828_at | 4.91 | 24.7 | 7.41 | 30.1 | 2.35 | 12.5 |
| 817 | 1398829_at | 3.68 | 27.9 | 5.25 | 33.7 | 2.08 | 14.8 |
| 818 | 1398833_at | −1.11 | 1.3 | 1.07 | 0.8 | 1.20 | 3.3 |
| 819 | 1398840_at | 1.80 | 8 | 2.19 | 11.8 | 1.52 | 4.9 |
| 820 | 1398851_at | 1.02 | 0.2 | −1.03 | 0.3 | −1.10 | 1.2 |
| 821 | 1398857_at | −1.06 | 0.5 | −1.12 | 1.3 | 1.03 | 0.2 |
| 822 | 1398873_at | 1.14 | 1.9 | 1.07 | 0.7 | −1.15 | 2 |
| 823 | 1398893_at | −1.02 | 0.1 | 1.21 | 3.5 | 1.03 | 0.3 |
| 824 | 1398962_at | 1.42 | 6.4 | 1.35 | 5.2 | 1.06 | 0.5 |
| 825 | 1398970_at | −1.15 | 1.7 | −1.05 | 0.4 | 1.11 | 1.1 |
| 826 | 1398994_at | 1.52 | 4.1 | 2.75 | 14.4 | 1.56 | 4.6 |
| 827 | 1399019_at | 1.30 | 8.6 | 1.34 | 9.8 | 1.16 | 3.5 |
| 828 | 1399045_at | 1.26 | 3 | 1.62 | 9 | 1.14 | 1.3 |
| 829 | 1399122_at | −1.46 | 5.8 | −1.26 | 2.8 | −1.06 | 0.3 |

TABLE 2
Time course of gene expression in cornea-sclera tissue

| | | | | | | Fold | | Fold | | Fold | | Fold | |
|---|---|---|---|---|---|---|---|---|---|---|---|---|---|
| | Affymetrix | Fold Change | NLogP | Fold Change | NLogP | Change | NLogP | Change | NLogP | Change | NLogP | Change | NLogP |
| No. | Probe Set ID | Day 1 | Day 1 | Day 2 | Day 2 | Day 4 | Day 4 | Day 7 | Day 7 | Day 15 | Day 15 | Day 38 | Day 38 |
| 1 | 1367453_at | 1.18 | 2.8 | 1.26 | 4.5 | 1.11 | 1.3 | 1.13 | 1.8 | 1.00 | 0 | −1.08 | 0.8 |
| 2 | 1367463_at | 1.53 | 12.9 | 1.12 | 1.8 | 1.21 | 4 | 1.09 | 1.3 | 1.06 | 0.8 | 1.04 | 0.4 |
| 3 | 1367538_at | −1.23 | 2.8 | −1.20 | 2.3 | −1.03 | 0.2 | 1.10 | 0.9 | 1.21 | 2.6 | 1.08 | 0.7 |
| 4 | 1367574_at | 1.78 | 5.9 | 2.54 | 11.7 | 2.72 | 12.8 | 2.91 | 13.9 | 1.83 | 6.3 | 1.20 | 1 |
| 5 | 1367575_at | 2.27 | 14.1 | 1.98 | 11.2 | 1.42 | 4.3 | 1.23 | 1.9 | −1.21 | 1.7 | −1.20 | 1.6 |
| 6 | 1367579_a_at | 1.33 | 5.4 | 1.09 | 0.9 | 1.16 | 1.9 | 1.09 | 0.8 | 1.00 | 0 | −1.07 | 0.6 |
| 7 | 1367581_a_at | 3.31 | 3.8 | 5.30 | 6.2 | 8.78 | 9.1 | 4.80 | 5.7 | 1.86 | 1.4 | 1.84 | 1.3 |
| 8 | 1367590_at | 1.92 | 16.7 | 1.38 | 6.5 | 1.33 | 5.3 | 1.22 | 3.1 | −1.05 | 0.4 | −1.15 | 1.8 |
| 9 | 1367594_at | −1.17 | 0.9 | 1.38 | 2.8 | 2.31 | 11.6 | 2.76 | 14.8 | 2.40 | 12.3 | 1.12 | 0.6 |
| 10 | 1367619_at | −1.39 | 4.2 | −1.10 | 0.6 | −1.01 | 0 | 1.17 | 1.3 | 1.23 | 2 | 1.10 | 0.7 |
| 11 | 1367627_at | 1.80 | 1.4 | 1.75 | 1.4 | 2.12 | 2.1 | 1.96 | 1.8 | −1.10 | 0.1 | −1.25 | 0.4 |
| 12 | 1367646_at | 1.20 | 3.4 | 1.46 | 10.1 | 1.44 | 9.5 | 1.27 | 5.3 | 1.21 | 3.6 | −1.03 | 0.2 |
| 13 | 1367652_at | 1.73 | 1.8 | 2.06 | 2.9 | 2.46 | 4 | 3.94 | 7.5 | 1.62 | 1.5 | 1.11 | 0.2 |
| 14 | 1367658_at | −1.88 | 7.2 | −1.33 | 2.1 | 1.16 | 0.8 | 1.09 | 0.4 | 1.02 | 0.1 | −1.03 | 0.1 |
| 15 | 1367661_at | 3.36 | 11.3 | 3.03 | 10 | 2.62 | 8.2 | 1.81 | 4 | 1.19 | 0.6 | 1.02 | 0.1 |
| 16 | 1367663_at | −1.07 | 0.6 | 1.04 | 0.3 | 1.04 | 0.4 | 1.03 | 0.3 | 1.06 | 0.5 | −1.04 | 0.3 |
| 17 | 1367671_at | 1.97 | 11 | 1.35 | 3.4 | 1.17 | 1.2 | 1.05 | 0.2 | 1.02 | 0.1 | −1.03 | 0.1 |
| 18 | 1367682_at | −1.78 | 5.9 | −1.37 | 2.3 | −1.11 | 0.5 | 1.03 | 0.1 | 1.14 | 0.6 | 1.03 | 0.1 |
| 19 | 1367693_at | 1.83 | 11.7 | 1.61 | 8.5 | 1.53 | 7.2 | 1.49 | 6.5 | 1.21 | 2 | 1.02 | 0.1 |
| 20 | 1367701_at | −1.28 | 1.2 | 1.11 | 0.4 | 2.19 | 7.3 | 1.94 | 5.7 | 1.33 | 1.6 | 1.10 | 0.3 |
| 21 | 1367716_at | −1.65 | 6 | −1.46 | 3.9 | −1.26 | 1.9 | −1.16 | 1 | 1.09 | 0.5 | 1.05 | 0.2 |
| 22 | 1367721_at | 1.90 | 14.7 | 1.37 | 5.4 | 1.24 | 3.1 | 1.07 | 0.6 | −1.05 | 0.4 | 1.11 | 1 |
| 23 | 1367722_at | −1.71 | 6.7 | −1.18 | 1.1 | 1.20 | 1.2 | 1.57 | 5.2 | 1.87 | 8.4 | 1.25 | 1.7 |
| 24 | 1367732_at | 1.20 | 3.1 | 1.12 | 1.4 | 1.18 | 2.6 | 1.13 | 1.6 | −1.05 | 0.5 | −1.08 | 0.9 |
| 25 | 1367733_at | −1.37 | 1.1 | −1.16 | 0.4 | 1.42 | 1.3 | 1.10 | 0.2 | −1.50 | 1.6 | −1.11 | 0.3 |
| 26 | 1367749_at | −1.65 | 6.1 | −1.12 | 0.6 | 1.23 | 1.6 | 1.38 | 3.1 | 1.44 | 3.7 | 1.00 | 0 |
| 27 | 1367760_at | 1.49 | 9.7 | 1.30 | 5.2 | 1.31 | 5.3 | 1.22 | 3.4 | 1.04 | 0.3 | −1.01 | 0.1 |
| 28 | 1367765_at | −1.10 | 0.5 | 1.18 | 1 | 1.53 | 4.4 | 1.61 | 5.2 | 1.27 | 1.7 | 1.02 | 0.1 |
| 29 | 1367774_at | 1.03 | 0.1 | −1.12 | 0.3 | −1.20 | 0.5 | 1.02 | 0 | 1.24 | 0.7 | 1.20 | 0.5 |
| 30 | 1367784_a_at | −1.02 | 0.1 | 1.29 | 1.1 | 1.55 | 2.5 | 1.53 | 2.3 | 1.12 | 0.4 | 1.08 | 0.2 |
| 31 | 1367786_at | 1.36 | 1.3 | 1.54 | 2.1 | 1.53 | 2.1 | 1.50 | 1.9 | 1.33 | 1.1 | −1.01 | 0 |
| 32 | 1367800_at | 1.94 | 7.7 | 1.55 | 4.1 | 1.86 | 7 | 2.23 | 10.1 | 1.18 | 0.9 | 1.54 | 4 |
| 33 | 1367801_at | −1.10 | 0.9 | −1.22 | 2.9 | 1.12 | 1.2 | 1.07 | 0.6 | −1.03 | 0.2 | −1.09 | 0.9 |
| 34 | 1367807_at | −1.40 | 5.7 | −1.00 | 0 | 1.26 | 3.2 | 1.34 | 4.6 | 1.47 | 6.9 | −1.02 | 0.1 |
| 35 | 1367814_at | −2.01 | 4.4 | −1.77 | 3.2 | −1.82 | 3.5 | −1.58 | 2.3 | −1.33 | 1.1 | −1.08 | 0.2 |
| 36 | 1367823_at | −1.49 | 4.7 | −1.36 | 3.1 | 1.07 | 0.3 | 1.25 | 1.9 | 1.51 | 4.8 | 1.16 | 1.1 |
| 37 | 1367846_at | 2.57 | 6.7 | 2.45 | 6.2 | 1.84 | 3.4 | 1.52 | 1.9 | 1.15 | 0.4 | −1.02 | 0.1 |
| 38 | 1367850_at | 6.93 | 15.2 | 7.91 | 16.4 | 7.28 | 15.7 | 5.61 | 13.2 | 1.98 | 3.5 | 1.92 | 3.2 |
| 39 | 1367859_at | −3.18 | 12.1 | −2.29 | 7.6 | −1.17 | 0.6 | −1.28 | 1.2 | −1.02 | 0.1 | −1.09 | 0.3 |
| 40 | 1367881_at | 1.57 | 8.8 | 1.29 | 3.7 | 1.43 | 6.3 | 1.37 | 5.2 | 1.17 | 1.8 | 1.01 | 0.1 |
| 41 | 1367902_at | −1.43 | 2 | 1.16 | 0.5 | 2.21 | 6.8 | 2.27 | 7.2 | 1.55 | 2.8 | 1.40 | 1.9 |
| 42 | 1367905_at | 7.88 | 20.7 | 5.22 | 16.3 | 3.03 | 9.8 | 2.77 | 8.7 | 1.20 | 0.7 | 1.24 | 0.8 |
| 43 | 1367914_at | 2.14 | 12 | 1.92 | 9.8 | 1.76 | 8 | 1.70 | 7.3 | 1.57 | 5.7 | −1.04 | 0.2 |
| 44 | 1367930_at | −1.05 | 0.1 | −1.58 | 2.4 | −1.55 | 2.3 | −1.90 | 4.1 | −1.37 | 1.4 | −1.23 | 0.8 |
| 45 | 1367940_at | −1.19 | 1.2 | −1.12 | 0.6 | −1.10 | 0.6 | −1.14 | 0.8 | −1.00 | 0 | 1.22 | 1.5 |
| 46 | 1367942_at | 2.22 | 8.2 | 3.02 | 12.8 | 2.03 | 6.9 | 2.19 | 8 | 1.47 | 2.7 | −1.00 | 0 |
| 47 | 1367948_a_at | −1.50 | 2.5 | −1.30 | 1.3 | 1.39 | 1.8 | 1.47 | 2.3 | 1.17 | 0.6 | 1.15 | 0.5 |
| 48 | 1367959_a_at | −2.48 | 2 | −1.24 | 0.3 | 1.36 | 0.4 | 1.82 | 1.1 | 1.68 | 0.9 | −1.20 | 0.2 |
| 49 | 1367973_at | 98.34 | 20.5 | 49.43 | 17.2 | 40.70 | 16.2 | 21.22 | 12.8 | 8.89 | 8.2 | 2.81 | 2.6 |
| 50 | 1367974_at | 1.09 | 0.6 | 1.11 | 0.7 | 1.24 | 2.1 | 1.12 | 0.8 | 1.11 | 0.7 | 1.10 | 0.6 |
| 51 | 1367998_at | 5.57 | 15.9 | 6.12 | 16.9 | 5.18 | 15.1 | 3.32 | 10.1 | −1.43 | 1.6 | −2.05 | 4.8 |
| 52 | 1368000_at | 2.74 | 6.1 | 3.46 | 8.3 | 4.79 | 11.5 | 3.74 | 9.1 | −1.23 | 0.6 | −2.25 | 4.4 |
| 53 | 1368049_at | 1.55 | 5.2 | 1.00 | 0 | 1.18 | 1.2 | 1.11 | 0.6 | −1.07 | 0.4 | −1.06 | 0.3 |
| 54 | 1368052_at | −1.65 | 4.2 | 1.14 | 0.6 | 1.31 | 1.6 | −1.10 | 0.4 | −1.11 | 0.4 | 1.18 | 0.8 |
| 55 | 1368057_at | −1.62 | 6 | −1.31 | 2.4 | −1.30 | 2.3 | −1.25 | 1.8 | −1.24 | 1.7 | 1.05 | 0.2 |
| 56 | 1368078_at | 2.69 | 3.5 | 2.28 | 2.6 | 4.51 | 6.6 | 3.97 | 5.8 | 1.03 | 0 | 1.08 | 0.1 |
| 57 | 1368079_at | 1.74 | 4.1 | 1.78 | 4.4 | 1.16 | 0.6 | −1.18 | 0.7 | −1.38 | 1.8 | −1.41 | 2 |
| 58 | 1368089_at | −1.57 | 3.1 | −1.14 | 0.5 | 1.87 | 5.2 | 1.71 | 4.1 | 1.14 | 0.5 | 1.21 | 0.9 |
| 59 | 1368097_a_at | −1.48 | 1.5 | −1.43 | 1.3 | −1.05 | 0.1 | 1.04 | 0.1 | −1.01 | 0 | 1.08 | 0.2 |
| 60 | 1368103_at | −1.23 | 1.7 | 1.03 | 0.2 | −1.00 | 0 | 1.06 | 0.3 | 1.25 | 1.9 | 1.06 | 0.3 |
| 61 | 1368106_at | −1.18 | 1 | −1.07 | 0.3 | 1.12 | 0.6 | −1.06 | 0.2 | −1.12 | 0.6 | −1.12 | 0.6 |
| 62 | 1368145_at | −2.64 | 2.4 | −1.91 | 1.3 | −2.44 | 2.1 | −2.16 | 1.7 | −1.33 | 0.4 | −1.18 | 0.2 |
| 63 | 1368173_at | 2.10 | 14.5 | 1.25 | 2.6 | 1.24 | 2.4 | 1.13 | 1 | 1.03 | 0.2 | 1.06 | 0.4 |
| 64 | 1368183_at | −1.39 | 3.5 | −1.14 | 0.9 | −1.03 | 0.1 | 1.18 | 1.2 | 1.00 | 0 | 1.08 | 0.4 |
| 65 | 1368200_at | −1.30 | 1.3 | 1.04 | 0.1 | 1.04 | 0.1 | 1.16 | 0.6 | 1.13 | 0.4 | 1.30 | 1.3 |
| 66 | 1368221_at | −1.53 | 5.7 | −1.53 | 5.6 | −1.32 | 3 | −1.08 | 0.5 | 1.19 | 1.4 | 1.03 | 0.1 |
| 67 | 1368223_at | 2.43 | 7 | 1.43 | 1.7 | 1.22 | 0.7 | 1.62 | 2.7 | 1.46 | 1.9 | 1.47 | 1.9 |
| 68 | 1368259_at | 3.63 | 16.5 | 2.39 | 10.1 | 2.13 | 8.3 | 1.67 | 4.7 | 1.10 | 0.4 | 1.21 | 1.1 |
| 69 | 1368280_at | 1.79 | 4.4 | 2.52 | 8.8 | 2.26 | 7.3 | 2.77 | 10 | 1.35 | 1.6 | 1.00 | 0 |
| 70 | 1368281_at | −1.57 | 3.1 | −1.32 | 1.5 | 1.49 | 2.6 | 1.45 | 2.3 | 1.23 | 0.9 | −1.14 | 0.5 |
| 71 | 1368322_at | −1.15 | 0.5 | −1.06 | 0.2 | −1.03 | 0.1 | 1.22 | 0.8 | 1.10 | 0.3 | −1.02 | 0.1 |
| 72 | 1368332_at | 2.06 | 4.6 | 4.21 | 12.4 | 3.61 | 10.7 | 2.85 | 8.1 | 1.18 | 0.5 | −1.46 | 1.7 |
| 73 | 1368347_at | −1.30 | 0.3 | 1.46 | 0.5 | 6.29 | 5.3 | 13.07 | 8.7 | 2.55 | 1.9 | 1.13 | 0.1 |
| 74 | 1368370_at | 1.77 | 4.4 | 1.70 | 3.9 | 2.17 | 7 | 2.70 | 9.9 | 1.58 | 3.1 | 1.28 | 1.2 |
| 75 | 1368393_at | 2.07 | 3.8 | 2.05 | 3.7 | 5.34 | 12.6 | 5.47 | 12.8 | 1.52 | 1.6 | 1.52 | 1.6 |
| 76 | 1368395_at | −2.40 | 8.7 | −3.11 | 12.3 | −2.33 | 8.2 | −1.86 | 5.2 | 1.01 | 0 | 1.11 | 0.4 |
| 77 | 1368404_at | −1.04 | 0.1 | 1.26 | 1.3 | 1.85 | 5.5 | 2.37 | 9 | 1.37 | 2 | 1.16 | 0.6 |
| 78 | 1368419_at | 1.14 | 0.3 | 1.19 | 0.4 | 1.54 | 1.6 | 2.21 | 4 | 1.59 | 1.8 | 1.55 | 1.6 |
| 79 | 1368420_at | 2.29 | 4.2 | 2.80 | 5.8 | 1.22 | 0.5 | 2.04 | 3.3 | −1.14 | 0.3 | −1.30 | 0.7 |
| 80 | 1368430_at | 2.73 | 12.1 | 4.07 | 18.1 | 3.53 | 16 | 3.67 | 16.6 | 1.55 | 3.7 | 1.07 | 0.2 |
| 81 | 1368448_at | −1.70 | 1.3 | −1.44 | 0.8 | 1.11 | 0.2 | −1.02 | 0 | −1.30 | 0.5 | −1.18 | 0.3 |
| 82 | 1368464_at | 2.74 | 5.5 | 4.45 | 9.8 | 3.44 | 7.5 | 3.70 | 8.2 | 1.51 | 1.4 | 1.20 | 0.4 |
| 83 | 1368474_at | 1.33 | 1.5 | 2.44 | 8.5 | 2.40 | 8.3 | 3.49 | 13.3 | 2.41 | 8.4 | 1.74 | 4.2 |
| 84 | 1368519_at | 9.92 | 9.2 | 10.47 | 9.5 | 13.39 | 10.9 | 11.31 | 9.9 | 3.45 | 3.7 | 1.29 | 0.4 |
| 85 | 1368612_at | 1.24 | 2.6 | 1.11 | 0.8 | 1.03 | 0.2 | −1.30 | 3.6 | −1.33 | 4.1 | −1.28 | 3.2 |
| 86 | 1368655_at | 5.79 | 13.5 | 5.23 | 12.5 | 4.30 | 10.6 | 3.04 | 7.2 | 1.28 | 0.7 | 1.03 | 0.1 |
| 87 | 1368657_at | 6.38 | 13.6 | 5.27 | 11.8 | 5.89 | 12.8 | 3.64 | 8.4 | 1.63 | 1.9 | 1.65 | 2 |
| 88 | 1368731_at | 1.58 | 1.8 | 2.50 | 5.1 | 1.38 | 1 | 1.44 | 1.2 | −1.23 | 0.6 | −1.02 | 0 |
| 89 | 1368771_at | 1.79 | 3.5 | 1.47 | 1.8 | 1.74 | 3.2 | 2.46 | 6.7 | 1.51 | 2 | 1.28 | 1 |
| 90 | 1368813_at | 6.74 | 14.9 | 5.23 | 12.5 | 4.04 | 10 | 4.59 | 11.3 | 2.02 | 3.6 | 2.05 | 3.8 |
| 91 | 1368851_at | 1.35 | 1.5 | 1.34 | 1.4 | 2.09 | 5.7 | 1.96 | 5 | 1.52 | 2.4 | 1.22 | 0.8 |
| 92 | 1368885_at | 1.44 | 2.9 | 1.61 | 4.4 | 2.21 | 9.3 | 1.98 | 7.6 | 1.19 | 0.9 | 1.10 | 0.4 |
| 93 | 1368989_at | −2.16 | 2.4 | −2.02 | 2.1 | −1.55 | 1 | −1.56 | 1.1 | −1.17 | 0.3 | 1.13 | 0.2 |
| 94 | 1369006_at | 1.81 | 5.9 | 1.32 | 1.9 | 1.14 | 0.6 | 1.25 | 1.3 | −1.18 | 0.9 | −1.08 | 0.3 |
| 95 | 1369044_a_at | 2.80 | 6.9 | 2.34 | 5.2 | 2.61 | 6.2 | 2.59 | 6.1 | 1.44 | 1.4 | 1.32 | 0.9 |
| 96 | 1369065_a_at | 1.19 | 1.7 | −1.10 | 0.7 | 1.05 | 0.3 | 1.25 | 2.5 | 1.27 | 2.7 | 1.00 | 0 |
| 97 | 1369087_at | −1.08 | 0.3 | −1.04 | 0.1 | 1.11 | 0.5 | 1.22 | 1 | 1.12 | 0.5 | −1.05 | 0.2 |
| 98 | 1369166_at | 4.27 | 3.4 | 6.59 | 5 | 16.91 | 9.1 | 11.32 | 7.3 | 1.22 | 0.2 | −1.62 | 0.7 |
| 99 | 1369269_at | 1.25 | 1.4 | 1.03 | 0.1 | 1.24 | 1.3 | 1.20 | 1 | 1.09 | 0.4 | 1.06 | 0.2 |
| 100 | 1369294_at | 5.45 | 5.9 | 4.71 | 5.2 | 4.06 | 4.4 | 3.15 | 3.2 | 1.33 | 0.4 | −1.90 | 1.3 |
| 101 | 1369313_at | 2.06 | 1.9 | 2.32 | 2.4 | 2.79 | 3.3 | 3.92 | 5.1 | 2.17 | 2.1 | 1.70 | 1.2 |
| 102 | 1369422_at | −1.22 | 1.6 | 1.11 | 0.6 | 1.74 | 7.8 | 1.76 | 8 | 1.52 | 5.1 | 1.08 | 0.4 |
| 103 | 1369425_at | −1.34 | 1.2 | −1.08 | 0.2 | 1.86 | 3.8 | 1.93 | 4.1 | 1.30 | 1 | −1.12 | 0.3 |
| 104 | 1369484_at | −1.62 | 2 | −1.12 | 0.3 | −1.04 | 0.1 | −1.07 | 0.2 | −1.04 | 0.1 | 1.04 | 0.1 |
| 105 | 1369621_s_at | 1.59 | 3.9 | 1.51 | 3.3 | 2.08 | 7.8 | 2.12 | 8 | 1.31 | 1.7 | −1.04 | 0.1 |
| 106 | 1369633_at | −4.01 | 18.5 | −2.44 | 10.8 | −1.23 | 1.2 | −1.18 | 0.9 | 1.23 | 1.2 | 1.24 | 1.3 |
| 107 | 1369651_at | −1.36 | 2.3 | 1.27 | 1.6 | 2.28 | 9.9 | 2.70 | 12.8 | 1.24 | 1.3 | −1.06 | 0.3 |
| 108 | 1369652_at | −1.25 | 0.7 | 1.70 | 2.5 | 3.26 | 8.3 | 3.69 | 9.5 | 1.75 | 2.7 | 1.13 | 0.3 |
| 109 | 1369703_at | −1.33 | 2.2 | −1.17 | 0.9 | −1.13 | 0.6 | −1.03 | 0.1 | −1.01 | 0 | −1.08 | 0.3 |
| 110 | 1369735_at | −1.52 | 4.5 | −1.38 | 3 | 1.05 | 0.2 | 1.10 | 0.5 | 1.24 | 1.7 | 1.14 | 0.8 |
| 111 | 1369815_at | 15.61 | 11.7 | 16.28 | 11.9 | 12.62 | 10.5 | 2.56 | 2.4 | 1.44 | 0.6 | 1.05 | 0.1 |
| 112 | 1369895_s_at | −1.26 | 0.7 | −1.44 | 1.3 | 1.05 | 0.1 | 1.04 | 0.1 | −1.11 | 0.2 | −1.28 | 0.7 |
| 113 | 1369897_s_at | 1.01 | 0.1 | 1.12 | 1.9 | 1.23 | 4.7 | 1.23 | 4.7 | 1.16 | 2.8 | 1.00 | 0 |
| 114 | 1369926_at | −1.60 | 3.2 | −1.68 | 3.7 | 1.21 | 0.8 | 1.68 | 3.7 | 1.67 | 3.6 | 1.56 | 2.9 |
| 115 | 1369931_at | 2.58 | 20.1 | 2.14 | 15.7 | 1.56 | 7.8 | 1.27 | 3 | −1.14 | 1.3 | −1.24 | 2.6 |
| 116 | 1369947_at | −2.35 | 9.2 | −1.59 | 3.6 | 1.58 | 3.6 | 1.76 | 5 | 1.91 | 6.1 | 1.18 | 0.8 |
| 117 | 1369950_at | 1.37 | 5 | 1.26 | 3.1 | 1.20 | 2.1 | 1.29 | 3.6 | 1.10 | 0.9 | −1.10 | 0.8 |
| 118 | 1369953_s_at | −1.58 | 9.3 | −1.09 | 0.8 | −1.14 | 1.5 | −1.31 | 4.4 | −1.20 | 2.4 | −1.03 | 0.2 |
| 119 | 1369955_at | −2.49 | 15.8 | −1.60 | 6.5 | −1.03 | 0.2 | 1.18 | 1.3 | 1.73 | 8.1 | −1.20 | 1.5 |
| 120 | 1369956_at | 2.26 | 16.2 | 2.21 | 15.7 | 2.05 | 13.9 | 1.76 | 10.3 | 1.31 | 3.5 | 1.05 | 0.3 |
| 121 | 1369958_at | 1.18 | 1.2 | 1.37 | 3.3 | 1.72 | 7.6 | 1.78 | 8.3 | 1.25 | 2 | 1.25 | 2 |
| 122 | 1369960_at | −2.01 | 5 | −1.90 | 4.4 | −1.39 | 1.6 | −1.44 | 1.8 | 1.08 | 0.2 | 1.05 | 0.1 |
| 123 | 1369961_at | 1.66 | 6.8 | 1.66 | 6.8 | 1.88 | 9.3 | 1.87 | 9.2 | 1.24 | 1.8 | 1.09 | 0.5 |
| 124 | 1369973_at | 2.63 | 9.1 | 3.08 | 11.2 | 2.43 | 8.1 | 2.21 | 6.8 | 1.40 | 1.8 | −1.04 | 0.1 |
| 125 | 1370007_at | 1.58 | 5.1 | 1.42 | 3.4 | 1.37 | 2.9 | 1.45 | 3.7 | 1.80 | 7.5 | 1.04 | 0.2 |
| 126 | 1370023_at | 1.33 | 2.6 | 1.33 | 2.5 | 2.07 | 10.4 | 2.42 | 13.3 | 1.28 | 2.1 | −1.09 | 0.4 |
| 127 | 1370048_at | −2.21 | 10.1 | −1.59 | 4.6 | −1.32 | 2.1 | −1.44 | 3.1 | −1.11 | 0.5 | 1.05 | 0.2 |
| 128 | 1370051_at | 2.35 | 7.1 | 1.17 | 0.6 | 1.09 | 0.3 | −1.19 | 0.7 | −1.57 | 2.7 | −1.84 | 4.3 |
| 129 | 1370057_at | −1.07 | 0.4 | 1.09 | 0.5 | 1.28 | 2.5 | 1.66 | 7.5 | 1.09 | 0.5 | −1.01 | 0 |
| 130 | 1370062_at | 2.45 | 13.7 | 1.52 | 4.7 | 1.22 | 1.5 | −1.17 | 1.1 | −1.05 | 0.2 | −1.20 | 1.3 |
| 131 | 1370073_at | 1.45 | 4.1 | 1.32 | 2.6 | 1.07 | 0.3 | 1.25 | 1.9 | 1.24 | 1.8 | −1.09 | 0.5 |
| 132 | 1370097_s_at | 3.64 | 11.9 | 4.21 | 13.7 | 6.01 | 17.7 | 7.12 | 19.5 | 2.73 | 8.5 | 1.28 | 1 |
| 133 | 1370172_at | 5.61 | 16.2 | 3.81 | 11.8 | 2.20 | 5.6 | 1.96 | 4.4 | 1.30 | 1 | −1.03 | 0.1 |
| 134 | 1370184_at | 2.44 | 12.5 | 2.29 | 11.3 | 2.94 | 15.8 | 3.00 | 16.1 | 1.46 | 3.6 | 1.10 | 0.5 |
| 135 | 1370221_at | −1.09 | 0.3 | −1.04 | 0.1 | 1.22 | 1 | 1.61 | 3.8 | 1.12 | 0.5 | −1.09 | 0.3 |
| 136 | 1370244_at | 1.30 | 4.6 | 1.75 | 13.4 | 1.40 | 6.7 | 1.30 | 4.6 | 1.25 | 3.5 | 1.11 | 1.1 |
| 137 | 1370248_at | −1.72 | 4.9 | −1.41 | 2.4 | −1.20 | 0.9 | −1.10 | 0.4 | 1.18 | 0.8 | 1.02 | 0.1 |
| 138 | 1370256_at | 1.83 | 8.8 | 1.52 | 5.1 | 1.81 | 8.5 | 2.26 | 13.1 | 1.32 | 2.7 | 1.04 | 0.2 |
| 139 | 1370290_at | 1.73 | 13.3 | 1.30 | 4.6 | 1.40 | 6.8 | 1.35 | 5.7 | 1.23 | 3.3 | −1.06 | 0.5 |
| 140 | 1370301_at | 1.12 | 0.6 | 1.43 | 3.4 | 2.22 | 11.3 | 2.12 | 10.3 | 1.77 | 7 | 1.10 | 0.5 |
| 141 | 1370309_s_at | 1.33 | 3.6 | 1.10 | 0.7 | 1.14 | 1 | 1.05 | 0.3 | −1.01 | 0 | 1.07 | 0.4 |
| 142 | 1370312_at | −1.26 | 1.6 | 1.23 | 1.4 | 1.93 | 8.2 | 2.09 | 9.6 | 1.60 | 5 | 1.15 | 0.8 |
| 143 | 1370328_at | −3.07 | 9.4 | −2.71 | 7.9 | −2.00 | 4.6 | −1.70 | 3.1 | −1.14 | 0.4 | 1.05 | 0.1 |
| 144 | 1370408_at | 1.57 | 5 | 1.51 | 4.3 | 1.55 | 4.8 | 1.73 | 6.7 | 1.09 | 0.4 | 1.33 | 2.5 |
| 145 | 1370442_at | 1.77 | 1.5 | 1.70 | 1.3 | 1.69 | 1.3 | 2.05 | 2.1 | 1.77 | 1.5 | −1.03 | 0 |
| 146 | 1370570_at | −1.25 | 1.5 | 1.07 | 0.3 | 1.61 | 4.8 | 1.80 | 6.6 | 1.37 | 2.6 | 1.05 | 0.2 |
| 147 | 1370613_s_at | 2.44 | 3.4 | 3.04 | 4.7 | 2.66 | 3.9 | 1.70 | 1.5 | −1.27 | 0.5 | −2.52 | 3.6 |
| 148 | 1370624_at | 2.88 | 6.9 | 1.99 | 3.6 | 2.00 | 3.6 | 1.80 | 2.8 | 1.12 | 0.3 | 1.15 | 0.4 |
| 149 | 1370633_at | 58.54 | 10.6 | 49.53 | 10 | 31.09 | 8.4 | 5.26 | 2.8 | 1.29 | 0.2 | −3.80 | 2 |
| 150 | 1370634_x_at | 23.06 | 10.6 | 17.27 | 9.3 | 12.75 | 8 | 3.02 | 2.2 | 1.15 | 0.1 | −1.08 | 0.1 |
| 151 | 1370642_s_at | −1.43 | 2.5 | 1.06 | 0.2 | 2.04 | 7.1 | 2.97 | 12.8 | 2.54 | 10.4 | 1.52 | 3.1 |
| 152 | 1370693_s_at | −1.75 | 4.5 | −1.17 | 0.7 | −1.24 | 1 | −1.20 | 0.8 | −1.06 | 0.2 | −1.10 | 0.4 |
| 153 | 1370802_at | −2.00 | 6.8 | −1.40 | 2.3 | −1.32 | 1.7 | −1.24 | 1.2 | 1.17 | 0.7 | 1.19 | 0.8 |
| 154 | 1370809_at | 1.14 | 0.8 | −1.03 | 0.1 | −1.03 | 0.1 | 1.02 | 0.1 | −1.07 | 0.3 | −1.21 | 1.4 |
| 155 | 1370828_at | −1.27 | 0.6 | 1.24 | 0.5 | 1.21 | 0.5 | 1.45 | 1.2 | 1.02 | 0 | 1.19 | 0.4 |
| 156 | 1370838_s_at | −1.53 | 8.8 | −1.38 | 5.8 | −1.39 | 6.1 | −1.32 | 4.7 | −1.03 | 0.2 | 1.00 | 0 |
| 157 | 1370845_at | 1.04 | 0.1 | 1.11 | 0.4 | 1.22 | 1 | 1.07 | 0.2 | 1.10 | 0.4 | −1.00 | 0 |
| 158 | 1370854_at | −1.83 | 2.7 | −1.22 | 0.5 | −1.15 | 0.3 | 1.10 | 0.2 | 1.45 | 1.3 | 1.42 | 1.2 |
| 159 | 1370855_at | −1.98 | 7.3 | −1.63 | 4.4 | −1.16 | 0.7 | −1.16 | 0.7 | 1.29 | 1.6 | 1.30 | 1.6 |
| 160 | 1370862_at | −2.30 | 15.4 | −1.60 | 7.1 | −1.11 | 0.8 | 1.05 | 0.3 | 1.20 | 1.7 | 1.13 | 0.9 |
| 161 | 1370892_at | 1.03 | 0 | 1.23 | 0.4 | 2.20 | 2.4 | 2.76 | 3.6 | 1.58 | 1.1 | 1.28 | 0.5 |
| 162 | 1370905_at | −1.47 | 6.1 | −1.46 | 5.9 | −1.11 | 0.8 | −1.33 | 3.9 | −1.18 | 1.7 | −1.15 | 1.2 |
| 163 | 1370927_at | −2.06 | 9.8 | −1.11 | 0.6 | 1.37 | 2.8 | 1.47 | 3.8 | 1.86 | 7.9 | 1.31 | 2.2 |
| 164 | 1370950_at | 1.31 | 2.4 | 1.08 | 0.4 | 1.52 | 4.9 | 1.72 | 7.2 | 1.40 | 3.5 | 1.43 | 3.8 |
| 165 | 1370954_at | 1.58 | 2.8 | 1.46 | 2.1 | 1.32 | 1.3 | 1.71 | 3.6 | 1.83 | 4.3 | 1.05 | 0.1 |
| 166 | 1370956_at | −1.57 | 12.2 | −1.35 | 7 | −1.04 | 0.4 | −1.08 | 1 | 1.18 | 2.8 | 1.16 | 2.3 |
| 167 | 1370963_at | 1.38 | 1.3 | 1.24 | 0.7 | 1.37 | 1.2 | 1.61 | 2.3 | 1.21 | 0.6 | 1.39 | 1.3 |
| 168 | 1371016_at | 2.13 | 3.9 | 1.78 | 2.6 | 1.67 | 2.1 | 1.73 | 2.4 | 1.54 | 1.7 | −1.08 | 0.2 |
| 169 | 1371037_at | 1.70 | 4.1 | 2.51 | 9.3 | 3.48 | 13.9 | 3.68 | 14.7 | 1.99 | 6.2 | −1.01 | 0 |
| 170 | 1371079_at | 15.31 | 14.5 | 15.34 | 14.5 | 9.18 | 11.2 | 8.94 | 11 | 3.78 | 5.4 | 2.03 | 2 |
| 171 | 1371166_at | 2.21 | 3 | 1.92 | 2.2 | 3.17 | 5.3 | 4.17 | 7.3 | 1.67 | 1.5 | 1.17 | 0.3 |
| 172 | 1371232_s_at | 6.52 | 5.2 | 4.30 | 3.5 | 3.29 | 2.6 | 5.87 | 4.7 | 1.47 | 0.5 | 1.40 | 0.4 |
| 173 | 1371246_at | 1.75 | 8.4 | 1.37 | 3.5 | 1.48 | 4.9 | 1.24 | 1.9 | 1.10 | 0.6 | −1.11 | 0.7 |
| 174 | 1371250_at | 2.50 | 6.5 | 3.39 | 9.8 | 3.80 | 11 | 3.51 | 10.1 | 1.76 | 3.1 | 1.20 | 0.6 |
| 175 | 1371320_at | −1.58 | 14.5 | −1.18 | 3.4 | −1.08 | 1 | −1.09 | 1.3 | 1.08 | 1.1 | 1.10 | 1.5 |
| 176 | 1371329_at | 1.78 | 9.2 | 1.39 | 4 | 1.19 | 1.5 | 1.03 | 0.2 | −1.16 | 1.2 | −1.15 | 1 |
| 177 | 1371341_at | 1.31 | 2.8 | 1.50 | 5.3 | 1.20 | 1.6 | 1.14 | 0.9 | 1.09 | 0.5 | 1.09 | 0.5 |
| 178 | 1371349_at | −2.37 | 14.2 | −1.71 | 7.5 | 1.04 | 0.2 | 1.13 | 0.8 | 1.39 | 3.6 | −1.02 | 0.1 |
| 179 | 1371356_at | −1.64 | 4.9 | −1.94 | 7.5 | −1.59 | 4.4 | −1.32 | 2 | 1.05 | 0.2 | 1.05 | 0.2 |
| 180 | 1371360_at | 2.23 | 9.3 | 2.17 | 8.9 | 2.18 | 9 | 1.51 | 3.4 | 1.06 | 0.2 | 1.07 | 0.3 |
| 181 | 1371369_at | −2.54 | 14.4 | −2.01 | 9.9 | −1.01 | 0.1 | 1.19 | 1.2 | 1.49 | 4.4 | −1.16 | 1 |
| 182 | 1371392_at | 1.51 | 7 | 1.51 | 6.9 | 1.09 | 0.7 | 1.20 | 2 | 1.05 | 0.3 | 1.01 | 0 |
| 183 | 1371440_at | 1.13 | 0.5 | 1.21 | 1 | 1.28 | 1.5 | 1.55 | 3.6 | 1.36 | 2.1 | 1.09 | 0.4 |
| 184 | 1371447_at | −1.60 | 2.9 | −1.61 | 3 | −1.57 | 2.7 | −1.75 | 3.8 | −1.32 | 1.3 | −1.15 | 0.5 |
| 185 | 1371487_at | 1.07 | 0.6 | 1.26 | 3.4 | 1.24 | 3.2 | 1.19 | 2.3 | 1.00 | 0 | −1.10 | 0.9 |
| 186 | 1371498_at | 1.60 | 10.8 | 1.09 | 0.9 | 1.11 | 1.1 | 1.15 | 1.7 | 1.03 | 0.2 | −1.11 | 1.1 |
| 187 | 1371507_at | −1.54 | 7.8 | −1.62 | 9.1 | −1.23 | 2.6 | −1.25 | 2.9 | 1.13 | 1.2 | −1.06 | 0.4 |
| 188 | 1371525_at | −1.02 | 0.1 | 1.04 | 0.2 | 1.10 | 0.7 | 1.28 | 2.7 | 1.06 | 0.3 | 1.01 | 0.1 |
| 189 | 1371541_at | −3.14 | 9.5 | −2.45 | 6.6 | −1.51 | 2 | −1.14 | 0.4 | 1.13 | 0.4 | 1.05 | 0.1 |
| 190 | 1371545_at | 1.17 | 0.3 | 2.12 | 3.2 | 4.17 | 8.4 | 3.94 | 7.9 | 1.72 | 1.9 | 1.53 | 1.3 |
| 191 | 1371572_at | −1.48 | 8.7 | −1.17 | 2.2 | −1.08 | 0.8 | −1.11 | 1.2 | 1.03 | 0.2 | −1.00 | 0 |
| 192 | 1371575_at | 3.26 | 15.9 | 2.98 | 14.5 | 3.72 | 17.9 | 3.71 | 17.9 | 1.77 | 5.9 | 1.21 | 1.1 |
| 193 | 1371596_at | 1.29 | 4.2 | −1.03 | 0.2 | 1.05 | 0.3 | 1.03 | 0.2 | 1.04 | 0.3 | 1.02 | 0.1 |
| 194 | 1371600_at | −1.14 | 0.7 | 1.01 | 0 | 1.32 | 2.3 | 1.49 | 4 | 1.35 | 2.6 | 1.13 | 0.7 |
| 195 | 1371602_at | −1.74 | 9.2 | −1.25 | 2.3 | −1.02 | 0.1 | −1.12 | 0.8 | −1.07 | 0.5 | −1.07 | 0.4 |
| 196 | 1371618_s_at | 2.43 | 3.3 | 2.35 | 3.2 | 3.22 | 5.1 | 3.50 | 5.7 | 1.98 | 2.2 | 2.22 | 2.8 |
| 197 | 1371625_at | −2.41 | 11.9 | −1.83 | 7.1 | −1.54 | 4.3 | −1.76 | 6.4 | −1.11 | 0.5 | 1.07 | 0.3 |
| 198 | 1371632_at | −1.25 | 4.4 | −1.19 | 2.8 | −1.16 | 2.3 | −1.33 | 6.2 | −1.20 | 3.2 | −1.03 | 0.3 |
| 199 | 1371641_at | 1.49 | 7.1 | 1.16 | 1.6 | 1.11 | 1 | 1.11 | 0.9 | 1.03 | 0.2 | −1.01 | 0.1 |
| 200 | 1371691_at | 3.64 | 12.1 | 4.24 | 13.9 | 4.35 | 14.2 | 4.52 | 14.7 | 2.42 | 7.2 | 1.35 | 1.4 |
| 201 | 1371694_at | 1.25 | 2.1 | 1.31 | 2.8 | 1.47 | 4.7 | 1.56 | 6 | 1.50 | 5.2 | 1.04 | 0.2 |
| 202 | 1371727_at | 1.07 | 0.5 | 1.26 | 2.8 | 1.34 | 4.2 | 1.06 | 0.4 | 1.02 | 0.1 | −1.04 | 0.3 |
| 203 | 1371790_at | 1.54 | 6.2 | 1.30 | 2.9 | 1.19 | 1.6 | 1.06 | 0.4 | 1.02 | 0.1 | 1.21 | 1.7 |
| 204 | 1371824_at | 2.13 | 6.2 | 1.31 | 1.3 | 1.45 | 2.1 | 1.28 | 1.1 | 1.16 | 0.6 | −1.00 | 0 |
| 205 | 1371840_at | 1.34 | 0.6 | 1.38 | 0.7 | 2.52 | 3.6 | 2.95 | 4.6 | 1.44 | 0.9 | 1.32 | 0.6 |
| 206 | 1371849_at | −1.43 | 2.5 | −1.45 | 2.6 | −1.61 | 3.9 | −1.54 | 3.3 | −1.02 | 0.1 | −1.16 | 0.7 |
| 207 | 1371894_at | −1.20 | 2.7 | 1.12 | 1.2 | 1.27 | 4 | 1.32 | 5 | 1.21 | 2.9 | 1.11 | 1.2 |
| 208 | 1371924_at | −2.59 | 15.4 | −1.28 | 2.2 | 1.22 | 1.6 | 1.35 | 2.9 | 1.62 | 6.1 | 1.05 | 0.2 |
| 209 | 1371926_at | 1.46 | 8.3 | 1.39 | 6.9 | 1.41 | 7.3 | 1.42 | 7.4 | 1.21 | 2.9 | 1.15 | 1.8 |
| 210 | 1371951_at | −1.06 | 0.1 | 1.33 | 0.7 | 1.95 | 2.7 | 2.65 | 4.9 | 1.58 | 1.5 | 1.20 | 0.4 |
| 211 | 1371986_at | −1.85 | 10.9 | −1.76 | 9.8 | −1.65 | 8.3 | −1.33 | 3.6 | −1.20 | 1.7 | 1.04 | 0.2 |
| 212 | 1371988_at | 1.36 | 2 | 1.44 | 2.7 | 1.72 | 4.8 | 2.02 | 7.1 | 1.13 | 0.5 | 1.12 | 0.5 |
| 213 | 1371999_at | 1.03 | 0.2 | −1.30 | 4.3 | −1.11 | 1 | −1.16 | 1.8 | 1.07 | 0.6 | 1.15 | 1.6 |
| 214 | 1372005_at | 1.06 | 0.2 | 1.22 | 0.9 | 1.39 | 1.7 | 1.40 | 1.8 | 1.40 | 1.9 | 1.08 | 0.3 |
| 215 | 1372006_at | −1.36 | 2.2 | 1.05 | 0.2 | 1.81 | 6.2 | 2.32 | 10.2 | 1.29 | 1.7 | −1.27 | 1.6 |
| 216 | 1372028_at | 1.51 | 7.7 | −1.09 | 0.7 | 1.09 | 0.8 | 1.15 | 1.4 | 1.25 | 2.9 | 1.04 | 0.2 |
| 217 | 1372031_at | 3.04 | 12.9 | 2.64 | 10.8 | 3.19 | 13.6 | 3.21 | 13.7 | 2.06 | 7.2 | 1.13 | 0.5 |
| 218 | 1372050_at | 1.36 | 2.4 | 1.50 | 3.8 | 1.54 | 4.1 | 1.84 | 7 | 1.50 | 3.7 | 1.16 | 0.8 |
| 219 | 1372068_at | −1.47 | 6.2 | −1.38 | 4.8 | −1.25 | 2.7 | −1.12 | 1 | 1.08 | 0.5 | 1.11 | 0.9 |
| 220 | 1372084_at | −2.69 | 7.2 | −1.16 | 0.5 | 1.35 | 1.2 | 1.77 | 3.1 | 1.35 | 1.1 | 1.07 | 0.2 |
| 221 | 1372084_at | −2.69 | 7.2 | −1.16 | 0.5 | 1.35 | 1.2 | 1.77 | 3.1 | 1.35 | 1.1 | 1.07 | 0.2 |
| 222 | 1372088_at | −1.53 | 5.8 | −1.42 | 4.2 | −1.19 | 1.4 | −1.12 | 0.8 | 1.12 | 0.7 | −1.03 | 0.2 |
| 223 | 1372101_at | 1.22 | 1.3 | −1.04 | 0.1 | 1.63 | 5.4 | 1.60 | 5.1 | 1.29 | 2 | 1.24 | 1.5 |
| 224 | 1372104_at | −1.32 | 2.3 | −1.48 | 3.9 | 1.03 | 0.1 | 1.38 | 2.8 | 1.10 | 0.5 | 1.05 | 0.2 |
| 225 | 1372111_at | −1.45 | 4.7 | 1.03 | 0.2 | 1.73 | 8.2 | 1.60 | 6.6 | 1.13 | 0.9 | 1.25 | 2.1 |
| 226 | 1372127_at | 1.28 | 5.3 | 1.02 | 0.2 | 1.07 | 0.8 | 1.14 | 2 | 1.02 | 0.2 | −1.03 | 0.3 |
| 227 | 1372136_at | 1.30 | 8.9 | 1.26 | 7.3 | 1.41 | 12.6 | 1.27 | 7.8 | −1.04 | 0.5 | 1.01 | 0.1 |
| 228 | 1372164_at | 1.45 | 6.5 | 1.44 | 6.4 | 1.82 | 12.5 | 1.68 | 10.4 | 1.25 | 3 | 1.25 | 2.9 |
| 229 | 1372213_at | −1.30 | 1.6 | −1.15 | 0.6 | 1.53 | 3.4 | 1.71 | 4.8 | −1.05 | 0.2 | 1.07 | 0.2 |
| 230 | 1372223_at | −1.61 | 6.9 | −1.49 | 5.3 | −1.37 | 3.7 | −1.40 | 4.2 | −1.15 | 1.1 | 1.03 | 0.2 |
| 231 | 1372234_at | −1.52 | 3.5 | −1.24 | 1.3 | 1.07 | 0.3 | 1.27 | 1.5 | 1.25 | 1.4 | 1.11 | 0.5 |
| 232 | 1372251_at | −1.22 | 5.4 | −1.21 | 4.9 | −1.12 | 2.2 | −1.02 | 0.2 | 1.05 | 0.6 | 1.09 | 1.4 |
| 233 | 1372254_at | 1.78 | 5.5 | 2.19 | 8.6 | 3.00 | 13.5 | 3.22 | 14.6 | 1.98 | 7.1 | 1.57 | 3.8 |
| 234 | 1372256_at | 1.53 | 1.9 | 1.94 | 3.8 | 2.13 | 4.7 | 2.02 | 4.2 | 1.69 | 2.6 | 1.16 | 0.4 |
| 235 | 1372266_at | −1.65 | 3.9 | −1.68 | 4.1 | −1.23 | 1 | −1.13 | 0.5 | 1.01 | 0 | 1.11 | 0.4 |
| 236 | 1372294_at | −1.48 | 3.5 | 1.15 | 0.7 | 2.39 | 11.4 | 2.83 | 14.2 | 1.87 | 7.2 | 1.06 | 0.2 |
| 237 | 1372311_at | 1.25 | 3.3 | 1.31 | 4.4 | 1.28 | 3.8 | 1.24 | 3 | 1.08 | 0.6 | −1.07 | 0.5 |
| 238 | 1372326_at | 1.20 | 1 | 1.09 | 0.3 | 1.57 | 3.8 | 1.69 | 4.8 | 1.14 | 0.6 | −1.07 | 0.2 |
| 239 | 1372410_at | −1.14 | 0.7 | 1.02 | 0.1 | 1.47 | 3.2 | 1.61 | 4.4 | 1.31 | 1.8 | −1.10 | 0.4 |
| 240 | 1372439_at | 1.63 | 3.9 | 1.87 | 5.8 | 4.47 | 18.2 | 5.51 | 20.9 | 2.00 | 6.7 | 1.08 | 0.3 |
| 241 | 1372466_at | 1.00 | 0 | −1.02 | 0.1 | 1.41 | 5.7 | 1.43 | 6.1 | 1.42 | 6 | 1.19 | 2.1 |
| 242 | 1372518_at | 1.49 | 3 | 1.51 | 3.2 | 1.36 | 2 | 1.86 | 5.9 | 1.02 | 0 | 1.13 | 0.6 |
| 243 | 1372549_at | −1.05 | 0.3 | 1.19 | 1.4 | 1.47 | 4.7 | 1.49 | 5 | −1.01 | 0 | −1.16 | 1.1 |
| 244 | 1372569_at | 2.48 | 15.5 | 2.31 | 14 | 2.48 | 15.4 | 2.41 | 14.9 | 1.31 | 2.7 | 1.06 | 0.3 |
| 245 | 1372579_at | 2.28 | 4.7 | 1.92 | 3.3 | 2.34 | 4.9 | 2.28 | 4.7 | 1.36 | 1.1 | −1.00 | 0 |
| 246 | 1372585_at | −1.71 | 2.9 | 1.08 | 0.2 | 1.30 | 1 | 1.28 | 0.9 | 1.12 | 0.3 | 1.04 | 0.1 |
| 247 | 1372587_at | −1.13 | 0.5 | −1.06 | 0.2 | 1.77 | 4.5 | 1.74 | 4.3 | 1.36 | 1.7 | 1.05 | 0.1 |
| 248 | 1372593_at | −1.34 | 3.6 | −1.46 | 5.3 | −1.41 | 4.6 | −1.16 | 1.3 | 1.16 | 1.2 | 1.08 | 0.5 |
| 249 | 1372610_at | −1.36 | 3.4 | −1.09 | 0.5 | 1.06 | 0.3 | 1.30 | 2.7 | 1.52 | 5.4 | 1.12 | 0.8 |
| 250 | 1372613_at | −3.28 | 9.9 | −2.16 | 5.3 | −1.18 | 0.5 | −1.23 | 0.7 | 1.35 | 1.3 | 1.14 | 0.4 |
| 251 | 1372615_at | −2.44 | 8.2 | −1.77 | 4.2 | 1.08 | 0.3 | 1.06 | 0.2 | 1.31 | 1.3 | 1.19 | 0.7 |
| 252 | 1372638_at | 1.10 | 0.6 | 1.27 | 2.1 | 1.62 | 6.3 | 1.44 | 4.2 | 1.14 | 0.9 | 1.05 | 0.3 |
| 253 | 1372727_at | 1.20 | 0.9 | 1.32 | 1.6 | 1.91 | 6.1 | 1.78 | 5.1 | 1.32 | 1.6 | 1.12 | 0.5 |
| 254 | 1372729_at | 2.45 | 8.7 | 1.75 | 4.3 | 1.87 | 5.2 | 1.71 | 4.1 | 1.09 | 0.3 | 1.04 | 0.1 |
| 255 | 1372761_at | 1.21 | 0.5 | 1.63 | 2.2 | 2.29 | 5 | 2.34 | 5.1 | 1.57 | 1.9 | 1.47 | 1.5 |
| 256 | 1372769_at | 1.34 | 4 | 1.28 | 3 | 1.17 | 1.5 | 1.18 | 1.6 | 1.14 | 1.1 | −1.00 | 0 |
| 257 | 1372776_at | 1.70 | 9.9 | 1.46 | 6.1 | 1.30 | 3.4 | 1.29 | 3.2 | 1.11 | 0.8 | 1.07 | 0.5 |
| 258 | 1372818_at | −1.32 | 2.3 | −1.14 | 0.7 | 1.35 | 2.5 | 1.52 | 4.3 | 1.44 | 3.4 | 1.28 | 1.8 |
| 259 | 1372835_at | 1.05 | 0.2 | −1.16 | 0.6 | 1.72 | 4 | 1.85 | 4.8 | 1.04 | 0.1 | 1.63 | 3.4 |
| 260 | 1372836_at | 1.09 | 0.7 | 1.07 | 0.4 | 1.15 | 1.2 | 1.24 | 2.4 | 1.06 | 0.3 | 1.04 | 0.2 |
| 261 | 1372838_at | −1.55 | 8.4 | −1.52 | 7.9 | −1.35 | 4.7 | −1.17 | 1.8 | −1.07 | 0.5 | −1.10 | 0.9 |
| 262 | 1372844_at | 1.31 | 1.6 | 1.54 | 3.2 | 2.28 | 8.5 | 1.91 | 6 | 1.23 | 1 | −1.00 | 0 |
| 263 | 1372861_at | −1.35 | 3.1 | −1.08 | 0.5 | 1.20 | 1.4 | 1.25 | 2 | 1.11 | 0.6 | −1.10 | 0.6 |
| 264 | 1372919_at | 1.32 | 3.3 | 1.13 | 0.9 | 1.15 | 1.2 | 1.23 | 2.1 | 1.21 | 1.8 | 1.11 | 0.8 |
| 265 | 1372947_at | −1.40 | 6.1 | −1.22 | 2.8 | −1.13 | 1.3 | −1.06 | 0.5 | 1.04 | 0.3 | −1.05 | 0.4 |
| 266 | 1373000_at | −2.50 | 8.8 | −1.13 | 0.5 | 1.63 | 3.5 | 2.63 | 9.5 | 3.10 | 11.8 | 1.69 | 3.8 |
| 267 | 1373062_at | 2.09 | 5.4 | 1.49 | 2.1 | 1.75 | 3.5 | 2.62 | 8 | 1.54 | 2.3 | 1.22 | 0.7 |
| 268 | 1373079_at | −1.61 | 3.6 | −1.82 | 5.1 | −1.32 | 1.6 | −1.18 | 0.7 | −1.03 | 0.1 | 1.11 | 0.4 |
| 269 | 1373102_at | −1.17 | 0.7 | 1.24 | 1 | 1.62 | 3.6 | 1.60 | 3.5 | 1.10 | 0.4 | −1.06 | 0.2 |
| 270 | 1373140_at | 1.85 | 9.1 | 2.14 | 12.2 | 2.41 | 14.6 | 2.80 | 17.5 | 2.11 | 11.9 | 1.51 | 5 |
| 271 | 1373151_at | −2.26 | 9.5 | −1.45 | 2.9 | 1.07 | 0.3 | 1.11 | 0.5 | 1.16 | 0.8 | 1.19 | 0.9 |
| 272 | 1373180_at | 1.04 | 0.2 | 1.16 | 1 | 1.20 | 1.3 | 1.28 | 2.1 | 1.12 | 0.7 | 1.10 | 0.5 |
| 273 | 1373181_at | −1.27 | 1.5 | −1.06 | 0.2 | 1.53 | 3.6 | 1.61 | 4.3 | 1.13 | 0.6 | 1.17 | 0.8 |
| 274 | 1373217_at | −1.72 | 9.1 | −1.31 | 3.2 | −1.24 | 2.2 | −1.16 | 1.3 | −1.02 | 0.1 | −1.01 | 0.1 |
| 275 | 1373222_at | −1.43 | 4.2 | −1.25 | 2 | 1.05 | 0.3 | 1.22 | 1.7 | 1.35 | 3.1 | 1.17 | 1.2 |
| 276 | 1373245_at | 1.61 | 3.3 | 2.11 | 6.5 | 5.29 | 18.5 | 6.08 | 20.2 | 2.49 | 8.7 | 1.20 | 0.8 |
| 277 | 1373258_at | −2.13 | 9.1 | −1.47 | 3.3 | −1.15 | 0.7 | 1.06 | 0.2 | 1.18 | 0.9 | 1.21 | 1.1 |
| 278 | 1373286_at | 3.06 | 12 | 2.85 | 11 | 4.02 | 15.6 | 4.27 | 16.4 | 1.83 | 5 | 1.30 | 1.4 |
| 279 | 1373301_at | −1.60 | 8.1 | −1.43 | 5.4 | −1.13 | 1 | −1.08 | 0.6 | 1.04 | 0.3 | 1.06 | 0.4 |
| 280 | 1373310_at | −1.34 | 2.5 | −1.25 | 1.6 | −1.42 | 3.2 | −1.28 | 1.9 | −1.04 | 0.2 | −1.00 | 0 |
| 281 | 1373346_at | −1.49 | 7.3 | −1.04 | 0.3 | 1.12 | 1.1 | 1.15 | 1.4 | 1.18 | 1.8 | 1.16 | 1.5 |
| 282 | 1373363_at | −1.13 | 0.5 | 1.01 | 0 | 1.37 | 2.1 | 1.65 | 4.3 | 1.44 | 2.6 | 1.22 | 1.1 |
| 283 | 1373374_at | −1.67 | 9.1 | −1.50 | 6.5 | −1.36 | 4.3 | −1.49 | 6.4 | −1.03 | 0.1 | 1.05 | 0.3 |
| 284 | 1373379_at | 1.25 | 4.9 | 1.16 | 2.5 | 1.25 | 4.9 | 1.13 | 2 | 1.04 | 0.4 | 1.06 | 0.7 |
| 285 | 1373401_at | 3.34 | 4.9 | 4.09 | 6.2 | 6.01 | 8.8 | 10.75 | 12.8 | 2.84 | 3.9 | 1.21 | 0.3 |
| 286 | 1373415_at | 1.22 | 1.2 | 1.15 | 0.7 | 1.31 | 1.9 | 1.40 | 2.6 | 1.28 | 1.6 | 1.03 | 0.1 |
| 287 | 1373463_at | −2.62 | 15.3 | −1.45 | 4 | 1.11 | 0.6 | 1.56 | 5.3 | 1.75 | 7.4 | −1.15 | 0.9 |
| 288 | 1373466_at | −1.58 | 7 | −1.46 | 5.3 | −1.47 | 5.4 | −1.58 | 6.9 | −1.25 | 2.3 | −1.05 | 0.3 |
| 289 | 1373483_at | −1.43 | 1.9 | −1.07 | 0.2 | 1.53 | 2.4 | 1.62 | 2.9 | 1.29 | 1.1 | 1.16 | 0.5 |
| 290 | 1373487_at | −1.72 | 3 | −1.39 | 1.4 | 1.07 | 0.2 | 1.16 | 0.5 | 1.14 | 0.4 | −1.14 | 0.4 |
| 291 | 1373488_at | 1.23 | 2.9 | 1.10 | 0.9 | 1.20 | 2.3 | 1.13 | 1.3 | 1.00 | 0 | 1.14 | 1.4 |
| 292 | 1373515_at | −1.47 | 4.4 | −1.47 | 4.4 | −1.38 | 3.3 | −1.26 | 2 | −1.13 | 0.8 | −1.02 | 0.1 |
| 293 | 1373533_at | −1.75 | 5 | −1.41 | 2.3 | −1.07 | 0.2 | 1.03 | 0.1 | 1.02 | 0.1 | 1.06 | 0.2 |
| 294 | 1373554_at | 2.59 | 5.1 | 2.38 | 4.4 | 2.42 | 4.5 | 3.79 | 8.3 | 3.53 | 7.7 | 2.10 | 3.4 |
| 295 | 1373557_at | 2.34 | 12.2 | 1.67 | 6 | 1.43 | 3.4 | 1.08 | 0.4 | 1.23 | 1.5 | 1.01 | 0.1 |
| 296 | 1373590_at | 2.23 | 8.8 | 1.67 | 4.5 | 1.71 | 4.9 | 1.51 | 3.3 | 1.38 | 2.2 | 1.18 | 0.8 |
| 297 | 1373592_at | −1.23 | 1 | −1.09 | 0.3 | 1.52 | 3.1 | 1.44 | 2.5 | 1.03 | 0.1 | 1.14 | 0.6 |
| 298 | 1373599_at | −1.60 | 4.6 | −1.27 | 1.6 | 1.12 | 0.6 | 1.18 | 0.9 | 1.39 | 2.7 | 1.28 | 1.8 |
| 299 | 1373615_at | 1.01 | 0 | −1.10 | 0.3 | 1.34 | 1.4 | 1.56 | 2.6 | 1.05 | 0.1 | 1.08 | 0.2 |
| 300 | 1373628_at | 1.09 | 0.3 | 1.12 | 0.3 | 1.67 | 3 | 2.07 | 5.3 | 2.32 | 6.5 | 1.17 | 0.5 |
| 301 | 1373661_a_at | 4.70 | 10.8 | 7.00 | 14.4 | 8.08 | 15.7 | 9.02 | 16.6 | 3.01 | 6.7 | 2.08 | 3.3 |
| 302 | 1373666_at | 1.13 | 1 | 1.16 | 1.2 | 1.21 | 1.9 | 1.49 | 5.8 | −1.01 | 0 | 1.12 | 0.9 |
| 303 | 1373683_at | 1.53 | 3.3 | 1.08 | 0.3 | 1.41 | 2.4 | 1.66 | 4.4 | −1.03 | 0.1 | −1.01 | 0 |
| 304 | 1373695_at | −1.15 | 1.5 | −1.19 | 2.1 | −1.06 | 0.4 | −1.12 | 1.2 | −1.02 | 0.2 | −1.00 | 0 |
| 305 | 1373696_at | −2.89 | 5.3 | −2.28 | 3.6 | −1.43 | 1 | −1.29 | 0.6 | 1.05 | 0.1 | −1.06 | 0.1 |
| 306 | 1373740_at | −5.03 | 7.6 | −4.43 | 6.7 | −2.74 | 3.7 | −1.87 | 1.8 | −1.04 | 0.1 | 1.34 | 0.6 |
| 307 | 1373751_at | 1.51 | 3.7 | 1.78 | 6.2 | 1.67 | 5.2 | 1.85 | 6.8 | 1.29 | 1.8 | −1.06 | 0.2 |
| 308 | 1373829_at | −1.52 | 6.3 | −1.64 | 8.1 | −1.67 | 8.5 | −1.59 | 7.4 | −1.27 | 2.7 | −1.04 | 0.2 |
| 309 | 1373847_at | 1.16 | 0.7 | 1.28 | 1.4 | 2.14 | 7.8 | 2.47 | 10 | 1.78 | 5.2 | 1.40 | 2.3 |
| 310 | 1373874_at | 1.12 | 0.9 | −1.05 | 0.3 | −1.08 | 0.5 | −1.16 | 1.3 | −1.07 | 0.4 | 1.02 | 0.1 |
| 311 | 1373881_at | 1.15 | 2.2 | 1.33 | 6.3 | 1.37 | 7.3 | 1.37 | 7.4 | 1.40 | 8.2 | 1.13 | 1.7 |
| 312 | 1373911_at | −2.10 | 3.5 | 1.54 | 1.6 | 2.34 | 4.6 | 3.81 | 9.1 | 1.84 | 2.8 | −1.30 | 0.8 |
| 313 | 1373957_at | −1.55 | 1.9 | −1.94 | 3.7 | −1.54 | 1.9 | −1.09 | 0.2 | 1.08 | 0.2 | −1.15 | 0.4 |
| 314 | 1373970_at | 1.23 | 1 | 1.16 | 0.6 | 1.16 | 0.6 | 1.41 | 2.2 | 1.00 | 0 | 1.09 | 0.3 |
| 315 | 1374013_at | −1.91 | 8.6 | −1.33 | 2.4 | 1.37 | 2.9 | 1.77 | 7.1 | 2.06 | 9.9 | 1.23 | 1.5 |
| 316 | 1374034_at | 2.22 | 12 | 1.79 | 7.8 | 1.83 | 8.2 | 1.24 | 1.7 | 1.39 | 3.2 | 1.33 | 2.6 |
| 317 | 1374057_at | 1.14 | 0.5 | 1.11 | 0.4 | 1.58 | 3.2 | 1.93 | 5.6 | 1.21 | 0.8 | −1.02 | 0.1 |
| 318 | 1374061_at | 1.82 | 5.5 | 2.14 | 7.9 | 2.00 | 6.9 | 2.26 | 8.7 | 1.65 | 4.2 | 1.63 | 4 |
| 319 | 1374104_at | −3.02 | 7.9 | −1.76 | 2.9 | −1.20 | 0.5 | 1.65 | 2.4 | 1.39 | 1.3 | 1.13 | 0.3 |
| 320 | 1374118_at | 1.32 | 5.2 | 1.16 | 2 | −1.01 | 0.1 | 1.02 | 0.2 | −1.08 | 0.8 | 1.05 | 0.4 |
| 321 | 1374129_at | −1.10 | 0.5 | 1.03 | 0.1 | 1.34 | 2.6 | 1.53 | 4.7 | 1.04 | 0.2 | −1.06 | 0.3 |
| 322 | 1374134_at | 1.58 | 5.7 | 1.44 | 4 | 1.81 | 8.3 | 1.83 | 8.6 | 1.36 | 3.1 | 1.09 | 0.5 |
| 323 | 1374171_at | 1.02 | 0 | 1.28 | 0.4 | 1.78 | 1.4 | 1.69 | 1.2 | 1.34 | 0.5 | 1.18 | 0.3 |
| 324 | 1374172_at | −2.29 | 9.3 | −1.21 | 1 | 1.16 | 0.7 | 1.31 | 1.7 | 2.18 | 8.5 | 1.29 | 1.6 |
| 325 | 1374176_at | −1.04 | 0.1 | 1.74 | 4.7 | 2.14 | 7.5 | 2.21 | 8 | 1.60 | 3.7 | 1.59 | 3.6 |
| 326 | 1374207_at | 1.25 | 0.9 | 1.02 | 0 | 2.13 | 5.8 | 2.42 | 7.3 | 1.30 | 1.2 | 1.15 | 0.5 |
| 327 | 1374247_at | 1.22 | 0.7 | 1.93 | 4 | 3.04 | 8.7 | 3.74 | 11 | 1.83 | 3.5 | 1.18 | 0.5 |
| 328 | 1374266_at | −1.55 | 4.7 | −1.32 | 2.3 | −1.44 | 3.5 | −1.69 | 6.1 | −1.21 | 1.3 | −1.20 | 1.2 |
| 329 | 1374273_at | −1.51 | 5.7 | −1.51 | 5.7 | −1.60 | 6.9 | −1.45 | 4.9 | −1.05 | 0.3 | 1.11 | 0.8 |
| 330 | 1374276_at | −1.49 | 2.4 | −1.61 | 3.2 | −1.38 | 1.7 | −1.20 | 0.7 | −1.34 | 1.5 | −1.20 | 0.8 |
| 331 | 1374284_at | 1.11 | 0.4 | 1.06 | 0.2 | −1.30 | 1.3 | −1.02 | 0 | 1.11 | 0.4 | 1.06 | 0.2 |
| 332 | 1374285_at | −1.37 | 4.6 | −1.60 | 8.3 | −1.45 | 5.9 | −1.21 | 2.1 | 1.04 | 0.2 | 1.12 | 0.9 |
| 333 | 1374306_at | 1.76 | 10.4 | 1.48 | 6.1 | 1.36 | 4.3 | 1.39 | 4.7 | 1.07 | 0.5 | 1.24 | 2.5 |
| 334 | 1374320_at | 1.18 | 0.5 | 1.02 | 0 | −1.09 | 0.2 | 1.02 | 0.1 | −1.77 | 3.1 | −1.27 | 0.9 |
| 335 | 1374399_at | 1.35 | 1.9 | 1.22 | 1 | 1.59 | 3.7 | 1.32 | 1.7 | −1.09 | 0.3 | 1.05 | 0.2 |
| 336 | 1374474_at | 1.43 | 7.6 | 1.36 | 6.2 | 1.30 | 4.9 | 1.29 | 4.6 | 1.01 | 0 | −1.04 | 0.3 |
| 337 | 1374477_at | 1.10 | 0.4 | 1.18 | 0.8 | 1.74 | 4.9 | 1.64 | 4.1 | 1.13 | 0.5 | 1.07 | 0.2 |
| 338 | 1374479_at | −1.16 | 1.2 | −1.33 | 3.3 | −1.41 | 4.4 | −1.47 | 5.3 | −1.14 | 1 | −1.01 | 0.1 |
| 339 | 1374529_at | 2.16 | 8.2 | 2.00 | 7.1 | 2.17 | 8.3 | 2.67 | 11.4 | 3.20 | 14.2 | 1.69 | 4.6 |
| 340 | 1374531_at | −1.32 | 1.5 | 1.02 | 0.1 | −1.14 | 0.5 | 1.05 | 0.1 | 1.27 | 1.2 | 1.14 | 0.5 |
| 341 | 1374575_at | −1.93 | 5.1 | −1.63 | 3.2 | −1.17 | 0.6 | 1.02 | 0.1 | −1.11 | 0.4 | −1.07 | 0.2 |
| 342 | 1374586_at | −1.23 | 1.4 | −1.28 | 1.9 | −1.17 | 1 | 1.14 | 0.7 | 1.06 | 0.3 | 1.31 | 2.2 |
| 343 | 1374626_at | 7.25 | 12.4 | 6.07 | 11 | 6.03 | 11 | 4.79 | 9.1 | 1.64 | 1.6 | 1.31 | 0.7 |
| 344 | 1374635_at | −2.42 | 1.9 | −1.84 | 1.1 | −1.30 | 0.4 | −1.01 | 0 | 1.03 | 0 | 1.12 | 0.1 |
| 345 | 1374678_at | −1.09 | 0.4 | −1.17 | 0.9 | −1.62 | 4.9 | −2.01 | 8.5 | −1.26 | 1.6 | −1.04 | 0.2 |
| 346 | 1374742_at | −1.09 | 0.5 | −1.25 | 1.7 | −1.22 | 1.4 | −1.06 | 0.3 | −1.08 | 0.4 | −1.79 | 7.5 |
| 347 | 1374771_at | −1.22 | 1.2 | −1.04 | 0.1 | 1.58 | 4.6 | 1.56 | 4.4 | 1.19 | 1 | 1.01 | 0 |
| 348 | 1374779_at | 1.20 | 0.4 | 1.53 | 1.4 | 1.30 | 0.7 | 1.20 | 0.4 | 1.09 | 0.2 | −1.19 | 0.4 |
| 349 | 1374816_at | −1.85 | 10 | −1.18 | 1.4 | 1.13 | 0.9 | −1.04 | 0.2 | −1.12 | 0.8 | −1.16 | 1.2 |
| 350 | 1374849_at | 1.06 | 0.2 | 1.21 | 0.6 | 1.10 | 0.3 | 1.43 | 1.5 | 1.00 | 0 | −1.15 | 0.4 |
| 351 | 1374868_at | −2.39 | 9.4 | −2.37 | 9.3 | −1.71 | 4.6 | −1.20 | 0.9 | 1.24 | 1.1 | 1.08 | 0.3 |
| 352 | 1374897_at | 1.80 | 9.6 | 1.30 | 2.9 | 1.27 | 2.5 | 1.10 | 0.6 | 1.02 | 0.1 | −1.10 | 0.7 |
| 353 | 1374953_at | 5.84 | 5 | 5.33 | 4.6 | 5.97 | 5 | 7.55 | 6.1 | 3.84 | 3.3 | 3.68 | 3.1 |
| 354 | 1375028_at | 1.50 | 5.5 | 1.43 | 4.6 | 1.10 | 0.7 | −1.30 | 2.9 | −1.10 | 0.6 | −1.19 | 1.5 |
| 355 | 1375051_at | 2.50 | 11 | 1.75 | 5.5 | 1.81 | 5.9 | 1.72 | 5.2 | 1.09 | 0.4 | −1.22 | 1.1 |
| 356 | 1375074_at | −1.03 | 0.1 | −1.15 | 0.8 | 1.27 | 1.7 | 1.26 | 1.6 | 1.15 | 0.8 | 1.06 | 0.2 |
| 357 | 1375138_at | −2.44 | 6.9 | −2.46 | 7 | −2.09 | 5.2 | −1.93 | 4.4 | −1.06 | 0.2 | 1.04 | 0.1 |
| 358 | 1375144_at | −1.64 | 5.2 | −1.26 | 1.6 | 1.21 | 1.2 | 1.28 | 1.7 | 1.59 | 4.7 | 1.24 | 1.5 |
| 359 | 1375267_at | 1.03 | 0.1 | 1.06 | 0.2 | 1.95 | 7.1 | 2.15 | 8.6 | 1.76 | 5.5 | −1.01 | 0 |
| 360 | 1375270_at | −1.63 | 7.9 | −1.53 | 6.4 | −1.20 | 1.7 | −1.34 | 3.7 | −1.05 | 0.3 | −1.11 | 0.8 |
| 361 | 1375277_at | 1.03 | 0.1 | −1.21 | 1.4 | −1.08 | 0.4 | −1.22 | 1.5 | −1.06 | 0.3 | −1.28 | 2 |
| 362 | 1375337_at | 1.76 | 5.7 | 1.76 | 5.8 | 1.63 | 4.6 | 1.89 | 6.9 | 1.11 | 0.5 | −1.00 | 0 |
| 363 | 1375368_at | 1.46 | 5.4 | 1.15 | 1.1 | 1.18 | 1.6 | 1.12 | 0.8 | 1.09 | 0.6 | 1.09 | 0.6 |
| 364 | 1375369_at | 1.28 | 3.2 | 1.30 | 3.4 | 1.46 | 6.1 | 1.48 | 6.4 | 1.22 | 2.3 | 1.21 | 2.1 |
| 365 | 1375377_at | 1.24 | 1.5 | 1.17 | 1 | −1.12 | 0.6 | 1.17 | 1 | −1.07 | 0.3 | −1.04 | 0.1 |
| 366 | 1375420_at | 1.21 | 0.7 | 1.57 | 2.5 | 3.13 | 9.9 | 2.91 | 9 | 1.18 | 0.6 | 1.01 | 0 |
| 367 | 1375424_at | 1.34 | 4.4 | 1.30 | 3.8 | 1.30 | 3.8 | 1.25 | 2.9 | −1.08 | 0.6 | −1.03 | 0.2 |
| 368 | 1375463_at | −1.17 | 1.7 | −1.02 | 0.1 | 1.19 | 2 | 1.09 | 0.8 | 1.16 | 1.6 | 1.09 | 0.8 |
| 369 | 1375473_at | 1.03 | 0.1 | −1.13 | 0.6 | 1.15 | 0.7 | 1.42 | 2.7 | 1.26 | 1.4 | −1.01 | 0 |
| 370 | 1375657_at | 1.40 | 3.3 | 1.34 | 2.6 | 1.66 | 6.2 | 1.64 | 5.9 | 1.18 | 1.1 | 1.11 | 0.6 |
| 371 | 1375719_s_at | −1.15 | 0.2 | 1.11 | 0.1 | 3.59 | 4.1 | 3.32 | 3.7 | 2.22 | 2 | 1.03 | 0 |
| 372 | 1375721_at | −1.15 | 0.9 | −1.05 | 0.2 | 1.06 | 0.3 | 1.06 | 0.3 | 1.13 | 0.7 | 1.22 | 1.5 |
| 373 | 1375739_at | 1.23 | 2.7 | 1.26 | 3.2 | 1.51 | 7.6 | 1.38 | 5.3 | 1.02 | 0.1 | −1.02 | 0.1 |
| 374 | 1375862_at | −1.46 | 2 | 1.04 | 0.1 | 1.94 | 4.8 | 2.73 | 9 | 1.63 | 3 | −1.09 | 0.3 |
| 375 | 1375898_at | −1.13 | 0.7 | −1.14 | 0.8 | 1.10 | 0.5 | 1.14 | 0.8 | 1.05 | 0.2 | 1.19 | 1.2 |
| 376 | 1375951_at | 1.06 | 0.2 | −1.23 | 0.8 | 1.15 | 0.5 | 1.24 | 0.9 | 1.37 | 1.5 | 1.44 | 1.9 |
| 377 | 1376004_at | −1.16 | 1.5 | −1.41 | 5.5 | −1.21 | 2.2 | −1.03 | 0.2 | 1.16 | 1.5 | 1.17 | 1.6 |
| 378 | 1376045_at | 1.40 | 2.7 | 1.40 | 2.7 | 1.92 | 7.4 | 2.28 | 10.3 | 1.70 | 5.4 | 1.40 | 2.7 |
| 379 | 1376055_at | 1.51 | 4.8 | 1.33 | 2.8 | 1.23 | 1.6 | 1.08 | 0.4 | 1.25 | 1.8 | −1.13 | 0.8 |
| 380 | 1376099_at | −1.97 | 7.6 | −2.02 | 8 | −1.20 | 1.1 | 1.06 | 0.2 | 1.77 | 6 | −1.35 | 2.2 |
| 381 | 1376106_at | −1.08 | 0.2 | 1.20 | 0.6 | 1.45 | 1.7 | 1.95 | 4.2 | 1.50 | 1.9 | 1.63 | 2.6 |
| 382 | 1376128_at | −2.18 | 12.8 | −1.83 | 9.1 | −1.42 | 4 | −1.51 | 5.2 | −1.04 | 0.2 | −1.05 | 0.2 |
| 383 | 1376182_at | −1.30 | 2.7 | −1.23 | 1.9 | −1.14 | 0.9 | 1.09 | 0.5 | 1.15 | 1.1 | 1.07 | 0.4 |
| 384 | 1376265_at | 1.89 | 6.8 | 1.63 | 4.5 | 1.44 | 2.9 | 1.62 | 4.4 | 1.28 | 1.6 | 1.09 | 0.4 |
| 385 | 1376570_at | 1.58 | 9.9 | 1.34 | 5.2 | 1.27 | 3.8 | 1.19 | 2.3 | 1.18 | 2.1 | 1.04 | 0.3 |
| 386 | 1376574_at | 1.87 | 4.7 | 1.73 | 3.8 | 3.52 | 12.7 | 3.46 | 12.4 | 1.28 | 1.2 | 1.22 | 0.9 |
| 387 | 1376583_at | −1.23 | 3.2 | −1.00 | 0 | 1.13 | 1.4 | 1.18 | 2.2 | 1.08 | 0.7 | −1.12 | 1.3 |
| 388 | 1376624_at | 2.40 | 8.7 | 2.07 | 6.7 | 2.06 | 6.6 | 1.92 | 5.7 | 1.26 | 1.2 | −1.05 | 0.2 |
| 389 | 1376631_at | 1.29 | 2.3 | 1.10 | 0.6 | 1.26 | 2 | 1.30 | 2.4 | 1.12 | 0.7 | −1.07 | 0.4 |
| 390 | 1376640_at | −1.02 | 0.1 | 1.22 | 1 | 2.11 | 7.9 | 2.00 | 7.1 | 1.57 | 3.7 | 1.15 | 0.6 |
| 391 | 1376655_at | −1.35 | 3.6 | −1.45 | 4.9 | −1.18 | 1.5 | −1.07 | 0.4 | −1.06 | 0.3 | −1.00 | 0 |
| 392 | 1376678_at | −1.15 | 0.3 | 1.11 | 0.2 | 1.54 | 1.6 | 1.74 | 2.4 | 1.34 | 0.9 | 1.01 | 0 |
| 393 | 1376754_at | 1.23 | 2.6 | 1.14 | 1.3 | 1.33 | 4.3 | 1.07 | 0.5 | 1.07 | 0.5 | −1.01 | 0.1 |
| 394 | 1376775_at | 1.31 | 1.3 | 1.70 | 3.5 | 2.17 | 6.2 | 2.13 | 6 | 1.51 | 2.4 | 1.15 | 0.5 |
| 395 | 1376786_as_at | −2.07 | 8.4 | −1.86 | 6.7 | −1.51 | 3.6 | −1.20 | 1 | −1.05 | 0.2 | −1.01 | 0 |
| 396 | 1376788_at | −1.64 | 6.3 | −1.58 | 5.6 | −1.39 | 3.4 | −1.28 | 2.1 | −1.31 | 2.5 | 1.10 | 0.6 |
| 397 | 1376848_at | −1.08 | 0.6 | −1.00 | 0 | 1.31 | 4 | 1.40 | 5.6 | 1.02 | 0.1 | 1.05 | 0.4 |
| 398 | 1376858_at | −1.65 | 9.6 | −1.66 | 9.8 | −1.06 | 0.4 | 1.10 | 0.8 | 1.20 | 2 | 1.08 | 0.6 |
| 399 | 1376919_at | −1.35 | 4.8 | 1.00 | 0 | −1.07 | 0.5 | −1.18 | 1.8 | 1.00 | 0 | 1.12 | 1.1 |
| 400 | 1377023_at | 2.16 | 1.5 | 3.30 | 3 | 3.80 | 3.6 | 4.22 | 4 | 1.98 | 1.3 | −1.24 | 0.3 |
| 401 | 1377369_at | −1.43 | 2.9 | −1.28 | 1.6 | −1.12 | 0.5 | −1.20 | 1 | 1.05 | 0.2 | −1.07 | 0.3 |
| 402 | 1377390_at | −2.27 | 11.2 | −1.92 | 8.2 | −1.83 | 7.3 | −1.71 | 6.2 | −1.33 | 2.4 | −1.15 | 0.8 |
| 403 | 1377595_at | −1.10 | 0.3 | −1.03 | 0.1 | −1.36 | 1.7 | −1.05 | 0.2 | −1.08 | 0.2 | 1.07 | 0.2 |
| 404 | 1377625_at | 1.67 | 4.3 | 1.23 | 1.1 | 1.20 | 0.9 | 1.12 | 0.5 | 1.06 | 0.2 | 1.01 | 0 |
| 405 | 1377630_at | 1.48 | 3.5 | 1.60 | 4.7 | 1.74 | 6 | 1.74 | 6 | 1.19 | 1 | 1.15 | 0.8 |
| 406 | 1377671_at | 10.42 | 21.1 | 7.69 | 18.3 | 6.08 | 15.9 | 4.97 | 13.8 | 1.74 | 3 | 1.09 | 0.2 |
| 407 | 1377697_at | −1.19 | 0.9 | −1.04 | 0.1 | 1.43 | 2.6 | 1.76 | 5.4 | 1.39 | 2.4 | 1.25 | 1.3 |
| 408 | 1377702_at | −1.34 | 3.9 | −1.16 | 1.3 | −1.16 | 1.4 | −1.25 | 2.5 | −1.23 | 2.2 | −1.10 | 0.7 |
| 409 | 1377723_at | −1.20 | 2.8 | −1.04 | 0.3 | −1.08 | 0.8 | 1.10 | 1.1 | 1.09 | 0.8 | 1.16 | 2 |
| 410 | 1377862_at | 1.20 | 0.4 | 1.06 | 0.1 | 1.83 | 2.3 | 2.35 | 4 | 1.12 | 0.2 | 1.27 | 0.6 |
| 411 | 1377869_at | 1.01 | 0.1 | 1.17 | 1.1 | 1.10 | 0.6 | 1.10 | 0.5 | 1.20 | 1.4 | 1.33 | 2.7 |
| 412 | 1377950_at | −1.46 | 0.8 | 1.33 | 0.5 | 1.11 | 0.1 | 1.89 | 1.7 | 1.47 | 0.8 | −1.08 | 0.1 |
| 413 | 1377994_at | 10.82 | 10.6 | 14.04 | 12.2 | 13.12 | 11.8 | 17.14 | 13.3 | 2.88 | 3.2 | 2.34 | 2.3 |
| 414 | 1378134_at | −1.20 | 1.6 | −1.32 | 3.1 | −1.24 | 2 | −1.17 | 1.3 | −1.05 | 0.2 | −1.11 | 0.7 |
| 415 | 1378282_at | 1.49 | 5.6 | 1.32 | 3.1 | 1.28 | 2.6 | 1.16 | 1.3 | 1.01 | 0 | 1.01 | 0 |
| 416 | 1378305_at | −1.15 | 0.3 | −1.17 | 0.3 | 1.71 | 1.9 | 2.03 | 3 | 1.82 | 2.3 | 1.48 | 1.2 |
| 417 | 1378321_at | −1.05 | 0.1 | 1.01 | 0 | −1.26 | 1 | −1.15 | 0.5 | −1.02 | 0.1 | −1.08 | 0.2 |
| 418 | 1378342_at | 1.57 | 1.4 | 1.95 | 2.5 | 2.43 | 3.9 | 2.76 | 4.8 | 1.83 | 2.2 | 1.26 | 0.5 |
| 419 | 1378440_at | 2.46 | 9.8 | 1.66 | 4.2 | 1.71 | 4.6 | 1.75 | 4.9 | 1.34 | 1.8 | 1.00 | 0 |
| 420 | 1378507_at | 1.54 | 4.1 | 1.38 | 2.6 | 1.34 | 2.3 | 1.12 | 0.5 | 1.24 | 1.4 | −1.10 | 0.4 |
| 421 | 1378526_at | −1.25 | 1.1 | −1.29 | 1.3 | 1.12 | 0.4 | 1.05 | 0.2 | −1.07 | 0.2 | 1.02 | 0 |
| 422 | 1378925_at | 8.81 | 12.7 | 3.84 | 6.5 | 4.00 | 6.8 | 2.78 | 4.3 | 1.79 | 1.8 | 1.50 | 1.1 |
| 423 | 1379294_at | −1.25 | 2.3 | 1.04 | 0.2 | 1.45 | 5.2 | 1.54 | 6.6 | 1.60 | 7.4 | 1.32 | 3.4 |
| 424 | 1379322_at | 1.38 | 5.8 | 1.17 | 1.9 | 1.15 | 1.5 | 1.17 | 1.9 | 1.02 | 0.1 | −1.00 | 0 |
| 425 | 1379331_at | −1.09 | 0.1 | 1.33 | 0.3 | 3.90 | 2.7 | 18.62 | 8.7 | 14.26 | 7.6 | −1.29 | 0.3 |
| 426 | 1379335_at | −1.28 | 5.1 | −1.24 | 4.1 | −1.23 | 3.7 | −1.16 | 2.2 | −1.04 | 0.3 | −1.00 | 0 |
| 427 | 1379345_at | 1.47 | 1.1 | 2.51 | 4.3 | 4.65 | 9.1 | 4.93 | 9.6 | 1.99 | 2.7 | −1.03 | 0 |
| 428 | 1379461_at | −1.47 | 4 | −1.30 | 2.1 | −1.33 | 2.5 | −1.30 | 2.1 | −1.12 | 0.6 | −1.05 | 0.2 |
| 429 | 1379497_at | 6.92 | 7.7 | 8.82 | 9.1 | 5.00 | 5.8 | 2.49 | 2.4 | 1.07 | 0.1 | 1.38 | 0.5 |
| 430 | 1379604_at | −1.10 | 0.5 | 1.28 | 1.8 | 1.71 | 6 | 1.76 | 6.4 | 1.40 | 2.9 | 1.16 | 0.9 |
| 431 | 1379772_at | −1.23 | 0.7 | 1.36 | 1.3 | 3.56 | 10.6 | 2.97 | 8.6 | 1.63 | 2.6 | 1.52 | 2 |
| 432 | 1379790_at | 1.22 | 0.9 | 1.28 | 1.3 | 1.57 | 3.3 | 1.58 | 3.3 | 1.34 | 1.7 | 1.24 | 1.1 |
| 433 | 1379799_at | −1.29 | 1.3 | 1.04 | 0.1 | 1.25 | 1.1 | 1.08 | 0.3 | 1.46 | 2.4 | 1.27 | 1.2 |
| 434 | 1379847_at | −1.24 | 0.6 | −1.00 | 0 | 1.28 | 0.8 | 1.54 | 1.7 | 1.10 | 0.2 | −1.08 | 0.2 |
| 435 | 1379882_a_at | −1.77 | 2 | −1.94 | 2.4 | −1.02 | 0 | 1.14 | 0.3 | 1.41 | 0.9 | 1.21 | 0.4 |
| 436 | 1379935_at | 20.88 | 11.3 | 13.58 | 9.2 | 11.66 | 8.5 | 8.24 | 6.8 | 1.33 | 0.4 | 1.18 | 0.2 |
| 437 | 1380089_at | −2.44 | 4.8 | −1.77 | 2.4 | −1.31 | 0.8 | 1.10 | 0.2 | 1.48 | 1.4 | 1.18 | 0.4 |
| 438 | 1380110_at | 3.57 | 13.3 | 2.55 | 8.9 | 2.77 | 10 | 2.58 | 9 | 1.34 | 1.5 | 1.11 | 0.3 |
| 439 | 1380318_at | 1.25 | 1.1 | 1.57 | 3.2 | 1.90 | 5.4 | 1.90 | 5.5 | 1.67 | 3.8 | 1.94 | 5.7 |
| 440 | 1380474_at | 1.02 | 0 | 1.49 | 1.1 | 2.37 | 3.4 | 3.11 | 5.2 | 1.36 | 0.7 | −1.03 | 0 |
| 441 | 1380617_at | 1.10 | 0.2 | 1.05 | 0.1 | 1.97 | 3.5 | 2.20 | 4.4 | 1.47 | 1.5 | 1.20 | 0.5 |
| 442 | 1380688_at | −2.44 | 9.5 | −2.27 | 8.5 | −1.73 | 4.7 | −1.50 | 2.9 | −1.0 | 10 | 1.17 | 0.7 |
| 443 | 1380728_at | 1.31 | 1.2 | 1.48 | 2.1 | 1.38 | 1.6 | 1.55 | 2.5 | 1.46 | 2 | 1.05 | 0.1 |
| 444 | 1380908_at | −1.03 | 0.1 | 1.16 | 0.5 | 1.19 | 0.6 | 1.42 | 1.7 | 1.40 | 1.7 | 1.09 | 0.2 |
| 445 | 1381190_at | −1.26 | 2.4 | 1.07 | 0.4 | −1.26 | 2.4 | −1.51 | 5.7 | −1.30 | 2.9 | −1.04 | 0.2 |
| 446 | 1381305_at | −1.11 | 0.8 | 1.07 | 0.4 | 1.39 | 4.3 | 1.81 | 10.2 | 1.82 | 10.2 | 1.27 | 2.6 |
| 447 | 1381311_at | 1.73 | 2 | 2.36 | 3.9 | 2.99 | 5.7 | 3.69 | 7.4 | 2.05 | 3 | 1.37 | 0.9 |
| 448 | 1381335_at | 1.68 | 1.8 | 1.87 | 2.4 | 1.34 | 0.8 | −1.08 | 0.1 | −1.13 | 0.3 | −1.07 | 0.1 |
| 449 | 1381410_a_at | −1.06 | 0.1 | 1.21 | 0.5 | 1.92 | 2.9 | 1.89 | 2.8 | 1.46 | 1.3 | 1.37 | 1 |
| 450 | 1381461_at | 1.45 | 3.4 | 1.56 | 4.6 | 1.44 | 3.4 | 1.51 | 4 | 1.15 | 0.8 | 1.20 | 1.2 |
| 451 | 1381470_at | −2.00 | 7.8 | −1.45 | 3 | −1.27 | 1.6 | −1.34 | 2.1 | −1.22 | 1.2 | 1.07 | 0.3 |
| 452 | 1381678_at | 1.81 | 4.3 | 1.83 | 4.4 | 2.12 | 6.1 | 1.86 | 4.6 | 1.00 | 0 | −1.26 | 1 |
| 453 | 1381915_at | −1.49 | 2 | −1.85 | 3.8 | −1.48 | 1.9 | −1.21 | 0.7 | −1.04 | 0.1 | 1.18 | 0.6 |
| 454 | 1381971_at | 1.15 | 0.3 | 1.45 | 1.2 | 3.81 | 8.7 | 3.37 | 7.6 | 1.54 | 1.6 | −1.17 | 0.4 |
| 455 | 1381996_at | −1.21 | 0.8 | −1.42 | 1.9 | 1.08 | 0.2 | 1.38 | 1.6 | 1.39 | 1.7 | 1.35 | 1.5 |
| 456 | 1382008_at | 1.16 | 2.4 | 1.42 | 9 | 1.22 | 3.8 | 1.12 | 1.7 | −1.12 | 1.6 | −1.21 | 3.6 |
| 457 | 1382017_at | −1.16 | 0.2 | 2.57 | 1.9 | 2.75 | 2.1 | 1.99 | 1.2 | −1.76 | 0.9 | −2.60 | 1.9 |
| 458 | 1382108_at | 1.58 | 6.6 | 1.63 | 7.3 | 1.81 | 9.6 | 1.79 | 9.3 | 1.29 | 2.7 | 1.01 | 0.1 |
| 459 | 1382130_at | −1.01 | 0 | 1.03 | 0.1 | 1.42 | 1.6 | 1.60 | 2.5 | 1.18 | 0.6 | 1.16 | 0.5 |
| 460 | 1382138_at | 1.19 | 1.3 | −1.27 | 2 | −1.29 | 2.3 | −1.08 | 0.4 | −1.15 | 0.9 | −1.02 | 0.1 |
| 461 | 1382181_at | 2.46 | 7 | 2.44 | 6.9 | 2.45 | 6.9 | 2.03 | 4.9 | 1.50 | 2.1 | 1.14 | 0.4 |
| 462 | 1382212_at | −1.67 | 3.2 | −1.45 | 1.9 | 1.01 | 0 | 1.15 | 0.5 | 1.20 | 0.7 | 1.06 | 0.2 |
| 463 | 1382296_at | 1.83 | 5.7 | 1.52 | 3.3 | 1.74 | 5.1 | 1.74 | 5.1 | 1.02 | 0.1 | 1.28 | 1.5 |
| 464 | 1382375_at | −1.43 | 4.6 | −1.10 | 0.7 | 1.12 | 0.8 | 1.29 | 2.7 | 1.25 | 2.2 | 1.17 | 1.3 |
| 465 | 1382404_at | 1.62 | 2.1 | 1.66 | 2.3 | 2.23 | 4.7 | 2.51 | 5.7 | 1.57 | 1.9 | 1.90 | 3.3 |
| 466 | 1382431_at | −1.07 | 0.1 | 1.21 | 0.5 | 2.26 | 4.1 | 2.62 | 5.2 | 1.63 | 1.8 | 1.59 | 1.7 |
| 467 | 1382482_at | −1.16 | 0.6 | 1.06 | 0.2 | 1.81 | 4.8 | 1.83 | 4.9 | 1.41 | 2.1 | −1.03 | 0.1 |
| 468 | 1382536_at | 1.47 | 5.1 | 1.28 | 2.4 | 1.45 | 4.7 | 1.49 | 5.2 | 1.29 | 2.6 | 1.18 | 1.3 |
| 469 | 1382571_at | −1.12 | 0.3 | −1.46 | 1.9 | 1.45 | 1.9 | 1.43 | 1.8 | −1.09 | 0.2 | −1.32 | 1.2 |
| 470 | 1382628_at | −1.05 | 0.3 | −1.02 | 0.1 | 1.39 | 4 | 1.10 | 0.6 | 1.23 | 1.9 | 1.19 | 1.5 |
| 471 | 1382680_at | 4.84 | 4.2 | 4.78 | 4.1 | 3.34 | 2.7 | 2.88 | 2.2 | 1.43 | 0.5 | 1.28 | 0.3 |
| 472 | 1382692_at | 9.75 | 20.1 | 10.10 | 20.4 | 7.39 | 17.5 | 5.17 | 13.9 | 2.38 | 5.7 | 1.56 | 2 |
| 473 | 1382818_at | −1.44 | 4.7 | −1.24 | 2 | −1.06 | 0.4 | −1.09 | 0.6 | 1.21 | 1.7 | 1.17 | 1.3 |
| 474 | 1382995_at | −1.08 | 0.4 | 1.10 | 0.6 | 1.36 | 3.1 | 1.50 | 4.8 | 1.45 | 4.2 | 1.02 | 0.1 |
| 475 | 1383019_at | 1.53 | 1.6 | 1.20 | 0.5 | 1.56 | 1.7 | 1.85 | 2.8 | 1.19 | 0.4 | 1.48 | 1.4 |
| 476 | 1383073_at | 1.44 | 9.4 | 1.26 | 4.8 | 1.34 | 6.7 | 1.34 | 6.7 | 1.11 | 1.4 | 1.06 | 0.6 |
| 477 | 1383080_at | 1.42 | 9 | 1.13 | 1.8 | 1.18 | 3 | 1.30 | 5.9 | 1.18 | 2.9 | 1.06 | 0.6 |
| 478 | 1383131_at | 4.36 | 13.2 | 4.05 | 12.3 | 3.43 | 10.4 | 3.31 | 10 | 1.65 | 2.7 | 1.04 | 0.1 |
| 479 | 1383147_at | −1.26 | 1.5 | −1.64 | 4.8 | −1.17 | 0.8 | 1.14 | 0.7 | 1.23 | 1.3 | 1.15 | 0.7 |
| 480 | 1383193_at | −1.29 | 2 | −1.14 | 0.8 | 1.10 | 0.5 | 1.21 | 1.3 | 1.24 | 1.6 | 1.04 | 0.2 |
| 481 | 1383201_at | −1.25 | 3 | −1.25 | 3 | −1.13 | 1.2 | −1.08 | 0.6 | 1.04 | 0.3 | 1.09 | 0.7 |
| 482 | 1383240_at | 1.84 | 11 | 1.30 | 3.2 | 1.53 | 6.7 | 1.16 | 1.3 | −1.22 | 2.1 | −1.21 | 1.9 |
| 483 | 1383247_a_at | −2.02 | 9.7 | −1.40 | 3.3 | −1.08 | 0.4 | −1.31 | 2.3 | −1.16 | 0.9 | −1.12 | 0.7 |
| 484 | 1383261_at | −1.70 | 5.8 | −1.39 | 2.7 | −1.29 | 1.9 | −1.09 | 0.4 | −1.17 | 0.9 | 1.16 | 0.9 |
| 485 | 1383328_x_at | −2.41 | 10.5 | −1.72 | 5.2 | −1.51 | 3.4 | −1.34 | 2 | −1.21 | 1.1 | −1.16 | 0.7 |
| 486 | 1383355_at | 1.29 | 2 | 1.67 | 6 | 1.76 | 7 | 1.79 | 7.3 | 1.43 | 3.5 | 1.33 | 2.4 |
| 487 | 1383382_at | −1.17 | 1.8 | −1.21 | 2.2 | −1.29 | 3.6 | −1.33 | 4.3 | −1.10 | 0.8 | −1.08 | 0.6 |
| 488 | 1383469_at | −1.45 | 3.8 | −1.20 | 1.3 | −1.22 | 1.5 | −1.58 | 5.3 | −1.63 | 5.9 | −1.27 | 2 |
| 489 | 1383484_at | −1.04 | 0.6 | −1.11 | 2.7 | −1.06 | 1.1 | −1.00 | 0 | 1.08 | 1.9 | 1.16 | 5 |
| 490 | 1383489_at | 1.44 | 3.3 | 1.68 | 5.7 | 1.93 | 8 | 2.41 | 11.9 | 1.78 | 6.6 | 1.43 | 3.2 |
| 491 | 1383498_at | −1.06 | 0.3 | 1.26 | 1.8 | 1.64 | 5.7 | 1.98 | 9.1 | 1.39 | 3 | 1.14 | 0.7 |
| 492 | 1383516_at | 4.54 | 11.1 | 2.39 | 5 | 2.49 | 5.3 | 2.62 | 5.8 | 1.44 | 1.3 | 1.53 | 1.6 |
| 493 | 1383533_at | −1.43 | 4.5 | −1.32 | 3 | 1.01 | 0.1 | 1.05 | 0.3 | 1.35 | 3.4 | 1.17 | 1.2 |
| 494 | 1383574_at | 1.51 | 4 | 1.82 | 7 | 2.00 | 8.5 | 1.93 | 7.9 | 1.20 | 1.1 | −1.03 | 0.1 |
| 495 | 1383641_at | 1.12 | 0.6 | 1.36 | 2.3 | 1.98 | 8 | 2.24 | 10 | 1.66 | 5.1 | 1.10 | 0.4 |
| 496 | 1383644_at | −5.38 | 3.5 | −4.15 | 2.7 | −4.07 | 2.6 | −2.69 | 1.5 | −1.34 | 0.3 | −1.27 | 0.2 |
| 497 | 1383662_at | 1.17 | 0.8 | 1.05 | 0.2 | 1.16 | 0.7 | 1.28 | 1.4 | 1.38 | 2.2 | 1.06 | 0.2 |
| 498 | 1383776_at | −1.81 | 11.4 | −1.29 | 3.2 | −1.28 | 3.1 | −1.26 | 2.9 | −1.14 | 1.2 | 1.09 | 0.7 |
| 499 | 1383785_at | −1.09 | 0.3 | −1.07 | 0.2 | 1.21 | 0.8 | 1.61 | 3.1 | 1.10 | 0.3 | 1.15 | 0.5 |
| 500 | 1383824_at | −1.60 | 1.8 | −1.18 | 0.4 | 1.97 | 3.2 | 2.09 | 3.7 | 1.56 | 1.7 | 1.17 | 0.4 |
| 501 | 1383844_at | −1.40 | 1.1 | −1.16 | 0.4 | 1.62 | 1.9 | 1.52 | 1.6 | −1.07 | 0.1 | −1.04 | 0.1 |
| 502 | 1383863_at | −1.10 | 0.5 | 1.19 | 1.1 | 1.56 | 4.5 | 1.77 | 6.5 | −1.01 | 0 | 1.06 | 0.2 |
| 503 | 1383914_at | −1.87 | 6.6 | −1.58 | 4.1 | −1.19 | 0.9 | 1.02 | 0.1 | 1.07 | 0.3 | 1.04 | 0.1 |
| 504 | 1383935_at | 1.14 | 0.2 | −1.14 | 0.2 | 1.41 | 0.9 | 1.72 | 1.8 | 1.14 | 0.3 | −1.05 | 0.1 |
| 505 | 1383949_at | 1.30 | 2.1 | 1.11 | 0.5 | 1.41 | 3.2 | 1.60 | 5.2 | 1.32 | 2.2 | 1.30 | 2 |
| 506 | 1384051_at | 1.04 | 0.1 | 1.08 | 0.2 | 1.30 | 1.2 | 1.65 | 3.3 | 1.27 | 1.1 | 1.01 | 0 |
| 507 | 1384165_at | 11.68 | 7.1 | 7.38 | 5.2 | 7.88 | 5.5 | 5.10 | 3.8 | 1.81 | 0.8 | −1.06 | 0.1 |
| 508 | 1384183_at | −2.08 | 5.1 | −1.83 | 3.9 | −1.80 | 3.6 | −1.29 | 1 | 1.05 | 0.1 | −1.10 | 0.3 |
| 509 | 1384188_at | −1.97 | 10.9 | −1.43 | 4.3 | −1.32 | 2.9 | −1.02 | 0.1 | 1.21 | 1.6 | 1.29 | 2.5 |
| 510 | 1384192_at | −1.13 | 0.3 | 1.11 | 0.2 | 1.96 | 3 | 2.22 | 4 | 1.53 | 1.5 | 1.02 | 0 |
| 511 | 1384227_at | 1.04 | 0.2 | 1.08 | 0.3 | 1.31 | 1.9 | 1.11 | 0.5 | −1.11 | 0.5 | 1.12 | 0.6 |
| 512 | 1384232_at | 1.50 | 2.6 | 1.25 | 1.1 | 1.52 | 2.7 | 1.67 | 3.8 | 1.27 | 1.1 | −1.18 | 0.7 |
| 513 | 1384302_at | −2.05 | 4.4 | −2.13 | 4.7 | −1.91 | 3.7 | −1.29 | 0.9 | 1.12 | 0.3 | 1.04 | 0.1 |
| 514 | 1384335_at | −1.41 | 7.5 | −1.28 | 4.6 | −1.17 | 2.2 | −1.07 | 0.7 | 1.03 | 0.2 | 1.03 | 0.3 |
| 515 | 1384381_at | 1.18 | 0.4 | 1.71 | 2.1 | 2.29 | 4.2 | 2.56 | 5.1 | 1.90 | 2.9 | 1.41 | 1.1 |
| 516 | 1384392_at | 1.18 | 0.6 | −1.39 | 1.8 | −1.21 | 0.8 | −1.25 | 1 | −1.36 | 1.6 | 1.22 | 0.8 |
| 517 | 1384415_at | 1.69 | 6.5 | 1.73 | 7 | 1.78 | 7.4 | 1.42 | 3.5 | 1.35 | 2.8 | −1.30 | 2.2 |
| 518 | 1384724_at | 2.21 | 10 | 1.61 | 4.7 | 1.87 | 7.1 | 1.70 | 5.6 | 1.29 | 1.8 | 1.17 | 0.9 |
| 519 | 1384899_at | −1.42 | 2.1 | −1.51 | 2.7 | −1.59 | 3.3 | −1.19 | 0.8 | 1.09 | 0.3 | −1.09 | 0.3 |
| 520 | 1384900_at | 1.75 | 5.1 | 1.64 | 4.2 | 1.67 | 4.4 | 1.49 | 3 | 1.36 | 2 | 1.10 | 0.4 |
| 521 | 1384907_at | −1.65 | 2 | −1.47 | 1.4 | −1.14 | 0.3 | 1.05 | 0.1 | −1.23 | 0.5 | −1.06 | 0.1 |
| 522 | 1385057_at | 1.32 | 2.8 | 1.47 | 4.6 | 1.01 | 0 | −1.16 | 1.1 | −1.29 | 2.4 | −1.24 | 1.9 |
| 523 | 1385359_at | 3.92 | 6.6 | 3.98 | 6.7 | 4.86 | 8.2 | 3.81 | 6.4 | 1.50 | 1 | 1.34 | 0.7 |
| 524 | 1385397_at | 1.70 | 5.3 | 1.81 | 6.2 | 1.78 | 5.9 | 1.73 | 5.5 | 1.14 | 0.6 | −1.07 | 0.3 |
| 525 | 1385444_at | −2.33 | 15.4 | −1.75 | 9 | −1.27 | 2.5 | −1.32 | 3.2 | 1.24 | 2.2 | 1.02 | 0.1 |
| 526 | 1385506_at | −2.01 | 8.4 | −1.44 | 3.2 | −1.42 | 3 | −1.51 | 3.8 | −1.31 | 2 | −1.01 | 0 |
| 527 | 1385925_at | 6.37 | 11.3 | 4.37 | 8.3 | 4.40 | 8.3 | 5.07 | 9.4 | 1.39 | 0.9 | 1.02 | 0 |
| 528 | 1385926_at | 3.17 | 12.2 | 2.23 | 7.4 | 2.25 | 7.5 | 2.42 | 8.5 | 1.21 | 0.9 | 1.01 | 0 |
| 529 | 1386041_s_at | 1.52 | 0.5 | 1.50 | 0.5 | 2.23 | 1.3 | 2.33 | 1.4 | 1.54 | 0.5 | 1.92 | 0.9 |
| 530 | 1386080_at | −1.06 | 0.2 | 1.05 | 0.2 | 1.43 | 2.1 | 1.61 | 3.3 | 1.25 | 1 | 1.17 | 0.6 |
| 531 | 1386540_at | −1.64 | 7.5 | −1.29 | 2.7 | −1.26 | 2.4 | −1.25 | 2.2 | −1.18 | 1.4 | 1.01 | 0 |
| 532 | 1386721_at | −1.62 | 3.8 | −1.51 | 3 | 1.00 | 0 | 1.58 | 3.5 | 1.54 | 3.3 | 1.34 | 1.7 |
| 533 | 1386833_at | −1.14 | 0.3 | 1.23 | 0.6 | 2.02 | 3.6 | 1.83 | 2.8 | 1.34 | 1 | 1.18 | 0.4 |
| 534 | 1386864_at | 2.07 | 16.1 | 1.64 | 10 | 1.44 | 6.5 | 1.22 | 2.5 | −1.01 | 0 | −1.14 | 1.3 |
| 535 | 1386866_at | 1.64 | 11.2 | 1.31 | 4.7 | 1.46 | 7.6 | 1.55 | 9.6 | 1.19 | 2.4 | −1.04 | 0.3 |
| 536 | 1386881_at | 2.31 | 3.5 | 2.85 | 4.9 | 3.91 | 7.3 | 4.50 | 8.4 | 1.89 | 2.3 | 1.30 | 0.6 |
| 537 | 1386899_at | −1.17 | 2.3 | 1.07 | 0.6 | 1.39 | 6.8 | 1.37 | 6.4 | 1.32 | 5.3 | 1.10 | 1.1 |
| 538 | 1386912_at | −1.86 | 7.7 | −1.40 | 3.1 | 1.22 | 1.3 | 1.31 | 2.1 | 1.40 | 3 | 1.06 | 0.3 |
| 539 | 1386913_at | 3.94 | 21.1 | 2.97 | 16.4 | 2.96 | 16.4 | 2.44 | 13 | 1.37 | 2.9 | 1.08 | 0.4 |
| 540 | 1386922_at | 1.02 | 0 | 1.11 | 0.3 | 1.64 | 2.9 | 1.36 | 1.4 | −1.45 | 1.8 | 1.10 | 0.3 |
| 541 | 1386937_at | −1.63 | 2.5 | −1.49 | 1.8 | −1.34 | 1.1 | −1.23 | 0.7 | −1.05 | 0.1 | 1.03 | 0.1 |
| 542 | 1386938_at | 1.20 | 1.7 | 1.51 | 5.8 | 1.82 | 10 | 1.58 | 6.8 | 1.53 | 6.2 | 1.27 | 2.5 |
| 543 | 1386940_at | −1.31 | 2.8 | −1.08 | 0.5 | 1.26 | 2.2 | 1.35 | 3.2 | 1.40 | 3.9 | 1.15 | 1 |
| 544 | 1386948_at | −1.29 | 1.4 | 1.19 | 0.8 | 1.62 | 3.8 | 2.09 | 7.2 | −1.07 | 0.2 | 1.16 | 0.6 |
| 545 | 1387001_at | 1.45 | 5.5 | 1.40 | 4.8 | 1.65 | 8.6 | 1.51 | 6.5 | 1.16 | 1.4 | −1.03 | 0.2 |
| 546 | 1387005_at | 1.78 | 4.4 | 2.12 | 6.5 | 2.26 | 7.3 | 2.56 | 9 | 1.75 | 4.2 | 1.21 | 0.8 |
| 547 | 1387018_at | −1.80 | 2.8 | −1.64 | 2.2 | 1.08 | 0.2 | 1.19 | 0.5 | −1.10 | 0.2 | −1.07 | 0.2 |
| 548 | 1387076_at | 2.05 | 11.1 | 1.42 | 3.8 | 1.55 | 5.5 | 1.51 | 4.9 | 1.15 | 1 | −1.03 | 0.1 |
| 549 | 1387125_at | 75.78 | 20.1 | 79.54 | 20.3 | 60.92 | 19 | 40.36 | 17 | 1.66 | 1 | −1.98 | 1.5 |
| 550 | 1387137_at | 1.15 | 0.3 | 1.85 | 2 | 2.42 | 3.4 | 1.54 | 1.1 | 1.02 | 0 | 1.08 | 0.1 |
| 551 | 1387153_at | −1.64 | 4.8 | −1.25 | 1.4 | 1.15 | 0.7 | 1.15 | 0.7 | 1.19 | 1 | 1.02 | 0.1 |
| 552 | 1387154_at | −3.03 | 5.5 | −4.92 | 9.3 | −10.32 | 15.2 | −6.42 | 11.4 | −2.98 | 5.3 | −1.66 | 1.6 |
| 553 | 1387160_at | 1.32 | 0.4 | 2.18 | 2 | 3.36 | 3.9 | 3.64 | 4.3 | 1.67 | 1.1 | −1.14 | 0.2 |
| 554 | 1387197_at | −4.21 | 15.4 | −2.62 | 9.2 | −1.64 | 3.4 | −1.66 | 3.5 | 1.24 | 0.9 | 1.40 | 1.9 |
| 555 | 1387202_at | 3.15 | 12.8 | 2.57 | 9.9 | 2.38 | 8.8 | 2.37 | 8.7 | 1.81 | 5 | −1.07 | 0.2 |
| 556 | 1387219_at | 1.93 | 3.1 | 1.57 | 1.7 | 1.95 | 3.2 | 1.65 | 2 | 1.26 | 0.6 | 1.06 | 0.1 |
| 557 | 1387259_at | −1.13 | 0.4 | −1.32 | 1.2 | 1.11 | 0.3 | 1.56 | 2.5 | 1.32 | 1.2 | 1.13 | 0.4 |
| 558 | 1387270_at | 1.56 | 2.1 | 1.98 | 4 | 2.45 | 6 | 2.36 | 5.7 | 1.47 | 1.7 | 1.23 | 0.7 |
| 559 | 1387273_at | 3.21 | 4.7 | 1.85 | 1.8 | 3.70 | 5.6 | 4.18 | 6.4 | 1.39 | 0.7 | −1.17 | 0.3 |
| 560 | 1387279_at | −1.70 | 9 | −1.06 | 0.3 | −1.06 | 0.3 | −1.19 | 1.7 | −1.23 | 2.1 | −1.03 | 0.2 |
| 561 | 1387282_at | 1.47 | 2 | 1.60 | 2.7 | 1.39 | 1.5 | 1.65 | 2.9 | 1.34 | 1.3 | 1.23 | 0.8 |
| 562 | 1387294_at | −1.04 | 0.1 | 1.34 | 1.9 | 1.86 | 6.3 | 2.32 | 9.6 | 1.41 | 2.5 | 1.30 | 1.7 |
| 563 | 1387306_a_at | 2.20 | 5.7 | 1.91 | 4.2 | 1.98 | 4.5 | 1.93 | 4.3 | 1.40 | 1.5 | 1.23 | 0.7 |
| 564 | 1387343_at | 6.18 | 13.4 | 4.65 | 10.8 | 2.49 | 5.1 | 4.33 | 10.1 | 1.23 | 0.5 | 1.47 | 1.4 |
| 565 | 1387344_at | −2.23 | 8.4 | −1.70 | 4.6 | −1.57 | 3.5 | −1.32 | 1.7 | −1.18 | 0.8 | −1.02 | 0.1 |
| 566 | 1387348_at | −1.52 | 2.1 | −1.38 | 1.4 | 1.08 | 0.2 | 1.04 | 0.1 | 1.11 | 0.3 | 1.12 | 0.3 |
| 567 | 1387389_at | 12.76 | 10.1 | 12.72 | 10.1 | 16.88 | 11.6 | 22.31 | 13.1 | 2.46 | 2.1 | 2.87 | 2.7 |
| 568 | 1387395_at | 2.02 | 9.3 | 1.56 | 4.7 | 1.54 | 4.5 | 1.26 | 1.7 | 1.10 | 0.5 | −1.14 | 0.8 |
| 569 | 1387402_at | 1.60 | 3.7 | 1.68 | 4.3 | 1.94 | 6.3 | 2.02 | 6.9 | 1.25 | 1.2 | 1.08 | 0.3 |
| 570 | 1387530_a_at | 1.03 | 0.1 | 1.04 | 0.2 | −1.09 | 0.4 | −1.15 | 0.8 | −1.29 | 1.9 | −1.43 | 3.2 |
| 571 | 1387547_a_at | 1.05 | 0.1 | 1.45 | 1.4 | 3.30 | 8.4 | 3.27 | 8.3 | 1.54 | 1.8 | 1.30 | 0.9 |
| 572 | 1387548_at | 1.64 | 3.1 | 1.55 | 2.6 | 1.67 | 3.3 | 2.01 | 5.3 | 1.19 | 0.7 | −1.03 | 0.1 |
| 573 | 1387630_at | 1.20 | 1.4 | 1.20 | 1.4 | 1.32 | 2.8 | 1.18 | 1.3 | −1.05 | 0.2 | −1.07 | 0.4 |
| 574 | 1387648_at | 22.75 | 14.9 | 16.94 | 13.2 | 24.96 | 15.4 | 15.47 | 12.6 | 3.10 | 3.5 | 1.08 | 0.1 |
| 575 | 1387675_at | 2.28 | 9.3 | 2.35 | 9.8 | 2.98 | 13.5 | 1.96 | 7 | 1.50 | 3.2 | 1.35 | 2 |
| 576 | 1387759_s_at | 1.63 | 4.3 | 2.06 | 7.8 | 1.85 | 6.1 | 1.57 | 3.8 | −1.18 | 0.9 | −1.55 | 3.6 |
| 577 | 1387789_at | −1.66 | 4.8 | −1.02 | 0.1 | 1.47 | 3.1 | 1.46 | 3 | 1.61 | 4.3 | 1.14 | 0.6 |
| 578 | 1387805_at | 1.47 | 2.3 | 1.42 | 2 | 1.32 | 1.4 | 1.22 | 0.9 | 1.03 | 0.1 | 1.04 | 0.1 |
| 579 | 1387808_at | 1.33 | 2.6 | 1.43 | 3.7 | 1.87 | 8.6 | 2.18 | 11.6 | 1.82 | 8.1 | 1.33 | 2.6 |
| 580 | 1387817_at | −1.63 | 2.1 | −1.36 | 1.1 | −1.04 | 0.1 | 1.37 | 1.1 | 1.04 | 0.1 | −1.09 | 0.2 |
| 581 | 1387843_at | 1.28 | 0.8 | 1.11 | 0.3 | 1.19 | 0.5 | 1.22 | 0.6 | −1.31 | 0.9 | 1.28 | 0.8 |
| 582 | 1387856_at | 1.04 | 0.2 | 1.06 | 0.5 | 1.33 | 4.8 | 1.40 | 6.2 | 1.18 | 2 | 1.03 | 0.2 |
| 583 | 1387870_at | 1.29 | 2.2 | 1.32 | 2.5 | 1.07 | 0.4 | −1.08 | 0.4 | −1.13 | 0.8 | 1.14 | 0.8 |
| 584 | 1387871_at | 1.80 | 7.4 | 1.63 | 5.7 | 2.16 | 10.8 | 2.14 | 10.6 | 1.12 | 0.6 | −1.04 | 0.1 |
| 585 | 1387885_at | −1.50 | 4.4 | 1.38 | 3.1 | 1.55 | 5 | 1.60 | 5.5 | 1.33 | 2.5 | 1.02 | 0.1 |
| 586 | 1387892_at | 1.92 | 13.6 | 1.55 | 8 | 1.49 | 6.9 | 1.50 | 7.1 | 1.04 | 0.3 | −1.08 | 0.6 |
| 587 | 1387893_at | 1.22 | 1.5 | 1.52 | 4.7 | 2.30 | 12.6 | 2.41 | 13.5 | 1.77 | 7.5 | 1.37 | 3 |
| 588 | 1387896_at | −1.45 | 4.4 | −1.42 | 4.1 | −1.45 | 4.4 | −1.28 | 2.4 | −1.11 | 0.7 | −1.08 | 0.4 |
| 589 | 1387897_at | −1.53 | 4.3 | 1.08 | 0.4 | −1.06 | 0.3 | 1.05 | 0.2 | 1.08 | 0.3 | 1.18 | 1 |
| 590 | 1387975_at | 1.36 | 2.5 | 1.31 | 2.1 | 1.52 | 4 | 1.64 | 5.2 | 1.07 | 0.3 | −1.05 | 0.2 |
| 591 | 1387976_at | −1.77 | 4.3 | −1.36 | 1.6 | 1.52 | 2.6 | 1.37 | 1.7 | 1.51 | 2.6 | −1.02 | 0 |
| 592 | 1388032_a_at | 17.94 | 8.9 | 14.69 | 8 | 10.69 | 6.7 | 2.48 | 1.5 | 1.33 | 0.3 | −1.99 | 1 |
| 593 | 1388054_a_at | 6.42 | 7.8 | 3.47 | 4.2 | 2.65 | 2.9 | 4.97 | 6.3 | 1.14 | 0.2 | −1.30 | 0.4 |
| 594 | 1388089_a_at | 1.18 | 1.7 | 1.23 | 2.3 | 1.14 | 1.2 | 1.11 | 0.9 | 1.01 | 0.1 | −1.14 | 1.1 |
| 595 | 1388119_at | 1.37 | 7.8 | 1.15 | 2.3 | 1.04 | 0.4 | −1.01 | 0.1 | −1.04 | 0.4 | −1.03 | 0.3 |
| 596 | 1388120_at | −1.25 | 3.1 | −1.24 | 3.1 | −1.13 | 1.3 | −1.33 | 4.7 | −1.12 | 1.1 | −1.25 | 3.3 |
| 597 | 1388131_at | 2.24 | 6.1 | 2.37 | 6.8 | 3.74 | 12.2 | 4.60 | 14.6 | 1.29 | 1.1 | 1.18 | 0.6 |
| 598 | 1388142_at | 5.13 | 5.6 | 2.79 | 2.8 | 2.41 | 2.2 | 4.36 | 4.8 | 1.32 | 0.4 | 1.03 | 0 |
| 599 | 1388143_at | 1.90 | 7.1 | 1.90 | 7.1 | 2.87 | 13.9 | 2.92 | 14.2 | 1.26 | 1.5 | −1.04 | 0.2 |
| 600 | 1388151_at | 1.01 | 0 | 1.16 | 2.1 | 1.27 | 4.1 | 1.30 | 4.8 | 1.02 | 0.2 | −1.04 | 0.3 |
| 601 | 1388155_at | −1.94 | 2.1 | −1.77 | 1.7 | −1.73 | 1.5 | −1.73 | 1.5 | −1.38 | 0.7 | −1.04 | 0.1 |
| 602 | 1388170_at | −1.08 | 0.4 | 1.09 | 0.5 | 1.19 | 1.3 | 1.22 | 1.6 | 1.20 | 1.4 | 1.02 | 0.1 |
| 603 | 1388201_at | −2.01 | 4 | −1.73 | 2.8 | −1.24 | 0.7 | −1.57 | 2.1 | 1.04 | 0.1 | −1.12 | 0.3 |
| 604 | 1388204_at | 23.83 | 13.5 | 25.19 | 13.8 | 14.22 | 10.7 | 5.66 | 5.8 | 2.29 | 1.9 | −1.15 | 0.2 |
| 605 | 1388218_at | 1.01 | 0 | −1.43 | 2.1 | −1.37 | 1.7 | −1.77 | 4.3 | −2.07 | 6.2 | −1.43 | 2.1 |
| 606 | 1388243_at | −1.07 | 0.2 | 1.34 | 1.7 | 2.06 | 6.9 | 2.84 | 11.6 | 2.84 | 11.6 | 1.46 | 2.6 |
| 607 | 1388271_at | 10.71 | 28.9 | 7.13 | 24.5 | 4.92 | 20 | 3.94 | 16.9 | 2.16 | 8 | 1.38 | 2.1 |
| 608 | 1388312_at | −1.68 | 4.5 | −1.31 | 1.6 | 1.22 | 1.1 | 1.35 | 1.9 | 1.61 | 4 | 1.26 | 1.3 |
| 609 | 1388318_at | 2.94 | 22.9 | 2.66 | 20.8 | 1.74 | 10.6 | 1.38 | 4.9 | 1.08 | 0.6 | 1.04 | 0.3 |
| 610 | 1388335_at | 1.69 | 11 | 1.68 | 10.8 | 1.84 | 13.3 | 1.77 | 12.3 | 1.05 | 0.4 | −1.02 | 0.1 |
| 611 | 1388348_at | 1.64 | 8.6 | 1.50 | 6.5 | 1.54 | 7.1 | 1.31 | 3.4 | 1.05 | 0.3 | 1.00 | 0 |
| 612 | 1388356_at | −1.07 | 0.5 | −1.06 | 0.4 | −1.20 | 2.2 | −1.40 | 5.6 | −1.23 | 2.6 | −1.18 | 1.9 |
| 613 | 1388392_at | 1.45 | 6.7 | 1.52 | 8.1 | 1.78 | 12.5 | 1.78 | 12.4 | 1.20 | 2.2 | 1.04 | 0.3 |
| 614 | 1388397_at | 1.96 | 8.8 | 1.11 | 0.5 | 1.15 | 0.8 | 1.12 | 0.6 | 1.04 | 0.2 | −1.02 | 0.1 |
| 615 | 1388401_at | 1.17 | 1.2 | 1.15 | 1 | 1.47 | 4.4 | 1.54 | 5.3 | 1.14 | 0.9 | 1.04 | 0.2 |
| 616 | 1388449_at | 1.36 | 9.8 | 1.51 | 14.3 | 1.25 | 6.2 | 1.11 | 1.9 | 1.12 | 2.2 | 1.09 | 1.4 |
| 617 | 1388459_at | 1.78 | 4.1 | 1.62 | 3.1 | 3.10 | 11 | 3.34 | 11.9 | 1.54 | 2.6 | −1.07 | 0.2 |
| 618 | 1388486_at | −1.58 | 9.1 | −1.47 | 7.1 | −1.30 | 4 | −1.27 | 3.5 | −1.08 | 0.7 | 1.05 | 0.4 |
| 619 | 1388492_at | 1.06 | 0.5 | 1.21 | 3 | 1.21 | 3.1 | 1.03 | 0.2 | 1.03 | 0.3 | −1.07 | 0.7 |
| 620 | 1388493_at | −1.53 | 2.8 | −1.29 | 1.3 | −1.02 | 0 | 1.29 | 1.3 | 1.40 | 2 | 1.19 | 0.7 |
| 621 | 1388494_at | 1.15 | 0.6 | 1.33 | 1.6 | 3.18 | 12.5 | 3.85 | 15.1 | 1.72 | 4.2 | −1.11 | 0.4 |
| 622 | 1388495_at | −1.11 | 0.8 | −1.36 | 4.4 | −1.18 | 1.6 | −1.16 | 1.4 | 1.04 | 0.2 | 1.05 | 0.3 |
| 623 | 1388521_at | 1.13 | 0.7 | −1.08 | 0.4 | 1.27 | 1.9 | 1.39 | 3.1 | 1.22 | 1.4 | −1.16 | 0.9 |
| 624 | 1388533_at | −1.46 | 9.2 | −1.35 | 6.6 | −1.23 | 3.7 | −1.19 | 2.8 | 1.02 | 0.2 | 1.05 | 0.5 |
| 625 | 1388534_at | 1.14 | 1.6 | 1.22 | 2.9 | 1.16 | 1.8 | 1.21 | 2.8 | −1.06 | 0.5 | −1.07 | 0.6 |
| 626 | 1388568_at | 1.33 | 7.9 | 1.07 | 0.9 | 1.06 | 0.8 | 1.02 | 0.2 | −1.04 | 0.5 | −1.05 | 0.6 |
| 627 | 1388583_at | −2.04 | 5 | −1.84 | 3.9 | 1.16 | 0.5 | 1.13 | 0.4 | −1.00 | 0 | 1.14 | 0.4 |
| 628 | 1388602_at | 1.06 | 0.2 | 1.79 | 3.5 | 2.73 | 7.9 | 2.87 | 8.5 | 1.48 | 1.9 | 1.20 | 0.6 |
| 629 | 1388618_at | −1.32 | 2.5 | −1.14 | 0.9 | 1.27 | 2 | 1.43 | 3.7 | 1.18 | 1.1 | 1.09 | 0.4 |
| 630 | 1388666_at | 1.74 | 6.5 | 1.49 | 3.9 | 1.33 | 2.3 | 1.87 | 7.8 | 1.06 | 0.2 | 1.45 | 3.6 |
| 631 | 1388695_at | 2.21 | 18.3 | 1.56 | 8.9 | 1.27 | 3.5 | 1.31 | 4.2 | 1.01 | 0 | 1.02 | 0.1 |
| 632 | 1388699_at | −1.14 | 1.1 | 1.12 | 0.9 | 1.37 | 4.3 | 1.45 | 5.7 | 1.42 | 5.2 | 1.29 | 3.2 |
| 633 | 1388700_at | −1.72 | 11 | −1.28 | 3.5 | −1.13 | 1.2 | −1.09 | 0.7 | 1.13 | 1.2 | 1.10 | 0.9 |
| 634 | 1388703_at | 1.65 | 2.5 | 2.05 | 4.4 | 2.94 | 8 | 3.42 | 9.6 | 1.51 | 1.8 | 1.14 | 0.4 |
| 635 | 1388705_at | −1.01 | 0 | 1.44 | 4 | 1.98 | 10.1 | 2.11 | 11.3 | 1.63 | 6.2 | 1.06 | 0.3 |
| 636 | 1388711_at | 2.15 | 14 | 2.29 | 15.4 | 1.77 | 9.5 | 1.50 | 5.8 | 1.29 | 2.8 | 1.09 | 0.6 |
| 637 | 1388715_at | 1.52 | 10.8 | 1.28 | 5.1 | 1.26 | 4.7 | 1.26 | 4.7 | 1.00 | 0 | −1.07 | 0.8 |
| 638 | 1388728_at | −1.52 | 8.8 | −1.25 | 3.4 | −1.17 | 2 | −1.25 | 3.5 | −1.09 | 0.8 | −1.03 | 0.2 |
| 639 | 1388742_at | 1.46 | 5.7 | 1.44 | 5.4 | 1.31 | 3.5 | 1.42 | 5.2 | 1.43 | 5.3 | 1.22 | 2.1 |
| 640 | 1388750_at | 3.58 | 14.6 | 1.25 | 1.1 | 1.09 | 0.3 | 1.33 | 1.6 | 1.03 | 0.1 | −1.19 | 0.8 |
| 641 | 1388773_at | −1.00 | 0 | −1.05 | 0.2 | −1.15 | 0.9 | −1.24 | 1.7 | −1.19 | 1.2 | −1.10 | 0.5 |
| 642 | 1388792_at | 3.59 | 9.1 | 2.31 | 4.9 | 2.27 | 4.7 | 3.16 | 7.9 | −1.07 | 0.2 | 1.37 | 1.1 |
| 643 | 1388836_at | −1.63 | 10.6 | −1.58 | 9.7 | −1.20 | 2.4 | −1.27 | 3.7 | −1.33 | 4.7 | −1.19 | 2.3 |
| 644 | 1388870_at | −1.26 | 4.1 | −1.43 | 7.9 | −1.21 | 3 | −1.16 | 2.1 | −1.01 | 0.1 | 1.07 | 0.6 |
| 645 | 1388878_at | 1.42 | 2.7 | 1.57 | 4 | 1.52 | 3.6 | 1.25 | 1.4 | 1.43 | 2.7 | 1.42 | 2.7 |
| 646 | 1388889_at | −1.40 | 5.5 | −1.19 | 1.9 | −1.31 | 4 | −1.43 | 6 | −1.17 | 1.7 | 1.02 | 0.1 |
| 647 | 1388926_at | −1.33 | 2 | 1.15 | 0.7 | 1.08 | 0.3 | 1.09 | 0.4 | 1.36 | 2.3 | 1.36 | 2.2 |
| 648 | 1388945_at | 2.24 | 9.7 | 1.73 | 5.6 | 2.05 | 8.2 | 2.07 | 8.4 | 1.44 | 2.9 | 1.41 | 2.7 |
| 649 | 1388955_at | 1.06 | 0.5 | 1.28 | 4.7 | 1.73 | 14.2 | 1.53 | 10.3 | 1.25 | 4 | 1.05 | 0.4 |
| 650 | 1388970_at | 1.17 | 0.4 | 1.68 | 2.2 | 2.75 | 6.2 | 2.23 | 4.4 | 1.35 | 1 | 1.15 | 0.3 |
| 651 | 1389018_at | 2.43 | 6.5 | 3.30 | 9.9 | 2.03 | 4.6 | 1.77 | 3.3 | −1.04 | 0.1 | −1.08 | 0.2 |
| 652 | 1389039_at | −1.09 | 0.5 | −1.04 | 0.2 | 1.31 | 2.5 | 1.48 | 4.3 | 1.52 | 4.8 | 1.18 | 1.1 |
| 653 | 1389040_at | −1.16 | 1.6 | −1.37 | 5 | −1.14 | 1.4 | −1.12 | 1 | 1.15 | 1.4 | 1.12 | 1 |
| 654 | 1389059_at | −2.02 | 7.2 | −1.51 | 3.2 | −1.14 | 0.6 | −1.15 | 0.7 | 1.02 | 0.1 | 1.08 | 0.3 |
| 655 | 1389103_at | 1.49 | 5 | 1.33 | 3 | 1.30 | 2.7 | 1.20 | 1.6 | −1.03 | 0.2 | −1.01 | 0.1 |
| 656 | 1389123_at | −1.05 | 0.1 | 1.33 | 1.5 | 1.17 | 0.6 | −1.13 | 0.5 | 1.06 | 0.2 | 1.12 | 0.4 |
| 657 | 1389151_at | 2.00 | 5.8 | 1.62 | 3.3 | 2.18 | 6.9 | 2.99 | 11.2 | 1.65 | 3.5 | 1.14 | 0.5 |
| 658 | 1389153_at | −1.19 | 2.1 | −1.54 | 8.3 | −1.41 | 6.1 | −1.41 | 6 | −1.10 | 0.9 | 1.01 | 0 |
| 659 | 1389157_at | −1.01 | 0.1 | −1.10 | 0.7 | 1.10 | 0.8 | 1.16 | 1.4 | 1.01 | 0.1 | 1.07 | 0.4 |
| 660 | 1389186_at | 1.14 | 0.8 | 1.17 | 1 | 1.59 | 5.5 | 1.87 | 8.4 | 1.61 | 5.7 | 1.18 | 1.1 |
| 661 | 1389207_at | 1.46 | 6.2 | 1.34 | 4.2 | 1.41 | 5.3 | 1.15 | 1.3 | 1.06 | 0.4 | 1.05 | 0.3 |
| 662 | 1389213_at | −1.14 | 0.8 | −1.16 | 0.9 | 1.14 | 0.8 | 1.21 | 1.4 | −1.02 | 0.1 | −1.24 | 1.6 |
| 663 | 1389214_at | 2.15 | 5 | 2.45 | 6.3 | 3.74 | 10.9 | 4.56 | 13 | 1.83 | 3.4 | 1.30 | 1 |
| 664 | 1389227_at | 1.36 | 5 | 1.61 | 9.3 | 1.49 | 7.2 | 1.35 | 4.7 | 1.22 | 2.4 | 1.04 | 0.3 |
| 665 | 1389244_x_at | 8.87 | 12.9 | 10.59 | 14.2 | 15.19 | 16.8 | 19.77 | 18.6 | 6.34 | 10.4 | 2.88 | 4.6 |
| 666 | 1389255_at | 1.09 | 0.2 | 1.79 | 2.8 | 3.81 | 9.9 | 3.94 | 10.2 | 1.88 | 3.2 | 1.56 | 1.9 |
| 667 | 1389297_at | 1.74 | 5 | 1.67 | 4.5 | 1.44 | 2.6 | 1.25 | 1.2 | 1.19 | 0.9 | 1.11 | 0.4 |
| 668 | 1389324_at | −1.31 | 1.5 | 1.13 | 0.5 | 2.16 | 7.6 | 1.93 | 6 | 1.09 | 0.3 | −1.02 | 0.1 |
| 669 | 1389341_at | −1.22 | 0.6 | 1.17 | 0.4 | 2.83 | 7.1 | 2.79 | 6.9 | 1.42 | 1.3 | 1.17 | 0.4 |
| 670 | 1389373_at | −1.27 | 4.1 | −1.06 | 0.5 | 1.14 | 1.6 | 1.13 | 1.5 | 1.00 | 0 | 1.02 | 0.1 |
| 671 | 1389464_at | −10.10 | 5.4 | −2.12 | 1 | −8.63 | 4.8 | −3.78 | 2.3 | −1.70 | 0.6 | −1.87 | 0.8 |
| 672 | 1389470_at | 6.43 | 8.4 | 4.38 | 6 | 1.99 | 1.9 | 2.55 | 3 | 1.11 | 0.2 | 1.12 | 0.2 |
| 673 | 1389476_at | −1.29 | 3.5 | −1.30 | 3.7 | −1.04 | 0.2 | −1.03 | 0.2 | −1.04 | 0.3 | 1.02 | 0.1 |
| 674 | 1389478_at | −1.12 | 0.7 | 1.02 | 0.1 | 1.16 | 1 | 1.21 | 1.6 | 1.21 | 1.5 | −1.11 | 0.6 |
| 675 | 1389483_at | 1.56 | 4.7 | 1.53 | 4.4 | 1.82 | 7.4 | 2.21 | 10.9 | 1.47 | 3.7 | 1.24 | 1.6 |
| 676 | 1389533_at | −1.56 | 2.6 | 1.11 | 0.3 | 1.45 | 2 | 2.53 | 8.1 | 2.11 | 5.9 | 1.17 | 0.6 |
| 677 | 1389546_at | −1.47 | 6.1 | −1.29 | 3.2 | −1.07 | 0.5 | 1.14 | 1.2 | 1.17 | 1.5 | 1.34 | 4 |
| 678 | 1389573_at | −1.72 | 5.1 | −1.03 | 0.1 | 1.18 | 0.8 | 1.04 | 0.2 | 1.35 | 2.1 | −1.14 | 0.6 |
| 679 | 1389590_at | −1.34 | 2.2 | −1.39 | 2.6 | −1.29 | 1.8 | −1.13 | 0.6 | −1.13 | 0.6 | 1.06 | 0.2 |
| 680 | 1389611_at | −1.55 | 3.7 | −1.73 | 5.2 | −1.67 | 4.7 | −1.28 | 1.5 | −1.16 | 0.7 | −1.10 | 0.4 |
| 681 | 1389617_at | 1.49 | 5 | 1.57 | 6.1 | 1.88 | 9.9 | 1.61 | 6.5 | 1.05 | 0.2 | −1.13 | 0.8 |
| 682 | 1389651_at | 1.51 | 1.9 | 1.56 | 2.1 | 2.60 | 6.7 | 2.43 | 6 | 1.18 | 0.5 | −1.10 | 0.2 |
| 683 | 1389787_at | 1.23 | 1.8 | 1.49 | 4.8 | 1.62 | 6.4 | 1.91 | 9.9 | 1.45 | 4.4 | 1.05 | 0.3 |
| 684 | 1389789_at | 1.15 | 1 | 1.06 | 0.3 | −1.20 | 1.6 | −1.28 | 2.6 | −1.20 | 1.7 | 1.02 | 0.1 |
| 685 | 1389836_a_at | −2.40 | 9.2 | −2.60 | 10.4 | −1.97 | 6.4 | −1.84 | 5.4 | −1.13 | 0.5 | −1.02 | 0.1 |
| 686 | 1389883_at | −1.45 | 6.2 | −1.24 | 2.6 | −1.06 | 0.4 | −1.08 | 0.6 | 1.00 | 0 | −1.10 | 0.8 |
| 687 | 1389966_at | −3.00 | 21.4 | −2.06 | 13.4 | −1.11 | 0.7 | 1.03 | 0.2 | 1.57 | 7 | 1.08 | 0.5 |
| 688 | 1390031_at | 1.38 | 3.5 | 1.36 | 3.3 | 1.22 | 1.7 | 1.09 | 0.5 | 1.06 | 0.3 | −1.01 | 0 |
| 689 | 1390112_at | −1.45 | 1.6 | −1.19 | 0.5 | −1.50 | 1.8 | −1.49 | 1.7 | −1.06 | 0.1 | 1.29 | 0.9 |
| 690 | 1390141_at | 2.74 | 7.5 | 1.48 | 1.8 | 1.32 | 1.1 | 1.35 | 1.1 | 1.48 | 1.7 | 1.03 | 0.1 |
| 691 | 1390159_at | −2.41 | 5.6 | −2.05 | 4.1 | −1.33 | 1 | 1.01 | 0 | −1.16 | 0.4 | −1.11 | 0.3 |
| 692 | 1390173_at | 1.77 | 11.3 | 1.25 | 2.8 | 1.36 | 4.6 | 1.10 | 0.8 | −1.03 | 0.2 | −1.05 | 0.3 |
| 693 | 1390300_at | 1.01 | 0 | 1.04 | 0.1 | 1.65 | 2.6 | 1.68 | 2.7 | 1.27 | 0.8 | 1.52 | 2 |
| 694 | 1390306_at | 1.10 | 0.4 | 1.20 | 0.8 | 1.52 | 2.9 | 1.54 | 3.1 | 1.14 | 0.5 | 1.04 | 0.1 |
| 695 | 1390311_at | −1.42 | 5.2 | −1.23 | 2.2 | −1.23 | 2.3 | 1.01 | 0 | −1.05 | 0.3 | 1.00 | 0 |
| 696 | 1390380_at | −1.36 | 5.2 | −1.25 | 3.2 | 1.02 | 0.1 | 1.06 | 0.4 | 1.05 | 0.4 | 1.05 | 0.4 |
| 697 | 1390383_at | 2.66 | 7.3 | 3.21 | 9.4 | 2.71 | 7.5 | 2.37 | 6 | 1.21 | 0.6 | 1.04 | 0.1 |
| 698 | 1390388_at | −1.54 | 2.4 | −1.03 | 0.1 | −1.22 | 0.8 | −1.36 | 1.5 | −1.09 | 0.3 | −1.12 | 0.4 |
| 699 | 1390403_at | 1.17 | 0.6 | 1.41 | 1.9 | 1.99 | 5.6 | 1.96 | 5.4 | 1.35 | 1.5 | 1.16 | 0.6 |
| 700 | 1390406_at | 1.11 | 0.7 | 1.28 | 2.5 | 1.77 | 9.1 | 1.44 | 4.7 | 1.15 | 1.1 | 1.12 | 0.8 |
| 701 | 1390415_at | 1.85 | 5.8 | 1.47 | 2.8 | 1.57 | 3.6 | 1.33 | 1.7 | −1.10 | 0.4 | −1.09 | 0.3 |
| 702 | 1390420_at | 2.31 | 4.9 | 2.53 | 5.7 | 3.04 | 7.5 | 2.47 | 5.5 | 2.76 | 6.5 | 1.36 | 1 |
| 703 | 1390536_at | −1.87 | 9.1 | −1.53 | 5.1 | −1.43 | 3.9 | −1.24 | 1.7 | −1.24 | 1.8 | −1.00 | 0 |
| 704 | 1390555_at | 1.11 | 0.7 | 1.09 | 0.5 | 1.16 | 1 | 1.38 | 3.5 | 1.08 | 0.4 | −1.09 | 0.5 |
| 705 | 1390647_at | 1.09 | 0.5 | 1.01 | 0.1 | 1.15 | 1 | 1.19 | 1.3 | 1.17 | 1.1 | 1.00 | 0 |
| 706 | 1390706_at | −1.37 | 3.2 | −1.21 | 1.4 | −1.15 | 0.9 | 1.08 | 0.4 | 1.08 | 0.4 | 1.11 | 0.6 |
| 707 | 1390738_at | 1.53 | 1.6 | 1.51 | 1.5 | 1.84 | 2.8 | 2.16 | 4 | 2.44 | 5 | 1.26 | 0.7 |
| 708 | 1390782_at | −1.99 | 4 | −1.43 | 1.5 | 1.92 | 3.6 | 2.18 | 4.8 | 1.06 | 0.1 | 1.24 | 0.7 |
| 709 | 1390828_at | −2.21 | 1.5 | 1.23 | 0.3 | 1.49 | 0.6 | 1.86 | 1.1 | 1.70 | 0.8 | 1.55 | 0.7 |
| 710 | 1390832_at | −1.28 | 2 | −1.34 | 2.7 | −1.30 | 2.3 | −1.34 | 2.7 | −1.23 | 1.6 | −1.14 | 0.8 |
| 711 | 1390835_at | 2.40 | 6.1 | 2.11 | 4.8 | 2.81 | 7.8 | 1.67 | 2.7 | 1.61 | 2.4 | 1.20 | 0.6 |
| 712 | 1390846_at | −1.47 | 5.4 | −1.28 | 2.8 | 1.09 | 0.6 | 1.09 | 0.6 | 1.41 | 4.5 | 1.03 | 0.1 |
| 713 | 1390914_at | 1.28 | 1.8 | 1.60 | 4.7 | 2.06 | 9 | 2.11 | 9.4 | 1.62 | 4.9 | 1.22 | 1.2 |
| 714 | 1391167_at | −1.35 | 4.3 | −1.06 | 0.4 | −1.11 | 0.9 | −1.17 | 1.6 | −1.04 | 0.3 | −1.03 | 0.2 |
| 715 | 1391421_at | −1.47 | 4.8 | −1.37 | 3.4 | −1.07 | 0.4 | −1.04 | 0.2 | 1.13 | 0.8 | −1.02 | 0.1 |
| 716 | 1391435_at | −1.70 | 3.5 | 1.11 | 0.3 | 1.91 | 4.7 | 2.10 | 5.8 | 1.50 | 2.3 | 1.18 | 0.6 |
| 717 | 1391442_at | −1.00 | 0 | 1.29 | 1.4 | 1.80 | 4.9 | 2.24 | 7.8 | 2.06 | 6.6 | 1.20 | 0.8 |
| 718 | 1391450_at | −1.39 | 1.8 | 1.11 | 0.3 | 1.87 | 4.9 | 2.18 | 6.8 | 1.50 | 2.5 | −1.21 | 0.8 |
| 719 | 1391458_at | 3.30 | 10.5 | 3.33 | 10.7 | 3.03 | 9.5 | 2.04 | 5 | 1.19 | 0.6 | 1.12 | 0.3 |
| 720 | 1391503_at | 1.20 | 0.7 | 1.26 | 1 | 1.71 | 3.7 | 1.82 | 4.4 | 1.28 | 1.2 | 1.01 | 0 |
| 721 | 1391505_x_at | 4.60 | 7.6 | 5.48 | 8.9 | 4.25 | 7.1 | 2.15 | 2.7 | 1.24 | 0.4 | 1.20 | 0.3 |
| 722 | 1391607_at | 1.35 | 2.9 | 1.49 | 4.5 | 1.12 | 0.7 | 1.17 | 1.1 | −1.04 | 0.2 | 1.03 | 0.1 |
| 723 | 1391827_at | −1.05 | 0.3 | 1.17 | 1.1 | 1.21 | 1.6 | 1.36 | 3.3 | 1.03 | 0.2 | 1.00 | 0 |
| 724 | 1391856_at | −1.26 | 1.1 | −1.28 | 1.2 | 1.04 | 0.1 | 1.31 | 1.4 | 1.09 | 0.3 | 1.05 | 0.1 |
| 725 | 1391871_at | −1.04 | 0.1 | −1.46 | 1.8 | 1.37 | 1.4 | 1.55 | 2.3 | 1.27 | 0.9 | 1.35 | 1.3 |
| 726 | 1391946_at | 4.42 | 8.7 | 2.92 | 5.4 | 2.72 | 4.8 | 1.70 | 1.8 | 1.39 | 0.9 | 1.07 | 0.1 |
| 727 | 1392044_at | −1.78 | 6.9 | −1.35 | 2.6 | −1.02 | 0.1 | 1.01 | 0 | 1.32 | 2.2 | 1.16 | 0.9 |
| 728 | 1392171_at | 21.90 | 17.4 | 17.41 | 15.9 | 8.92 | 11.4 | 5.84 | 8.5 | 1.37 | 0.7 | −1.18 | 0.3 |
| 729 | 1392220_at | −1.85 | 5.2 | −1.13 | 0.5 | 1.76 | 4.5 | 1.77 | 4.6 | 1.74 | 4.4 | 1.27 | 1.2 |
| 730 | 1392264_s_at | 4.14 | 4.4 | 6.74 | 6.8 | 6.71 | 6.8 | 6.23 | 6.4 | 3.26 | 3.3 | 1.91 | 1.3 |
| 731 | 1392280_at | 2.17 | 6.6 | 1.94 | 5.2 | 1.45 | 2.1 | 1.15 | 0.5 | 1.09 | 0.3 | 1.01 | 0 |
| 732 | 1392471_at | −1.27 | 3.8 | −1.27 | 3.8 | −1.15 | 1.6 | −1.11 | 1.1 | −1.08 | 0.7 | 1.08 | 0.6 |
| 733 | 1392489_at | −1.16 | 0.9 | 1.21 | 1.4 | 1.95 | 9 | 2.31 | 12.2 | 1.80 | 7.5 | 1.43 | 3.6 |
| 734 | 1392490_at | −1.18 | 2.6 | −1.12 | 1.5 | −1.13 | 1.7 | −1.07 | 0.7 | 1.11 | 1.3 | −1.03 | 0.3 |
| 735 | 1392497_at | −1.40 | 4.1 | −1.41 | 4.2 | −1.05 | 0.3 | 1.02 | 0.1 | 1.05 | 0.3 | 1.05 | 0.3 |
| 736 | 1392578_at | 2.29 | 7.2 | 2.66 | 9.1 | 2.66 | 9.1 | 3.94 | 14.1 | 2.98 | 10.5 | 1.87 | 4.7 |
| 737 | 1392597_at | −1.47 | 4.6 | −1.12 | 0.7 | −1.13 | 0.8 | −1.12 | 0.7 | 1.05 | 0.2 | −1.16 | 1 |
| 738 | 1392648_at | 2.53 | 8 | 3.34 | 11.4 | 3.04 | 10.2 | 3.19 | 10.8 | 1.49 | 2.2 | 1.04 | 0.1 |
| 739 | 1392736_at | 5.66 | 7.2 | 6.46 | 8 | 4.32 | 5.6 | 1.55 | 0.9 | −1.15 | 0.2 | 1.14 | 0.2 |
| 740 | 1392784_at | 1.04 | 0.2 | 1.03 | 0.1 | 1.23 | 1.5 | 1.26 | 1.8 | 1.24 | 1.6 | 1.11 | 0.6 |
| 741 | 1392786_at | 1.33 | 1.1 | 1.28 | 0.9 | 1.97 | 4.3 | 1.96 | 4.2 | 1.51 | 1.9 | 1.32 | 1.1 |
| 742 | 1392905_at | 3.44 | 6.8 | 3.61 | 7.2 | 4.27 | 8.6 | 3.96 | 7.9 | 2.04 | 2.9 | 1.28 | 0.6 |
| 743 | 1392917_at | −1.04 | 0.3 | −1.15 | 2 | −1.11 | 1.2 | −1.08 | 0.8 | −1.02 | 0.2 | −1.07 | 0.7 |
| 744 | 1392934_at | −1.05 | 0.5 | −1.04 | 0.5 | 1.04 | 0.4 | 1.15 | 2.6 | 1.13 | 2.2 | −1.01 | 0.1 |
| 745 | 1392948_at | −1.29 | 0.8 | −1.66 | 2.2 | −1.73 | 2.5 | −1.44 | 1.4 | −1.28 | 0.8 | −1.07 | 0.1 |
| 746 | 1392953_at | 1.09 | 0.2 | 1.10 | 0.2 | 1.32 | 0.9 | 1.42 | 1.2 | 1.02 | 0 | −1.12 | 0.3 |
| 747 | 1392953_at | 1.09 | 0.2 | 1.10 | 0.2 | 1.32 | 0.9 | 1.42 | 1.2 | 1.02 | 0 | −1.12 | 0.3 |
| 748 | 1392990_at | −1.90 | 2 | −1.67 | 1.4 | 1.61 | 1.3 | 2.11 | 2.5 | 1.13 | 0.2 | −1.10 | 0.2 |
| 749 | 1393003_at | 1.40 | 2.6 | 1.12 | 0.5 | 1.20 | 1 | 1.47 | 3.1 | 1.22 | 1.1 | 1.05 | 0.2 |
| 750 | 1393067_at | −1.32 | 2.1 | −1.00 | 0 | 1.46 | 3.4 | 1.52 | 4.1 | 1.22 | 1.3 | 1.29 | 1.9 |
| 751 | 1393210_at | −4.53 | 10.3 | −3.93 | 9 | −1.47 | 1.3 | −1.51 | 1.5 | 1.15 | 0.3 | 1.23 | 0.5 |
| 752 | 1393235_at | 1.46 | 1.6 | 1.22 | 0.6 | 1.94 | 3.7 | 2.26 | 5 | 1.54 | 1.9 | 1.05 | 0.1 |
| 753 | 1393240_at | 1.87 | 8.7 | 1.88 | 8.8 | 2.17 | 11.6 | 2.33 | 13 | 1.55 | 5.2 | 1.13 | 0.7 |
| 754 | 1393249_at | 1.54 | 2.3 | 1.66 | 3 | 2.68 | 8.3 | 3.38 | 11 | 1.55 | 2.4 | 1.05 | 0.1 |
| 755 | 1393252_at | 1.36 | 1.5 | 1.51 | 2.4 | 1.73 | 3.7 | 1.97 | 5.2 | 1.39 | 1.7 | 1.06 | 0.2 |
| 756 | 1393281_at | −1.27 | 2 | 1.09 | 0.5 | 1.91 | 8.8 | 1.83 | 7.9 | 1.19 | 1.2 | 1.22 | 1.5 |
| 757 | 1393282_at | −1.99 | 9.8 | −1.54 | 5 | −1.14 | 0.9 | −1.37 | 3.1 | −1.14 | 0.8 | −1.23 | 1.6 |
| 758 | 1393316_at | 2.65 | 6.1 | 2.49 | 5.5 | 2.86 | 6.8 | 3.20 | 7.9 | 1.45 | 1.4 | 1.06 | 0.1 |
| 759 | 1393401_at | 1.71 | 1.1 | 1.54 | 0.8 | 3.61 | 4 | 3.69 | 4.1 | 1.55 | 0.8 | −1.39 | 0.5 |
| 760 | 1393427_s_at | 1.18 | 1 | 1.25 | 1.6 | 1.68 | 5.6 | 1.61 | 5 | 1.30 | 1.9 | 1.08 | 0.3 |
| 761 | 1393460_at | 2.02 | 8.7 | 2.13 | 9.6 | 2.26 | 10.6 | 2.15 | 9.7 | 1.46 | 3.4 | 1.07 | 0.3 |
| 762 | 1393706_at | 2.39 | 7.7 | 1.74 | 3.9 | 1.63 | 3.2 | 1.72 | 3.8 | 1.31 | 1.3 | 1.07 | 0.2 |
| 763 | 1393730_at | 6.72 | 11.5 | 4.36 | 8.1 | 6.12 | 10.8 | 8.74 | 13.6 | 2.43 | 3.8 | 2.14 | 3 |
| 764 | 1393799_at | −1.00 | 0 | −1.09 | 0.3 | 1.27 | 1.2 | 1.68 | 4 | 1.20 | 0.8 | 1.03 | 0.1 |
| 765 | 1393883_at | −1.07 | 0.2 | 1.02 | 0 | 1.14 | 0.4 | 1.45 | 1.7 | 1.03 | 0.1 | 1.09 | 0.2 |
| 766 | 1393917_at | 2.89 | 5.1 | 4.22 | 8 | 3.45 | 6.4 | 2.84 | 4.9 | 1.23 | 0.5 | 1.18 | 0.3 |
| 767 | 1393944_at | −1.51 | 2.2 | −1.17 | 0.5 | 1.94 | 4.8 | 1.92 | 4.6 | 2.00 | 5.1 | 1.26 | 0.9 |
| 768 | 1393987_s_at | 1.40 | 7.2 | 1.43 | 7.8 | 1.33 | 5.7 | 1.22 | 3.2 | 1.13 | 1.5 | 1.10 | 1.1 |
| 769 | 1394109_at | 2.10 | 6.4 | 1.96 | 5.6 | 2.24 | 7.3 | 3.00 | 11.2 | 3.57 | 13.5 | 1.48 | 2.4 |
| 770 | 1394315_at | 1.92 | 3.8 | 1.92 | 3.8 | 2.51 | 6.4 | 1.85 | 3.5 | 1.07 | 0.2 | −1.02 | 0 |
| 771 | 1394375_x_at | −1.27 | 0.9 | 1.01 | 0 | 1.87 | 3.8 | 1.90 | 4 | 1.11 | 0.3 | −1.01 | 0 |
| 772 | 1394459_at | −2.52 | 10.8 | −1.86 | 6.2 | −2.12 | 8.2 | −1.48 | 3.1 | −1.01 | 0 | −1.01 | 0 |
| 773 | 1394473_at | 1.26 | 1.2 | 1.43 | 2.3 | 1.55 | 3.2 | 2.03 | 6.7 | 1.45 | 2.5 | 1.18 | 0.8 |
| 774 | 1394483_at | 2.22 | 3.7 | 1.71 | 2 | 1.93 | 2.7 | 2.79 | 5.4 | 1.30 | 0.7 | −1.04 | 0.1 |
| 775 | 1394490_at | −1.02 | 0 | 1.54 | 1.8 | 2.47 | 5.4 | 2.66 | 6.1 | 1.93 | 3.3 | 1.76 | 2.6 |
| 776 | 1394597_at | 1.03 | 0.2 | 1.10 | 0.7 | 1.08 | 0.5 | 1.22 | 2.3 | 1.18 | 1.7 | 1.18 | 1.8 |
| 777 | 1394612_at | −1.36 | 2.3 | −1.42 | 2.8 | −1.29 | 1.7 | −1.14 | 0.7 | −1.08 | 0.3 | 1.16 | 0.8 |
| 778 | 1395079_at | 1.15 | 0.5 | 1.17 | 0.5 | 1.39 | 1.5 | 1.56 | 2.3 | 1.33 | 1.2 | −1.16 | 0.5 |
| 779 | 1395197_at | −1.04 | 0.1 | −1.26 | 1.1 | 1.04 | 0.1 | 1.17 | 0.6 | −1.07 | 0.2 | −1.20 | 0.8 |
| 780 | 1395265_at | −1.17 | 0.9 | 1.23 | 1.4 | 1.27 | 1.7 | 1.47 | 3.6 | 1.17 | 0.9 | 1.03 | 0.1 |
| 781 | 1395357_at | 1.03 | 0.1 | −1.02 | 0 | 1.22 | 0.7 | 1.48 | 1.9 | 1.37 | 1.4 | 1.16 | 0.5 |
| 782 | 1395508_at | 1.66 | 5.1 | 1.46 | 3.3 | 1.36 | 2.4 | 1.38 | 2.6 | 1.11 | 0.5 | −1.02 | 0.1 |
| 783 | 1395635_at | 1.49 | 1.7 | 1.04 | 0.1 | 1.67 | 2.6 | 1.90 | 3.7 | 1.20 | 0.6 | −1.02 | 0 |
| 784 | 1395663_at | −1.32 | 4.8 | −1.16 | 1.8 | −1.17 | 2 | −1.32 | 4.8 | −1.09 | 0.8 | −1.09 | 0.8 |
| 785 | 1395730_at | 1.50 | 3.3 | 1.30 | 1.6 | 1.33 | 1.9 | 1.40 | 2.5 | −1.03 | 0.1 | −1.10 | 0.4 |
| 786 | 1396055_at | −2.92 | 11.1 | −2.20 | 7.3 | −1.37 | 1.7 | −1.06 | 0.2 | 1.46 | 2.4 | −1.04 | 0.1 |
| 787 | 1396208_at | 1.93 | 4.5 | 1.64 | 2.9 | 1.56 | 2.4 | 1.39 | 1.6 | −1.01 | 0 | 1.11 | 0.3 |
| 788 | 1397272_at | −1.56 | 4.4 | −1.71 | 5.8 | −1.60 | 4.7 | −1.66 | 5.3 | −1.07 | 0.3 | −1.30 | 1.9 |
| 789 | 1397317_at | 1.86 | 2.4 | 1.81 | 2.3 | 2.24 | 3.7 | 3.28 | 6.7 | 1.63 | 1.7 | −1.03 | 0.1 |
| 790 | 1397644_at | 1.96 | 6.3 | 1.35 | 1.8 | 1.88 | 5.8 | 2.51 | 9.9 | 1.12 | 0.4 | −1.11 | 0.4 |
| 791 | 1397674_at | 1.37 | 2.4 | 1.23 | 1.3 | 1.03 | 0.1 | 1.23 | 1.3 | −1.06 | 0.2 | −1.12 | 0.6 |
| 792 | 1397808_at | 2.42 | 6 | 2.62 | 6.8 | 3.06 | 8.4 | 3.76 | 10.6 | 1.67 | 2.6 | 1.27 | 0.8 |
| 793 | 1398131_at | −1.02 | 0.1 | −1.14 | 0.6 | 1.30 | 1.7 | 1.45 | 2.9 | 1.17 | 0.8 | 1.07 | 0.3 |
| 794 | 1398246_s_at | 5.99 | 10.9 | 6.83 | 12 | 6.77 | 11.9 | 4.74 | 9 | 1.71 | 1.8 | 1.68 | 1.7 |
| 795 | 1398256_at | 37.28 | 16.8 | 27.02 | 15.1 | 21.59 | 13.9 | 7.03 | 7.5 | 2.02 | 1.6 | −1.01 | 0 |
| 796 | 1398275_at | 30.47 | 8.8 | 41.12 | 9.8 | 150.57 | 14.5 | 80.59 | 12.3 | 2.94 | 1.5 | −1.44 | 0.3 |
| 797 | 1398286_at | −2.12 | 10 | −1.70 | 6.1 | −1.25 | 1.6 | −1.25 | 1.6 | −1.05 | 0.2 | 1.06 | 0.2 |
| 798 | 1398287_at | 1.62 | 3.6 | 1.69 | 4.1 | 2.17 | 7.4 | 1.41 | 2.2 | 1.17 | 0.7 | 1.02 | 0.1 |
| 799 | 1398303_s_at | 1.73 | 6.9 | 1.56 | 5 | 1.82 | 7.7 | 1.58 | 5.2 | 1.21 | 1.3 | −1.17 | 1 |
| 800 | 1398332_at | −1.02 | 0.1 | 1.08 | 0.5 | 1.41 | 4.1 | 1.45 | 4.6 | 1.10 | 0.7 | 1.04 | 0.2 |
| 801 | 1398333_at | −1.13 | 1.5 | −1.01 | 0 | 1.20 | 2.8 | 1.09 | 0.9 | 1.18 | 2.3 | 1.18 | 2.4 |
| 802 | 1398335_at | −1.34 | 4.3 | 1.01 | 0.1 | 1.28 | 3.2 | 1.34 | 4.3 | 1.19 | 1.9 | −1.03 | 0.2 |
| 803 | 1398348_at | 2.10 | 2.8 | 1.33 | 0.7 | 1.33 | 0.7 | 1.59 | 1.3 | −1.03 | 0 | −1.31 | 0.6 |
| 804 | 1398350_at | 1.36 | 1.4 | 1.25 | 0.9 | 1.46 | 1.9 | 2.42 | 7.1 | 1.55 | 2.4 | 1.10 | 0.3 |
| 805 | 1398356_at | 1.59 | 14.6 | 1.60 | 14.8 | 1.46 | 11.3 | 1.29 | 6.5 | −1.17 | 3 | −1.06 | 0.7 |
| 806 | 1398380_at | 2.73 | 13.7 | 2.53 | 12.5 | 2.84 | 14.4 | 2.99 | 15.3 | 1.86 | 7.1 | 1.11 | 0.5 |
| 807 | 1398390_at | 2.81 | 4.1 | 2.57 | 3.6 | 1.64 | 1.3 | 2.62 | 3.7 | 1.51 | 1 | −1.32 | 0.6 |
| 808 | 1398397_at | −1.03 | 0.1 | 1.09 | 0.3 | 2.06 | 7.1 | 2.79 | 11.5 | 1.86 | 5.7 | 1.43 | 2.4 |
| 809 | 1398597_at | −1.19 | 0.9 | −1.35 | 1.9 | 1.61 | 3.8 | 1.50 | 2.9 | 1.30 | 1.5 | 1.35 | 1.9 |
| 810 | 1398626_s_at | 1.36 | 10.6 | 1.35 | 10.3 | 1.31 | 8.9 | 1.28 | 7.8 | 1.01 | 0.1 | 1.00 | 0 |
| 811 | 1398656_at | −1.58 | 0.8 | −5.58 | 5.9 | −2.10 | 1.6 | −4.53 | 4.9 | −2.11 | 1.6 | −1.30 | 0.4 |
| 812 | 1398664_at | −1.76 | 5.4 | −1.26 | 1.4 | −1.15 | 0.6 | −1.09 | 0.4 | −1.12 | 0.5 | −1.12 | 0.5 |
| 813 | 1398727_at | 1.11 | 0.2 | 1.28 | 0.7 | 1.31 | 0.8 | 1.43 | 1.1 | 1.10 | 0.2 | −1.14 | 0.3 |
| 814 | 1398757_at | 2.66 | 19.3 | 2.18 | 14.9 | 2.01 | 13 | 1.87 | 11.4 | 1.35 | 3.9 | 1.10 | 0.7 |
| 815 | 1398765_at | 1.24 | 6 | 1.42 | 11.9 | 1.37 | 10.1 | 1.39 | 11 | 1.13 | 2.5 | 1.01 | 0.1 |
| 816 | 1398828_at | 2.10 | 10.4 | 2.23 | 11.4 | 2.92 | 16.4 | 2.92 | 16.4 | 1.69 | 6.4 | 1.12 | 0.6 |
| 817 | 1398829_at | 1.76 | 11.4 | 1.74 | 11.1 | 2.32 | 18.3 | 2.37 | 18.9 | 1.43 | 6 | 1.08 | 0.6 |
| 818 | 1398833_at | −1.26 | 4.9 | −1.20 | 3.3 | 1.00 | 0 | −1.04 | 0.3 | 1.10 | 1.2 | 1.09 | 1.1 |
| 819 | 1398840_at | 1.02 | 0.1 | 1.09 | 0.5 | 1.52 | 4.8 | 1.57 | 5.4 | 1.30 | 2.3 | 1.11 | 0.6 |
| 820 | 1398851_at | 1.25 | 4.5 | 1.03 | 0.3 | 1.01 | 0.1 | 1.02 | 0.2 | −1.12 | 1.6 | −1.12 | 1.6 |
| 821 | 1398857_at | −1.39 | 6.3 | −1.11 | 1.1 | −1.15 | 1.7 | −1.14 | 1.4 | −1.09 | 0.8 | 1.04 | 0.3 |
| 822 | 1398873_at | 1.20 | 3.2 | −1.01 | 0 | 1.07 | 0.7 | −1.05 | 0.4 | −1.26 | 4.6 | 1.03 | 0.3 |
| 823 | 1398893_at | −1.03 | 0.3 | −1.15 | 2.2 | −1.02 | 0.1 | 1.04 | 0.3 | −1.01 | 0.1 | −1.05 | 0.5 |
| 824 | 1398962_at | 1.15 | 1.6 | 1.12 | 1.1 | 1.34 | 5 | 1.35 | 5.1 | −1.01 | 0.1 | 1.07 | 0.6 |
| 825 | 1398970_at | −1.28 | 4.1 | −1.14 | 1.5 | −1.14 | 1.6 | −1.07 | 0.6 | 1.16 | 1.9 | 1.00 | 0 |
| 826 | 1398994_at | 1.11 | 0.5 | 1.22 | 1.3 | 1.41 | 3 | 1.99 | 8.6 | 1.36 | 2.5 | −1.03 | 0.1 |
| 827 | 1399019_at | −1.10 | 1.9 | 1.08 | 1.3 | 1.10 | 1.8 | 1.14 | 3 | 1.03 | 0.3 | 1.02 | 0.2 |
| 828 | 1399045_at | 1.40 | 5.4 | 1.29 | 3.4 | 1.46 | 6.3 | 1.35 | 4.5 | 1.22 | 2.3 | 1.02 | 0.1 |
| 829 | 1399122_at | −1.11 | 0.9 | −1.16 | 1.4 | −1.20 | 1.9 | −1.19 | 1.7 | 1.03 | 0.2 | 1.04 | 0.2 |

TABLE 3
Time course of gene expression in aortic ring assay

| | | Fold | | Fold | | Fold | | Fold |
|---|---|---|---|---|---|---|---|---|
| | Affymetrix | Change | NLogP | Change | NLogP | Change | NLogP | Change |
| No. | Probe Set ID | Day 0 | Day 0 | Day 1 | Day 1 | Day 2 | Day 2 | Day 4 |
| 1 | 1367453_at | −1.03 | 0.2 | −1.11 | 1.1 | −1.01 | 0 | 1.26 |
| 2 | 1367463_at | −1.03 | 0.3 | −1.26 | 4.7 | −1.12 | 1.6 | 1.07 |
| 3 | 1367538_at | 1.23 | 2 | 1.36 | 4 | 1.24 | 2.2 | 1.30 |
| 4 | 1367574_at | 1.02 | 0.1 | −2.22 | 11.8 | −1.42 | 3.5 | −1.41 |
| 5 | 1367575_at | 1.17 | 0.9 | −1.11 | 0.6 | −1.02 | 0.1 | 1.22 |
| 6 | 1367579_a_at | 1.10 | 0.7 | −1.53 | 6.9 | −1.39 | 4.7 | −1.22 |
| 7 | 1367581_a_at | 1.41 | 1.8 | −1.30 | 1.4 | −1.26 | 1.2 | −1.73 |
| 8 | 1367590_at | 1.11 | 0.9 | −1.32 | 4.4 | −1.25 | 3.1 | −1.23 |
| 9 | 1367594_at | −1.23 | 1.7 | 1.18 | 1.4 | −1.24 | 2 | 1.05 |
| 10 | 1367619_at | 1.31 | 2.3 | 1.23 | 1.7 | 1.10 | 0.5 | −1.11 |
| 11 | 1367627_at | 2.82 | 1.6 | −1.02 | 0 | 1.08 | 0.1 | 1.70 |
| 12 | 1367646_at | 1.18 | 1.4 | −1.26 | 2.5 | −1.30 | 3.1 | −1.46 |
| 13 | 1367652_at | 2.21 | 2 | 1.02 | 0 | 2.99 | 3.5 | 5.14 |
| 14 | 1367658_at | −1.17 | 0.2 | 1.43 | 0.6 | 1.74 | 1.1 | 3.56 |
| 15 | 1367661_at | 1.71 | 2.9 | −1.94 | 4.4 | −1.58 | 2.4 | −1.59 |
| 16 | 1367663_at | 1.06 | 0.3 | 1.02 | 0.1 | 1.14 | 1.2 | 1.36 |
| 17 | 1367671_at | 1.15 | 1.1 | −1.88 | 11.3 | −1.22 | 2 | −1.40 |
| 18 | 1367682_at | 1.05 | 0.2 | 1.73 | 8.1 | 1.46 | 4.6 | 1.55 |
| 19 | 1367693_at | 1.10 | 0.8 | −1.68 | 10.5 | −1.35 | 4.8 | −1.26 |
| 20 | 1367701_at | 2.32 | 9.1 | 2.36 | 10 | 3.01 | 13.9 | 2.81 |
| 21 | 1367716_at | 1.04 | 0.3 | 1.01 | 0.1 | −1.00 | 0 | −1.01 |
| 22 | 1367721_at | 1.02 | 0.1 | 1.41 | 4.7 | 1.40 | 4.5 | 1.68 |
| 23 | 1367722_at | 1.17 | 0.9 | 1.23 | 1.5 | −1.07 | 0.3 | 1.10 |
| 24 | 1367732_at | 1.02 | 0.2 | −1.21 | 3.1 | −1.13 | 1.6 | −1.23 |
| 25 | 1367733_at | 1.39 | 0.4 | 1.75 | 0.9 | −1.15 | 0.2 | 1.29 |
| 26 | 1367749_at | 1.08 | 0.3 | 1.86 | 5.9 | 1.47 | 2.8 | 1.63 |
| 27 | 1367760_at | 1.08 | 0.9 | −1.05 | 0.5 | 1.08 | 0.9 | 1.07 |
| 28 | 1367765_at | 1.12 | 0.5 | 1.58 | 4 | 1.31 | 1.8 | 1.32 |
| 29 | 1367774_at | 1.14 | 0.5 | 2.15 | 7.5 | 1.37 | 1.9 | −1.01 |
| 30 | 1367784_a_at | 1.82 | 5 | 1.64 | 4 | 1.55 | 3.3 | 3.10 |
| 31 | 1367786_at | 1.03 | 0.1 | 1.50 | 1.6 | 1.76 | 2.7 | 2.14 |
| 32 | 1367800_at | 1.43 | 2.6 | 2.90 | 13.6 | 1.48 | 3.2 | 1.51 |
| 33 | 1367801_at | 1.96 | 10 | 1.77 | 8.5 | 1.65 | 7 | 2.21 |
| 34 | 1367807_at | 1.24 | 1.5 | 1.71 | 6.3 | 1.07 | 0.3 | 1.38 |
| 35 | 1367814_at | −1.02 | 0.1 | 1.10 | 0.3 | −1.12 | 0.4 | −1.20 |
| 36 | 1367823_at | 1.10 | 0.4 | 1.28 | 1.4 | 1.04 | 0.1 | 1.08 |
| 37 | 1367846_at | −1.08 | 0.3 | −1.57 | 3.8 | −1.92 | 6.6 | −2.29 |
| 38 | 1367850_at | 1.28 | 0.6 | 2.19 | 3.9 | 2.72 | 5.7 | 4.07 |
| 39 | 1367859_at | 1.06 | 0.2 | −1.19 | 0.8 | −1.25 | 1.2 | 1.07 |
| 40 | 1367881_at | 1.10 | 0.5 | 1.75 | 8 | 1.40 | 3.7 | 1.89 |
| 41 | 1367902_at | −1.09 | 0.5 | 1.31 | 2.9 | 1.20 | 1.6 | 1.52 |
| 42 | 1367905_at | 1.02 | 0 | −1.14 | 0.4 | −1.01 | 0 | 1.37 |
| 43 | 1367914_at | −1.01 | 0 | −1.80 | 8.4 | −1.66 | 6.7 | −1.49 |
| 44 | 1367930_at | 1.97 | 1 | −2.18 | 1.3 | 1.80 | 0.9 | 1.29 |
| 45 | 1367940_at | 1.75 | 4.5 | 2.25 | 8.5 | 1.89 | 5.9 | 1.40 |
| 46 | 1367942_at | 1.06 | 0.1 | 1.27 | 0.8 | 1.97 | 4 | 1.23 |
| 47 | 1367948_a_at | −1.81 | 2 | 1.60 | 1.5 | 2.15 | 3.2 | 4.20 |
| 48 | 1367959_a_at | 1.24 | 0.8 | 2.43 | 6.9 | 1.75 | 3.4 | 1.61 |
| 49 | 1367973_at | 2.07 | 4.2 | 1.64 | 2.7 | 1.22 | 0.7 | −1.35 |
| 50 | 1367974_at | 1.32 | 0.8 | 1.17 | 0.4 | 1.53 | 1.6 | 2.03 |
| 51 | 1367998_at | 1.34 | 0.3 | 8.01 | 5.9 | 14.64 | 8.6 | 21.25 |
| 52 | 1368000_at | −1.39 | 0.8 | 6.11 | 11.1 | 6.68 | 11.9 | 5.20 |
| 53 | 1368049_at | −1.07 | 0.4 | −1.52 | 6 | −1.18 | 1.4 | 1.18 |
| 54 | 1368052_at | −1.00 | 0 | −1.11 | 0.1 | 3.25 | 2.1 | 3.77 |
| 55 | 1368057_at | −1.01 | 0 | 1.08 | 0.3 | 1.30 | 1.8 | 1.10 |
| 56 | 1368078_at | 1.13 | 0.2 | −3.65 | 4.6 | −2.85 | 3.3 | −6.70 |
| 57 | 1368079_at | 1.56 | 1.7 | −1.02 | 0 | 1.89 | 3.4 | 1.73 |
| 58 | 1368089_at | 1.02 | 0.1 | 1.31 | 1.4 | 2.00 | 5.9 | 2.44 |
| 59 | 1368097_a_at | −1.10 | 0.2 | 1.70 | 1.9 | 1.44 | 1.1 | 3.22 |
| 60 | 1368103_at | 1.25 | 0.6 | 4.52 | 11.4 | 2.34 | 5 | 2.38 |
| 61 | 1368106_at | 1.14 | 0.3 | 1.16 | 0.3 | 2.09 | 3 | 2.59 |
| 62 | 1368145_at | −2.50 | 2.3 | −1.01 | 0 | −1.11 | 0.1 | −2.38 |
| 63 | 1368173_at | −1.32 | 2.7 | −1.50 | 5.2 | −1.16 | 1.1 | −1.15 |
| 64 | 1368183_at | −1.19 | 1.2 | 1.22 | 1.5 | 1.53 | 4.9 | 1.24 |
| 65 | 1368200_at | 1.65 | 2.6 | −1.26 | 0.8 | −1.85 | 3.8 | −1.22 |
| 66 | 1368221_at | 1.18 | 0.8 | 1.11 | 0.4 | 1.29 | 1.6 | 1.21 |
| 67 | 1368223_at | −1.13 | 0.3 | 2.80 | 7.3 | 2.20 | 4.8 | 1.55 |
| 68 | 1368259_at | 1.22 | 0.6 | 1.75 | 3.1 | 1.61 | 2.4 | −1.01 |
| 69 | 1368280_at | 1.21 | 0.6 | 1.67 | 2.8 | 1.36 | 1.3 | 1.14 |
| 70 | 1368281_at | −1.29 | 1.1 | 1.00 | 0 | 1.24 | 0.9 | 1.29 |
| 71 | 1368322_at | 1.04 | 0.1 | 2.76 | 10.8 | 1.36 | 1.7 | 1.55 |
| 72 | 1368332_at | 1.05 | 0.1 | −2.15 | 2.5 | 1.18 | 0.3 | 2.88 |
| 73 | 1368347_at | 1.42 | 0.5 | 1.70 | 1 | 3.15 | 3.2 | 10.74 |
| 74 | 1368370_at | 1.04 | 0.1 | 1.13 | 0.4 | 1.11 | 0.3 | 1.34 |
| 75 | 1368393_at | 2.23 | 3.6 | 6.83 | 13.6 | 4.44 | 9.7 | 3.83 |
| 76 | 1368395_at | 2.51 | 1.7 | 4.46 | 3.9 | 4.05 | 3.6 | 4.95 |
| 77 | 1368404_at | 1.01 | 0 | −1.15 | 0.7 | −1.07 | 0.3 | 1.40 |
| 78 | 1368419_at | 1.42 | 1.4 | 2.32 | 5.9 | 1.67 | 2.7 | 1.89 |
| 79 | 1368420_at | −1.14 | 0.4 | 1.09 | 0.2 | −1.04 | 0.1 | −1.02 |
| 80 | 1368430_at | 1.12 | 0.8 | 1.57 | 6.7 | 1.41 | 4.4 | 1.67 |
| 81 | 1368448_at | 1.38 | 2.5 | 1.44 | 3.2 | 1.36 | 2.4 | 1.56 |
| 82 | 1368464_at | 2.98 | 2.3 | 1.54 | 0.6 | 3.36 | 2.9 | 7.11 |
| 83 | 1368474_at | −1.01 | 0 | 1.29 | 1.8 | 1.17 | 0.9 | −1.04 |
| 84 | 1368519_at | 1.89 | 0.8 | 8.51 | 5.7 | 11.18 | 6.8 | 8.19 |
| 85 | 1368612_at | 1.26 | 0.7 | −1.02 | 0.1 | 1.30 | 0.9 | 1.36 |
| 86 | 1368655_at | 1.31 | 2 | 1.59 | 5.1 | 1.08 | 0.4 | 1.10 |
| 87 | 1368657_at | −1.15 | 0.4 | 1.51 | 2 | −1.08 | 0.2 | −1.33 |
| 88 | 1368731_at | −1.68 | 0.9 | 12.95 | 10.4 | 3.30 | 3.3 | 3.16 |
| 89 | 1368771_at | 1.49 | 2.4 | 2.21 | 7.4 | 1.95 | 5.8 | 2.63 |
| 90 | 1368813_at | 1.48 | 1.6 | 2.44 | 5.9 | 1.18 | 0.5 | 2.11 |
| 91 | 1368851_at | 1.19 | 0.9 | −1.08 | 0.3 | 1.06 | 0.2 | 1.41 |
| 92 | 1368885_at | 1.20 | 0.6 | 2.37 | 6.6 | 1.58 | 2.5 | 1.44 |
| 93 | 1368989_at | 1.15 | 0.4 | −2.05 | 4.4 | −1.74 | 2.9 | −1.85 |
| 94 | 1369006_at | −1.13 | 0.3 | −1.10 | 0.2 | 1.27 | 0.8 | 1.24 |
| 95 | 1369044_a_at | 1.10 | 0.2 | 2.50 | 4.6 | 2.28 | 3.9 | 3.33 |
| 96 | 1369065_a_at | −1.03 | 0.1 | 1.66 | 7.2 | 1.26 | 2.1 | 1.15 |
| 97 | 1369087_at | −1.19 | 0.6 | 1.57 | 2.6 | 1.34 | 1.3 | 1.11 |
| 98 | 1369166_at | 1.31 | 0.3 | 3.08 | 2.4 | 6.04 | 5 | 3.05 |
| 99 | 1369269_at | −1.20 | 0.9 | 1.06 | 0.2 | 1.23 | 1.2 | 1.19 |
| 100 | 1369294_at | 2.87 | 4.8 | 9.37 | 14.8 | 3.19 | 5.6 | 3.48 |
| 101 | 1369313_at | −1.19 | 0.4 | −1.40 | 1.1 | 1.19 | 0.4 | 1.06 |
| 102 | 1369422_at | 1.13 | 0.4 | −1.04 | 0.1 | −1.15 | 0.5 | 1.03 |
| 103 | 1369425_at | −1.33 | 1.3 | −1.13 | 0.4 | −1.13 | 0.4 | 1.04 |
| 104 | 1369484_at | 1.08 | 0.3 | −1.81 | 5.3 | −1.51 | 2.8 | −1.14 |
| 105 | 1369621_s_at | 1.28 | 2.1 | −1.10 | 0.6 | −1.06 | 0.3 | −1.18 |
| 106 | 1369633_at | 1.22 | 0.6 | −2.11 | 4.5 | −1.07 | 0.2 | 1.91 |
| 107 | 1369651_at | −1.01 | 0 | −1.19 | 0.6 | −1.20 | 0.7 | 1.15 |
| 108 | 1369652_at | 1.16 | 0.3 | 1.19 | 0.4 | −1.36 | 0.8 | 1.33 |
| 109 | 1369703_at | −1.30 | 1.4 | 1.78 | 5.2 | 1.48 | 2.9 | 1.06 |
| 110 | 1369735_at | −1.28 | 1.5 | 1.29 | 1.8 | −1.01 | 0 | −1.13 |
| 111 | 1369815_at | −1.46 | 1.2 | 3.37 | 7.9 | 2.42 | 4.9 | 3.38 |
| 112 | 1369895_s_at | 2.79 | 3.2 | 2.21 | 2.3 | 2.76 | 3.4 | 4.01 |
| 113 | 1369897_s_at | 1.22 | 3 | −1.06 | 0.6 | −1.10 | 1.1 | 1.01 |
| 114 | 1369926_at | 1.10 | 0.3 | 1.80 | 4.9 | 1.48 | 2.6 | 1.70 |
| 115 | 1369931_at | 1.20 | 1.8 | −1.17 | 1.6 | −1.07 | 0.5 | 1.24 |
| 116 | 1369947_at | 1.35 | 1.8 | 1.23 | 1.1 | −1.10 | 0.4 | −1.05 |
| 117 | 1369950_at | 1.08 | 0.5 | −1.39 | 5.1 | −1.09 | 0.7 | 1.14 |
| 118 | 1369953_a_at | 2.44 | 3.2 | 2.96 | 4.6 | 1.75 | 1.7 | 1.49 |
| 119 | 1369955_at | 1.25 | 1.1 | −1.01 | 0 | −1.02 | 0.1 | 2.52 |
| 120 | 1369956_at | 1.13 | 1.3 | 1.55 | 9.8 | 1.29 | 4.4 | 1.34 |
| 121 | 1369958_at | 1.03 | 0.1 | 1.30 | 2.2 | 1.24 | 1.6 | 1.54 |
| 122 | 1369960_at | −1.16 | 1.2 | 1.09 | 0.6 | −1.11 | 0.8 | −1.07 |
| 123 | 1369961_at | 1.23 | 1.2 | 3.66 | 18.4 | 2.12 | 9.2 | 1.76 |
| 124 | 1369973_at | 1.50 | 3.4 | 1.69 | 5.4 | 1.27 | 1.6 | 1.81 |
| 125 | 1370007_at | 1.47 | 4.3 | −1.09 | 0.5 | −1.05 | 0.3 | 1.13 |
| 126 | 1370023_at | 1.05 | 0.2 | −1.29 | 1.7 | 1.05 | 0.2 | −1.18 |
| 127 | 1370048_at | −1.06 | 0.1 | 1.66 | 2.9 | 1.91 | 4.2 | 1.48 |
| 128 | 1370051_at | 2.01 | 1.6 | 2.12 | 1.9 | 1.16 | 0.2 | 2.58 |
| 129 | 1370057_at | −1.06 | 0.3 | 1.18 | 1.1 | 1.10 | 0.5 | 1.26 |
| 130 | 1370062_at | 1.08 | 0.3 | 1.04 | 0.2 | 1.39 | 2.5 | 1.53 |
| 131 | 1370073_at | 1.04 | 0.2 | 1.01 | 0 | −1.08 | 0.4 | −1.16 |
| 132 | 1370097_a_at | 1.13 | 0.4 | 1.87 | 3.9 | 2.12 | 5.2 | 1.35 |
| 133 | 1370172_at | 1.20 | 1.4 | 1.57 | 6 | 1.08 | 0.5 | 1.13 |
| 134 | 1370184_at | 1.05 | 0.4 | −1.22 | 3.3 | −1.29 | 4.8 | −1.10 |
| 135 | 1370221_at | 1.16 | 0.4 | 2.24 | 4.6 | 1.79 | 2.8 | 1.83 |
| 136 | 1370244_at | 1.51 | 4.2 | −1.11 | 0.6 | −1.18 | 1.1 | −1.49 |
| 137 | 1370248_at | −1.21 | 0.5 | 3.15 | 7.1 | 5.25 | 11.9 | 3.74 |
| 138 | 1370256_at | 1.01 | 0 | 1.42 | 2.4 | 1.21 | 1 | 2.39 |
| 139 | 1370290_at | 1.12 | 0.8 | −1.43 | 4.5 | −1.21 | 1.7 | 1.31 |
| 140 | 1370301_at | −1.07 | 0.3 | 1.83 | 9.3 | 1.46 | 4.7 | 1.66 |
| 141 | 1370309_a_at | 1.15 | 0.8 | −1.47 | 4.3 | −1.23 | 1.6 | −1.33 |
| 142 | 1370312_at | 1.33 | 0.9 | 2.60 | 6 | 2.81 | 6.7 | 3.80 |
| 143 | 1370328_at | −1.08 | 0.3 | −1.40 | 2.4 | −1.50 | 3.2 | 1.04 |
| 144 | 1370408_at | 1.73 | 5.2 | 1.41 | 2.7 | 1.03 | 0.1 | 1.13 |
| 145 | 1370442_at | −1.06 | 0.1 | 1.58 | 1.2 | 1.06 | 0.1 | 1.33 |
| 146 | 1370570_at | −1.16 | 0.5 | −1.08 | 0.3 | 1.19 | 0.7 | 1.23 |
| 147 | 1370613_s_at | 3.20 | 4.7 | 3.58 | 5.8 | 2.34 | 3.1 | 3.76 |
| 148 | 1370624_at | 1.03 | 0 | 1.20 | 0.3 | 1.12 | 0.2 | 1.39 |
| 149 | 1370633_at | −1.21 | 0.2 | 5.48 | 4.9 | 6.00 | 5.4 | 4.07 |
| 150 | 1370634_x_at | 1.46 | 0.5 | 5.31 | 5.4 | 3.71 | 3.7 | 3.14 |
| 151 | 1370642_s_at | 1.05 | 0.2 | −1.41 | 3.9 | −1.36 | 3.3 | −1.24 |
| 152 | 1370693_a_at | −1.22 | 1 | 1.69 | 4.7 | 1.56 | 3.7 | 1.38 |
| 153 | 1370802_at | −1.03 | 0.1 | −1.14 | 0.7 | −1.09 | 0.4 | −1.32 |
| 154 | 1370809_at | 1.22 | 1.7 | −1.54 | 5.9 | −1.18 | 1.3 | −1.25 |
| 155 | 1370828_at | −1.86 | 2.6 | −1.31 | 0.8 | −1.07 | 0.1 | −1.35 |
| 156 | 1370838_s_at | 1.02 | 0.1 | 1.74 | 13.5 | 1.19 | 2.5 | 1.28 |
| 157 | 1370845_at | −1.06 | 0.2 | 1.39 | 2.8 | 1.35 | 2.4 | 1.86 |
| 158 | 1370854_at | 1.09 | 0.2 | 1.03 | 0.1 | 1.20 | 0.7 | −1.55 |
| 159 | 1370855_at | 1.13 | 0.8 | −1.22 | 1.8 | −1.49 | 5.3 | −1.31 |
| 160 | 1370862_at | 1.63 | 2.6 | 2.57 | 7.6 | 2.24 | 6 | 3.27 |
| 161 | 1370892_at | 1.35 | 0.9 | 2.53 | 5.4 | 2.04 | 3.6 | 3.48 |
| 162 | 1370905_at | 1.24 | 1.8 | 1.64 | 7.2 | 1.41 | 4.1 | 1.85 |
| 163 | 1370927_at | 1.46 | 1.5 | 1.28 | 0.9 | 1.93 | 3.7 | 1.75 |
| 164 | 1370950_at | 1.25 | 0.8 | 2.11 | 5.7 | 1.97 | 4.9 | 1.99 |
| 165 | 1370954_at | 1.05 | 0.2 | 1.03 | 0.1 | 1.24 | 1.6 | 1.58 |
| 166 | 1370956_at | 1.73 | 3.9 | 2.64 | 9.9 | 1.77 | 4.5 | 1.30 |
| 167 | 1370963_at | 1.14 | 0.6 | −1.15 | 0.7 | −1.25 | 1.2 | −1.18 |
| 168 | 1371016_at | −1.24 | 0.8 | 3.28 | 11 | 2.48 | 7.6 | 1.78 |
| 169 | 1371037_at | −1.12 | 0.6 | 1.45 | 4.1 | 1.01 | 0.1 | 1.40 |
| 170 | 1371079_at | 1.08 | 0.1 | 5.07 | 4 | 7.69 | 5.7 | 11.73 |
| 171 | 1371166_at | 3.10 | 4.2 | 4.39 | 6.8 | 11.18 | 13.5 | 9.26 |
| 172 | 1371232_a_at | 1.04 | 0.1 | −1.03 | 0.1 | −1.04 | 0.1 | −1.13 |
| 173 | 1371246_at | 1.05 | 0.2 | −1.59 | 5.5 | −1.34 | 2.7 | −1.03 |
| 174 | 1371250_at | 2.13 | 3.4 | 1.42 | 1.1 | 2.54 | 5 | 3.71 |
| 175 | 1371320_at | −1.13 | 1.1 | 1.25 | 3 | 1.12 | 1 | 1.43 |
| 176 | 1371329_at | 1.08 | 0.5 | −1.50 | 6.3 | −1.23 | 2.3 | −1.02 |
| 177 | 1371341_at | 1.29 | 1.7 | −1.35 | 2.4 | −1.24 | 1.5 | −1.01 |
| 178 | 1371349_at | 1.01 | 0 | −1.06 | 0.2 | −1.14 | 0.6 | 1.14 |
| 179 | 1371356_at | 1.18 | 0.8 | 1.17 | 0.8 | 1.28 | 1.6 | 2.12 |
| 180 | 1371360_at | 1.08 | 0.2 | 1.84 | 5.1 | 1.85 | 5.2 | 2.76 |
| 181 | 1371369_at | 1.17 | 0.7 | −1.08 | 0.3 | −1.41 | 2.3 | 1.35 |
| 182 | 1371392_at | −1.00 | 0 | 1.10 | 0.5 | 1.30 | 2 | 1.69 |
| 183 | 1371440_at | 1.01 | 0 | 1.14 | 0.6 | 1.08 | 0.3 | 1.60 |
| 184 | 1371447_at | 1.88 | 1.7 | 5.44 | 8.1 | 3.60 | 5.3 | 6.31 |
| 185 | 1371487_at | 1.19 | 1.1 | −1.31 | 2.3 | −1.20 | 1.3 | 1.11 |
| 186 | 1371498_at | 1.03 | 0.2 | −1.42 | 6.1 | −1.16 | 1.7 | −1.04 |
| 187 | 1371507_at | 1.02 | 0.1 | 1.03 | 0.2 | −1.03 | 0.1 | −1.03 |
| 188 | 1371525_at | −1.05 | 0.2 | 1.48 | 4.4 | 1.34 | 2.8 | 1.20 |
| 189 | 1371541_at | −1.06 | 0.3 | 2.14 | 10.7 | 1.33 | 2.4 | 1.42 |
| 190 | 1371545_at | 4.02 | 10.2 | 6.05 | 15.1 | 6.82 | 16.3 | 8.61 |
| 191 | 1371572_at | 1.23 | 1.7 | 1.39 | 3.8 | 1.53 | 5.6 | 1.95 |
| 192 | 1371575_at | 1.11 | 0.8 | −1.49 | 6.3 | −1.45 | 5.5 | −1.23 |
| 193 | 1371596_at | 1.11 | 1.1 | −1.15 | 1.9 | −1.04 | 0.3 | 1.10 |
| 194 | 1371600_at | 1.05 | 0.3 | −1.17 | 1.2 | 1.04 | 0.2 | 1.11 |
| 195 | 1371602_at | 1.10 | 0.6 | 1.66 | 8.9 | 1.31 | 3.5 | 1.13 |
| 196 | 1371618_s_at | −1.34 | 0.4 | 2.41 | 2.2 | 1.30 | 0.4 | 1.97 |
| 197 | 1371625_at | 1.19 | 1.1 | 1.34 | 2.6 | 1.23 | 1.5 | 1.53 |
| 198 | 1371632_at | 1.10 | 0.5 | −1.07 | 0.3 | −1.10 | 0.5 | 1.31 |
| 199 | 1371641_at | 1.11 | 1.2 | −1.29 | 5.1 | −1.11 | 1.3 | 1.13 |
| 200 | 1371691_at | −1.19 | 0.9 | 1.61 | 4.4 | 1.19 | 1 | 1.05 |
| 201 | 1371694_at | 1.13 | 0.9 | −1.08 | 0.5 | 1.03 | 0.2 | 1.40 |
| 202 | 1371727_at | 1.46 | 7.3 | −1.24 | 3.3 | −1.24 | 3.4 | −1.36 |
| 203 | 1371790_at | −1.36 | 4.2 | −1.07 | 0.5 | 1.00 | 0 | −1.03 |
| 204 | 1371824_at | 1.25 | 0.7 | 1.02 | 0 | 1.66 | 2.6 | 1.78 |
| 205 | 1371840_at | 1.21 | 0.7 | 1.87 | 4.6 | 1.94 | 5.1 | 2.75 |
| 206 | 1371849_at | 1.19 | 1 | −1.59 | 4.6 | −1.42 | 3 | −1.50 |
| 207 | 1371894_at | −1.03 | 0.2 | 1.23 | 2.7 | 1.15 | 1.5 | 1.49 |
| 208 | 1371924_at | 1.08 | 0.1 | 1.23 | 0.5 | 1.92 | 2.9 | 2.50 |
| 209 | 1371926_at | 1.14 | 1.4 | 1.69 | 12.2 | 1.50 | 8.7 | 1.81 |
| 210 | 1371951_at | 1.66 | 1.9 | 1.05 | 0.1 | 1.45 | 1.3 | 1.50 |
| 211 | 1371986_at | −1.23 | 1.5 | −1.18 | 1.1 | 1.12 | 0.7 | 1.59 |
| 212 | 1371988_at | 1.31 | 1.5 | 1.85 | 5.7 | 1.71 | 4.6 | 1.81 |
| 213 | 1371999_at | −1.02 | 0.1 | −1.40 | 3.3 | 1.02 | 0.1 | −1.06 |
| 214 | 1372005_at | −1.44 | 2.9 | −1.60 | 4.7 | 1.02 | 0.1 | −1.06 |
| 215 | 1372006_at | 1.22 | 1 | 1.16 | 0.7 | 1.46 | 2.9 | 1.62 |
| 216 | 1372028_at | 1.06 | 0.3 | −1.05 | 0.3 | 1.12 | 0.9 | 1.42 |
| 217 | 1372031_at | 1.50 | 4.5 | 1.16 | 1.1 | 1.05 | 0.2 | 1.21 |
| 218 | 1372050_at | −1.02 | 0.1 | 1.05 | 0.3 | −1.00 | 0 | 1.15 |
| 219 | 1372068_at | 1.08 | 0.6 | 1.03 | 0.2 | 1.02 | 0.1 | −1.08 |
| 220 | 1372084_at | 1.30 | 0.9 | −1.55 | 2.2 | 1.08 | 0.2 | 1.30 |
| 221 | 1372084_at | 1.30 | 0.9 | −1.55 | 2.2 | 1.08 | 0.2 | 1.30 |
| 222 | 1372088_at | 1.30 | 2 | 1.01 | 0 | −1.06 | 0.3 | −1.08 |
| 223 | 1372101_at | 1.73 | 2.7 | 2.02 | 4.2 | 1.87 | 3.5 | 2.65 |
| 224 | 1372104_at | 1.03 | 0.1 | −1.08 | 0.3 | 1.00 | 0 | −1.03 |
| 225 | 1372111_at | −1.03 | 0.1 | −2.81 | 10.9 | −1.52 | 2.8 | −1.02 |
| 226 | 1372127_at | −1.09 | 0.8 | −1.24 | 3.1 | −1.00 | 0 | −1.00 |
| 227 | 1372136_at | 1.07 | 0.7 | 1.32 | 6.5 | 1.28 | 5.3 | 1.41 |
| 228 | 1372164_at | 1.03 | 0.2 | 1.62 | 9.2 | 1.20 | 2.1 | 1.41 |
| 229 | 1372213_at | −1.75 | 3.1 | 1.04 | 0.1 | 1.15 | 0.5 | 1.12 |
| 230 | 1372223_at | 1.30 | 1.1 | 1.85 | 4.5 | 1.01 | 0 | −1.13 |
| 231 | 1372234_at | 1.05 | 0.3 | −1.12 | 1.2 | 1.00 | 0 | 1.08 |
| 232 | 1372251_at | 1.06 | 0.6 | 1.06 | 0.6 | 1.03 | 0.3 | 1.06 |
| 233 | 1372254_at | 1.44 | 2.2 | 3.46 | 14.3 | 2.19 | 7.7 | 3.16 |
| 234 | 1372256_at | 1.17 | 0.6 | −1.29 | 1.2 | −1.23 | 0.9 | −1.17 |
| 235 | 1372266_at | 1.11 | 0.3 | 1.99 | 4.6 | 1.59 | 2.5 | 1.07 |
| 236 | 1372294_at | 1.06 | 0.4 | −1.05 | 0.3 | −1.04 | 0.3 | 1.12 |
| 237 | 1372311_at | −1.13 | 0.9 | −1.38 | 4.5 | −1.22 | 2.1 | −1.10 |
| 238 | 1372326_at | −1.38 | 1.6 | 1.21 | 0.8 | 1.81 | 4.4 | 2.39 |
| 239 | 1372410_at | −1.07 | 0.2 | 1.04 | 0.1 | 1.06 | 0.2 | 1.15 |
| 240 | 1372439_at | 1.07 | 0.3 | 2.13 | 10.5 | 1.42 | 3.3 | 1.75 |
| 241 | 1372466_at | 1.35 | 3.2 | 1.30 | 2.8 | 1.17 | 1.3 | 1.53 |
| 242 | 1372518_at | 1.47 | 1.4 | 2.92 | 7.2 | 2.87 | 7 | 5.62 |
| 243 | 1372549_at | −1.52 | 1.5 | 1.07 | 0.1 | 1.83 | 2.9 | 2.19 |
| 244 | 1372569_at | 1.02 | 0.1 | −1.40 | 4.6 | −1.20 | 1.8 | 1.07 |
| 245 | 1372579_at | 1.43 | 1.6 | 2.12 | 5.5 | 2.38 | 6.9 | 2.45 |
| 246 | 1372585_at | 1.01 | 0 | −1.05 | 0.1 | 1.40 | 1.1 | 2.53 |
| 247 | 1372587_at | −1.45 | 1.3 | 1.20 | 0.5 | 1.25 | 0.7 | −1.07 |
| 248 | 1372593_at | 1.25 | 1.4 | 1.55 | 4.3 | 1.29 | 1.9 | 1.22 |
| 249 | 1372610_at | 1.09 | 0.4 | −1.03 | 0.1 | 1.02 | 0.1 | 1.26 |
| 250 | 1372613_at | 1.10 | 0.2 | −1.30 | 1 | −1.10 | 0.3 | −1.04 |
| 251 | 1372615_at | 1.19 | 0.6 | 2.59 | 7.6 | 1.49 | 2 | 1.65 |
| 252 | 1372638_at | −1.17 | 1.3 | 1.11 | 0.8 | 1.22 | 2 | 1.58 |
| 253 | 1372727_at | 1.16 | 0.4 | 1.79 | 2.8 | 1.60 | 2 | 1.62 |
| 254 | 1372729_at | 3.87 | 10.4 | 1.76 | 3 | 1.53 | 1.9 | −1.02 |
| 255 | 1372761_at | −1.08 | 0.1 | 1.61 | 1.1 | 3.29 | 4.3 | 3.24 |
| 256 | 1372769_at | −1.16 | 1.3 | −1.01 | 0 | 1.15 | 1.3 | 1.28 |
| 257 | 1372776_at | 1.12 | 0.6 | 1.75 | 6.2 | 1.64 | 5.1 | 1.88 |
| 258 | 1372818_at | 1.09 | 0.4 | 1.34 | 2.7 | 1.23 | 1.5 | 1.22 |
| 259 | 1372835_at | 1.17 | 0.9 | 1.68 | 6.1 | 1.62 | 5.4 | 1.97 |
| 260 | 1372836_at | −1.01 | 0 | 1.52 | 4.8 | 1.20 | 1.3 | 1.25 |
| 261 | 1372838_at | −1.40 | 4.3 | −1.02 | 0.1 | 1.18 | 1.5 | −1.07 |
| 262 | 1372844_at | 1.85 | 2.7 | 1.87 | 3 | 1.87 | 3 | 2.20 |
| 263 | 1372861_at | 1.14 | 0.7 | −1.09 | 0.4 | 1.04 | 0.2 | 1.58 |
| 264 | 1372919_at | −1.30 | 3 | 1.15 | 1.3 | 1.02 | 0.1 | 1.01 |
| 265 | 1372947_at | −1.04 | 0.2 | −1.35 | 3.3 | −1.26 | 2.2 | −1.31 |
| 266 | 1373000_at | 1.10 | 0.3 | −2.64 | 7.8 | −1.37 | 1.4 | −1.22 |
| 267 | 1373062_at | 1.45 | 2.1 | 2.51 | 8.8 | 2.38 | 8.1 | 3.05 |
| 268 | 1373079_at | 1.02 | 0.1 | 1.52 | 4.6 | 1.57 | 5.2 | 1.90 |
| 269 | 1373102_at | −1.02 | 0 | 1.01 | 0 | −1.04 | 0.1 | 1.06 |
| 270 | 1373140_at | 1.44 | 2.9 | 1.89 | 7.3 | 1.95 | 7.8 | 2.23 |
| 271 | 1373151_at | 1.03 | 0.2 | 1.02 | 0.1 | −1.12 | 0.7 | −1.22 |
| 272 | 1373180_at | 1.10 | 0.4 | −1.47 | 3.4 | −1.17 | 0.9 | −1.44 |
| 273 | 1373181_at | 1.18 | 0.6 | 1.31 | 1.3 | 1.41 | 1.9 | 1.40 |
| 274 | 1373217_at | −1.05 | 0.3 | 1.42 | 4.5 | 1.31 | 3 | 1.07 |
| 275 | 1373222_at | −1.03 | 0.2 | 1.08 | 0.6 | −1.08 | 0.5 | 1.14 |
| 276 | 1373245_at | 1.09 | 0.6 | 2.39 | 18 | 1.82 | 12.1 | 2.24 |
| 277 | 1373258_at | −1.06 | 0.3 | 1.39 | 3.5 | 1.26 | 2 | 1.49 |
| 278 | 1373286_at | −1.18 | 1.1 | 1.20 | 1.4 | −1.10 | 0.5 | −1.35 |
| 279 | 1373301_at | 1.12 | 0.6 | 1.21 | 1.3 | 1.61 | 5.3 | 1.93 |
| 280 | 1373310_at | 2.06 | 5.9 | 1.57 | 3.3 | 1.63 | 3.7 | 2.09 |
| 281 | 1373346_at | 1.15 | 1 | 1.10 | 0.6 | 1.07 | 0.4 | −1.19 |
| 282 | 1373363_at | −1.08 | 0.4 | −1.08 | 0.4 | −1.60 | 5.3 | −1.96 |
| 283 | 1373374_at | 1.01 | 0.1 | 1.37 | 4.1 | 1.12 | 0.9 | 1.49 |
| 284 | 1373379_at | 1.04 | 0.2 | −1.05 | 0.3 | −1.15 | 1.3 | −1.16 |
| 285 | 1373401_at | −1.15 | 0.2 | 3.10 | 4.2 | 8.17 | 10.5 | 11.91 |
| 286 | 1373415_at | 3.43 | 4.4 | 1.85 | 1.6 | 2.10 | 2.2 | 3.49 |
| 287 | 1373463_at | 1.10 | 0.5 | 1.01 | 0.1 | 1.17 | 1.1 | 1.63 |
| 288 | 1373466_at | 1.15 | 0.7 | −1.12 | 0.6 | 1.00 | 0 | −1.22 |
| 289 | 1373483_at | −1.41 | 1.3 | 1.10 | 0.2 | 1.44 | 1.5 | 1.46 |
| 290 | 1373487_at | 1.20 | 0.4 | 1.76 | 2.3 | 2.03 | 3.2 | 2.60 |
| 291 | 1373488_at | 1.14 | 1.2 | −1.55 | 8 | −1.27 | 3.1 | −1.22 |
| 292 | 1373515_at | −1.03 | 0.1 | 1.16 | 1.3 | 1.11 | 0.8 | −1.07 |
| 293 | 1373533_at | −1.08 | 0.4 | 1.31 | 2.7 | 1.40 | 3.8 | 2.14 |
| 294 | 1373554_at | 1.38 | 2.4 | 1.25 | 1.5 | −1.07 | 0.3 | 1.10 |
| 295 | 1373557_at | 1.21 | 1.2 | −1.87 | 7.9 | −1.17 | 1 | −1.28 |
| 296 | 1373590_at | −1.04 | 0.2 | 1.49 | 4.2 | 1.23 | 1.5 | 1.13 |
| 297 | 1373592_at | −1.23 | 1 | 1.24 | 1.2 | 1.63 | 4.1 | 1.10 |
| 298 | 1373599_at | 1.09 | 0.3 | −1.07 | 0.2 | 1.10 | 0.3 | 1.08 |
| 299 | 1373615_at | 1.07 | 0.2 | 2.84 | 9.7 | 1.54 | 2.5 | 1.40 |
| 300 | 1373628_at | 1.89 | 4 | −1.78 | 3.6 | 1.09 | 0.3 | −1.48 |
| 301 | 1373661_a_at | −1.95 | 2.2 | 1.97 | 2.4 | 2.33 | 3.4 | 1.69 |
| 302 | 1373666_at | 1.02 | 0 | 2.21 | 4.4 | 2.04 | 3.7 | 2.50 |
| 303 | 1373683_at | 1.36 | 2.4 | 2.03 | 9.4 | 1.90 | 8.2 | 1.81 |
| 304 | 1373695_at | −1.01 | 0.1 | 1.26 | 2.7 | 1.21 | 2 | 1.50 |
| 305 | 1373696_at | 1.07 | 0.2 | 1.04 | 0.1 | 1.27 | 1 | 1.52 |
| 306 | 1373740_at | 1.11 | 0.1 | 6.22 | 4 | 1.35 | 0.3 | 2.41 |
| 307 | 1373751_at | −1.10 | 0.3 | 1.74 | 4 | 1.35 | 1.5 | 1.53 |
| 308 | 1373829_at | −1.02 | 0 | 1.18 | 0.5 | −1.20 | 0.5 | −1.67 |
| 309 | 1373847_at | 3.18 | 9.9 | 1.37 | 1.5 | 2.01 | 5 | 1.78 |
| 310 | 1373874_at | 1.02 | 0.1 | 1.13 | 0.7 | 1.05 | 0.2 | 1.03 |
| 311 | 1373881_at | −1.09 | 0.4 | 3.13 | 17.7 | 2.66 | 14.8 | 2.90 |
| 312 | 1373911_at | 1.19 | 0.6 | −2.76 | 8.8 | −1.24 | 0.8 | −1.27 |
| 313 | 1373957_at | −1.14 | 0.4 | 1.07 | 0.2 | −1.18 | 0.5 | −1.91 |
| 314 | 1373970_at | 5.03 | 3.8 | 30.48 | 12.2 | 8.29 | 6.2 | 10.63 |
| 315 | 1374013_at | 1.09 | 0.5 | 1.09 | 0.5 | 1.25 | 1.7 | 1.24 |
| 316 | 1374034_at | 1.08 | 0.4 | −1.58 | 4.6 | −1.38 | 2.7 | 1.32 |
| 317 | 1374057_at | −1.08 | 0.4 | −1.18 | 1.1 | −1.04 | 0.2 | −1.11 |
| 318 | 1374061_at | 1.22 | 1 | 2.13 | 8.1 | 1.79 | 5.5 | 1.73 |
| 319 | 1374104_at | 1.36 | 1.1 | 1.34 | 1.1 | 2.21 | 5.2 | 2.84 |
| 320 | 1374118_at | −1.24 | 2.2 | −1.42 | 5 | −1.12 | 0.9 | −1.06 |
| 321 | 1374129_at | 1.61 | 4.1 | 1.18 | 0.9 | 1.14 | 0.6 | −1.06 |
| 322 | 1374134_at | −1.22 | 1.1 | −1.06 | 0.2 | 1.28 | 1.6 | 1.13 |
| 323 | 1374171_at | 1.00 | 0 | 3.78 | 7.2 | 2.27 | 3.4 | 1.88 |
| 324 | 1374172_at | −1.40 | 0.6 | 1.08 | 0.1 | 1.99 | 1.9 | 3.24 |
| 325 | 1374176_at | −1.12 | 0.3 | 1.47 | 1.6 | −1.47 | 1.6 | 1.16 |
| 326 | 1374207_at | 1.12 | 0.3 | 2.75 | 8 | 6.19 | 17.3 | 5.11 |
| 327 | 1374247_at | 3.60 | 3.1 | 2.25 | 1.6 | 4.11 | 3.9 | 3.26 |
| 328 | 1374266_at | 1.09 | 0.1 | 1.13 | 0.2 | 1.76 | 1.3 | 2.96 |
| 329 | 1374273_at | −1.56 | 1.1 | −1.02 | 0 | 1.57 | 1.2 | 1.18 |
| 330 | 1374276_at | −1.07 | 0.1 | 1.01 | 0 | −1.20 | 0.6 | −1.08 |
| 331 | 1374284_at | −1.19 | 0.6 | 1.30 | 1.1 | −1.11 | 0.3 | −1.27 |
| 332 | 1374285_at | −1.02 | 0.1 | −1.59 | 5 | −1.18 | 1 | −1.24 |
| 333 | 1374306_at | 1.15 | 0.4 | 1.27 | 0.8 | 1.12 | 0.3 | 1.18 |
| 334 | 1374320_at | 1.19 | 0.5 | 2.57 | 7.1 | 3.92 | 11.9 | 2.68 |
| 335 | 1374399_at | 1.30 | 1.5 | −1.41 | 2.4 | −1.05 | 0.2 | −1.16 |
| 336 | 1374474_at | 1.31 | 2.4 | 2.16 | 12.3 | 1.85 | 9 | 2.05 |
| 337 | 1374477_at | 1.19 | 1.3 | −1.05 | 0.2 | 1.35 | 3.1 | 1.29 |
| 338 | 1374479_at | −1.03 | 0.1 | −1.11 | 0.5 | 1.14 | 0.7 | −1.23 |
| 339 | 1374529_at | 1.24 | 0.5 | 6.62 | 12.2 | 4.09 | 8.2 | 3.87 |
| 340 | 1374531_at | 1.08 | 0.2 | 1.83 | 5 | 1.51 | 2.7 | 2.01 |
| 341 | 1374575_at | −1.04 | 0.1 | 1.31 | 1.1 | 1.08 | 0.2 | 1.21 |
| 342 | 1374586_at | 1.07 | 0.2 | 1.21 | 0.9 | 1.41 | 2.1 | 1.72 |
| 343 | 1374626_at | 1.06 | 0.1 | 4.75 | 5.5 | 2.48 | 2.4 | 4.11 |
| 344 | 1374635_at | −1.77 | 0.7 | 3.70 | 2.5 | 1.56 | 0.5 | 2.19 |
| 345 | 1374678_at | −1.16 | 0.4 | −1.12 | 0.3 | 1.09 | 0.2 | 2.33 |
| 346 | 1374742_at | −1.15 | 0.2 | 1.14 | 0.2 | 1.23 | 0.4 | 1.73 |
| 347 | 1374771_at | −1.25 | 1.7 | −1.23 | 1.6 | −1.29 | 2.3 | −1.13 |
| 348 | 1374779_at | 1.23 | 0.3 | 2.25 | 2.3 | 2.11 | 2 | 1.89 |
| 349 | 1374816_at | 1.93 | 2.8 | 1.02 | 0 | 1.68 | 2.1 | 1.30 |
| 350 | 1374849_at | −1.20 | 0.5 | 2.41 | 6.2 | 2.46 | 6.4 | 1.89 |
| 351 | 1374868_at | 1.30 | 1.2 | 1.18 | 0.7 | 1.01 | 0 | −1.24 |
| 352 | 1374897_at | 1.10 | 0.5 | −1.54 | 5.7 | −1.06 | 0.3 | 1.10 |
| 353 | 1374953_at | 2.37 | 4.1 | 1.53 | 1.5 | 1.27 | 0.7 | −1.04 |
| 354 | 1375028_at | 2.95 | 10.1 | 1.77 | 4.5 | 1.82 | 4.8 | 1.31 |
| 355 | 1375051_at | −1.28 | 1.3 | 1.06 | 0.2 | 1.00 | 0 | −1.03 |
| 356 | 1375074_at | −1.46 | 2.8 | 2.26 | 9.7 | 1.83 | 6.3 | 1.75 |
| 357 | 1375138_at | 1.03 | 0.1 | −1.66 | 3.7 | −1.48 | 2.5 | −1.32 |
| 358 | 1375144_at | 1.02 | 0.1 | 1.28 | 2.3 | −1.00 | 0 | 1.15 |
| 359 | 1375267_at | 1.48 | 3 | 1.19 | 0.9 | 1.00 | 0 | 1.06 |
| 360 | 1375270_at | −1.14 | 0.6 | 1.76 | 5.5 | 1.82 | 6 | 2.03 |
| 361 | 1375277_at | −1.06 | 0.2 | 1.31 | 1.8 | 1.76 | 5.6 | 1.64 |
| 362 | 1375337_at | 1.17 | 0.8 | 1.36 | 2.5 | 1.31 | 2.1 | 1.56 |
| 363 | 1375368_at | −1.16 | 1.1 | −1.04 | 0.2 | −1.05 | 0.3 | 1.01 |
| 364 | 1375369_at | −1.08 | 0.4 | 1.89 | 9.7 | 1.20 | 1.4 | 1.69 |
| 365 | 1375377_at | 3.66 | 10.4 | −1.05 | 0.1 | −1.28 | 1 | 1.49 |
| 366 | 1375420_at | 1.67 | 3 | −1.25 | 0.9 | 1.29 | 1.1 | 1.82 |
| 367 | 1375424_at | 1.16 | 1.2 | −1.43 | 5 | −1.27 | 2.6 | −1.45 |
| 368 | 1375463_at | −1.15 | 0.8 | −1.02 | 0.1 | −1.01 | 0 | 1.07 |
| 369 | 1375473_at | −1.17 | 0.5 | 1.46 | 1.9 | 2.01 | 4.8 | 1.72 |
| 370 | 1375657_at | 1.50 | 2.6 | 1.31 | 1.5 | 1.47 | 2.6 | 1.57 |
| 371 | 1375719_s_at | 1.51 | 1.6 | −1.12 | 0.3 | −1.11 | 0.3 | 1.33 |
| 372 | 1375721_at | −1.06 | 0.2 | 1.66 | 5.3 | 1.20 | 1.1 | 1.26 |
| 373 | 1375739_at | 1.17 | 1.5 | −1.35 | 4.5 | −1.13 | 1.1 | −1.20 |
| 374 | 1375862_at | 1.00 | 0 | −1.14 | 0.9 | −1.03 | 0.1 | 1.17 |
| 375 | 1375898_at | 1.15 | 1.2 | 1.10 | 0.8 | 1.05 | 0.4 | 1.21 |
| 376 | 1375951_at | 1.40 | 1.3 | 6.70 | 17.6 | 2.66 | 7.3 | 2.13 |
| 377 | 1376004_at | 1.17 | 0.9 | 1.05 | 0.2 | 1.20 | 1.2 | 1.20 |
| 378 | 1376045_at | 1.06 | 0.5 | −1.11 | 1 | 1.12 | 1.1 | 1.48 |
| 379 | 1376055_at | −1.27 | 1.4 | −1.79 | 6.1 | −1.13 | 0.6 | −1.23 |
| 380 | 1376099_at | 1.19 | 0.7 | 1.13 | 0.5 | 1.06 | 0.2 | 2.00 |
| 381 | 1376106_at | −1.27 | 0.7 | 1.10 | 0.2 | 1.30 | 0.9 | 1.65 |
| 382 | 1376128_at | 1.47 | 3.2 | 1.85 | 7 | 1.58 | 4.5 | 1.67 |
| 383 | 1376182_at | 1.06 | 0.2 | 1.21 | 1.2 | 1.24 | 1.4 | 1.70 |
| 384 | 1376265_at | −1.39 | 2.1 | 1.60 | 4 | 1.53 | 3.4 | 1.94 |
| 385 | 1376570_at | −1.00 | 0 | −1.17 | 2.7 | −1.07 | 0.7 | −1.07 |
| 386 | 1376574_at | 1.81 | 2.8 | 4.48 | 11.8 | 3.67 | 9.7 | 3.94 |
| 387 | 1376583_at | −1.13 | 0.6 | −1.06 | 0.2 | 1.19 | 1 | −1.04 |
| 388 | 1376624_at | −1.81 | 3 | 1.62 | 2.3 | 2.02 | 4.2 | 1.58 |
| 389 | 1376631_at | 1.34 | 2.8 | 1.25 | 2 | 1.19 | 1.4 | 1.30 |
| 390 | 1376640_at | 1.25 | 1.2 | 2.13 | 8.5 | 1.23 | 1.2 | 1.54 |
| 391 | 1376655_at | −1.06 | 0.3 | −1.30 | 2.4 | 1.02 | 0.1 | 1.15 |
| 392 | 1376678_at | 1.09 | 0.2 | 1.01 | 0 | −1.02 | 0 | 1.01 |
| 393 | 1376754_at | 1.02 | 0.1 | −1.53 | 6.1 | −1.17 | 1.3 | −1.06 |
| 394 | 1376775_at | −1.55 | 2.5 | −1.06 | 0.2 | 1.23 | 0.8 | 1.48 |
| 395 | 1376786_a_at | −1.07 | 0.3 | 1.48 | 3.8 | 1.31 | 2.1 | 1.54 |
| 396 | 1376788_at | 1.30 | 0.8 | 2.58 | 6.9 | 3.28 | 9.5 | 2.58 |
| 397 | 1376848_at | −1.01 | 0 | 1.13 | 0.4 | 1.27 | 1 | 1.11 |
| 398 | 1376858_at | −1.48 | 1.6 | −1.04 | 0.1 | 1.01 | 0 | −1.08 |
| 399 | 1376919_at | 1.03 | 0.1 | 1.16 | 1 | −1.19 | 1.4 | −1.14 |
| 400 | 1377023_at | −1.05 | 0.1 | 3.48 | 4.2 | 2.03 | 1.7 | 2.57 |
| 401 | 1377369_at | 1.41 | 3.5 | 1.28 | 2.3 | −1.01 | 0 | −1.06 |
| 402 | 1377390_at | 1.46 | 2.5 | 1.69 | 4.4 | 1.01 | 0 | 1.16 |
| 403 | 1377595_at | 1.72 | 3.8 | 1.20 | 0.9 | 1.00 | 0 | −1.06 |
| 404 | 1377625_at | 1.18 | 0.8 | 1.12 | 0.6 | −1.10 | 0.5 | −1.16 |
| 405 | 1377630_at | 1.32 | 0.6 | 1.20 | 0.4 | 2.54 | 4 | 2.57 |
| 406 | 1377671_at | −1.41 | 1.5 | 3.34 | 11.6 | 3.72 | 13 | 5.48 |
| 407 | 1377697_at | 1.08 | 0.2 | −1.51 | 2.8 | 1.06 | 0.2 | 1.80 |
| 408 | 1377702_at | −1.19 | 0.6 | 1.15 | 0.5 | 1.33 | 1.4 | 1.36 |
| 409 | 1377723_at | −1.10 | 0.5 | 1.13 | 0.8 | 1.10 | 0.6 | 1.19 |
| 410 | 1377862_at | 1.63 | 1.3 | 2.42 | 3.9 | 2.07 | 2.8 | 2.12 |
| 411 | 1377869_at | −1.64 | 3.6 | 1.04 | 0.1 | 1.10 | 0.4 | −1.33 |
| 412 | 1377950_at | 1.05 | 0.1 | −1.34 | 0.8 | 1.51 | 1.3 | 2.49 |
| 413 | 1377994_at | −2.06 | 1.9 | 2.93 | 4.1 | 1.80 | 1.6 | 1.20 |
| 414 | 1378134_at | 1.04 | 0.1 | 1.03 | 0 | 1.90 | 2.9 | −1.03 |
| 415 | 1378282_at | −1.15 | 0.7 | −1.05 | 0.3 | 1.22 | 1.7 | 1.38 |
| 416 | 1378305_at | 2.76 | 4.5 | 1.17 | 0.4 | 2.08 | 3 | 2.10 |
| 417 | 1378321_at | −1.02 | 0 | 1.20 | 0.6 | −1.22 | 0.7 | −1.53 |
| 418 | 1378342_at | −1.04 | 0.1 | 1.46 | 2.3 | 1.17 | 0.6 | 1.32 |
| 419 | 1378440_at | −1.47 | 2.5 | 1.04 | 0.2 | −1.07 | 0.3 | −1.03 |
| 420 | 1378507_at | −1.25 | 0.9 | −1.98 | 6.2 | −1.28 | 1.3 | −1.25 |
| 421 | 1378526_at | 1.02 | 0 | 1.14 | 0.5 | 1.10 | 0.3 | 1.05 |
| 422 | 1378925_at | −1.12 | 0.3 | 2.68 | 7 | 1.14 | 0.4 | −1.02 |
| 423 | 1379294_at | −1.24 | 1.4 | −1.08 | 0.4 | −1.09 | 0.4 | 1.05 |
| 424 | 1379322_at | 1.06 | 0.4 | 1.00 | 0 | 1.07 | 0.5 | −1.33 |
| 425 | 1379331_at | −2.04 | 1.2 | −1.74 | 0.9 | 1.05 | 0.1 | 2.93 |
| 426 | 1379335_at | −1.08 | 0.5 | −1.03 | 0.2 | 1.21 | 1.9 | −1.02 |
| 427 | 1379345_at | 1.15 | 0.6 | −1.44 | 2.8 | −1.40 | 2.4 | −1.31 |
| 428 | 1379461_at | −1.06 | 0.2 | 1.06 | 0.2 | 1.09 | 0.4 | 1.01 |
| 429 | 1379497_at | −2.40 | 0.9 | 1.27 | 0.2 | −2.15 | 0.8 | −6.64 |
| 430 | 1379604_at | 1.07 | 0.2 | 1.45 | 2.3 | 1.63 | 3.4 | 1.73 |
| 431 | 1379772_at | −1.52 | 0.7 | −1.09 | 0.1 | 1.03 | 0 | 2.85 |
| 432 | 1379790_at | −1.38 | 1.4 | 1.09 | 0.3 | 1.13 | 0.4 | 1.33 |
| 433 | 1379799_at | 1.37 | 0.7 | 1.02 | 0 | 1.43 | 0.9 | 1.92 |
| 434 | 1379847_at | −1.08 | 0.2 | −1.16 | 0.7 | −1.10 | 0.4 | 1.26 |
| 435 | 1379882_a_at | 1.03 | 0.1 | −1.06 | 0.2 | 1.15 | 0.4 | 1.67 |
| 436 | 1379935_at | 1.66 | 1.9 | 2.63 | 6.2 | 1.40 | 1.2 | 1.50 |
| 437 | 1380089_at | 1.30 | 0.5 | −1.35 | 0.8 | −1.12 | 0.2 | −1.55 |
| 438 | 1380110_at | 1.01 | 0 | 2.72 | 8.9 | 2.02 | 5.3 | 2.09 |
| 439 | 1380318_at | −1.10 | 0.1 | 2.64 | 3.5 | 5.00 | 7.5 | 3.69 |
| 440 | 1380474_at | 2.45 | 3.5 | −1.12 | 0.2 | 1.06 | 0.1 | 1.44 |
| 441 | 1380617_at | 1.82 | 1.1 | 5.79 | 6.9 | 8.86 | 9.4 | 5.67 |
| 442 | 1380688_at | 1.57 | 0.9 | 1.45 | 0.8 | 2.16 | 2.3 | 2.39 |
| 443 | 1380728_at | 1.16 | 0.4 | 1.18 | 0.5 | 1.03 | 0.1 | 1.22 |
| 444 | 1380908_at | −1.37 | 0.6 | −1.57 | 1.2 | 1.05 | 0.1 | −1.20 |
| 445 | 1381190_at | 1.69 | 2.3 | −2.00 | 4.2 | −1.38 | 1.3 | −1.01 |
| 446 | 1381305_at | 1.04 | 0.2 | 1.28 | 2.9 | 1.32 | 3.6 | 1.51 |
| 447 | 1381311_at | 1.48 | 0.6 | 2.21 | 2.1 | 2.06 | 1.8 | 4.48 |
| 448 | 1381335_at | −1.62 | 0.5 | −1.76 | 0.8 | −2.08 | 1.2 | −8.63 |
| 449 | 1381410_a_at | 2.25 | 2.2 | 1.76 | 1.5 | 1.52 | 1 | 2.60 |
| 450 | 1381461_at | 4.42 | 17 | 2.19 | 8.6 | 1.60 | 4 | 1.65 |
| 451 | 1381470_at | −1.48 | 1.7 | 2.19 | 5.8 | 3.57 | 11.6 | 5.02 |
| 452 | 1381678_at | −1.13 | 0.4 | 1.06 | 0.2 | 1.31 | 1.3 | 1.52 |
| 453 | 1381915_at | 1.03 | 0.1 | 1.39 | 1.6 | 1.44 | 1.9 | 1.88 |
| 454 | 1381971_at | 3.57 | 6.2 | 2.07 | 3.1 | 3.77 | 7.7 | 3.67 |
| 455 | 1381996_at | −1.64 | 1.1 | −1.85 | 1.8 | −1.08 | 0.1 | 1.31 |
| 456 | 1382008_at | −1.00 | 0 | −1.39 | 6.2 | −1.27 | 3.8 | −1.39 |
| 457 | 1382017_at | −1.42 | 0.4 | −1.91 | 1 | 1.29 | 0.3 | 2.19 |
| 458 | 1382108_at | 1.50 | 3 | 1.22 | 1.2 | 1.02 | 0.1 | 1.36 |
| 459 | 1382130_at | 1.16 | 0.2 | −1.51 | 0.9 | 1.03 | 0 | 1.38 |
| 460 | 1382138_at | −1.60 | 1.6 | 1.19 | 0.5 | 1.23 | 0.6 | 1.63 |
| 461 | 1382181_at | −1.14 | 0.6 | 1.25 | 1.5 | −1.07 | 0.3 | −1.00 |
| 462 | 1382212_at | 1.05 | 0.1 | 1.74 | 3.8 | 1.32 | 1.3 | 1.13 |
| 463 | 1382296_at | 1.12 | 0.2 | 3.91 | 7.8 | 1.99 | 3 | 1.67 |
| 464 | 1382375_at | 1.12 | 0.4 | 1.44 | 2 | 1.48 | 2.1 | 1.15 |
| 465 | 1382404_at | −1.42 | 0.9 | 1.63 | 1.7 | 2.56 | 4.8 | 2.38 |
| 466 | 1382431_at | −1.31 | 0.4 | 3.64 | 5.1 | 2.56 | 3.1 | 2.72 |
| 467 | 1382482_at | 1.54 | 4 | 1.11 | 0.5 | 1.33 | 2.3 | 1.53 |
| 468 | 1382536_at | 1.08 | 0.3 | 1.46 | 2.8 | 1.63 | 4.2 | 2.17 |
| 469 | 1382571_at | 1.80 | 1.3 | 1.62 | 1.2 | 1.26 | 0.4 | 1.99 |
| 470 | 1382628_at | 1.27 | 1.1 | 1.30 | 1.5 | 1.47 | 2.7 | 1.39 |
| 471 | 1382680_at | 1.38 | 1.4 | 1.10 | 0.3 | −1.39 | 1.5 | −1.86 |
| 472 | 1382692_at | −1.45 | 1.2 | −1.11 | 0.3 | −1.41 | 1.3 | −2.20 |
| 473 | 1382818_at | −1.05 | 0.1 | 1.37 | 1.6 | 1.14 | 0.4 | −1.12 |
| 474 | 1382995_at | −1.44 | 3 | 1.05 | 0.2 | −1.06 | 0.3 | −1.02 |
| 475 | 1383019_at | 1.17 | 0.2 | 2.35 | 1.9 | 5.52 | 5.5 | 3.43 |
| 476 | 1383073_at | 1.10 | 1 | −1.25 | 4.6 | −1.16 | 2.3 | −1.09 |
| 477 | 1383080_at | 1.05 | 0.5 | 1.24 | 4.2 | 1.14 | 1.9 | 1.12 |
| 478 | 1383131_at | −1.26 | 0.8 | 1.05 | 0.1 | 1.22 | 0.8 | 1.08 |
| 479 | 1383147_at | −1.01 | 0 | −1.35 | 3.8 | 1.13 | 1 | 1.08 |
| 480 | 1383193_at | 1.03 | 0.1 | −1.35 | 3.8 | −1.19 | 1.6 | −1.45 |
| 481 | 1383201_at | 1.03 | 0.1 | 1.16 | 1.3 | 1.15 | 1.1 | 1.11 |
| 482 | 1383240_at | 1.11 | 0.2 | 1.04 | 0.1 | −1.03 | 0 | 1.45 |
| 483 | 1383247_a_at | −2.24 | 1.8 | 1.14 | 0.2 | 6.92 | 8.2 | 3.89 |
| 484 | 1383261_at | 1.52 | 2.4 | −1.19 | 0.7 | 1.14 | 0.5 | 1.11 |
| 485 | 1383328_x_at | 1.34 | 1.5 | 1.33 | 1.6 | 1.47 | 2.6 | 1.74 |
| 486 | 1383355_at | −1.16 | 0.7 | 2.49 | 12.3 | 1.87 | 7.3 | 1.91 |
| 487 | 1383382_at | −1.11 | 0.4 | 1.41 | 2.7 | 1.05 | 0.2 | 1.39 |
| 488 | 1383469_at | 3.80 | 4 | −1.05 | 0.1 | 1.68 | 1.1 | 1.98 |
| 489 | 1383484_at | −1.01 | 0.1 | 1.11 | 1 | 1.15 | 1.6 | 1.06 |
| 490 | 1383489_at | 1.35 | 1.6 | 1.68 | 4.6 | 2.07 | 7.7 | 2.38 |
| 491 | 1383498_at | 1.09 | 0.4 | 1.52 | 5 | 1.30 | 2.4 | 1.39 |
| 492 | 1383516_at | −1.31 | 1.4 | −1.18 | 0.8 | 1.00 | 0 | −1.14 |
| 493 | 1383533_at | −1.01 | 0 | 1.16 | 1.1 | 1.07 | 0.4 | 1.22 |
| 494 | 1383574_at | 1.10 | 0.6 | 1.56 | 6.4 | 1.24 | 2.1 | 1.31 |
| 495 | 1383641_at | −1.25 | 1.1 | 1.61 | 3.9 | 1.14 | 0.6 | 1.12 |
| 496 | 1383644_at | −2.08 | 0.6 | 1.08 | 0 | −1.41 | 0.2 | −2.13 |
| 497 | 1383662_at | −1.13 | 0.7 | 1.57 | 5.5 | 1.20 | 1.3 | 1.65 |
| 498 | 1383776_at | 1.40 | 3.1 | 1.12 | 0.7 | −1.24 | 1.9 | −1.45 |
| 499 | 1383785_at | −1.43 | 1.5 | 1.06 | 0.2 | 1.20 | 0.7 | 1.04 |
| 500 | 1383824_at | −1.31 | 0.4 | −1.34 | 0.6 | 1.14 | 0.2 | 1.85 |
| 501 | 1383844_at | 1.22 | 0.3 | 2.51 | 3.1 | 3.60 | 5.1 | 2.47 |
| 502 | 1383863_at | 1.56 | 3.6 | 1.88 | 7.4 | 2.08 | 9.1 | 2.11 |
| 503 | 1383914_at | −1.09 | 0.4 | 1.13 | 0.7 | −1.29 | 2 | −1.46 |
| 504 | 1383935_at | −1.26 | 0.4 | −1.28 | 0.4 | 1.41 | 0.7 | 1.96 |
| 505 | 1383949_at | 1.16 | 0.9 | 1.47 | 3.8 | 1.64 | 5.7 | 1.81 |
| 506 | 1384051_at | 1.08 | 0.2 | 2.05 | 5.3 | 1.56 | 2.5 | 1.71 |
| 507 | 1384165_at | 2.01 | 0.8 | 3.60 | 2.5 | 1.81 | 0.8 | 1.63 |
| 508 | 1384183_at | 2.12 | 4.7 | −1.54 | 2.4 | −1.29 | 1.1 | −1.84 |
| 509 | 1384188_at | −1.04 | 0.1 | 1.08 | 0.3 | 1.18 | 0.9 | 1.17 |
| 510 | 1384192_at | −1.04 | 0.1 | 2.14 | 3.1 | 2.58 | 4.3 | 3.28 |
| 511 | 1384227_at | 1.80 | 3.2 | 1.63 | 2.9 | 1.72 | 3.4 | 2.05 |
| 512 | 1384232_at | −1.08 | 0.2 | −1.18 | 0.6 | −1.08 | 0.2 | 1.06 |
| 513 | 1384302_at | −1.11 | 0.2 | 4.05 | 8.9 | 1.60 | 1.6 | 1.59 |
| 514 | 1384335_at | 1.22 | 1.6 | 1.20 | 1.7 | −1.02 | 0.1 | 1.52 |
| 515 | 1384381_at | −1.47 | 1.1 | 4.10 | 8.8 | 2.55 | 5.2 | 2.59 |
| 516 | 1384392_at | 2.79 | 3.7 | 2.71 | 4.2 | 2.29 | 3.1 | −1.04 |
| 517 | 1384415_at | 1.22 | 0.9 | 1.24 | 1.2 | 1.48 | 3 | 2.59 |
| 518 | 1384724_at | 1.14 | 0.4 | 2.53 | 9 | 1.81 | 4.6 | 1.45 |
| 519 | 1384899_at | 1.18 | 0.6 | −1.25 | 1.1 | 1.03 | 0.1 | 1.11 |
| 520 | 1384900_at | −1.07 | 0.3 | 1.06 | 0.2 | −1.24 | 1.4 | −1.00 |
| 521 | 1384907_at | −1.21 | 0.3 | 1.80 | 1.9 | 1.26 | 0.5 | 1.31 |
| 522 | 1385057_at | −2.01 | 1.6 | −1.08 | 0.1 | −1.13 | 0.2 | 1.48 |
| 523 | 1385359_at | 1.55 | 0.9 | 2.05 | 2.4 | 1.73 | 1.5 | 2.91 |
| 524 | 1385397_at | −1.31 | 0.5 | 5.42 | 8.6 | 3.44 | 5.4 | 4.40 |
| 525 | 1385444_at | 2.06 | 0.7 | 4.67 | 2.1 | 7.07 | 3.1 | 7.91 |
| 526 | 1385506_at | 1.14 | 0.3 | −1.29 | 0.9 | −1.29 | 0.9 | −1.83 |
| 527 | 1385925_at | 1.34 | 1.3 | 1.75 | 4.1 | 1.43 | 2.1 | 1.28 |
| 528 | 1385926_at | 1.11 | 0.6 | 1.42 | 4.3 | 1.29 | 2.6 | 1.47 |
| 529 | 1386041_a_at | 6.17 | 6.3 | −3.15 | 3.3 | 1.55 | 0.8 | 2.55 |
| 530 | 1386080_at | −1.37 | 1.5 | −1.04 | 0.1 | −1.14 | 0.5 | −1.23 |
| 531 | 1386540_at | −1.03 | 0.1 | 1.23 | 1 | 1.49 | 2.8 | 1.95 |
| 532 | 1386721_at | 1.30 | 1.2 | 1.41 | 2.2 | 1.37 | 1.9 | 2.04 |
| 533 | 1386833_at | −1.26 | 0.9 | 1.38 | 1.7 | 2.45 | 8.2 | 2.41 |
| 534 | 1386864_at | 1.03 | 0.2 | −1.07 | 0.6 | 1.05 | 0.4 | 1.05 |
| 535 | 1386866_at | 1.14 | 1.8 | 1.00 | 0 | −1.11 | 1.3 | 1.08 |
| 536 | 1386881_at | 1.95 | 1.7 | 1.07 | 0.1 | 3.17 | 4.3 | 4.65 |
| 537 | 1386899_at | 1.44 | 4.9 | 1.06 | 0.4 | 1.15 | 1.2 | 1.54 |
| 538 | 1386912_at | 1.48 | 2.2 | 1.22 | 0.9 | 1.10 | 0.3 | 2.07 |
| 539 | 1386913_at | 1.29 | 1.1 | 2.12 | 6.2 | 1.98 | 5.4 | 1.70 |
| 540 | 1386922_at | −1.06 | 0.1 | −1.02 | 0 | 1.31 | 0.4 | 1.88 |
| 541 | 1386937_at | −1.00 | 0 | 1.03 | 0.1 | −1.23 | 1 | −1.02 |
| 542 | 1386938_at | 1.61 | 2.8 | 1.35 | 1.5 | 1.37 | 1.6 | 1.35 |
| 543 | 1386940_at | 1.08 | 0.5 | 1.33 | 3.4 | −1.17 | 1.4 | −1.18 |
| 544 | 1386948_at | −1.06 | 0.2 | −1.88 | 5.8 | −1.49 | 2.9 | −1.27 |
| 545 | 1387001_at | −1.02 | 0.1 | −1.23 | 2.1 | 1.03 | 0.2 | 1.19 |
| 546 | 1387005_at | 1.05 | 0.1 | 2.10 | 4.2 | 1.63 | 2.2 | 1.74 |
| 547 | 1387018_at | −1.06 | 0.2 | −1.42 | 1.6 | −1.09 | 0.2 | −1.15 |
| 548 | 1387076_at | 1.45 | 4 | 2.00 | 10.7 | 1.89 | 9.5 | 1.81 |
| 549 | 1387125_at | −1.17 | 0.2 | 14.70 | 8.5 | 11.41 | 7.3 | 12.60 |
| 550 | 1387137_at | −1.81 | 2.8 | 1.22 | 0.6 | 1.03 | 0.1 | −1.34 |
| 551 | 1387153_at | 1.11 | 0.4 | 1.55 | 3.7 | 1.47 | 3 | 1.65 |
| 552 | 1387154_at | 1.16 | 0.2 | 2.45 | 3.3 | 2.35 | 3.1 | 1.84 |
| 553 | 1387160_at | 1.71 | 1 | 1.55 | 0.8 | 2.12 | 1.7 | 3.30 |
| 554 | 1387197_at | 1.02 | 0 | −1.23 | 0.7 | 1.35 | 1.1 | 1.04 |
| 555 | 1387202_at | 1.71 | 3.4 | 1.42 | 2.1 | −1.18 | 0.7 | 1.10 |
| 556 | 1387219_at | 4.53 | 3.2 | 3.91 | 3 | 4.57 | 3.5 | 7.93 |
| 557 | 1387259_at | 1.35 | 2.1 | 1.52 | 3.9 | 1.42 | 2.9 | 1.82 |
| 558 | 1387270_at | 1.22 | 0.3 | 4.87 | 5.4 | 4.95 | 5.5 | 5.05 |
| 559 | 1387273_at | −1.67 | 1.7 | −2.26 | 3.7 | −3.21 | 6.4 | −1.66 |
| 560 | 1387279_at | 1.83 | 5.6 | 2.66 | 12 | 3.65 | 16.9 | 3.42 |
| 561 | 1387282_at | 1.27 | 1.1 | 1.59 | 3.6 | 1.24 | 1.1 | 1.10 |
| 562 | 1387294_at | 1.74 | 4.1 | 2.12 | 7 | 2.28 | 8 | 3.87 |
| 563 | 1387306_a_at | −1.09 | 0.2 | −2.06 | 4.4 | −1.65 | 2.5 | −1.70 |
| 564 | 1387343_at | −1.08 | 0.2 | 2.88 | 9.7 | 1.89 | 4.6 | 2.82 |
| 565 | 1387344_at | 1.12 | 0.5 | 1.28 | 1.7 | 1.16 | 0.8 | 1.41 |
| 566 | 1387348_at | −1.03 | 0 | 3.67 | 4.9 | 2.09 | 2 | −1.09 |
| 567 | 1387389_at | −1.50 | 0.5 | −1.89 | 1 | −1.33 | 0.3 | −2.08 |
| 568 | 1387395_at | −1.37 | 2.5 | 1.77 | 6.6 | 1.23 | 1.4 | 2.06 |
| 569 | 1387402_at | 1.60 | 2.9 | −1.26 | 1 | −1.16 | 0.6 | 1.10 |
| 570 | 1387530_a_at | −1.52 | 3.5 | 1.59 | 4.2 | 1.19 | 1 | 1.07 |
| 571 | 1387547_a_at | −1.87 | 1.8 | 1.51 | 1 | 2.80 | 4.2 | 3.66 |
| 572 | 1387548_at | −1.30 | 1.2 | −1.18 | 0.7 | 1.04 | 0.1 | 1.14 |
| 573 | 1387630_at | 1.06 | 0.2 | 1.65 | 4 | 1.07 | 0.3 | −1.10 |
| 574 | 1387648_at | 2.74 | 2.2 | 6.04 | 5.8 | 8.50 | 7.5 | 11.79 |
| 575 | 1387675_at | −1.12 | 0.2 | 2.48 | 5.2 | 3.54 | 8.5 | 5.14 |
| 576 | 1387759_s_at | 1.12 | 0.4 | 1.87 | 5 | 1.42 | 2 | 1.54 |
| 577 | 1387789_at | 1.07 | 0.2 | 1.05 | 0.2 | 1.22 | 0.9 | 1.14 |
| 578 | 1387805_at | 1.24 | 1.1 | 1.04 | 0.1 | 1.60 | 3.9 | 1.60 |
| 579 | 1387808_at | −1.27 | 1.6 | 1.64 | 5.9 | 1.39 | 3.1 | 1.42 |
| 580 | 1387817_at | 1.20 | 0.5 | 1.02 | 0 | 2.05 | 4.1 | 1.55 |
| 581 | 1387843_at | 1.89 | 1.5 | 4.57 | 6.3 | 2.73 | 3.3 | 3.83 |
| 582 | 1387856_at | 1.07 | 0.4 | 1.22 | 1.9 | 1.10 | 0.6 | 1.43 |
| 583 | 1387870_at | 1.10 | 0.3 | 2.20 | 6.3 | 1.92 | 4.7 | 3.27 |
| 584 | 1387871_at | −1.07 | 0.5 | −1.35 | 5.1 | −1.17 | 1.8 | 1.01 |
| 585 | 1387885_at | 1.27 | 2.7 | 1.52 | 6.9 | 1.13 | 1.1 | 1.40 |
| 586 | 1387892_at | 1.12 | 0.8 | −1.34 | 3.3 | −1.22 | 1.8 | −1.05 |
| 587 | 1387893_at | 1.46 | 2.9 | 2.02 | 7.9 | 1.61 | 4.4 | 1.74 |
| 588 | 1387896_at | 1.10 | 0.5 | −1.16 | 1 | 1.10 | 0.5 | −1.11 |
| 589 | 1387897_at | −1.14 | 0.5 | 2.11 | 7.4 | 1.71 | 4.5 | 2.34 |
| 590 | 1387975_at | −1.10 | 0.4 | 1.61 | 4.7 | 1.65 | 5.1 | 1.66 |
| 591 | 1387976_at | 1.82 | 3.4 | 1.02 | 0 | 1.02 | 0 | 1.25 |
| 592 | 1388032_a_at | −2.23 | 1.7 | 6.28 | 6.7 | 3.97 | 4.3 | 4.28 |
| 593 | 1388054_a_at | 1.16 | 0.5 | −1.32 | 1.3 | −1.13 | 0.4 | −1.05 |
| 594 | 1388089_a_at | −1.06 | 0.3 | −1.01 | 0 | −1.13 | 0.8 | −1.14 |
| 595 | 1388119_at | 1.02 | 0.2 | −1.39 | 9.7 | −1.28 | 6.5 | −1.00 |
| 596 | 1388120_at | 1.18 | 2.1 | −1.13 | 1.5 | −1.35 | 5.8 | −1.00 |
| 597 | 1388131_at | −1.17 | 0.3 | 1.08 | 0.1 | 1.38 | 0.9 | 1.72 |
| 598 | 1388142_at | 1.21 | 0.8 | −1.35 | 1.7 | −1.05 | 0.2 | −1.26 |
| 599 | 1388143_at | 1.03 | 0.1 | −1.06 | 0.2 | −1.41 | 2.9 | −1.75 |
| 600 | 1388151_at | −1.03 | 0.1 | −1.09 | 0.4 | 1.15 | 0.7 | 1.70 |
| 601 | 1388155_at | 1.27 | 0.6 | −1.89 | 2.8 | −2.20 | 4 | −2.84 |
| 602 | 1388170_at | −1.40 | 1.6 | 2.21 | 6.9 | 1.44 | 2.1 | 1.81 |
| 603 | 1388201_at | 4.96 | 5.4 | 3.98 | 4.7 | 6.32 | 7.2 | 5.33 |
| 604 | 1388204_at | 1.20 | 0.3 | −1.13 | 0.2 | −1.44 | 1 | −1.47 |
| 605 | 1388218_at | 1.16 | 0.4 | −1.43 | 1.5 | −1.18 | 0.5 | 1.02 |
| 606 | 1388243_at | −1.15 | 0.7 | 1.46 | 3 | −1.17 | 0.8 | 1.25 |
| 607 | 1388271_at | 2.76 | 5.3 | 1.19 | 0.4 | 1.03 | 0.1 | −1.09 |
| 608 | 1388312_at | 1.28 | 1.3 | 1.21 | 0.9 | 1.04 | 0.1 | 1.45 |
| 609 | 1388318_at | 1.03 | 0.1 | −1.12 | 0.3 | −1.06 | 0.1 | 1.29 |
| 610 | 1388335_at | 1.19 | 2 | −1.19 | 2.1 | −1.21 | 2.4 | 1.08 |
| 611 | 1388348_at | 1.16 | 1.2 | 1.47 | 5.3 | 1.17 | 1.4 | 1.15 |
| 612 | 1388356_at | 1.22 | 1.1 | 1.03 | 0.1 | 1.49 | 3.4 | 1.89 |
| 613 | 1388392_at | −1.03 | 0.2 | −1.10 | 1.1 | −1.05 | 0.4 | 1.11 |
| 614 | 1388397_at | −1.13 | 0.7 | −1.60 | 5.8 | −1.18 | 1.1 | −1.14 |
| 615 | 1388401_at | 1.38 | 1.7 | −1.06 | 0.2 | 1.33 | 1.5 | 1.30 |
| 616 | 1388449_at | 1.14 | 1.3 | −1.40 | 6 | −1.37 | 5.5 | −1.38 |
| 617 | 1388459_at | −1.14 | 0.8 | −1.14 | 0.9 | −1.50 | 4.8 | −1.83 |
| 618 | 1388486_at | −1.06 | 0.5 | 1.06 | 0.5 | 1.06 | 0.5 | −1.17 |
| 619 | 1388492_at | 1.03 | 0.1 | 1.80 | 9.3 | 1.24 | 1.9 | 1.43 |
| 620 | 1388493_at | −1.04 | 0.2 | 1.12 | 1 | −1.11 | 0.9 | −1.08 |
| 621 | 1388494_at | 1.10 | 0.4 | 2.70 | 12.9 | 1.73 | 5.5 | 2.62 |
| 622 | 1388495_at | 1.03 | 0.1 | −1.21 | 1.6 | −1.11 | 0.6 | −1.22 |
| 623 | 1388521_at | 1.04 | 0.2 | −1.53 | 6.8 | −1.16 | 1.3 | −1.05 |
| 624 | 1388533_at | 1.07 | 0.4 | 1.23 | 2.1 | 1.20 | 1.7 | 1.52 |
| 625 | 1388534_at | 1.16 | 1.8 | 1.39 | 6.8 | 1.19 | 2.5 | 1.21 |
| 626 | 1388568_at | −1.00 | 0 | −1.12 | 1.4 | −1.03 | 0.2 | 1.01 |
| 627 | 1388583_at | −1.41 | 1 | −1.77 | 2.4 | 1.12 | 0.3 | 2.72 |
| 628 | 1388602_at | 1.35 | 0.6 | 2.08 | 2.4 | 2.45 | 3.3 | 5.91 |
| 629 | 1388618_at | −1.09 | 0.3 | −1.45 | 2.5 | 1.19 | 0.8 | 1.36 |
| 630 | 1388666_at | 1.40 | 2.1 | 2.19 | 8 | 1.47 | 2.7 | 1.20 |
| 631 | 1388695_at | 1.28 | 1.5 | −1.15 | 0.7 | 1.01 | 0 | 1.43 |
| 632 | 1388699_at | 1.08 | 0.4 | 1.24 | 1.8 | 1.14 | 0.9 | 1.62 |
| 633 | 1388700_at | 1.09 | 0.5 | 1.18 | 1.4 | 1.06 | 0.3 | 1.19 |
| 634 | 1388703_at | 1.91 | 6.6 | 2.72 | 14 | 2.61 | 13.4 | 2.72 |
| 635 | 1388705_at | −1.04 | 0.2 | −1.61 | 6.8 | −1.29 | 2.6 | −1.20 |
| 636 | 1388711_at | 1.08 | 0.4 | −1.05 | 0.2 | 1.15 | 0.9 | 1.25 |
| 637 | 1388715_at | 1.04 | 0.3 | −1.37 | 6.9 | −1.15 | 1.9 | 1.16 |
| 638 | 1388728_at | −1.10 | 0.8 | 1.03 | 0.2 | 1.03 | 0.2 | −1.01 |
| 639 | 1388742_at | −1.35 | 1.9 | 1.51 | 3.3 | 1.56 | 3.8 | 1.32 |
| 640 | 1388750_at | −1.06 | 0.1 | 2.49 | 5.7 | 2.03 | 3.9 | 1.76 |
| 641 | 1388773_at | −1.24 | 1 | 1.75 | 4.4 | 1.25 | 1.1 | 1.53 |
| 642 | 1388792_at | 1.44 | 1.8 | 3.95 | 13.9 | 2.09 | 5.8 | 1.88 |
| 643 | 1388836_at | −1.11 | 0.3 | 1.68 | 3.5 | 1.82 | 4.3 | 1.77 |
| 644 | 1388870_at | 1.13 | 0.7 | 1.32 | 2.7 | 1.30 | 2.4 | 1.15 |
| 645 | 1388878_at | 1.02 | 0.1 | 1.20 | 0.8 | 1.03 | 0.1 | 1.71 |
| 646 | 1388889_at | −1.17 | 0.8 | 1.25 | 1.4 | 1.34 | 2.1 | 1.29 |
| 647 | 1388926_at | 1.86 | 1.8 | 2.09 | 2.5 | 1.55 | 1.1 | 3.81 |
| 648 | 1388945_at | −1.00 | 0 | −1.10 | 0.6 | −1.28 | 2.4 | −1.45 |
| 649 | 1388955_at | −1.10 | 0.7 | −1.20 | 2.2 | −1.09 | 0.7 | 1.19 |
| 650 | 1388970_at | 1.27 | 0.5 | 3.87 | 6.9 | 4.39 | 7.9 | 4.41 |
| 651 | 1389018_at | −1.07 | 0.1 | 1.26 | 0.5 | 1.45 | 1 | 2.74 |
| 652 | 1389039_at | −1.15 | 0.9 | 1.07 | 0.4 | 1.04 | 0.2 | 1.19 |
| 653 | 1389040_at | 1.20 | 1.1 | 1.10 | 0.5 | 1.08 | 0.4 | −1.04 |
| 654 | 1389059_at | −1.15 | 0.8 | −1.01 | 0 | 1.04 | 0.2 | 1.34 |
| 655 | 1389103_at | −1.25 | 1.4 | −2.00 | 8.7 | −1.23 | 1.4 | −1.53 |
| 656 | 1389123_at | 3.24 | 1.4 | 8.71 | 3.8 | 10.93 | 4.4 | 20.27 |
| 657 | 1389151_at | 1.16 | 0.6 | −1.17 | 0.7 | 1.02 | 0.1 | 1.74 |
| 658 | 1389153_at | −1.23 | 2.1 | −1.46 | 5.9 | −1.08 | 0.5 | −1.12 |
| 659 | 1389157_at | −1.27 | 3 | −1.09 | 0.7 | 1.21 | 2.2 | 1.04 |
| 660 | 1389186_at | 1.29 | 0.8 | 1.57 | 2 | 2.16 | 4.6 | 2.87 |
| 661 | 1389207_at | 1.10 | 0.5 | −1.20 | 1.4 | 1.30 | 2.4 | 1.40 |
| 662 | 1389213_at | −1.27 | 3 | 1.01 | 0.1 | −1.02 | 0.1 | −1.02 |
| 663 | 1389214_at | 1.10 | 0.6 | 2.49 | 16.3 | 1.81 | 9.4 | 2.23 |
| 664 | 1389227_at | −1.15 | 0.8 | 1.22 | 1.4 | 1.07 | 0.3 | 1.55 |
| 665 | 1389244_x_at | −1.27 | 0.5 | 2.17 | 3.5 | 2.57 | 4.8 | 1.74 |
| 666 | 1389255_at | 1.58 | 1 | 1.66 | 1.2 | 3.04 | 4.1 | 5.33 |
| 667 | 1389297_at | 1.06 | 0.2 | 1.15 | 0.8 | 1.26 | 1.8 | 1.47 |
| 668 | 1389324_at | 1.55 | 1.9 | 2.84 | 7.9 | 3.21 | 9.3 | 4.12 |
| 669 | 1389341_at | 1.01 | 0 | 1.42 | 2.8 | 1.88 | 7 | 3.15 |
| 670 | 1389373_at | 1.40 | 5.5 | 1.32 | 4.4 | 1.26 | 3.3 | 1.54 |
| 671 | 1389464_at | 1.48 | 0.9 | 3.37 | 5.4 | 2.98 | 4.3 | 2.78 |
| 672 | 1389470_at | 1.92 | 1.8 | 9.24 | 13.4 | 4.34 | 7.6 | 7.03 |
| 673 | 1389476_at | 1.02 | 0.1 | 1.55 | 4.8 | 1.20 | 1.3 | 1.25 |
| 674 | 1389478_at | −1.06 | 0.5 | 1.07 | 0.7 | 1.07 | 0.7 | 1.29 |
| 675 | 1389483_at | 1.30 | 2.4 | 1.04 | 0.2 | 1.20 | 1.5 | 1.14 |
| 676 | 1389533_at | 1.74 | 3.8 | 1.10 | 0.3 | 1.65 | 3.5 | 2.75 |
| 677 | 1389546_at | 1.35 | 1.1 | 1.14 | 0.4 | 1.28 | 0.9 | 2.14 |
| 678 | 1389573_at | −1.29 | 0.9 | −1.41 | 1.6 | −1.07 | 0.2 | 1.12 |
| 679 | 1389590_at | 1.13 | 0.6 | −1.00 | 0 | −1.02 | 0.1 | 1.03 |
| 680 | 1389611_at | 1.12 | 0.5 | 1.69 | 4.9 | 1.41 | 2.5 | 1.95 |
| 681 | 1389617_at | 1.19 | 1.6 | 1.02 | 0.1 | 1.09 | 0.7 | 1.50 |
| 682 | 1389651_at | −1.89 | 1.8 | 1.14 | 0.2 | 2.83 | 4.1 | 2.71 |
| 683 | 1389787_at | 1.81 | 1.3 | 1.46 | 0.7 | 2.85 | 3.2 | 3.90 |
| 684 | 1389789_at | 1.83 | 1.5 | 2.22 | 2.5 | 3.31 | 4.6 | 3.26 |
| 685 | 1389836_a_at | −1.11 | 0.3 | −1.76 | 4.5 | −1.50 | 2.7 | −1.48 |
| 686 | 1389883_at | 1.22 | 1.7 | −1.26 | 2.3 | −1.13 | 0.9 | −1.16 |
| 687 | 1389966_at | 1.46 | 2.3 | −1.07 | 0.2 | −1.01 | 0 | 1.40 |
| 688 | 1390031_at | −1.29 | 1.7 | 1.03 | 0.1 | 1.25 | 1.5 | 1.22 |
| 689 | 1390112_at | 1.36 | 0.3 | 1.76 | 0.7 | 5.73 | 3.9 | 6.89 |
| 690 | 1390141_at | −1.84 | 2.8 | −2.09 | 4.5 | 1.01 | 0 | 1.14 |
| 691 | 1390159_at | −2.23 | 1.9 | −1.17 | 0.2 | 1.73 | 1.2 | 2.15 |
| 692 | 1390173_at | −1.27 | 1 | −1.07 | 0.2 | 1.16 | 0.6 | 1.02 |
| 693 | 1390300_at | −1.07 | 0.1 | −1.24 | 0.5 | 1.31 | 0.7 | −1.62 |
| 694 | 1390306_at | 1.08 | 0.2 | 1.33 | 1.4 | 1.90 | 5 | 1.71 |
| 695 | 1390311_at | 1.07 | 0.4 | −1.05 | 0.3 | −1.09 | 0.6 | −1.40 |
| 696 | 1390380_at | −1.03 | 0.2 | −1.10 | 0.9 | −1.03 | 0.2 | 1.14 |
| 697 | 1390383_at | −1.18 | 0.9 | −1.70 | 5.4 | −1.58 | 4.3 | −1.78 |
| 698 | 1390388_at | 3.81 | 7.2 | 1.05 | 0.1 | −1.11 | 0.2 | 1.13 |
| 699 | 1390403_at | 1.11 | 0.1 | 1.71 | 1.1 | 1.76 | 1.2 | 4.66 |
| 700 | 1390406_at | −1.08 | 0.2 | 1.09 | 0.3 | 1.00 | 0 | 1.06 |
| 701 | 1390415_at | 1.04 | 0.1 | −1.91 | 6.4 | −1.28 | 1.5 | −1.29 |
| 702 | 1390420_at | 1.10 | 0.2 | 2.69 | 4.3 | 4.50 | 8.2 | 7.21 |
| 703 | 1390536_at | 1.87 | 8.1 | 1.21 | 1.4 | 1.28 | 2.1 | 1.42 |
| 704 | 1390555_at | −1.12 | 0.6 | −1.05 | 0.2 | −1.07 | 0.3 | −1.21 |
| 705 | 1390647_at | −1.02 | 0.1 | −1.84 | 6.2 | −1.54 | 3.6 | −1.69 |
| 706 | 1390706_at | 1.36 | 3.5 | −1.01 | 0.1 | 1.10 | 0.6 | 1.09 |
| 707 | 1390738_at | −1.15 | 0.5 | 1.99 | 5.6 | 1.38 | 1.7 | 1.71 |
| 708 | 1390782_at | 1.18 | 0.2 | 1.88 | 1.8 | 3.22 | 4.7 | 2.58 |
| 709 | 1390828_at | −3.30 | 1.7 | −1.05 | 0 | 1.26 | 0.2 | −1.41 |
| 710 | 1390832_at | −1.34 | 0.7 | 1.02 | 0 | 1.41 | 1.1 | 5.16 |
| 711 | 1390835_at | −1.00 | 0 | −2.61 | 2.6 | −3.34 | 3.6 | −8.43 |
| 712 | 1390846_at | −1.02 | 0.1 | 1.58 | 6.1 | 1.54 | 5.5 | 1.70 |
| 713 | 1390914_at | −1.05 | 0.1 | 1.02 | 0 | 1.36 | 1.5 | 1.75 |
| 714 | 1391167_at | −1.10 | 0.5 | −1.20 | 1.6 | 1.02 | 0.1 | −1.16 |
| 715 | 1391421_at | −1.19 | 1.6 | 1.04 | 0.2 | 1.08 | 0.6 | −1.03 |
| 716 | 1391435_at | 3.08 | 5.5 | 4.38 | 9 | 2.46 | 4.6 | 2.38 |
| 717 | 1391442_at | −1.67 | 2.4 | 1.25 | 0.8 | 1.13 | 0.4 | 1.31 |
| 718 | 1391450_at | 1.21 | 0.5 | 1.74 | 2.5 | 1.41 | 1.2 | 1.74 |
| 719 | 1391458_at | 1.26 | 0.6 | 1.65 | 2 | 1.68 | 2.2 | 2.33 |
| 720 | 1391503_at | −1.16 | 0.6 | 1.07 | 0.2 | 1.44 | 2.5 | 1.40 |
| 721 | 1391505_x_at | −1.74 | 0.5 | 1.12 | 0.1 | −1.48 | 0.4 | −8.19 |
| 722 | 1391607_at | −1.25 | 0.7 | 1.23 | 0.7 | −1.05 | 0.1 | 1.09 |
| 723 | 1391827_at | 1.12 | 0.6 | −1.26 | 1.8 | 1.15 | 0.9 | 1.12 |
| 724 | 1391856_at | 1.49 | 0.8 | 1.69 | 1.4 | 1.66 | 1.3 | 3.22 |
| 725 | 1391871_at | −1.02 | 0 | −1.01 | 0 | 1.20 | 0.7 | −1.04 |
| 726 | 1391946_at | −1.04 | 0.1 | 5.57 | 9.7 | 3.05 | 5.1 | 2.07 |
| 727 | 1392044_at | −1.01 | 0 | 1.04 | 0.1 | 1.04 | 0.1 | −1.05 |
| 728 | 1392171_at | 1.96 | 1.8 | 2.73 | 4.1 | 2.89 | 4.5 | 5.78 |
| 729 | 1392220_at | −1.14 | 0.3 | 1.20 | 0.6 | 1.28 | 0.9 | 1.74 |
| 730 | 1392264_s_at | 1.27 | 0.2 | 8.11 | 6.3 | 8.84 | 6.7 | 6.44 |
| 731 | 1392280_at | −1.25 | 1 | 1.28 | 1.4 | −1.08 | 0.3 | −1.02 |
| 732 | 1392471_at | 1.17 | 1.2 | 1.20 | 1.9 | 1.26 | 2.7 | 1.20 |
| 733 | 1392489_at | 1.26 | 2.6 | 1.31 | 4 | 1.20 | 2.1 | 1.14 |
| 734 | 1392490_at | 1.10 | 0.7 | 1.39 | 4.7 | 1.25 | 2.5 | 1.19 |
| 735 | 1392497_at | 1.02 | 0.1 | 1.20 | 0.9 | −1.04 | 0.1 | 1.00 |
| 736 | 1392578_at | 2.02 | 1.4 | 2.76 | 2.9 | 2.28 | 2.1 | 1.19 |
| 737 | 1392597_at | 1.03 | 0.1 | −1.07 | 0.2 | 1.25 | 1.1 | 1.32 |
| 738 | 1392648_at | 1.70 | 1.5 | 1.66 | 1.6 | 2.11 | 3 | 3.90 |
| 739 | 1392736_at | −1.50 | 0.4 | 3.11 | 1.8 | −2.29 | 1.1 | −11.85 |
| 740 | 1392784_at | −1.26 | 1.4 | 1.10 | 0.4 | 1.02 | 0.1 | −1.13 |
| 741 | 1392786_at | 1.00 | 0 | −1.08 | 0.3 | 1.09 | 0.3 | −1.03 |
| 742 | 1392905_at | −1.67 | 1.3 | 1.85 | 2 | 1.59 | 1.3 | 3.27 |
| 743 | 1392917_at | 1.04 | 0.3 | −1.07 | 0.6 | −1.10 | 1 | −1.20 |
| 744 | 1392934_at | 1.09 | 0.6 | 1.20 | 2.2 | 1.15 | 1.4 | 1.08 |
| 745 | 1392948_at | −1.22 | 0.4 | 3.66 | 7.9 | 1.71 | 2 | 2.58 |
| 746 | 1392953_at | 1.11 | 0.5 | −2.57 | 14.1 | −2.19 | 11.2 | −3.11 |
| 747 | 1392953_at | 1.11 | 0.5 | −2.57 | 14.1 | −2.19 | 11.2 | −3.11 |
| 748 | 1392990_at | 1.20 | 0.3 | 2.68 | 4.3 | 5.91 | 10.2 | 6.39 |
| 749 | 1393003_at | −1.14 | 0.5 | 1.74 | 4.9 | 1.21 | 1 | 1.24 |
| 750 | 1393067_at | 1.92 | 4.6 | 2.93 | 11.3 | 2.79 | 10.6 | 3.37 |
| 751 | 1393210_at | 1.03 | 0.1 | 1.25 | 0.8 | 1.18 | 0.5 | 1.64 |
| 752 | 1393235_at | 1.41 | 0.5 | 4.89 | 5.4 | 6.63 | 7 | 10.10 |
| 753 | 1393240_at | 1.00 | 0 | 1.45 | 4.8 | 1.32 | 3 | 1.44 |
| 754 | 1393249_at | −1.00 | 0 | 1.85 | 2.9 | 1.26 | 0.7 | 2.57 |
| 755 | 1393252_at | 1.19 | 0.3 | 4.17 | 8.7 | 2.71 | 5.1 | 3.17 |
| 756 | 1393281_at | 1.28 | 0.9 | −2.91 | 9.2 | −1.63 | 2.8 | −1.30 |
| 757 | 1393282_at | −1.31 | 1.3 | −1.03 | 0.1 | 1.42 | 2.2 | 1.22 |
| 758 | 1393316_at | 2.59 | 5.7 | 2.41 | 6.3 | 2.02 | 4.5 | 2.83 |
| 759 | 1393401_at | 1.42 | 0.5 | 3.81 | 4.5 | 8.18 | 8.8 | 9.47 |
| 760 | 1393427_s_at | 1.00 | 0 | 1.21 | 1.5 | 1.64 | 6.4 | 1.89 |
| 761 | 1393460_at | −1.06 | 0.2 | 1.08 | 0.3 | 1.30 | 1.7 | 1.24 |
| 762 | 1393706_at | −1.28 | 1.1 | 3.09 | 12.3 | 2.73 | 10.5 | 3.03 |
| 763 | 1393730_at | −1.00 | 0 | 1.92 | 5 | 1.42 | 1.9 | 1.68 |
| 764 | 1393799_at | −1.08 | 0.2 | −1.22 | 0.7 | 1.05 | 0.1 | −1.11 |
| 765 | 1393883_at | −1.43 | 0.7 | −1.14 | 0.2 | 1.67 | 1.4 | 1.24 |
| 766 | 1393917_at | 2.33 | 1 | −1.20 | 0.2 | −1.27 | 0.2 | 4.12 |
| 767 | 1393944_at | 5.56 | 5.8 | 5.63 | 6.9 | 10.60 | 10.7 | 8.46 |
| 768 | 1393987_s_at | −1.12 | 0.8 | −1.17 | 1.4 | −1.13 | 1 | 1.35 |
| 769 | 1394109_at | −1.14 | 0.2 | 5.86 | 8.2 | 4.33 | 6.2 | 3.60 |
| 770 | 1394315_at | −1.26 | 0.8 | 1.11 | 0.3 | 1.32 | 1.2 | 1.33 |
| 771 | 1394375_x_at | −1.23 | 0.8 | 1.59 | 3.3 | 2.05 | 6.4 | 2.38 |
| 772 | 1394459_at | −1.10 | 0.4 | 1.05 | 0.2 | 1.03 | 0.1 | −1.33 |
| 773 | 1394473_at | −1.12 | 0.3 | −1.01 | 0 | 1.31 | 1.4 | 1.05 |
| 774 | 1394483_at | 1.69 | 1.3 | 4.20 | 7.4 | 2.55 | 3.9 | 2.09 |
| 775 | 1394490_at | −1.24 | 0.3 | 3.81 | 5.1 | 2.80 | 3.7 | 5.85 |
| 776 | 1394597_at | −1.22 | 0.8 | 2.00 | 6.2 | 2.08 | 6.7 | 1.54 |
| 777 | 1394612_at | −1.10 | 0.3 | 1.05 | 0.2 | 1.13 | 0.6 | −1.16 |
| 778 | 1395079_at | 1.19 | 0.5 | 1.38 | 1.6 | 1.39 | 1.6 | 1.47 |
| 779 | 1395197_at | −1.17 | 0.4 | 1.25 | 0.7 | 1.45 | 1.4 | 1.31 |
| 780 | 1395265_at | 1.02 | 0.1 | 1.45 | 3.9 | 1.28 | 2.1 | 1.62 |
| 781 | 1395357_at | 1.31 | 1 | −1.13 | 0.4 | −1.57 | 2.5 | −2.11 |
| 782 | 1395508_at | 1.06 | 0.2 | −1.16 | 0.9 | −1.08 | 0.4 | −1.01 |
| 783 | 1395635_at | −1.34 | 0.9 | 1.72 | 2.7 | 2.03 | 4.2 | 1.46 |
| 784 | 1395663_at | −1.17 | 0.2 | 1.76 | 1.7 | 3.24 | 5.3 | 2.93 |
| 785 | 1395730_at | 1.32 | 2 | −1.27 | 1.8 | −1.14 | 0.8 | −1.22 |
| 786 | 1396055_at | 1.01 | 0 | −1.18 | 0.7 | −1.18 | 0.7 | −1.22 |
| 787 | 1396208_at | 1.00 | 0 | 2.03 | 5.5 | 1.82 | 4.3 | 1.46 |
| 788 | 1397272_at | 1.12 | 0.4 | −1.96 | 5.8 | −1.39 | 1.9 | −1.59 |
| 789 | 1397317_at | −1.03 | 0.1 | −1.01 | 0 | −1.13 | 0.5 | −1.06 |
| 790 | 1397644_at | −1.02 | 0.1 | −1.52 | 4.7 | −1.13 | 0.8 | −1.14 |
| 791 | 1397674_at | 1.17 | 0.7 | 1.09 | 0.4 | −1.01 | 0 | 1.42 |
| 792 | 1397808_at | 1.53 | 1.5 | 1.09 | 0.2 | 1.30 | 0.9 | 1.63 |
| 793 | 1398131_at | −2.67 | 2.8 | −1.53 | 0.9 | −1.12 | 0.2 | −1.70 |
| 794 | 1398246_s_at | −1.00 | 0 | 2.12 | 3.9 | 2.89 | 6.6 | 4.07 |
| 795 | 1398256_at | 1.23 | 0.3 | 3.44 | 4.6 | 1.15 | 0.2 | 1.46 |
| 796 | 1398275_at | −1.22 | 0.2 | 4.52 | 3 | 12.23 | 6.5 | 4.87 |
| 797 | 1398286_at | −1.07 | 0.3 | −1.21 | 1.5 | 1.03 | 0.1 | 1.27 |
| 798 | 1398287_at | −1.12 | 0.3 | 2.01 | 5 | 3.13 | 10.3 | 3.75 |
| 799 | 1398303_s_at | 1.49 | 2.9 | −1.36 | 2.1 | −1.07 | 0.2 | −1.23 |
| 800 | 1398332_at | −1.15 | 1.6 | −1.09 | 0.8 | 1.05 | 0.4 | 1.21 |
| 801 | 1398333_at | 1.60 | 4.1 | 4.84 | 21.9 | 2.88 | 13.9 | 3.09 |
| 802 | 1398335_at | −1.24 | 1.6 | 1.30 | 2.3 | 1.55 | 5.1 | 1.59 |
| 803 | 1398348_at | −1.14 | 0.4 | −1.05 | 0.1 | 1.21 | 0.8 | 1.61 |
| 804 | 1398350_at | 1.28 | 1.4 | −1.25 | 1.3 | −1.17 | 0.8 | 1.59 |
| 805 | 1398356_at | 1.25 | 4.1 | −1.14 | 1.8 | −1.06 | 0.6 | −1.02 |
| 806 | 1398380_at | −1.29 | 1 | 1.14 | 0.4 | 1.37 | 1.4 | 1.44 |
| 807 | 1398390_at | −1.20 | 0.3 | 3.78 | 6.6 | 5.71 | 9.8 | 5.62 |
| 808 | 1398397_at | 1.24 | 0.9 | 1.53 | 2.7 | 2.53 | 8.9 | 3.60 |
| 809 | 1398597_at | −1.12 | 0.6 | 1.28 | 2.3 | 1.29 | 2.5 | 1.67 |
| 810 | 1398626_s_at | −1.02 | 0.1 | −1.26 | 3 | −1.25 | 2.8 | −1.59 |
| 811 | 1398656_at | 4.45 | 2.3 | 13.93 | 6.6 | 5.88 | 3.6 | 3.10 |
| 812 | 1398664_at | 1.11 | 0.5 | 1.45 | 3.5 | 1.15 | 0.8 | −1.25 |
| 813 | 1398727_at | −1.07 | 0.1 | 1.17 | 0.6 | 1.31 | 1.2 | 1.71 |
| 814 | 1398757_at | 1.03 | 0.2 | −1.34 | 5.5 | −1.12 | 1.2 | −1.24 |
| 815 | 1398765_at | 1.10 | 1 | −1.04 | 0.4 | −1.13 | 1.6 | 1.26 |
| 816 | 1398828_at | 1.24 | 3.9 | −1.12 | 1.5 | −1.14 | 2 | −1.15 |
| 817 | 1398829_at | 1.02 | 0.1 | −1.18 | 2.4 | −1.17 | 2.4 | −1.06 |
| 818 | 1398833_at | 1.28 | 4.1 | −1.12 | 1.3 | −1.04 | 0.3 | 1.09 |
| 819 | 1398840_at | −1.17 | 0.8 | 1.30 | 1.7 | −1.03 | 0.1 | 1.09 |
| 820 | 1398851_at | 1.36 | 6.7 | −1.11 | 1.3 | −1.04 | 0.4 | 1.11 |
| 821 | 1398857_at | −1.13 | 0.7 | 1.05 | 0.2 | 1.00 | 0 | −1.17 |
| 822 | 1398873_at | 1.18 | 1.6 | −1.10 | 0.8 | 1.10 | 0.8 | 1.37 |
| 823 | 1398893_at | 1.18 | 1.7 | −1.39 | 5.3 | −1.33 | 4.3 | −1.61 |
| 824 | 1398962_at | 1.08 | 0.4 | 1.05 | 0.2 | 1.14 | 0.8 | 1.16 |
| 825 | 1398970_at | 1.10 | 0.7 | 1.09 | 0.7 | −1.04 | 0.3 | −1.13 |
| 826 | 1398994_at | 1.09 | 0.4 | 1.29 | 1.9 | 1.15 | 0.8 | 1.10 |
| 827 | 1399019_at | −1.02 | 0.1 | 1.37 | 5.9 | 1.10 | 1 | 1.31 |
| 828 | 1399045_at | 1.07 | 0.3 | 1.20 | 1.2 | 1.33 | 2.4 | 1.34 |
| 829 | 1399122_at | 1.05 | 0.2 | 1.13 | 0.6 | 1.01 | 0 | −1.06 |

| | | Fold | | Fold | NLogP | Fold | NLogP |
|---|---|---|---|---|---|---|---|
| | NLogP | Change | NLogP | Change | Day | Change | Day |
| No. | Day 4 | Day 7 | Day 7 | Day 10 | 10 | Day 14 | 14 |
| 1 | 3.5 | 1.40 | 6.7 | 1.61 | 10.3 | 1.46 | 8.1 |
| 2 | 0.7 | 1.20 | 3.3 | 1.25 | 4.1 | 1.13 | 1.8 |
| 3 | 2.9 | −1.37 | 4.1 | −1.36 | 3.8 | −1.42 | 4.8 |
| 4 | 3.2 | −1.04 | 0.2 | −1.03 | 0.1 | 1.16 | 0.9 |
| 5 | 1.3 | 1.60 | 5.4 | 1.66 | 5.6 | 1.76 | 7.1 |
| 6 | 2 | 1.18 | 1.6 | 1.36 | 3.9 | 1.33 | 3.7 |
| 7 | 4.1 | −1.42 | 2.2 | −1.23 | 0.9 | −1.12 | 0.4 |
| 8 | 2.5 | 1.28 | 3.7 | 1.25 | 2.9 | 1.10 | 0.9 |
| 9 | 0.2 | −1.84 | 9.7 | −1.51 | 5.1 | −1.35 | 3.4 |
| 10 | 0.6 | −1.16 | 1 | −1.75 | 7.2 | −1.43 | 3.9 |
| 11 | 0.6 | 6.41 | 4.2 | 2.38 | 1.2 | 1.74 | 0.7 |
| 12 | 5.2 | −1.40 | 4.6 | −1.17 | 1.3 | −1.17 | 1.5 |
| 13 | 6.1 | 35.18 | 18.3 | 46.89 | 18.9 | 24.23 | 16.1 |
| 14 | 3.8 | 13.16 | 11.6 | 9.10 | 8.8 | 5.39 | 6.3 |
| 15 | 2.3 | −1.37 | 1.4 | −1.34 | 1.2 | −1.05 | 0.1 |
| 16 | 4 | 1.05 | 0.3 | 1.14 | 1.1 | −1.16 | 1.3 |
| 17 | 4.1 | 1.05 | 0.3 | 1.10 | 0.6 | 1.05 | 0.3 |
| 18 | 5.4 | 1.23 | 1.8 | 1.22 | 1.6 | −1.18 | 1.3 |
| 19 | 2.9 | −1.01 | 0.1 | 1.08 | 0.5 | 1.10 | 0.8 |
| 20 | 12 | 6.53 | 24.9 | 9.45 | 28.1 | 5.85 | 23.5 |
| 21 | 0 | −1.34 | 5 | −1.30 | 3.8 | −1.39 | 5.9 |
| 22 | 8 | 1.01 | 0.1 | 1.01 | 0.1 | 1.03 | 0.2 |
| 23 | 0.5 | −1.56 | 5.1 | −1.48 | 3.8 | −1.56 | 5.1 |
| 24 | 3.3 | 1.42 | 8 | 1.46 | 8.4 | 1.37 | 6.9 |
| 25 | 0.3 | 7.31 | 6 | 6.82 | 5.3 | 4.95 | 4.3 |
| 26 | 3.8 | −1.04 | 0.1 | −1.25 | 1.2 | 1.07 | 0.3 |
| 27 | 0.8 | 1.35 | 7.6 | 1.32 | 6.2 | 1.38 | 8.3 |
| 28 | 1.7 | 1.08 | 0.3 | 1.26 | 1.3 | 1.49 | 3.3 |
| 29 | 0 | 1.09 | 0.3 | −1.17 | 0.7 | −1.21 | 0.9 |
| 30 | 12.4 | 1.07 | 0.2 | −1.27 | 1.2 | −1.64 | 4 |
| 31 | 4 | 1.58 | 2 | 1.06 | 0.1 | −1.15 | 0.4 |
| 32 | 3.2 | 2.11 | 8.4 | 2.25 | 8.9 | 1.84 | 6.2 |
| 33 | 12.4 | 2.34 | 14.4 | 2.50 | 14.9 | 2.24 | 13.4 |
| 34 | 2.7 | 1.43 | 3.4 | 1.65 | 5.4 | 1.67 | 6 |
| 35 | 0.6 | −1.37 | 1.5 | −1.47 | 1.9 | −1.82 | 4.1 |
| 36 | 0.3 | −1.29 | 1.5 | −1.83 | 5.1 | −1.74 | 4.8 |
| 37 | 8.7 | −1.85 | 6 | −1.87 | 5.7 | −1.77 | 5.3 |
| 38 | 8.8 | 1.75 | 2.3 | 1.21 | 0.5 | −1.06 | 0.1 |
| 39 | 0.2 | −1.72 | 4.7 | −1.20 | 0.9 | −1.16 | 0.7 |
| 40 | 9 | −1.05 | 0.2 | −1.05 | 0.2 | −1.30 | 2.5 |
| 41 | 5.3 | 1.95 | 11.2 | 1.94 | 10.4 | 1.78 | 9.1 |
| 42 | 1.4 | 1.14 | 0.4 | 1.40 | 1.5 | 1.85 | 4.2 |
| 43 | 4.4 | −1.40 | 3.6 | −1.04 | 0.2 | −1.10 | 0.6 |
| 44 | 0.3 | 6.37 | 5 | 11.94 | 7.2 | 5.62 | 4.5 |
| 45 | 2.1 | 1.24 | 1.2 | 1.06 | 0.2 | −1.32 | 1.6 |
| 46 | 0.6 | 2.07 | 4.4 | 2.25 | 4.9 | 2.89 | 7.8 |
| 47 | 7.7 | 14.29 | 18.2 | 17.75 | 18.8 | 11.24 | 16.3 |
| 48 | 2.4 | 1.17 | 0.5 | 1.07 | 0.2 | −1.05 | 0.1 |
| 49 | 1.1 | −2.03 | 4.7 | −2.85 | 7.8 | −2.37 | 6.3 |
| 50 | 3.4 | 3.90 | 9.7 | 3.59 | 8.3 | 2.54 | 5.6 |
| 51 | 9.7 | 5.40 | 4.3 | 3.66 | 2.7 | 4.24 | 3.4 |
| 52 | 9.2 | −2.29 | 3.5 | −4.18 | 7.5 | −13.07 | 17.2 |
| 53 | 1.3 | 1.22 | 1.8 | 1.09 | 0.5 | 1.07 | 0.4 |
| 54 | 2.3 | 22.52 | 9.2 | 48.51 | 11.6 | 11.66 | 6.5 |
| 55 | 0.4 | −1.48 | 3.3 | −1.59 | 4 | −1.58 | 4.2 |
| 56 | 7.7 | −5.53 | 7 | −3.19 | 3.6 | 1.97 | 1.7 |
| 57 | 2.5 | 2.46 | 5.9 | 2.50 | 5.6 | 2.46 | 5.9 |
| 58 | 7.9 | 3.99 | 15.2 | 3.70 | 13.4 | 2.59 | 9.3 |
| 59 | 6 | 8.06 | 14.3 | 7.76 | 13.2 | 3.52 | 7.2 |
| 60 | 4.8 | 2.03 | 3.8 | 2.09 | 3.7 | 2.40 | 5.2 |
| 61 | 4.1 | 7.19 | 12.7 | 9.53 | 14.1 | 5.10 | 9.8 |
| 62 | 2.1 | −5.15 | 6 | −8.78 | 8.5 | −8.14 | 8.6 |
| 63 | 0.9 | 1.13 | 0.8 | −1.11 | 0.6 | 1.06 | 0.3 |
| 64 | 1.6 | 1.55 | 5.1 | 1.89 | 8.4 | 1.68 | 6.7 |
| 65 | 0.6 | −2.62 | 7.5 | −1.82 | 3.4 | −1.82 | 3.6 |
| 66 | 1 | −1.45 | 2.8 | −1.61 | 4 | −1.30 | 1.7 |
| 67 | 1.8 | 1.23 | 0.6 | 1.09 | 0.2 | 1.22 | 0.6 |
| 68 | 0 | 1.80 | 3.3 | 2.04 | 4.2 | 2.85 | 8.1 |
| 69 | 0.4 | 1.33 | 1.1 | −1.20 | 0.6 | −1.96 | 4.3 |
| 70 | 1.1 | −1.56 | 2.7 | −2.52 | 7.8 | −1.93 | 5 |
| 71 | 2.9 | −2.03 | 6.4 | −2.02 | 5.9 | −1.83 | 5 |
| 72 | 3.8 | 2.91 | 4.2 | 1.87 | 1.7 | −1.12 | 0.2 |
| 73 | 8.9 | 13.30 | 10.8 | 10.27 | 8.7 | 11.96 | 10.2 |
| 74 | 1.2 | 2.02 | 4.7 | 2.34 | 5.8 | 3.45 | 10.7 |
| 75 | 7.8 | 12.39 | 18.7 | 18.38 | 20.8 | 13.06 | 19.1 |
| 76 | 4.1 | 5.77 | 5 | 1.22 | 0.2 | −1.44 | 0.5 |
| 77 | 2.4 | 1.53 | 3.7 | 1.47 | 2.9 | 1.69 | 5.1 |
| 78 | 3.6 | −1.28 | 0.9 | −1.68 | 2.6 | −1.89 | 3.8 |
| 79 | 0 | −2.03 | 4.4 | −2.64 | 6.7 | −2.34 | 5.9 |
| 80 | 7.4 | 1.31 | 3 | 1.36 | 3.4 | 1.41 | 4.4 |
| 81 | 4 | −2.79 | 14.3 | −2.19 | 9.5 | −1.91 | 7.7 |
| 82 | 5.6 | 2.14 | 1.4 | 3.62 | 2.9 | 3.39 | 2.9 |
| 83 | 0.1 | −1.88 | 7.1 | −1.69 | 5.1 | −1.99 | 8.1 |
| 84 | 5.1 | 4.90 | 3.6 | 2.49 | 1.4 | 3.81 | 2.7 |
| 85 | 1 | 1.79 | 3 | 2.75 | 6.6 | 2.44 | 5.8 |
| 86 | 0.5 | −1.10 | 0.5 | −1.51 | 3.9 | 1.05 | 0.2 |
| 87 | 1 | −2.01 | 4.4 | −1.83 | 3.3 | −2.55 | 6.9 |
| 88 | 2.9 | −2.64 | 2.4 | −4.69 | 4.6 | −6.38 | 6.5 |
| 89 | 9.2 | 2.13 | 6.9 | 1.82 | 4.5 | 1.47 | 2.5 |
| 90 | 4.2 | 1.33 | 1 | 1.06 | 0.1 | 1.10 | 0.3 |
| 91 | 2.5 | 2.10 | 8.8 | 2.35 | 9.9 | 1.74 | 5.7 |
| 92 | 1.6 | 1.69 | 3.1 | 2.41 | 6.3 | 2.60 | 7.7 |
| 93 | 3.2 | −1.62 | 2.2 | −1.07 | 0.2 | −1.05 | 0.1 |
| 94 | 0.7 | 2.61 | 6.8 | 1.92 | 3.5 | 1.40 | 1.3 |
| 95 | 6.5 | 2.57 | 4.8 | 1.56 | 1.4 | 1.46 | 1.2 |
| 96 | 1 | −1.35 | 3.2 | −1.26 | 2 | −1.25 | 2.1 |
| 97 | 0.3 | 2.35 | 6.9 | 2.46 | 6.9 | 2.15 | 5.9 |
| 98 | 2.2 | 3.45 | 2.8 | 4.09 | 3.2 | 1.69 | 0.8 |
| 99 | 0.8 | 1.84 | 6 | 1.12 | 0.5 | −1.33 | 1.8 |
| 100 | 6.2 | 1.22 | 0.4 | −1.44 | 1 | 1.00 | 0 |
| 101 | 0.1 | 1.60 | 1.7 | 2.03 | 3 | 2.26 | 4 |
| 102 | 0.1 | −1.41 | 1.8 | −1.69 | 3.2 | −1.87 | 4.6 |
| 103 | 0.1 | 1.53 | 2.5 | 2.36 | 7 | 2.76 | 9.5 |
| 104 | 0.5 | −1.65 | 4.2 | −1.36 | 1.8 | −1.66 | 4.2 |
| 105 | 1.1 | 1.57 | 5.6 | 1.92 | 9 | 1.83 | 8.7 |
| 106 | 3.3 | 1.02 | 0 | −1.16 | 0.4 | −2.28 | 5.2 |
| 107 | 0.5 | 4.63 | 15.7 | 6.06 | 17.9 | 5.77 | 18.3 |
| 108 | 0.7 | 4.46 | 9.1 | 5.91 | 10.8 | 8.30 | 14.4 |
| 109 | 0.2 | 2.41 | 9.7 | 2.33 | 8.6 | 2.17 | 8.1 |
| 110 | 0.6 | −1.72 | 5.6 | −2.04 | 7.9 | −1.69 | 5.4 |
| 111 | 7.4 | −1.02 | 0 | −1.75 | 2.2 | −2.58 | 5.5 |
| 112 | 5.2 | 8.96 | 10.8 | 11.59 | 11.7 | 7.04 | 9.2 |
| 113 | 0.1 | 1.27 | 4.3 | 1.29 | 4.4 | 1.29 | 4.8 |
| 114 | 3.9 | 1.26 | 1.2 | 1.76 | 4.3 | 1.83 | 5.1 |
| 115 | 2.3 | 1.21 | 2.1 | 1.30 | 3.1 | 1.41 | 5.3 |
| 116 | 0.1 | −1.48 | 2.9 | −1.64 | 3.8 | −2.41 | 9.7 |
| 117 | 1.1 | 1.28 | 3.3 | 1.28 | 3 | 1.07 | 0.5 |
| 118 | 0.9 | 3.34 | 5.5 | 2.25 | 2.7 | 3.03 | 4.8 |
| 119 | 9.8 | 1.68 | 4.5 | 1.29 | 1.4 | 1.79 | 5.3 |
| 120 | 5 | 1.24 | 3.4 | 1.34 | 5.1 | 1.01 | 0 |
| 121 | 4.3 | 1.21 | 1.3 | 1.12 | 0.6 | 1.16 | 0.9 |
| 122 | 0.4 | −1.48 | 5.4 | −1.40 | 4 | −1.40 | 4.4 |
| 123 | 5.7 | 1.08 | 0.4 | −1.27 | 1.5 | −1.57 | 4.3 |
| 124 | 6 | −1.27 | 1.6 | −1.09 | 0.3 | 1.01 | 0 |
| 125 | 0.7 | 1.26 | 2.1 | 1.15 | 1 | 1.22 | 1.7 |
| 126 | 0.8 | 5.14 | 23 | 11.15 | 31.3 | 12.54 | 33.7 |
| 127 | 1.8 | 1.25 | 0.8 | −1.21 | 0.6 | −1.30 | 1.1 |
| 128 | 2.5 | 1.29 | 0.4 | 1.60 | 0.9 | 3.62 | 4.4 |
| 129 | 1.8 | 1.74 | 7.4 | 1.73 | 6.8 | 1.71 | 7 |
| 130 | 3.5 | 2.05 | 8.2 | 1.63 | 4.3 | 1.31 | 1.9 |
| 131 | 1 | −1.44 | 4 | −1.34 | 2.7 | −1.27 | 2.1 |
| 132 | 1.2 | 7.48 | 19.6 | 7.36 | 18.4 | 5.66 | 16.5 |
| 133 | 0.8 | −1.13 | 0.8 | −1.26 | 2 | −1.26 | 2.1 |
| 134 | 1 | 1.22 | 3.3 | 1.47 | 8.1 | 1.35 | 6.2 |
| 135 | 2.7 | 1.65 | 2.2 | 1.13 | 0.3 | 1.99 | 3.6 |
| 136 | 4 | 1.16 | 0.9 | 1.12 | 0.6 | 1.51 | 4.6 |
| 137 | 8.1 | 10.91 | 17.5 | 8.49 | 15.4 | 3.47 | 8 |
| 138 | 9.2 | 1.04 | 0.2 | −1.48 | 2.8 | −1.47 | 2.9 |
| 139 | 2.8 | 1.40 | 4.2 | 1.45 | 4.5 | 1.33 | 3.2 |
| 140 | 6.7 | −1.21 | 1.6 | −1.23 | 1.7 | −1.14 | 0.9 |
| 141 | 2.5 | 1.25 | 1.9 | 1.02 | 0.1 | 1.10 | 0.5 |
| 142 | 9.1 | 1.67 | 2.3 | 1.31 | 0.8 | −1.33 | 0.9 |
| 143 | 0.1 | −2.39 | 10 | −1.87 | 5.7 | −2.37 | 9.8 |
| 144 | 0.6 | 1.26 | 1.5 | 1.11 | 0.4 | 1.18 | 0.9 |
| 145 | 0.6 | 2.73 | 4 | 1.46 | 0.8 | 2.53 | 3.6 |
| 146 | 0.8 | 1.69 | 3.8 | 2.01 | 5.5 | 1.58 | 3.1 |
| 147 | 5.7 | 2.91 | 4.4 | 3.51 | 5.3 | 2.55 | 3.6 |
| 148 | 0.6 | 3.17 | 4.1 | 2.62 | 2.9 | −1.11 | 0.2 |
| 149 | 3.4 | 1.57 | 0.7 | −2.23 | 1.4 | −4.09 | 3.7 |
| 150 | 2.8 | 1.98 | 1.4 | −2.61 | 2.1 | −3.55 | 3.5 |
| 151 | 1.7 | −1.62 | 6.6 | −1.21 | 1.5 | −1.11 | 0.7 |
| 152 | 2.1 | −1.10 | 0.4 | −1.75 | 4.9 | −1.84 | 6 |
| 153 | 2 | −1.66 | 5.3 | −1.69 | 5.2 | −1.63 | 5 |
| 154 | 2 | −1.20 | 1.5 | 1.19 | 1.4 | 1.32 | 3 |
| 155 | 0.9 | 2.16 | 3.9 | 2.46 | 4.7 | 1.92 | 3.1 |
| 156 | 4 | −1.10 | 1 | 1.05 | 0.4 | 1.02 | 0.1 |
| 157 | 6.7 | −1.37 | 2.6 | −1.32 | 2 | −1.43 | 3.1 |
| 158 | 2.3 | −1.68 | 3.3 | −2.17 | 5.7 | −2.28 | 6.7 |
| 159 | 2.6 | −1.72 | 8.3 | −1.63 | 6.7 | −1.48 | 5.1 |
| 160 | 9.9 | 1.92 | 4.4 | 1.98 | 4.4 | 1.21 | 0.7 |
| 161 | 7.8 | −1.13 | 0.3 | −1.36 | 0.9 | −1.40 | 1.1 |
| 162 | 9.1 | 2.16 | 13.2 | 2.25 | 13.2 | 2.30 | 14.5 |
| 163 | 2.6 | 2.29 | 5.3 | 1.48 | 1.5 | 2.89 | 7.7 |
| 164 | 4.7 | 1.34 | 1.3 | −1.12 | 0.4 | −1.26 | 1 |
| 165 | 4.8 | 1.57 | 5 | 1.33 | 2.3 | 1.49 | 4.1 |
| 166 | 1.3 | 1.03 | 0.1 | −1.05 | 0.2 | 1.09 | 0.3 |
| 167 | 0.8 | −1.72 | 5 | −1.24 | 1.1 | −1.10 | 0.4 |
| 168 | 3.5 | 1.76 | 3.7 | 1.61 | 2.6 | 1.23 | 0.8 |
| 169 | 3.3 | −1.11 | 0.6 | −1.02 | 0.1 | 1.26 | 2 |
| 170 | 7.1 | 4.24 | 3.4 | 1.81 | 0.8 | 1.06 | 0.1 |
| 171 | 11.4 | 28.07 | 19.7 | 32.03 | 19.5 | 23.11 | 18.4 |
| 172 | 0.4 | −2.23 | 7 | −2.31 | 6.9 | −1.59 | 3 |
| 173 | 0.1 | 1.33 | 2.6 | 1.29 | 2 | 1.14 | 0.8 |
| 174 | 7.8 | 3.53 | 7.9 | 2.45 | 4.4 | 1.47 | 1.3 |
| 175 | 5.7 | −1.02 | 0.1 | 1.05 | 0.3 | −1.09 | 0.7 |
| 176 | 0.1 | 1.27 | 2.9 | 1.38 | 4.2 | 1.24 | 2.4 |
| 177 | 0 | 1.78 | 6.6 | 1.69 | 5.3 | 1.35 | 2.4 |
| 178 | 0.6 | 1.52 | 3.6 | 2.21 | 8.7 | 1.74 | 5.5 |
| 179 | 8.2 | −1.39 | 2.5 | 1.09 | 0.4 | −1.41 | 2.7 |
| 180 | 10 | 2.00 | 6.2 | 2.39 | 8 | 2.07 | 6.6 |
| 181 | 1.7 | 1.37 | 2 | 2.26 | 8 | 2.06 | 7.2 |
| 182 | 5.2 | 1.76 | 6.3 | 2.01 | 8 | 1.98 | 8.3 |
| 183 | 4 | −1.15 | 0.7 | −1.28 | 1.4 | −1.51 | 3.5 |
| 184 | 7.8 | 2.26 | 2.6 | 1.19 | 0.3 | −1.02 | 0 |
| 185 | 0.5 | 1.46 | 3.9 | 1.86 | 7.7 | 1.62 | 5.7 |
| 186 | 0.3 | 1.27 | 3.4 | 1.28 | 3.2 | 1.10 | 0.8 |
| 187 | 0.1 | −1.38 | 4 | −1.34 | 3.2 | −1.27 | 2.5 |
| 188 | 1.3 | −1.31 | 2.5 | −1.44 | 3.7 | −1.38 | 3.3 |
| 189 | 3.2 | −1.29 | 2.1 | −1.36 | 2.5 | −1.59 | 5.2 |
| 190 | 17.7 | 12.09 | 21.8 | 15.12 | 22.6 | 9.12 | 19.2 |
| 191 | 10 | 1.62 | 6.7 | 1.47 | 4.5 | 1.39 | 3.8 |
| 192 | 2 | −1.07 | 0.4 | 1.12 | 0.8 | 1.43 | 5.2 |
| 193 | 1.1 | 1.26 | 4.1 | 1.31 | 5 | 1.27 | 4.3 |
| 194 | 0.7 | 1.61 | 6.7 | 1.78 | 8.3 | 2.20 | 13.6 |
| 195 | 1 | 1.58 | 7.7 | 2.26 | 15.6 | 2.26 | 16.5 |
| 196 | 1.4 | 3.43 | 3.8 | 4.13 | 4.4 | 3.15 | 3.4 |
| 197 | 4.3 | −1.15 | 0.9 | 1.04 | 0.2 | −1.03 | 0.1 |
| 198 | 2.3 | 1.30 | 2.3 | 1.65 | 6 | 1.55 | 5.3 |
| 199 | 1.4 | 1.18 | 2.6 | 1.33 | 5.8 | 1.32 | 6 |
| 200 | 0.2 | −1.61 | 4.4 | −1.22 | 1.1 | −1.64 | 4.7 |
| 201 | 4.2 | 1.25 | 2.4 | 1.02 | 0.1 | 1.13 | 1 |
| 202 | 5.4 | −1.40 | 6.6 | −1.29 | 4 | −1.18 | 2.3 |
| 203 | 0.2 | 1.10 | 0.8 | 1.25 | 2.5 | 1.03 | 0.2 |
| 204 | 3 | 2.12 | 4.8 | 1.68 | 2.5 | 1.44 | 1.6 |
| 205 | 8.9 | 5.20 | 17.7 | 4.40 | 14.7 | 3.35 | 12.1 |
| 206 | 3.5 | 1.10 | 0.5 | 1.03 | 0.1 | −1.06 | 0.2 |
| 207 | 6.8 | −1.14 | 1.3 | 1.00 | 0 | −1.03 | 0.2 |
| 208 | 4.6 | 1.19 | 0.4 | 1.12 | 0.2 | 1.11 | 0.2 |
| 209 | 13.6 | 1.16 | 1.9 | −1.05 | 0.4 | −1.12 | 1.2 |
| 210 | 1.4 | 1.70 | 2.2 | 2.03 | 3.2 | 2.87 | 6.4 |
| 211 | 5.2 | 1.20 | 1.3 | 1.09 | 0.4 | −1.08 | 0.4 |
| 212 | 5 | −1.14 | 0.6 | −1.31 | 1.5 | −1.34 | 1.8 |
| 213 | 0.3 | 1.04 | 0.2 | −1.53 | 4.4 | −1.23 | 1.6 |
| 214 | 0.2 | 1.25 | 1.5 | 1.27 | 1.5 | 1.22 | 1.2 |
| 215 | 3.9 | 2.83 | 12.9 | 2.21 | 8.4 | 2.58 | 11.4 |
| 216 | 4.5 | 1.44 | 5.1 | 1.46 | 5.1 | 1.26 | 2.6 |
| 217 | 1.4 | −1.26 | 2 | −1.12 | 0.7 | −1.07 | 0.3 |
| 218 | 1.1 | 1.38 | 4.2 | 1.57 | 6.5 | 1.28 | 2.7 |
| 219 | 0.6 | −1.33 | 4.4 | −1.38 | 5 | −1.28 | 3.6 |
| 220 | 0.9 | 3.17 | 9.5 | 3.67 | 10.5 | 2.95 | 8.7 |
| 221 | 0.9 | 3.17 | 9.5 | 3.67 | 10.5 | 2.95 | 8.7 |
| 222 | 0.4 | −1.40 | 3.1 | −1.74 | 6.3 | −1.21 | 1.3 |
| 223 | 6.4 | 1.33 | 1.1 | −1.24 | 0.7 | −1.21 | 0.6 |
| 224 | 0.1 | 2.26 | 10.5 | 2.04 | 8.2 | 2.19 | 10 |
| 225 | 0.1 | 1.43 | 2.2 | 1.90 | 5 | 1.96 | 5.9 |
| 226 | 0 | 1.19 | 2.2 | 1.33 | 4.4 | 1.31 | 4.3 |
| 227 | 8.3 | 2.38 | 26.3 | 2.39 | 25.3 | 2.17 | 23.7 |
| 228 | 5.2 | −1.16 | 1.6 | −1.03 | 0.1 | 1.03 | 0.2 |
| 229 | 0.3 | 2.74 | 8.2 | 2.89 | 8.3 | 2.17 | 5.6 |
| 230 | 0.4 | −1.80 | 4.2 | −1.96 | 4.8 | −1.36 | 1.5 |
| 231 | 0.6 | −1.62 | 9.8 | −1.66 | 9.8 | −1.55 | 8.6 |
| 232 | 0.5 | −1.13 | 1.6 | −1.11 | 1.2 | −1.40 | 7.6 |
| 233 | 12.2 | 1.05 | 0.1 | −1.03 | 0.1 | −1.12 | 0.4 |
| 234 | 0.6 | 1.95 | 5.4 | 2.61 | 8.6 | 1.99 | 5.7 |
| 235 | 0.2 | −1.06 | 0.2 | −1.33 | 1.1 | −1.09 | 0.2 |
| 236 | 0.9 | 1.42 | 5.8 | 1.59 | 8.2 | 1.83 | 12.5 |
| 237 | 0.7 | −1.26 | 2.7 | −1.20 | 1.8 | −1.30 | 3.3 |
| 238 | 7.3 | 3.40 | 12.5 | 4.88 | 16.3 | 4.80 | 17 |
| 239 | 0.7 | 1.93 | 7.4 | 1.90 | 6.6 | 1.84 | 6.6 |
| 240 | 6.4 | 3.50 | 19.5 | 4.50 | 22.4 | 3.90 | 21.3 |
| 241 | 5.6 | 1.68 | 8.1 | 1.96 | 10.8 | 1.38 | 3.9 |
| 242 | 13 | 1.62 | 2.1 | 1.42 | 1.2 | 2.04 | 3.9 |
| 243 | 4.1 | 4.04 | 10.4 | 3.25 | 7.6 | 2.39 | 5.2 |
| 244 | 0.4 | 1.53 | 6.5 | 1.83 | 10.1 | 1.71 | 9.2 |
| 245 | 6.8 | 1.43 | 1.8 | 1.23 | 0.7 | 1.91 | 4.4 |
| 246 | 4.8 | 2.70 | 5.8 | 3.07 | 6.5 | 1.93 | 3 |
| 247 | 0.1 | 2.75 | 6.7 | 3.26 | 7.8 | 2.84 | 7 |
| 248 | 1.2 | −1.09 | 0.4 | −1.21 | 1.1 | −1.36 | 2.5 |
| 249 | 1.6 | 1.12 | 0.6 | 1.52 | 3.9 | 1.63 | 5.3 |
| 250 | 0.1 | −1.69 | 2.9 | −1.70 | 2.8 | −1.94 | 4.2 |
| 251 | 2.6 | −1.42 | 1.6 | −1.59 | 2.3 | −1.32 | 1.1 |
| 252 | 6.8 | 1.65 | 8.3 | 1.86 | 10.6 | 1.81 | 10.5 |
| 253 | 2 | 2.82 | 6.9 | 2.79 | 6.3 | 2.48 | 5.7 |
| 254 | 0 | 2.15 | 4.8 | 3.05 | 7.9 | 2.36 | 5.7 |
| 255 | 3.9 | 6.52 | 8.6 | 9.30 | 10.2 | 5.21 | 7.1 |
| 256 | 2.9 | 1.82 | 11.5 | 1.92 | 12 | 1.75 | 10.5 |
| 257 | 6.9 | −1.06 | 0.3 | −1.52 | 3.6 | −1.12 | 0.6 |
| 258 | 1.4 | −1.04 | 0.2 | −1.30 | 2.1 | −1.53 | 4.9 |
| 259 | 8.4 | 1.54 | 4.6 | 1.39 | 2.8 | 1.31 | 2.2 |
| 260 | 1.7 | 1.15 | 0.9 | 1.33 | 2.4 | 1.38 | 3.1 |
| 261 | 0.4 | −1.04 | 0.2 | 1.05 | 0.3 | −1.15 | 1.3 |
| 262 | 4 | 2.64 | 5.9 | 2.71 | 5.7 | 2.91 | 6.8 |
| 263 | 4.7 | 1.19 | 1.2 | 1.47 | 3.7 | 1.61 | 5.5 |
| 264 | 0 | 1.28 | 2.9 | 1.67 | 8.2 | 1.32 | 3.4 |
| 265 | 2.6 | −1.87 | 9.8 | −1.64 | 6.5 | −1.39 | 3.7 |
| 266 | 0.7 | −1.55 | 2.3 | −1.69 | 2.8 | −1.09 | 0.2 |
| 267 | 10.8 | 2.35 | 8 | 1.79 | 4.2 | 1.70 | 3.9 |
| 268 | 8.2 | −1.09 | 0.5 | −1.59 | 5 | −1.38 | 3.1 |
| 269 | 0.1 | 1.20 | 0.6 | 1.49 | 1.8 | 2.02 | 4.7 |
| 270 | 9.5 | −1.02 | 0.1 | −1.33 | 2 | −1.43 | 3 |
| 271 | 1.5 | −1.77 | 8.1 | −1.77 | 7.7 | −1.52 | 5.1 |
| 272 | 2.9 | −1.52 | 3.9 | −1.70 | 5.2 | −1.40 | 2.8 |
| 273 | 1.7 | 4.31 | 15.5 | 4.02 | 13.8 | 3.58 | 13.1 |
| 274 | 0.4 | 1.12 | 0.8 | −1.09 | 0.5 | −1.01 | 0 |
| 275 | 1.1 | −1.50 | 6.3 | −1.37 | 3.9 | −1.39 | 4.6 |
| 276 | 16.5 | 3.72 | 28.3 | 4.51 | 30.5 | 3.45 | 26.9 |
| 277 | 4.4 | 1.07 | 0.3 | 1.25 | 1.8 | 1.17 | 1.1 |
| 278 | 2.8 | −1.84 | 8.8 | −1.26 | 1.9 | −1.10 | 0.5 |
| 279 | 8 | 2.88 | 16 | 2.81 | 14.7 | 2.34 | 12.2 |
| 280 | 6.5 | −1.10 | 0.3 | −1.23 | 1 | −1.29 | 1.4 |
| 281 | 1.3 | −1.45 | 4.5 | −1.34 | 2.9 | −1.37 | 3.5 |
| 282 | 8.4 | −1.10 | 0.5 | 1.09 | 0.4 | 1.18 | 1 |
| 283 | 5.6 | 1.22 | 2.1 | 1.05 | 0.3 | 1.16 | 1.3 |
| 284 | 1.4 | −1.43 | 6 | −1.48 | 6.3 | −1.32 | 4 |
| 285 | 12.3 | 2.72 | 3.5 | 1.80 | 1.4 | 2.68 | 3.4 |
| 286 | 4.5 | 6.11 | 8.5 | 6.78 | 8.6 | 10.91 | 12.4 |
| 287 | 6 | 1.22 | 1.6 | −1.03 | 0.1 | 1.19 | 1.3 |
| 288 | 1.2 | −1.57 | 4.6 | −1.91 | 7.3 | −1.57 | 4.6 |
| 289 | 1.5 | 2.47 | 6 | 2.84 | 7 | 2.53 | 6.3 |
| 290 | 4.8 | 1.89 | 2.7 | 1.63 | 1.7 | −1.29 | 0.7 |
| 291 | 2.2 | −1.17 | 1.7 | −1.32 | 3.7 | 1.04 | 0.2 |
| 292 | 0.4 | −1.47 | 5.6 | −1.30 | 2.9 | −1.21 | 1.8 |
| 293 | 11.6 | 2.74 | 17.4 | 1.98 | 10 | 1.87 | 9.5 |
| 294 | 0.4 | −1.66 | 5.2 | −1.43 | 2.9 | 1.03 | 0.1 |
| 295 | 1.7 | −1.06 | 0.2 | −1.29 | 1.8 | −1.10 | 0.5 |
| 296 | 0.7 | 1.05 | 0.2 | 1.25 | 1.6 | 1.07 | 0.4 |
| 297 | 0.4 | 1.41 | 2.4 | 1.50 | 2.9 | −1.23 | 1.1 |
| 298 | 0.2 | 1.39 | 1.9 | 1.45 | 2.1 | 1.79 | 4.7 |
| 299 | 1.6 | −1.35 | 1.5 | −1.22 | 0.8 | −1.31 | 1.2 |
| 300 | 1.8 | −1.64 | 2.9 | −1.50 | 2 | 1.05 | 0.1 |
| 301 | 1.5 | 8.54 | 13.2 | 9.72 | 13.4 | 6.28 | 10.8 |
| 302 | 5.1 | 5.16 | 12.6 | 7.26 | 15.1 | 3.79 | 9.6 |
| 303 | 6.8 | 1.62 | 5.3 | 1.28 | 1.7 | 1.49 | 3.9 |
| 304 | 6.2 | 1.37 | 4.5 | 1.07 | 0.4 | −1.01 | 0.1 |
| 305 | 2.1 | −1.69 | 3.2 | −2.10 | 5.1 | −2.24 | 6.2 |
| 306 | 1.2 | −1.31 | 0.3 | 1.47 | 0.4 | −2.35 | 1.2 |
| 307 | 2.4 | 2.30 | 7.4 | 1.94 | 4.9 | 2.20 | 6.9 |
| 308 | 2.1 | −1.60 | 2 | −1.65 | 2 | −2.68 | 6.2 |
| 309 | 3.5 | 3.49 | 11.7 | 3.00 | 9.2 | 1.81 | 3.9 |
| 310 | 0.1 | −1.53 | 4.6 | −1.58 | 4.9 | −1.31 | 2.3 |
| 311 | 15.4 | 2.33 | 12.3 | 1.96 | 8.4 | 1.49 | 4.1 |
| 312 | 0.9 | −1.10 | 0.3 | −1.17 | 0.5 | 1.31 | 1.2 |
| 313 | 3.7 | 1.29 | 1 | 1.79 | 3.1 | 2.72 | 7.7 |
| 314 | 6.8 | 12.99 | 8.2 | 2.64 | 1.7 | 1.49 | 0.5 |
| 315 | 1.7 | −1.34 | 2.9 | −1.33 | 2.5 | −1.47 | 4.4 |
| 316 | 2 | 1.39 | 2.7 | 1.36 | 2.3 | 1.66 | 5.3 |
| 317 | 0.5 | 1.39 | 3.1 | 1.42 | 3.2 | 1.50 | 4.2 |
| 318 | 4.6 | −1.25 | 1.2 | −1.91 | 6 | −1.95 | 6.8 |
| 319 | 6.7 | 12.31 | 23.1 | 14.98 | 23.7 | 9.38 | 20.6 |
| 320 | 0.3 | 1.16 | 1.3 | 1.11 | 0.8 | 1.16 | 1.3 |
| 321 | 0.2 | 1.02 | 0.1 | −1.13 | 0.5 | 1.03 | 0.1 |
| 322 | 0.5 | 1.39 | 2.4 | 1.19 | 0.9 | 1.57 | 4.1 |
| 323 | 2.1 | −1.37 | 0.8 | −1.45 | 1 | −1.46 | 1.1 |
| 324 | 4.1 | 1.68 | 1.3 | −1.37 | 0.6 | −1.42 | 0.7 |
| 325 | 0.4 | 2.11 | 4.5 | 1.58 | 1.9 | 1.61 | 2.2 |
| 326 | 14.3 | 4.60 | 14 | 4.32 | 12.5 | 3.43 | 10.6 |
| 327 | 2.7 | 5.72 | 5.4 | 9.46 | 7.3 | 3.04 | 2.7 |
| 328 | 3.3 | 4.26 | 5.5 | 5.35 | 6.4 | 5.11 | 6.6 |
| 329 | 0.3 | −1.87 | 2 | −3.76 | 5.9 | −1.85 | 1.9 |
| 330 | 0.2 | 2.34 | 5.8 | 2.70 | 6.8 | 1.75 | 3 |
| 331 | 0.9 | −1.40 | 1.6 | −2.21 | 5.5 | −2.82 | 8.7 |
| 332 | 1.4 | −1.21 | 1.3 | −1.25 | 1.5 | −1.15 | 0.8 |
| 333 | 0.4 | 1.33 | 1 | 2.12 | 4.1 | 1.34 | 1 |
| 334 | 7 | 3.73 | 11.3 | 2.00 | 4.1 | 1.82 | 3.5 |
| 335 | 0.7 | 1.65 | 4.3 | 1.71 | 4.5 | 1.74 | 5 |
| 336 | 10.6 | 1.10 | 0.5 | 1.08 | 0.4 | 1.10 | 0.6 |
| 337 | 2.3 | −1.71 | 7.6 | −1.73 | 7.2 | −1.89 | 9.7 |
| 338 | 1.2 | 1.07 | 0.3 | −1.24 | 1.3 | −1.53 | 4.1 |
| 339 | 7.2 | 1.16 | 0.3 | −1.55 | 1.3 | 1.01 | 0 |
| 340 | 5.7 | −1.03 | 0.1 | −1.54 | 2.7 | −1.39 | 1.9 |
| 341 | 0.7 | 1.87 | 4.1 | 1.62 | 2.5 | 1.57 | 2.4 |
| 342 | 4 | 1.51 | 2.8 | 1.04 | 0.1 | 1.55 | 3.1 |
| 343 | 4.4 | 9.86 | 9.6 | 12.76 | 10.5 | 9.31 | 9.3 |
| 344 | 1.1 | 4.63 | 3.2 | 10.85 | 5.8 | 2.94 | 1.8 |
| 345 | 4.8 | 1.80 | 2.9 | 2.95 | 7 | 2.46 | 5.7 |
| 346 | 1.3 | 7.05 | 9.3 | 5.55 | 7.2 | 5.26 | 7.4 |
| 347 | 0.7 | 1.92 | 9.3 | 2.68 | 15 | 1.83 | 8.4 |
| 348 | 1.5 | 2.45 | 2.6 | 2.32 | 2.2 | 8.63 | 9.9 |
| 349 | 0.7 | 2.37 | 4.7 | 2.17 | 3.7 | 2.08 | 3.6 |
| 350 | 3.5 | 1.56 | 2.2 | 1.54 | 1.9 | 1.44 | 1.6 |
| 351 | 0.9 | −1.80 | 4.5 | −1.68 | 3.5 | −1.22 | 0.9 |
| 352 | 0.6 | 1.27 | 2.3 | 1.18 | 1.2 | 1.28 | 2.4 |
| 353 | 0.1 | 1.15 | 0.3 | −1.32 | 0.7 | −1.07 | 0.1 |
| 354 | 1.3 | 1.34 | 1.6 | 1.31 | 1.3 | 1.30 | 1.4 |
| 355 | 0.1 | −1.04 | 0.1 | 1.32 | 1.7 | 1.61 | 4.2 |
| 356 | 5.2 | 2.13 | 8.7 | 1.82 | 5.7 | 1.75 | 5.6 |
| 357 | 1.4 | −2.17 | 7.2 | −1.28 | 1.1 | −1.10 | 0.3 |
| 358 | 0.9 | −1.61 | 6.3 | −1.67 | 6.5 | −1.41 | 3.9 |
| 359 | 0.2 | −1.18 | 0.8 | −1.55 | 3.5 | −1.63 | 4.5 |
| 360 | 7.2 | 2.67 | 12.1 | 2.79 | 12 | 2.85 | 13.1 |
| 361 | 3.8 | 3.88 | 18 | 3.98 | 17.4 | 3.66 | 17.1 |
| 362 | 4.2 | −1.03 | 0.1 | −1.07 | 0.3 | 1.01 | 0 |
| 363 | 0.1 | 1.12 | 0.9 | 1.44 | 4.5 | 1.25 | 2.3 |
| 364 | 6.8 | −1.11 | 0.7 | −1.03 | 0.1 | 1.16 | 1.1 |
| 365 | 1.9 | 2.16 | 5.7 | 1.49 | 1.9 | 1.87 | 4.1 |
| 366 | 3.8 | 5.91 | 18.5 | 6.12 | 18 | 5.96 | 18.7 |
| 367 | 4.9 | −1.24 | 2.2 | −1.06 | 0.3 | −1.09 | 0.6 |
| 368 | 0.3 | −1.48 | 4.1 | −1.52 | 4.2 | −1.25 | 1.7 |
| 369 | 3 | 4.08 | 13.1 | 2.94 | 8.6 | 2.79 | 8.6 |
| 370 | 3.1 | 1.62 | 3.7 | 1.63 | 3.5 | 1.82 | 5.2 |
| 371 | 0.9 | 2.22 | 4.9 | 2.92 | 7.1 | 4.14 | 11.4 |
| 372 | 1.5 | 1.01 | 0 | −1.29 | 1.7 | −1.06 | 0.3 |
| 373 | 1.9 | 1.34 | 4.3 | 1.31 | 3.4 | 1.43 | 5.7 |
| 374 | 1.2 | 1.46 | 4.6 | 1.86 | 9.1 | 2.17 | 13.1 |
| 375 | 2 | −1.17 | 1.6 | −1.23 | 2.3 | −1.36 | 4.5 |
| 376 | 4.6 | 3.06 | 8.9 | 2.84 | 7.5 | 2.81 | 7.9 |
| 377 | 1.1 | 1.03 | 0.1 | −1.54 | 4 | −1.25 | 1.5 |
| 378 | 6.8 | −1.22 | 2.6 | −1.24 | 2.7 | −1.39 | 5.5 |
| 379 | 1.2 | 1.16 | 0.7 | 1.01 | 0 | 1.18 | 0.9 |
| 380 | 5.8 | 1.62 | 3.6 | 1.27 | 1.2 | 1.80 | 4.9 |
| 381 | 2.2 | 2.48 | 5.8 | 2.43 | 5.2 | 1.70 | 2.5 |
| 382 | 5 | −1.51 | 3.8 | −1.61 | 4.4 | −1.76 | 6.1 |
| 383 | 5.3 | 1.14 | 0.7 | −1.05 | 0.2 | −1.13 | 0.6 |
| 384 | 6.2 | 1.33 | 1.9 | −1.05 | 0.2 | −1.17 | 0.8 |
| 385 | 0.7 | 1.26 | 5 | 1.28 | 5.3 | 1.24 | 4.5 |
| 386 | 9.8 | 9.22 | 19 | 13.10 | 21.1 | 7.77 | 17.3 |
| 387 | 0.1 | 1.60 | 4.4 | 1.91 | 6.7 | 1.47 | 3.3 |
| 388 | 2 | 4.40 | 12.3 | 3.12 | 8 | 3.28 | 9.1 |
| 389 | 2.4 | 1.15 | 1 | 1.28 | 2.2 | 1.45 | 4.5 |
| 390 | 3.4 | 1.24 | 1.2 | 1.61 | 4 | 1.57 | 3.9 |
| 391 | 0.9 | −1.11 | 0.6 | −1.51 | 4.5 | −1.15 | 0.9 |
| 392 | 0 | 1.80 | 2.7 | 2.35 | 4.4 | 1.85 | 2.8 |
| 393 | 0.3 | 1.20 | 1.6 | 1.30 | 2.8 | 1.46 | 5.1 |
| 394 | 2.1 | 2.41 | 7.7 | 1.98 | 4.9 | 1.91 | 4.9 |
| 395 | 4.1 | −1.56 | 4.6 | −1.48 | 3.5 | −1.20 | 1.2 |
| 396 | 6.4 | 1.62 | 2.4 | 1.39 | 1.3 | −1.14 | 0.4 |
| 397 | 0.3 | 2.65 | 8.6 | 3.02 | 9.5 | 3.25 | 11.1 |
| 398 | 0.2 | 1.84 | 3.2 | 2.15 | 4.5 | 1.96 | 4 |
| 399 | 0.8 | −1.60 | 6.1 | −1.22 | 1.6 | −1.22 | 1.6 |
| 400 | 2.5 | 4.18 | 5.2 | 9.20 | 9.3 | 3.32 | 4 |
| 401 | 0.3 | −1.19 | 1.4 | −1.72 | 7.1 | −1.35 | 3 |
| 402 | 0.6 | 1.03 | 0.1 | 1.24 | 1 | −1.14 | 0.6 |
| 403 | 0.2 | −1.03 | 0.1 | −1.47 | 2.5 | −1.98 | 6.4 |
| 404 | 0.8 | 1.57 | 5 | 1.11 | 0.5 | 1.14 | 0.8 |
| 405 | 3.7 | 3.07 | 5.3 | 2.93 | 4.6 | 2.08 | 2.7 |
| 406 | 16.8 | 5.67 | 18.1 | 5.47 | 16.8 | 2.36 | 7.3 |
| 407 | 4.6 | 1.99 | 6.3 | 2.23 | 7.3 | 1.84 | 5.3 |
| 408 | 1.4 | 2.66 | 8.9 | 1.96 | 4.8 | 1.85 | 4.5 |
| 409 | 1.3 | −1.20 | 1.5 | −1.47 | 4.4 | −1.39 | 3.7 |
| 410 | 2.8 | 3.51 | 6.6 | 7.10 | 11.6 | 5.68 | 10.5 |
| 411 | 1.7 | 1.22 | 1.1 | 1.28 | 1.3 | 1.70 | 4.8 |
| 412 | 4.2 | 1.08 | 0.1 | −1.28 | 0.6 | −1.52 | 1.3 |
| 413 | 0.3 | 1.37 | 0.7 | −1.09 | 0.1 | 2.09 | 2.3 |
| 414 | 0.1 | 2.18 | 3.9 | 2.31 | 4 | 3.11 | 6.9 |
| 415 | 3.2 | 1.51 | 5.2 | 1.57 | 5.6 | 1.52 | 5.3 |
| 416 | 3 | 2.38 | 4.2 | 1.89 | 2.4 | 1.31 | 0.7 |
| 417 | 2.1 | −1.26 | 0.9 | −2.15 | 5.2 | −2.36 | 6.6 |
| 418 | 1.4 | 1.66 | 3.7 | 2.15 | 6.4 | 1.78 | 4.5 |
| 419 | 0.1 | 1.67 | 4.7 | 1.95 | 6.5 | 1.91 | 6.6 |
| 420 | 1.1 | 1.08 | 0.3 | 1.07 | 0.2 | −1.02 | 0.1 |
| 421 | 0.1 | 1.95 | 5 | 1.34 | 1.3 | 1.46 | 2.1 |
| 422 | 0 | −1.10 | 0.3 | −1.12 | 0.3 | 1.00 | 0 |
| 423 | 0.2 | 1.54 | 4.7 | 1.85 | 7.4 | 1.50 | 4.3 |
| 424 | 4.1 | −1.04 | 0.3 | −1.10 | 0.8 | 1.00 | 0 |
| 425 | 2.2 | −2.81 | 2.3 | −6.12 | 5.1 | −3.36 | 2.9 |
| 426 | 0.1 | −1.04 | 0.2 | −1.59 | 7.2 | −1.36 | 4.1 |
| 427 | 1.6 | 1.21 | 1 | 1.48 | 2.8 | 2.25 | 9.2 |
| 428 | 0 | −1.36 | 2.4 | −1.63 | 4.7 | −1.33 | 2.2 |
| 429 | 3 | −6.36 | 3.1 | −23.02 | 6.5 | −6.32 | 3.1 |
| 430 | 3.9 | 2.62 | 9.6 | 2.46 | 8.1 | 1.76 | 4.4 |
| 431 | 3.3 | 3.92 | 5.3 | 6.65 | 8.1 | 3.64 | 4.9 |
| 432 | 1.3 | 2.45 | 8 | 3.49 | 11.8 | 4.12 | 14.7 |
| 433 | 2.2 | 3.61 | 6.7 | 3.93 | 6.9 | 2.96 | 5.2 |
| 434 | 1.2 | 1.72 | 4.6 | 2.40 | 8.7 | 2.71 | 11.2 |
| 435 | 2.9 | −2.02 | 5.1 | −1.69 | 3 | −2.01 | 5 |
| 436 | 1.5 | −1.76 | 2.7 | −2.64 | 5.8 | −2.61 | 6.2 |
| 437 | 1.2 | −3.59 | 6.8 | −3.79 | 6.8 | −5.74 | 10.7 |
| 438 | 5.3 | 1.16 | 0.5 | −1.26 | 0.9 | −1.40 | 1.7 |
| 439 | 5.1 | −1.22 | 0.4 | −1.22 | 0.3 | −1.46 | 0.8 |
| 440 | 1 | 2.62 | 4.6 | 1.81 | 2 | 2.10 | 3.1 |
| 441 | 6.3 | 18.09 | 13.7 | 15.00 | 11.8 | 8.66 | 9.3 |
| 442 | 2.6 | 5.04 | 7.2 | 5.93 | 7.7 | 3.26 | 4.4 |
| 443 | 0.6 | 2.18 | 5.1 | 1.47 | 1.6 | 1.00 | 0 |
| 444 | 0.3 | −2.52 | 3.5 | −4.09 | 6.3 | −2.94 | 4.5 |
| 445 | 0 | −1.32 | 1.1 | −2.28 | 5.1 | −1.92 | 3.9 |
| 446 | 6.1 | 1.63 | 8.5 | 1.42 | 4.8 | 1.37 | 4.4 |
| 447 | 5.1 | 2.15 | 2 | 1.72 | 1.1 | −1.69 | 1.1 |
| 448 | 5.6 | −2.87 | 2 | −3.15 | 2.1 | −1.97 | 1 |
| 449 | 3.2 | 6.94 | 9.8 | 7.74 | 9.9 | 8.93 | 11.6 |
| 450 | 4.1 | 1.31 | 1.7 | 1.24 | 1.1 | 1.24 | 1.2 |
| 451 | 14.8 | 5.60 | 16.9 | 4.63 | 13.9 | 4.65 | 14.7 |
| 452 | 2.4 | 3.71 | 13.6 | 3.38 | 11.6 | 3.03 | 10.9 |
| 453 | 4.1 | −1.12 | 0.4 | −1.58 | 2.5 | −1.25 | 0.9 |
| 454 | 7 | 17.15 | 20.3 | 19.71 | 20.3 | 9.81 | 15.9 |
| 455 | 0.5 | −1.61 | 1.2 | −2.78 | 3.8 | −3.02 | 4.6 |
| 456 | 5.8 | −1.26 | 3.6 | −1.10 | 0.9 | −1.16 | 1.7 |
| 457 | 1.2 | 5.60 | 4.4 | 5.22 | 3.8 | 3.54 | 2.7 |
| 458 | 2.2 | −1.35 | 2.3 | −1.51 | 3.5 | −1.60 | 4.6 |
| 459 | 0.6 | 3.46 | 5.1 | 1.93 | 1.8 | 1.93 | 1.9 |
| 460 | 1.9 | 3.85 | 9.6 | 4.14 | 9.6 | 4.92 | 12 |
| 461 | 0 | −1.35 | 2.3 | −1.43 | 2.8 | −1.67 | 5.2 |
| 462 | 0.4 | 1.53 | 2.5 | 1.59 | 2.6 | 1.78 | 4 |
| 463 | 1.8 | −1.59 | 1.6 | −2.26 | 3.6 | −2.28 | 3.9 |
| 464 | 0.5 | 2.93 | 10 | 2.41 | 7 | 1.58 | 2.6 |
| 465 | 4 | 5.72 | 11.8 | 3.73 | 7.5 | 2.85 | 5.7 |
| 466 | 3.2 | 1.24 | 0.4 | 2.06 | 1.9 | 1.15 | 0.2 |
| 467 | 3.9 | 1.86 | 7.4 | 1.69 | 5.4 | 2.04 | 9 |
| 468 | 7.8 | 1.13 | 0.5 | 1.17 | 0.7 | 1.23 | 1.1 |
| 469 | 1.9 | 5.17 | 7.7 | 5.80 | 8 | 4.56 | 6.9 |
| 470 | 2 | 1.63 | 3.9 | 1.73 | 4.4 | 1.44 | 2.5 |
| 471 | 3.7 | −2.69 | 8 | −1.66 | 2.5 | −1.52 | 2.1 |
| 472 | 4.4 | −1.17 | 0.4 | −1.91 | 3.2 | −1.25 | 0.7 |
| 473 | 0.3 | −1.06 | 0.2 | −1.84 | 4.1 | −1.94 | 5 |
| 474 | 0.1 | −1.69 | 6.3 | −1.64 | 5.3 | −1.34 | 2.5 |
| 475 | 3.1 | 9.94 | 8.5 | 7.09 | 6.3 | 8.76 | 7.9 |
| 476 | 0.9 | 1.00 | 0 | −1.07 | 0.7 | −1.11 | 1.3 |
| 477 | 1.4 | −1.22 | 3.7 | −1.43 | 8.4 | −1.30 | 5.8 |
| 478 | 0.2 | −1.18 | 0.6 | −1.36 | 1.4 | −1.96 | 5 |
| 479 | 0.5 | −1.05 | 0.3 | −1.43 | 4.6 | −1.05 | 0.3 |
| 480 | 4.8 | −1.61 | 7.5 | −1.73 | 8.6 | −1.37 | 4 |
| 481 | 0.7 | −1.23 | 2.2 | −1.40 | 4.1 | −1.08 | 0.5 |
| 482 | 0.9 | 3.22 | 5.4 | 3.80 | 6.2 | 3.02 | 5 |
| 483 | 4.5 | 5.84 | 7.2 | 3.14 | 3.4 | 3.11 | 3.7 |
| 484 | 0.4 | 1.84 | 5.1 | 1.56 | 2.9 | 1.45 | 2.3 |
| 485 | 4.2 | −1.02 | 0.1 | −1.47 | 2.4 | −1.45 | 2.5 |
| 486 | 7.2 | 1.48 | 3.5 | 1.48 | 3.3 | 1.26 | 1.6 |
| 487 | 2.4 | 1.41 | 2.7 | 1.56 | 3.8 | 1.93 | 7.4 |
| 488 | 1.5 | 4.82 | 6 | 4.58 | 5.3 | 7.04 | 8.3 |
| 489 | 0.5 | −1.06 | 0.4 | −1.31 | 4 | −1.22 | 2.7 |
| 490 | 9.2 | 1.26 | 1.3 | −1.11 | 0.4 | −1.58 | 3.8 |
| 491 | 3.2 | 1.18 | 1.2 | −1.02 | 0.1 | 1.21 | 1.4 |
| 492 | 0.5 | −1.62 | 4.1 | −1.56 | 3.3 | −1.34 | 1.9 |
| 493 | 1.6 | −1.31 | 2.7 | −1.40 | 3.5 | −1.61 | 6.5 |
| 494 | 2.8 | 1.09 | 0.5 | 1.04 | 0.2 | −1.12 | 0.8 |
| 495 | 0.4 | 1.40 | 2.3 | 1.75 | 4.7 | 1.45 | 2.6 |
| 496 | 0.6 | −1.21 | 0.1 | −18.25 | 4.7 | −29.79 | 6.4 |
| 497 | 5.9 | −1.01 | 0 | −1.18 | 1.1 | −1.09 | 0.5 |
| 498 | 4 | −1.36 | 3.2 | −1.42 | 3.6 | −1.60 | 6.1 |
| 499 | 0.1 | 1.85 | 4.2 | 1.41 | 1.6 | 1.98 | 4.9 |
| 500 | 1.5 | 3.01 | 4 | 3.47 | 4.5 | 2.91 | 3.8 |
| 501 | 2.5 | 7.91 | 10.3 | 5.85 | 7.7 | 5.13 | 7.4 |
| 502 | 8.8 | 4.16 | 20.7 | 3.30 | 16.1 | 2.45 | 12 |
| 503 | 3.4 | −1.79 | 7.1 | −1.61 | 4.8 | −1.69 | 6 |
| 504 | 1.7 | 5.41 | 7.5 | 10.36 | 11.2 | 8.33 | 10.4 |
| 505 | 6.9 | 1.13 | 0.7 | 1.13 | 0.6 | 1.15 | 0.9 |
| 506 | 3.1 | 1.58 | 2.6 | −1.01 | 0 | 1.43 | 1.8 |
| 507 | 0.6 | 6.58 | 4.5 | 7.76 | 4.7 | 1.97 | 0.9 |
| 508 | 3.8 | −1.66 | 3.1 | −1.56 | 2.3 | −1.64 | 2.7 |
| 509 | 0.7 | −1.39 | 2.4 | −1.54 | 3.4 | −1.74 | 5.4 |
| 510 | 5.7 | 7.04 | 12.3 | 9.76 | 14.1 | 10.87 | 15.8 |
| 511 | 4.9 | 1.29 | 1.1 | 1.32 | 1.2 | 1.09 | 0.3 |
| 512 | 0.1 | 1.43 | 1.6 | 1.49 | 1.8 | 2.09 | 5 |
| 513 | 1.6 | 1.39 | 1 | 1.34 | 0.8 | 1.35 | 0.9 |
| 514 | 5.7 | −1.16 | 1.2 | −1.10 | 0.7 | −1.23 | 2 |
| 515 | 4.9 | 2.01 | 3.3 | 1.46 | 1.2 | 1.04 | 0.1 |
| 516 | 0.1 | 1.03 | 0 | 1.24 | 0.4 | 1.06 | 0.1 |
| 517 | 10.6 | 5.07 | 21.2 | 5.80 | 21.9 | 7.64 | 26.4 |
| 518 | 2.1 | −1.49 | 2.5 | −2.13 | 6.2 | −2.10 | 6.4 |
| 519 | 0.4 | −1.33 | 1.6 | −2.11 | 6.6 | −1.30 | 1.4 |
| 520 | 0 | 1.71 | 5.9 | 1.60 | 4.5 | 1.14 | 0.7 |
| 521 | 0.6 | 2.30 | 3.2 | 2.10 | 2.5 | 2.78 | 4.4 |
| 522 | 0.8 | 2.29 | 2.5 | 4.34 | 5.7 | 4.97 | 6.9 |
| 523 | 4 | 6.20 | 9.6 | 6.09 | 8.9 | 4.17 | 6.8 |
| 524 | 6.7 | −1.08 | 0.1 | −3.13 | 4.5 | −3.39 | 5.3 |
| 525 | 3.2 | 24.42 | 6.6 | 8.82 | 3.4 | 2.25 | 0.8 |
| 526 | 3.2 | −2.02 | 4.3 | −2.14 | 4.5 | −2.53 | 6.5 |
| 527 | 1.1 | 1.89 | 5 | 1.49 | 2.2 | 1.43 | 2.1 |
| 528 | 4.6 | 1.61 | 6.9 | 1.40 | 3.7 | 1.29 | 2.7 |
| 529 | 2.3 | 1.94 | 1.4 | 2.10 | 1.6 | 2.50 | 2.3 |
| 530 | 0.9 | 2.41 | 8.3 | 3.11 | 11.1 | 2.87 | 10.7 |
| 531 | 5.7 | 2.24 | 8.1 | 2.03 | 6.2 | 2.41 | 9.2 |
| 532 | 6.3 | 1.57 | 3.3 | 1.46 | 2.4 | 1.41 | 2.2 |
| 533 | 7.4 | 7.15 | 21.6 | 6.13 | 18.9 | 5.76 | 19.1 |
| 534 | 0.4 | 1.32 | 5.1 | 1.28 | 4 | 1.42 | 7.2 |
| 535 | 0.8 | 1.17 | 2.3 | 1.14 | 1.7 | 1.25 | 4.1 |
| 536 | 6.2 | 23.99 | 17.4 | 30.83 | 18 | 18.90 | 15.9 |
| 537 | 6.4 | 1.08 | 0.5 | −1.14 | 1 | −1.35 | 3.9 |
| 538 | 5.7 | −1.01 | 0 | −1.22 | 0.8 | −1.12 | 0.4 |
| 539 | 3.4 | −1.13 | 0.4 | −1.13 | 0.4 | −1.01 | 0 |
| 540 | 1.4 | 10.50 | 10.6 | 7.29 | 7.8 | 6.65 | 7.8 |
| 541 | 0.1 | −1.42 | 2.2 | −1.84 | 4.8 | −1.58 | 3.3 |
| 542 | 1.3 | 1.37 | 1.6 | 1.74 | 3.5 | 1.80 | 4.1 |
| 543 | 1.4 | −1.75 | 9.4 | −1.49 | 5.3 | −1.48 | 5.6 |
| 544 | 1.2 | 1.63 | 3.9 | 2.08 | 6.7 | 2.26 | 8.4 |
| 545 | 1.6 | 1.96 | 12.3 | 2.26 | 14.8 | 1.86 | 11.1 |
| 546 | 2.4 | 1.22 | 0.6 | −1.04 | 0.1 | −1.48 | 1.5 |
| 547 | 0.4 | 1.75 | 3.4 | 1.58 | 2.3 | 1.91 | 4.3 |
| 548 | 8.1 | 1.28 | 2.3 | 1.03 | 0.1 | 1.15 | 1 |
| 549 | 7.3 | 13.29 | 8 | 10.19 | 6.4 | 4.63 | 3.6 |
| 550 | 1 | −2.52 | 5.9 | −5.84 | 13.6 | −9.73 | 18.4 |
| 551 | 4.2 | 1.76 | 5.5 | 1.08 | 0.3 | 1.42 | 2.6 |
| 552 | 1.7 | 2.36 | 3.1 | 2.51 | 3.2 | 3.46 | 5.5 |
| 553 | 3.3 | 8.78 | 8.8 | 5.79 | 6 | 5.69 | 6.3 |
| 554 | 0.1 | −1.38 | 1.2 | −2.07 | 4 | −1.87 | 3.4 |
| 555 | 0.3 | −1.86 | 5.1 | −1.49 | 2.4 | −1.57 | 3.1 |
| 556 | 5.3 | 7.45 | 5.4 | 3.48 | 2.4 | 1.16 | 0.2 |
| 557 | 6.2 | 2.29 | 10.6 | 1.67 | 4.9 | 1.43 | 3 |
| 558 | 5.2 | 7.34 | 7.7 | 7.50 | 7.3 | 4.03 | 4.5 |
| 559 | 1.6 | 2.89 | 5.5 | 2.24 | 3.4 | 3.32 | 6.6 |
| 560 | 15.1 | 7.25 | 26.3 | 10.14 | 29 | 7.30 | 26.4 |
| 561 | 0.4 | 1.32 | 1.6 | 1.44 | 2.3 | 1.94 | 6.2 |
| 562 | 14.6 | 4.42 | 17.2 | 4.51 | 16.6 | 2.56 | 9.6 |
| 563 | 2.5 | 1.17 | 0.5 | 1.14 | 0.4 | 1.57 | 2.1 |
| 564 | 8.8 | −1.02 | 0.1 | −1.08 | 0.2 | 1.09 | 0.3 |
| 565 | 2.6 | −1.31 | 1.9 | −1.66 | 4.8 | −1.58 | 4.4 |
| 566 | 0.1 | −1.12 | 0.2 | −1.60 | 1 | −2.56 | 2.9 |
| 567 | 1.1 | 2.78 | 2 | 5.47 | 4.1 | 4.36 | 3.5 |
| 568 | 8.7 | 1.75 | 6.5 | 1.52 | 3.9 | 1.37 | 2.7 |
| 569 | 0.3 | 1.87 | 4.8 | 1.67 | 3.3 | 1.47 | 2.3 |
| 570 | 0.3 | 1.26 | 1.5 | 1.05 | 0.2 | 1.22 | 1.2 |
| 571 | 5.5 | 8.83 | 12.4 | 11.20 | 13.3 | 6.15 | 9.7 |
| 572 | 0.5 | 1.89 | 5.1 | 1.39 | 1.7 | 1.50 | 2.5 |
| 573 | 0.3 | 1.53 | 3.1 | 1.39 | 1.9 | 1.19 | 0.8 |
| 574 | 8.6 | 1.52 | 0.6 | −2.48 | 1.9 | −3.64 | 3.5 |
| 575 | 11.4 | 7.13 | 15.2 | 8.19 | 15.6 | 4.55 | 10.9 |
| 576 | 2.6 | 2.40 | 8.2 | 2.16 | 6.3 | 1.96 | 5.5 |
| 577 | 0.5 | 1.52 | 2.6 | 1.57 | 2.7 | 1.86 | 4.5 |
| 578 | 3.7 | 1.92 | 6.5 | 1.80 | 5.2 | 1.41 | 2.4 |
| 579 | 3.2 | 1.14 | 0.8 | 1.34 | 2.4 | 1.49 | 4.1 |
| 580 | 1.8 | 1.87 | 3.3 | 1.37 | 1.1 | 2.09 | 4.2 |
| 581 | 4.9 | 1.29 | 0.5 | −1.50 | 0.8 | −2.71 | 3.3 |
| 582 | 4.3 | 1.40 | 4.2 | 1.27 | 2.3 | 1.17 | 1.4 |
| 583 | 10.5 | 1.52 | 2.4 | 1.27 | 1 | 1.20 | 0.7 |
| 584 | 0 | 1.82 | 13.4 | 1.75 | 11.6 | 1.88 | 14.3 |
| 585 | 4.6 | 1.09 | 0.6 | 1.24 | 2.4 | 1.25 | 2.6 |
| 586 | 0.3 | 1.33 | 3.2 | 1.50 | 5.1 | 1.23 | 2 |
| 587 | 5.1 | −1.28 | 1.6 | −1.36 | 2 | −1.26 | 1.5 |
| 588 | 0.5 | −1.47 | 4.2 | −2.00 | 9.4 | −2.10 | 11 |
| 589 | 8.3 | −1.39 | 2.1 | −1.54 | 3 | −1.66 | 4 |
| 590 | 4.7 | 1.25 | 1.5 | 1.30 | 1.7 | 1.30 | 1.8 |
| 591 | 0.8 | 1.59 | 2.5 | 2.37 | 6 | 1.77 | 3.4 |
| 592 | 4.4 | 1.72 | 1 | −2.64 | 2.3 | −3.68 | 4 |
| 593 | 0.1 | −2.21 | 6.9 | −2.09 | 5.7 | −1.54 | 2.7 |
| 594 | 0.8 | 1.49 | 4.2 | 1.14 | 0.8 | 1.14 | 0.8 |
| 595 | 0 | 1.10 | 1.5 | −1.01 | 0.1 | −1.00 | 0 |
| 596 | 0 | 1.14 | 1.6 | −1.03 | 0.2 | −1.05 | 0.4 |
| 597 | 1.8 | 2.79 | 5.1 | 2.92 | 5.1 | 1.99 | 2.8 |
| 598 | 1.1 | −1.89 | 5.5 | −1.97 | 5.7 | −1.50 | 2.7 |
| 599 | 5.8 | −1.03 | 0.1 | 1.77 | 6 | 1.86 | 7.2 |
| 600 | 5.3 | 1.80 | 6.6 | 1.73 | 5.5 | 1.31 | 2 |
| 601 | 5.7 | −3.69 | 8.5 | −1.69 | 2 | −2.31 | 4.3 |
| 602 | 4.1 | 1.28 | 1.2 | 1.15 | 0.5 | −1.05 | 0.2 |
| 603 | 5.8 | 5.35 | 6.3 | 1.61 | 0.9 | 1.18 | 0.2 |
| 604 | 1 | −2.04 | 2.6 | −2.66 | 4 | −2.63 | 4.2 |
| 605 | 0 | 2.63 | 6.8 | 2.17 | 4.5 | 1.56 | 2 |
| 606 | 1.3 | −1.20 | 1 | −1.65 | 4.4 | −1.44 | 2.8 |
| 607 | 0.2 | −1.26 | 0.6 | −1.04 | 0.1 | 1.12 | 0.2 |
| 608 | 2.4 | −2.12 | 7.5 | −1.95 | 5.9 | −1.77 | 5 |
| 609 | 0.9 | 1.54 | 2 | 1.88 | 3.4 | 1.98 | 4.2 |
| 610 | 0.6 | 1.13 | 1.2 | 1.28 | 3.3 | 1.36 | 5.1 |
| 611 | 1 | 1.33 | 3.2 | 1.11 | 0.7 | 1.14 | 1 |
| 612 | 6.4 | 3.06 | 14.9 | 3.67 | 16.9 | 2.37 | 10.7 |
| 613 | 1.2 | 1.52 | 10.6 | 1.51 | 9.5 | 1.27 | 4.6 |
| 614 | 0.7 | 1.19 | 1.2 | 1.11 | 0.6 | 1.06 | 0.3 |
| 615 | 1.2 | 2.37 | 8.1 | 2.19 | 6.6 | 1.53 | 2.7 |
| 616 | 5.3 | 1.08 | 0.6 | 1.14 | 1.3 | 1.14 | 1.5 |
| 617 | 8.1 | −1.16 | 1.1 | 1.70 | 6.7 | 1.89 | 9.4 |
| 618 | 1.7 | −1.30 | 4.1 | −1.27 | 3.3 | −1.18 | 2 |
| 619 | 4.1 | −1.14 | 1 | −1.13 | 0.8 | −1.43 | 4.4 |
| 620 | 0.6 | −1.36 | 4.6 | −1.44 | 5.6 | −1.82 | 12.2 |
| 621 | 11.6 | 2.91 | 14.1 | 3.72 | 17.1 | 3.27 | 16 |
| 622 | 1.6 | −1.30 | 2.6 | −1.37 | 3.2 | −1.49 | 4.9 |
| 623 | 0.3 | 1.11 | 0.8 | 1.18 | 1.5 | 1.19 | 1.7 |
| 624 | 5.9 | −1.14 | 1.1 | −1.01 | 0 | −1.07 | 0.4 |
| 625 | 2.8 | 1.27 | 4.2 | 1.12 | 1.2 | 1.05 | 0.4 |
| 626 | 0 | 1.14 | 1.8 | 1.34 | 5.8 | 1.25 | 4.2 |
| 627 | 5.3 | 1.06 | 0.1 | −1.48 | 1.3 | −2.95 | 6.4 |
| 628 | 8.6 | 3.13 | 4.8 | 3.64 | 5.4 | 2.27 | 2.9 |
| 629 | 1.7 | 2.01 | 6.7 | 1.66 | 3.7 | 1.93 | 6.1 |
| 630 | 0.8 | 1.38 | 2.1 | 1.33 | 1.6 | 1.33 | 1.7 |
| 631 | 2.6 | 1.80 | 6.1 | 1.70 | 4.8 | 2.01 | 7.8 |
| 632 | 5.7 | −1.21 | 1.4 | −1.21 | 1.4 | −1.11 | 0.6 |
| 633 | 1.4 | −1.42 | 4.5 | −1.37 | 3.5 | −1.19 | 1.5 |
| 634 | 13.2 | 7.98 | 29.8 | 8.93 | 29.9 | 6.14 | 26.5 |
| 635 | 1.4 | 1.26 | 2.3 | 1.50 | 5 | 1.77 | 8.8 |
| 636 | 1.7 | −1.30 | 2.3 | −1.60 | 5.2 | −1.55 | 5.1 |
| 637 | 2 | 1.55 | 11.2 | 1.79 | 15 | 1.93 | 18.3 |
| 638 | 0 | −1.35 | 4.6 | −1.31 | 3.5 | −1.38 | 5.1 |
| 639 | 1.7 | 2.03 | 7.6 | 1.38 | 2.1 | 1.42 | 2.6 |
| 640 | 2.6 | 4.57 | 11.9 | 3.25 | 7.8 | 1.95 | 3.6 |
| 641 | 2.6 | 1.31 | 1.4 | 1.24 | 0.9 | 1.02 | 0 |
| 642 | 4.2 | 1.42 | 1.8 | 1.29 | 1.1 | 1.44 | 1.9 |
| 643 | 3.7 | 3.10 | 11 | 2.21 | 6.2 | 2.53 | 8.3 |
| 644 | 0.9 | −1.26 | 2 | −1.67 | 6.4 | −1.41 | 3.8 |
| 645 | 4 | 1.12 | 0.4 | 1.36 | 1.7 | 1.30 | 1.4 |
| 646 | 1.6 | 1.69 | 5.2 | 1.35 | 2 | 1.40 | 2.6 |
| 647 | 5.8 | 1.88 | 2 | 2.40 | 3 | 2.13 | 2.6 |
| 648 | 4.1 | −1.22 | 1.6 | 1.26 | 2 | 1.14 | 0.9 |
| 649 | 1.9 | 1.42 | 5.9 | 1.74 | 10.6 | 1.68 | 10.3 |
| 650 | 7.4 | 9.17 | 13.6 | 9.19 | 12.8 | 5.91 | 10.2 |
| 651 | 4.4 | 5.33 | 10 | 5.19 | 9.1 | 6.31 | 11.4 |
| 652 | 1.1 | −1.43 | 3.6 | −1.60 | 5.2 | −1.40 | 3.4 |
| 653 | 0.1 | −1.08 | 0.4 | −1.59 | 4.6 | −1.38 | 2.8 |
| 654 | 2.4 | −1.74 | 6.8 | −1.47 | 3.6 | −1.38 | 2.9 |
| 655 | 3.9 | 1.10 | 0.5 | −1.02 | 0.1 | 1.07 | 0.3 |
| 656 | 5.8 | 6.64 | 3.1 | 2.64 | 1 | 1.57 | 0.4 |
| 657 | 4.1 | 2.87 | 11.2 | 3.16 | 11.8 | 2.96 | 11.7 |
| 658 | 0.9 | −1.25 | 2.6 | −1.31 | 3.2 | −1.18 | 1.6 |
| 659 | 0.2 | 1.26 | 3 | 1.35 | 4.2 | 1.37 | 4.9 |
| 660 | 7 | 2.08 | 4.3 | 1.78 | 2.8 | 1.93 | 3.6 |
| 661 | 3.3 | 1.51 | 5 | 1.51 | 4.5 | 1.57 | 5.6 |
| 662 | 0.1 | 1.84 | 12.9 | 2.08 | 15.3 | 1.96 | 14.6 |
| 663 | 13.2 | 1.76 | 8.7 | 1.60 | 6.2 | 1.64 | 7.2 |
| 664 | 4.4 | 1.29 | 2.1 | 1.44 | 3.3 | 1.23 | 1.5 |
| 665 | 1.9 | 8.96 | 15.5 | 9.61 | 15.2 | 6.52 | 12.7 |
| 666 | 7 | 9.56 | 11.5 | 12.48 | 12.5 | 8.20 | 10.4 |
| 667 | 3.6 | 2.03 | 9.6 | 2.47 | 12.5 | 2.20 | 11.2 |
| 668 | 11.3 | 9.71 | 21.1 | 14.98 | 23.9 | 8.02 | 19.2 |
| 669 | 14.8 | 4.90 | 22.5 | 4.64 | 20.6 | 2.50 | 11.8 |
| 670 | 8 | 2.09 | 17 | 2.31 | 18.6 | 2.43 | 20.8 |
| 671 | 3.9 | −1.54 | 1.1 | −2.33 | 2.9 | −3.05 | 4.8 |
| 672 | 10.6 | −1.47 | 1 | −2.97 | 4.5 | −7.79 | 12.1 |
| 673 | 1.5 | −1.05 | 0.2 | −1.12 | 0.6 | −1.26 | 1.7 |
| 674 | 4.8 | 1.27 | 4.7 | 1.14 | 1.6 | 1.10 | 1.1 |
| 675 | 0.8 | −1.16 | 1.1 | −1.53 | 5 | −1.05 | 0.2 |
| 676 | 9.3 | 1.95 | 5.4 | 1.50 | 2.3 | 1.97 | 5.6 |
| 677 | 4.6 | 1.21 | 0.6 | −1.25 | 0.7 | −1.12 | 0.3 |
| 678 | 0.3 | 1.13 | 0.4 | 1.28 | 0.9 | 3.08 | 9.4 |
| 679 | 0.1 | −1.37 | 2.5 | −1.70 | 5.2 | −1.33 | 2.1 |
| 680 | 6.5 | 2.10 | 8.1 | 1.71 | 4.7 | 2.12 | 8.3 |
| 681 | 6.2 | 2.30 | 17.3 | 2.09 | 14.2 | 2.32 | 17.5 |
| 682 | 3.6 | 1.98 | 2.2 | 1.19 | 0.3 | 1.10 | 0.2 |
| 683 | 4.5 | 2.56 | 2.7 | 1.94 | 1.5 | 2.33 | 2.3 |
| 684 | 4.2 | 1.13 | 0.2 | −1.03 | 0 | −1.09 | 0.1 |
| 685 | 2.4 | −2.20 | 7.4 | −1.12 | 0.4 | −1.13 | 0.5 |
| 686 | 1.1 | −1.27 | 2.4 | −1.38 | 3.6 | −1.54 | 6.1 |
| 687 | 1.9 | 1.92 | 5.8 | 1.83 | 4.9 | 2.36 | 8.8 |
| 688 | 1.2 | 2.10 | 9.6 | 2.71 | 13.3 | 1.94 | 8.2 |
| 689 | 4.2 | 26.57 | 9.9 | 12.65 | 6.4 | 11.38 | 6.4 |
| 690 | 0.3 | 1.48 | 1.7 | 1.43 | 1.4 | 1.48 | 1.7 |
| 691 | 1.8 | 6.92 | 8 | 3.96 | 4.4 | 2.24 | 2.1 |
| 692 | 0 | 1.86 | 4.6 | 1.70 | 3.3 | 1.84 | 4.5 |
| 693 | 1.5 | −2.35 | 3.9 | −2.80 | 4.9 | −1.68 | 1.8 |
| 694 | 3.5 | −1.26 | 1.1 | −1.93 | 4.9 | −1.55 | 2.8 |
| 695 | 4.2 | −1.37 | 4.1 | −1.43 | 4.7 | −1.20 | 1.8 |
| 696 | 1.3 | −1.52 | 8.2 | −1.46 | 6.5 | −1.10 | 0.9 |
| 697 | 5.7 | −1.20 | 1 | −1.09 | 0.3 | 1.39 | 2.6 |
| 698 | 0.2 | −1.02 | 0 | −1.16 | 0.3 | −1.25 | 0.6 |
| 699 | 5.2 | 3.67 | 4.3 | 2.93 | 3 | 2.50 | 2.5 |
| 700 | 0.2 | 2.97 | 12 | 2.49 | 8.9 | 2.23 | 7.9 |
| 701 | 1.4 | 1.05 | 0.2 | 1.03 | 0.1 | 1.07 | 0.2 |
| 702 | 11.2 | 2.65 | 4.2 | 1.67 | 1.5 | 1.38 | 0.8 |
| 703 | 3.3 | −1.42 | 3.7 | −1.73 | 6.7 | −1.84 | 8.3 |
| 704 | 1.1 | 1.35 | 2.3 | 1.18 | 0.9 | 1.71 | 5.8 |
| 705 | 4.5 | −2.07 | 7.9 | −1.85 | 5.8 | −1.95 | 7.1 |
| 706 | 0.6 | 1.40 | 4.3 | 1.39 | 3.9 | 1.54 | 6.4 |
| 707 | 3.5 | 1.18 | 0.6 | 1.01 | 0 | 1.26 | 1 |
| 708 | 2.9 | 5.20 | 7.9 | 5.76 | 8 | 2.29 | 2.8 |
| 709 | 0.3 | 11.19 | 5.9 | 8.87 | 4.7 | 6.15 | 3.8 |
| 710 | 10.6 | 2.42 | 4.6 | 1.56 | 1.5 | 1.34 | 0.9 |
| 711 | 8 | −12.24 | 10.8 | −6.60 | 6.7 | −6.88 | 7.5 |
| 712 | 7 | 1.05 | 0.2 | 1.12 | 0.7 | 1.22 | 1.7 |
| 713 | 3.5 | 2.72 | 9.1 | 2.96 | 9.5 | 2.38 | 7.4 |
| 714 | 1.1 | −1.29 | 2.7 | −1.15 | 1 | −1.44 | 4.7 |
| 715 | 0.2 | −1.20 | 2 | −1.38 | 4.5 | −1.30 | 3.5 |
| 716 | 4.1 | −1.11 | 0.2 | 1.06 | 0.1 | −1.02 | 0 |
| 717 | 1 | 2.00 | 4.5 | 2.11 | 4.7 | 1.79 | 3.4 |
| 718 | 2.3 | 2.63 | 5.9 | 1.89 | 2.9 | 2.35 | 4.9 |
| 719 | 4.3 | 2.31 | 4.5 | 2.62 | 5.2 | 1.83 | 2.7 |
| 720 | 2.1 | 2.45 | 9.9 | 3.14 | 12.9 | 2.32 | 9.1 |
| 721 | 3.9 | −6.07 | 3.3 | −11.32 | 4.9 | −3.14 | 1.6 |
| 722 | 0.2 | −1.04 | 0.1 | 1.16 | 0.5 | 2.04 | 4.7 |
| 723 | 0.6 | 1.70 | 6.6 | 1.54 | 4.6 | 1.40 | 3.3 |
| 724 | 4.6 | 3.33 | 5.2 | 3.15 | 4.5 | 2.72 | 3.9 |
| 725 | 0.1 | 1.71 | 3.4 | 1.55 | 2.3 | 2.22 | 6.4 |
| 726 | 2.5 | 1.58 | 1.3 | 1.33 | 0.6 | 1.10 | 0.2 |
| 727 | 0.1 | −1.93 | 4.3 | −1.80 | 3.3 | −1.93 | 4.3 |
| 728 | 8.8 | 1.00 | 0 | −1.37 | 0.7 | −1.38 | 0.7 |
| 729 | 2.8 | 1.74 | 3 | 1.63 | 2.3 | 2.40 | 6 |
| 730 | 4.9 | 4.69 | 4 | 2.60 | 1.8 | 4.03 | 3.4 |
| 731 | 0.1 | −1.49 | 3 | −1.82 | 5.2 | −1.79 | 5.3 |
| 732 | 1.7 | −1.11 | 0.8 | −1.41 | 4.7 | −1.21 | 2 |
| 733 | 1.1 | −1.22 | 2.4 | −1.38 | 4.9 | −1.32 | 4.1 |
| 734 | 1.7 | 1.03 | 0.2 | 1.05 | 0.3 | −1.01 | 0 |
| 735 | 0 | −1.15 | 0.7 | −1.11 | 0.4 | −1.62 | 4 |
| 736 | 0.2 | −2.02 | 1.6 | −3.77 | 4 | −1.59 | 0.9 |
| 737 | 1.4 | 3.09 | 12.5 | 4.03 | 15.3 | 3.43 | 13.9 |
| 738 | 7 | 2.15 | 3.1 | 2.08 | 2.7 | 1.76 | 2 |
| 739 | 5.5 | −3.96 | 2.4 | −4.94 | 2.8 | −1.80 | 0.7 |
| 740 | 0.6 | −1.97 | 8.1 | −2.44 | 11.1 | −4.03 | 19.1 |
| 741 | 0.1 | 1.35 | 1.6 | 2.27 | 7.1 | 2.89 | 10.8 |
| 742 | 5 | 2.79 | 4.3 | 2.34 | 3 | 1.49 | 1 |
| 743 | 2.4 | −1.21 | 2.7 | −1.38 | 5.7 | −1.36 | 5.7 |
| 744 | 0.5 | −1.34 | 4.5 | −1.29 | 3.4 | −1.45 | 6.5 |
| 745 | 4.6 | 1.66 | 1.9 | −1.04 | 0.1 | 1.04 | 0.1 |
| 746 | 16.6 | −2.02 | 9.6 | −1.78 | 6.8 | −1.09 | 0.4 |
| 747 | 16.6 | −2.02 | 9.6 | −1.78 | 6.8 | −1.09 | 0.4 |
| 748 | 10.1 | 21.58 | 19.8 | 24.29 | 19.6 | 17.93 | 18.6 |
| 749 | 1.1 | −1.05 | 0.2 | −1.14 | 0.5 | 1.56 | 3.5 |
| 750 | 12.5 | 5.28 | 19.2 | 6.51 | 20.7 | 3.90 | 15.2 |
| 751 | 2.2 | −1.45 | 1.5 | −2.00 | 3.8 | −3.39 | 9.4 |
| 752 | 8.8 | 15.58 | 11.8 | 6.23 | 6.2 | 12.60 | 10.6 |
| 753 | 4.4 | −1.39 | 3.9 | −1.47 | 4.7 | −1.52 | 5.8 |
| 754 | 4.7 | 4.97 | 11.9 | 5.56 | 12.2 | 2.89 | 6.7 |
| 755 | 6 | 1.71 | 2 | 1.40 | 0.9 | 1.54 | 1.4 |
| 756 | 1 | 1.87 | 4.1 | 2.05 | 4.7 | 2.15 | 5.6 |
| 757 | 0.9 | 1.73 | 4.2 | 1.77 | 4.4 | 2.01 | 6.4 |
| 758 | 7.6 | 1.70 | 2.9 | 1.67 | 2.5 | 1.57 | 2.2 |
| 759 | 9 | 31.73 | 16.7 | 35.81 | 16.5 | 15.91 | 12.7 |
| 760 | 8.7 | 2.73 | 16.7 | 2.62 | 15 | 1.75 | 7.7 |
| 761 | 1.1 | 2.94 | 13.6 | 4.18 | 17.9 | 2.66 | 12 |
| 762 | 11.3 | 1.91 | 5.6 | 2.01 | 5.9 | 1.52 | 2.8 |
| 763 | 3.2 | 2.32 | 7.4 | 3.09 | 10.4 | 3.12 | 11.2 |
| 764 | 0.3 | 1.80 | 3.8 | 2.30 | 6.1 | 2.63 | 8.1 |
| 765 | 0.4 | 3.85 | 6.4 | 7.98 | 11 | 5.96 | 9.6 |
| 766 | 2.3 | 3.81 | 2.2 | 5.71 | 3.2 | 21.36 | 8 |
| 767 | 8.8 | 2.31 | 2.3 | 1.42 | 0.6 | 1.31 | 0.4 |
| 768 | 3.4 | 1.47 | 5.8 | 1.51 | 6 | 1.42 | 5 |
| 769 | 4.7 | 1.47 | 0.8 | −1.77 | 1.4 | −1.22 | 0.3 |
| 770 | 1.1 | 3.46 | 11.2 | 3.40 | 10.3 | 3.85 | 12.4 |
| 771 | 7.9 | 5.74 | 20.3 | 6.90 | 21.4 | 5.02 | 18.6 |
| 772 | 1.9 | −1.48 | 3.4 | −1.63 | 4.4 | −1.58 | 4.3 |
| 773 | 0.1 | 2.07 | 6.1 | 1.42 | 1.8 | 1.50 | 2.4 |
| 774 | 2.5 | −1.41 | 0.8 | −1.82 | 1.8 | −2.61 | 4 |
| 775 | 7.8 | 1.39 | 0.7 | 1.49 | 0.8 | 1.13 | 0.2 |
| 776 | 2.8 | −1.04 | 0.1 | −1.11 | 0.4 | −1.23 | 1 |
| 777 | 0.7 | −1.60 | 4.2 | −1.86 | 5.9 | −1.46 | 3 |
| 778 | 1.9 | 2.00 | 5.1 | 2.24 | 6 | 2.21 | 6.2 |
| 779 | 0.8 | 2.26 | 4.8 | 2.21 | 4.3 | 3.04 | 7.6 |
| 780 | 5.5 | 1.06 | 0.3 | 1.02 | 0.1 | −1.05 | 0.3 |
| 781 | 5.1 | −1.08 | 0.2 | −1.02 | 0 | −1.06 | 0.2 |
| 782 | 0 | 1.68 | 5.5 | 1.52 | 3.7 | 1.25 | 1.5 |
| 783 | 1.5 | 3.35 | 9.2 | 3.45 | 8.9 | 3.28 | 9 |
| 784 | 4.3 | 5.68 | 9.4 | 5.99 | 9.1 | 7.10 | 11 |
| 785 | 1.3 | 2.08 | 9.5 | 2.07 | 9.4 | 1.97 | 9.1 |
| 786 | 0.8 | 2.39 | 8.2 | 1.91 | 4.9 | 1.90 | 5.2 |
| 787 | 2 | 1.16 | 0.5 | 1.50 | 2.2 | 1.55 | 2.6 |
| 788 | 3 | −1.34 | 1.6 | −1.19 | 0.7 | −1.14 | 0.5 |
| 789 | 0.2 | −1.41 | 2.2 | 1.54 | 2.8 | 2.14 | 6.8 |
| 790 | 0.7 | −1.08 | 0.4 | −1.22 | 1.4 | −1.07 | 0.3 |
| 791 | 2.4 | 1.62 | 4.3 | 1.65 | 4.2 | 1.58 | 3.9 |
| 792 | 2.1 | 1.21 | 0.6 | −1.72 | 2.4 | −2.13 | 4.3 |
| 793 | 1.2 | −2.78 | 3.6 | −3.66 | 4.8 | −3.23 | 4.4 |
| 794 | 9.2 | 1.92 | 3.1 | 1.27 | 0.7 | −1.08 | 0.2 |
| 795 | 0.7 | 1.09 | 0.1 | −1.94 | 1.6 | −2.72 | 3.3 |
| 796 | 3 | 13.06 | 6.8 | 6.49 | 3.9 | 2.12 | 1 |
| 797 | 2 | −1.57 | 5.7 | −1.71 | 7 | −1.58 | 5.8 |
| 798 | 11.1 | 7.84 | 20.9 | 7.22 | 19.1 | 4.08 | 13.5 |
| 799 | 1.1 | 1.38 | 2.2 | 1.65 | 4.1 | 1.26 | 1.4 |
| 800 | 2.6 | 1.47 | 8.1 | 1.53 | 8.8 | 1.48 | 8.5 |
| 801 | 14.2 | 2.57 | 12.1 | 2.52 | 11 | 2.95 | 14.3 |
| 802 | 5.2 | 1.62 | 6 | 1.41 | 3.3 | 1.31 | 2.4 |
| 803 | 2.8 | 2.14 | 6.2 | 2.62 | 8.1 | 3.26 | 11.6 |
| 804 | 3.7 | 1.80 | 5.8 | 1.10 | 0.4 | 1.19 | 0.9 |
| 805 | 0.1 | 1.25 | 4.4 | 1.24 | 3.9 | 1.17 | 2.5 |
| 806 | 1.6 | 1.32 | 1.2 | 2.02 | 4.5 | 2.36 | 6.6 |
| 807 | 9 | −1.20 | 0.4 | −2.42 | 3.3 | −2.26 | 3.1 |
| 808 | 12.9 | 7.72 | 23.2 | 5.96 | 19.1 | 4.77 | 17.4 |
| 809 | 6.6 | 1.61 | 6.4 | 1.62 | 6.1 | 1.93 | 10.1 |
| 810 | 8.2 | −1.28 | 3.3 | −1.11 | 0.9 | −1.10 | 0.8 |
| 811 | 1.7 | 2.02 | 0.9 | 7.38 | 4.1 | 3.25 | 1.9 |
| 812 | 1.5 | −1.15 | 0.8 | −1.33 | 2.2 | −1.55 | 4.4 |
| 813 | 3.2 | −1.67 | 3.2 | −1.68 | 3 | −2.09 | 5.6 |
| 814 | 3 | 1.14 | 1.6 | 1.12 | 1.1 | 1.18 | 2.3 |
| 815 | 4.1 | 1.01 | 0.1 | 1.09 | 0.9 | −1.00 | 0 |
| 816 | 2 | 1.47 | 10.1 | 1.49 | 9.8 | 1.40 | 8.2 |
| 817 | 0.5 | 1.38 | 7 | 1.58 | 10.9 | 1.65 | 13 |
| 818 | 0.9 | 1.02 | 0.1 | 1.08 | 0.8 | −1.17 | 2.1 |
| 819 | 0.4 | 1.86 | 6.8 | 2.30 | 9.6 | 1.88 | 6.9 |
| 820 | 1.2 | 1.22 | 3.5 | 1.19 | 2.7 | 1.23 | 3.8 |
| 821 | 1 | −1.48 | 4.3 | −1.07 | 0.3 | −1.26 | 1.9 |
| 822 | 4.3 | 1.06 | 0.4 | 1.26 | 2.6 | 1.11 | 0.9 |
| 823 | 8.5 | −1.00 | 0 | 1.00 | 0 | 1.14 | 1.2 |
| 824 | 0.8 | 1.50 | 4.3 | 1.94 | 8.3 | 1.63 | 5.6 |
| 825 | 0.9 | −1.27 | 2.8 | −1.50 | 5.9 | −1.22 | 2.1 |
| 826 | 0.5 | 1.66 | 5.7 | 1.54 | 4.1 | 1.79 | 6.9 |
| 827 | 4.3 | 1.02 | 0.1 | 1.16 | 1.7 | 1.09 | 0.8 |
| 828 | 2.3 | 1.55 | 4.6 | 1.03 | 0.1 | −1.19 | 1.1 |
| 829 | 0.2 | −1.54 | 4.5 | −1.68 | 5.5 | −1.69 | 6 |

To assign annotation to the differentially expressed genes for the better understanding of their functional roles in the angiogenic processes, a variety of public resources as well as proprietary tools are used including: Affymetrix Netaffx analysis database, GeneCard (Weizmann Institute of Science Crown Human Genome Center, UniGene, RefSeq and LocusLink (Wheeler, D. L., et al., (2001) Nucleic Acids Res., 29, 11-16); SwissProt/TrEMBL (Bairoch, A. and Apweiler, R. (2000) Nucleic Acids Res., 28, 45-48); FANTOM2 (Bono H, et al., Nucleic Acids Res. 2002 Jan. 1; 30(1):116-8); and The Institute of Genomics Research (TIGR) Gene Index databases (Nucleic Acids Res. 2000 Jan. 1; 28(1):141-5).

For those uncharacterized genes or ESTs, a semi-automatic annotation strategy is used combining the following steps: i) Homology search against the major nucleotide and protein databases, including NCBI-nr, Ensembl, SwissProt/SPTreMBL, and GenPept using BLASTX and BLASTP; ii) Function derivation from homolog/orthlog databases, including HomoloGene, TIGR Resourcerer, and TOGA databases (Wheeler, D. L., et al, supra; Tsai J, et al. (2001) Genome Biology 2-11; Genome Res. 2002, 493-502). The results of the annotation process are edited and additional curation is performed to identify the homologous human and mouse DNA sequences and encoded protein sequences.

Based on the annotation and curation process, the 829 probe sets correspond to 736 rat genes. The Affymetrix probe set accession numbers and the corresponding rat, human and mouse representative (RefSeq or GenBank) accession numbers are shown in Table 4. Table 4, being longer than 51-pages, is submitted in duplicate (in compliance with 37 CFR § 1.52(e)(4), separately on one CD-R each, in accordance with 37 CFR §1.52(e)(1)(iii) and 37 CFR §1.58.

### Example 4

### Construction of Vectors that Express a Gene of the Present Invention

The cDNA sequence for a gene of the present invention is retrieved from suitable public database and two oligonucleotides including one containing the 5′ end of the gene (5′ oligonucleotide) and one containing the 3′ end of the gene (3′ oligonucleotide) are synthesized. Using the above 5′ and 3′ oligonucleotides, the cDNA is amplified by PCR from a suitable animal cDNA library available commercially using a PCR kit. The PCR product is purified and cloned into a vector (e.g. pIRESneo vector (Clonetech Inc., Palo Alto, Calif., USA) by commercially available PCR cloning kit according to the manufacturer's recommendations. The cloned gene is then used to transform competent E. coli cells. Plasmid DNA is isolated and the insert from at least one clone is sequenced to ensure that the gene sequence is correct. Suitable vertebrate cells (e.g. HEK293 cells containing a stably integrated Mercury CRE-LUC plasmid (Clonetech Inc., Palo Alto, Calif., USA) are transfected with purified plasmid DNA. Cells stably transfected with plasmid DNA are selected by culturing the cells in G418. The stably transfected cells are propagated in DMEM (Life Technologies, Rockville, Md.) containing 10% fetal bovine serum at 37° C. in a 5% CO₂. The clones are then characterized to ensure they have the correct gene activity. Cells expressing the gene at an appropriate level may then be utilized for further analysis.

### Example 5

### Binding Assays

Binding analysis of compounds is performed in whole cells by plating the cells from Example 2 expressing a gene of the present invention in a 96 well plate. Cells are seeded in DMEM medium containing 10% fetal bovine serum at 37° C. in a 5% CO₂ and incubated overnight. The culture medium is removed and the appropriate amount of suitably labeled compound is added. The cells are incubated for 90 minutes at room temperature then washed 4 times with phosphate buffered saline. Following the final wash the plate is analyzed for binding. For saturation binding analysis, log doses of a compound ranging from 10⁻¹² to 10⁻³ M are added to the cells and binding analyzed both in the absence and in the presence of a saturating concentration of unlabeled compound for evaluation of non-specific binding. The binding analysis may differentiate various compounds based on the binding affinities.

### Example 6

### Activation Assay

Activation analysis is performed by seeding the cells of Example 2 into Packard View Plate-96 (Packard Inc., CA). Cells are seeded in DMEM containing 10% fetal bovine serum at 37° C. in a 5% CO₂ and incubated overnight. The medium is then removed and replaced with DMEM containing 0.01% bovine albumin fraction V containing the compound of interest. The cells are further incubated for four hours at 37° C. after which the medium is removed and the cells are washed twice with Hanks Balanced Salt Solution (HBSS). Lysis Reagent is then added to the washed cells and incubated for 20 minutes at 37° C. The cells are then placed at −80° C. for 20 minutes followed by a 20-minute incubation at 37° C. After this incubation, Luciferase Assay Buffer and Luciferase Assay Substrate (Promega Inc., Madison, Wis.) are added to the cell lysates and luciferase activity quantitated using a luminometer. Relative activity of a compound is evaluated by comparing the increase following exposure to compound to the level of luciferase in HEK cells that contain the CRE-LUC construct without the gene of interest following exposure to compound. Specificity of response is also checked by evaluating the luciferase response of hCR/CRE-LUC HEK cells to compound in the presence and absence of a 10-fold excess of an antagonist.

### Example 7

Proteins of the inventions could be further characterized in functional assays and agonists or antagonists of these proteins may be screened using assays known to one skilled in the art. Following are some exemplary assays that could be used.

### Cell Proliferation Assay Using NIH 3T3 Cells

To measure cell proliferation in response to protein growth and differentiation factor, NIH 3T3 cells are plated at a seeding density of 5×10³ cells per well in 96-well plate. After 24 h incubation at 37° C. in 5% CO₂, cell culture medium is aspirated and cells are washed with serum-free DMEM. After washing, cells are treated with different doses of growth and differentiation factor or vehicle suspended in serum-free medium for 24 h. Cell number is measured by a colorimetric method using CellTiter reagent from Promega. CellTitre assay is performed by adding 20 μl of the CellTiter Reagent to each well of 96-well culture plate. Cells are incubated for 3 hours at 37° C. and then absorbance is measured at 490 nm with a 96-well plate reader. Absorbance value obtained in samples is converted into cell numbers extrapolated from established standard curve.

### Ion Channels

Single HEK cells expressing the human potassium current HERG ion channel are used to test the effects of compounds on HERG. Only cells displaying stable currents with biophysical characteristics typical of HERG are used. Current—voltage relationships for each cell are recorded three times before and after exposure to test compounds. Compounds are prepared freshly as stock solutions and diluted in standard Tyrodes buffer. Compounds are gravity perfused through the recording chamber at a rate of 3-5 mls/min. Cells are exposed to each concentration of drug for at least 5 minutes. Deactivating tail currents (time 500 to 1000 msec) are measured at their peak amplitudes and used to assess the effects of these compounds on HERG.

HEK cells transfected with HERG are recorded with an Axopatch 1-D patch-clamp amplifier in the whole-cell configuration of the patch-clamp technique. Data acquisition and command potentials are controlled with a commercial software program (PCLAMP, Axon Instruments). The external solution is normal Tyrode's solution and contained (mM): NaCl 130, KCl 4, CaCl₂ 1.8, MgCl₂ 1, Hepes 10 and glucose 10 (pH adjusted to 7.35 with NaOH). The internal (pipette) solution is in mM: KCl 110, K₂ATP 5, K₄BAPTA 5, MgCl₂ 1, and Hepes 10 (pH adjusted to 7.2 with KOH). Microelectrodes are pulled from borosilicate glass and heat-polished (pipette tip resistance, 3-6 M). Ion currents are recorded at room temperature (22-23° C.). Command potentials are applied for 1.5 sec from a holding potential of −65 mV. Current is recorded from 500 msec depolarizing pulses ranging from −40 mV to +80 mV in 20 mV increments. Deactivating tail currents (500 msec) are recorded from preceding depolarizing pulses by returning to −45 mV.

### Screening for a Kinase Inhibitor

In order to screen for kinase inhibitors, the Promega Kinase-Glo™ Luminescent Kinase Assay is used according to the methods recommended by the manufacturer. Briefly, to each well in a 96 well plate, add the mixture containing 2× the optimal concentration of kinase and kinase substrate. Then add an optimal concentration of the test compound, mix, add the optimal concentration of ATP. Mix the plate and incubate for the optimal amount of time to ensure complete reaction. Add the optimal amount of Kinase-Glo™ reagent to all wells, mix and incubate at room temperature for 10 minutes to stabilize the luminescent signal and record luminescence reading.

### Screening for a Phosphatase Inhibitor

To screen for the PTPase inhibitory activity, testing of the subject compounds is carried using various assays known to those skilled in the art. For example, a DiFMUP Phosphatase Assay is described. DiFMUP (“6,8-difluoro-4-methylumbelliferyl phosphate”) (Molecular Probes) (10 mM) is incubated for 15 minutes with nM concentrations of phosphatase in buffer containing 50 mM Tris (pH 7), 150 mM NaCl, 5 mM DTT, 1 mM EDTA, 0.01% BSA. The resulting phosphatase product is measured at 355/460 nm (ex/em) using a Victor V plate reader (Wallac). Inhibitors (0.002-40 mM) are pre-incubated with phosphatase for 10 minutes prior to addition of DiFMUP substrate. IC50 curves are generated using Excel-Fit®.

### Example 8

### Cornea Micropocket Assay

### Material and Methods:

### The Mouse Micropocket Corneal Assay

The mouse micropocket corneal assay is carried out according to procedures described (Kenyon, B. M., E. E. Voest, C. Chen, E. Flynn, J. Folkman and R. J. D'Amato, 1996. Invest. Ophthal. Visula Sci. 37: 1625-1632). Mice (strain C57BL/6) weighing 25-35 grams are used for the study. ETL k/o and control mice are purchased from Deltagen, Inc (San Carlos, Calif.). Animals are anesthetized with intraperitoneal injection of ketamine (100 mg/kg) and xylazine (25 mg/kg) and the eyes are topically anesthetized with 0.5% tetracaine HCl ophthalmic solution. Under an operating microscope, a central intrastromal linear keratotomy is performed with a surgical blade parallel to the insertion of the lateral rectus muscle. Using a modified von Graefe cataract knife, a corneal micropocket is created. Into the eye, a 0.4 mm×0.4 mm×0.2 mm sucrose aluminum sulfate pellet coated with 12% hydron polymer (Interferon Sciences, New Brunswick, N.J.) containing 60 ng of VEGF₁₆₅ is placed into the pocket 0.5-0.7 mm from the temporal limbus. Vetropolycin ophthalmic ointment is applied and animals are allowed to recover. Corneal angiogenesis is evaluated 5 days after the implantation using vascular FITC-dextran fluorescent imaging technique. Animals are anesthetized as described previously and FITC-dextran (molecular weight=500 K) dissolved in the saline is injected intravenously into the jugular vein at a dose of 50 mg/kg. Fluorescent images are acquired with a digital (Micropublisher RTV 5.0) image acquisition system. The filter wavelengths are set for excitation and emission at 480 nm and 515 nm, respectively.

### Gene Expression in Human Cell Lines

Human vascular cell lines are obtained from Cambrex/BioWhittaker Cell Biology Products (Walkersville, Md.), including, aortic smooth muscle cells (AoSMC; n=2), coronary artery smooth muscle cells (CaSMC; n=3), pulmonary artery smooth muscle cells (PASMC; n=3), aortic endothelial cells (HAEC; n=3), coronary artery endothelial cells (HCAEC; n=3), pulmonary artery endothelial cells (HPAEC; n=2), umbilical vein endothelial cells (HUVEC; n=3), dermal microvascular endothelial cells (HMVEC-D; n=3) and lung microvascular endothelial cells (HMVEC-L; n=3). Cells are cultured as described by the manufacturer. For the final passage, cells are plated in 100 mm plates with 5 ml of culture media. The media is removed and replaced with 1 mL of Trizol Reagent (Gibco BRL, Carlsbad Calif.) and stored at −80° C. mRNA is isolated as described for the aortic ring and cornea samples. For the human cell lines, Human U133A/B Affymetrix Genechips are used. The statistical analysis is based on the Affy signal (MAS 5.0) and Affy Absent/Present calls.

### Zebrafish Studies

List of Abbreviations: dpf (days post-fertilization); hpf (hours post-fertilization); ISVs (intersegmental vessels); MO (morpholino).

To assess gene function in zebrafish (Danio rario) angiogenesis, MOs directed against ETL, GPR (or GPR176) and their respective mismatch controls are microinjected into zebrafish at the 1-2 cell stage. Blood circulation through the intersegmental vessels (ISVs) is observed in live zebrafish at 48 hpf and morphology of ISVs in the tail at 48 hpf using Phy-V antibody staining is also examined as described (Seng W. L. et al., Angiogenesis. 2004; 7(3):243-53.). The sequence for the ETL morpholino is: GCAGGAGTTTCATTGGAGAACTGTG. The sequence for the mismatch ETL morpholino (small case letters indicate mismatches) is: GCAcGAcTTTgATTGGAcAACTcTG. The sequence for the GPR morpholino is: AGCTCTCCGCGTTATCCGCCTCCAT. The sequence for the mismatch ETL morpholino (small case letters indicate mismatches) is: AGgTCTgCGCcTTATCCcCCTCgAT.

### Results

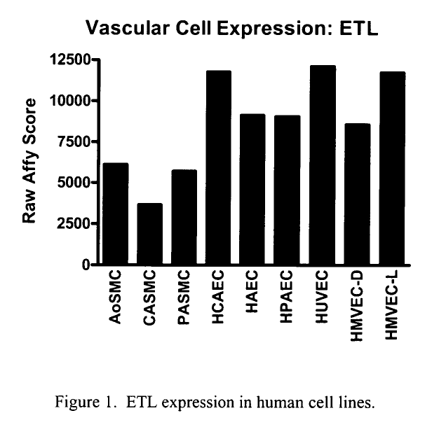

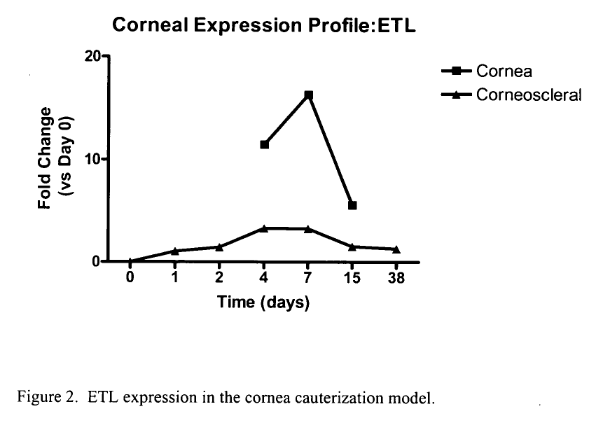

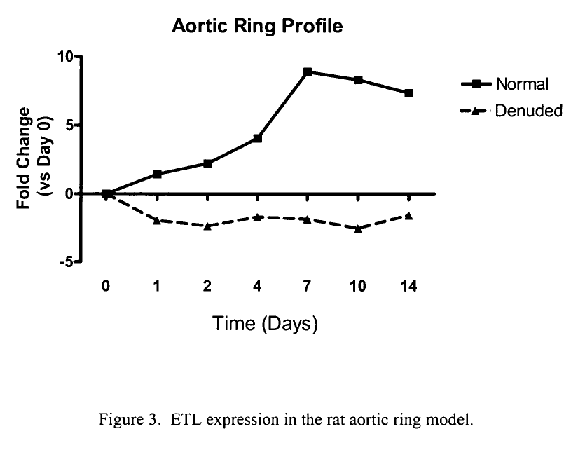

The gene expression profile of EGF-TM7-latrophilin-related protein (ETL) indicates that this may be a gene of interest for angiogenesis. ETL is a recently discovered receptor that has been classified into the adhesion family of G-protein coupled receptors (GPCRs). ETL's large extracellular domain contains EGF modules, a Ser/Thr rich linker region and a Cys-rich proteolysis domain (Nechiporuk T. J. Biol. Chem. 276, 6, 4150-4157, 2001). Transcriptional profiling of ETL demonstrates that it is present in cultured, human smooth muscle and endothelial cells with higher expression noted in the endothelial cells examined (FIG. 1). These data indicate that ETL is expressed in the vascular cells and may contribute to blood vessel regulation. In addition, data show that ETL transcript levels are significantly increased in the rat cornea cauterization model (FIG. 2) and the rat aortic ring model (FIG. 3). The expression of ETL in these models parallels the changes in vascularity, supporting the contention that ETL may play a role in angiogenesis and/or vascular remodeling. In the cornea, ETL expression increases dramatically in the sections that include the neovessels (“cornea”), indicating the ETL expression is higher in newly formed and developing blood vessels. In the aortic ring, ETL expression increases throughout the study and its expression does not respond in the aortic ring samples that have had the endothelial cell removed (“denuded”), suggesting that ETL is expressed and transcriptionally regulated in the rat endothelial cells during angiogenesis. Alternatively, these data suggest that ETL expression levels may be a marker of blood vessel growth and/or development. ETL is reportedly present in the heart and is developmentally regulated, suggesting that ETL may have a role in the quiescent adult vasculature and during angiogenesis (Nechiporuk T. JBC, 276, 6, 4150-4157 2001).

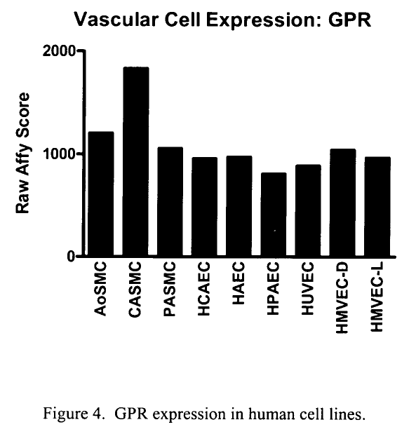

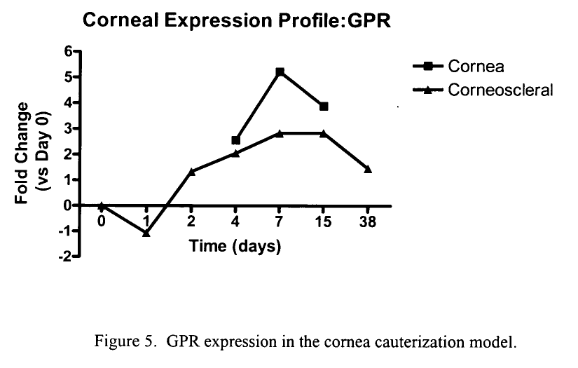

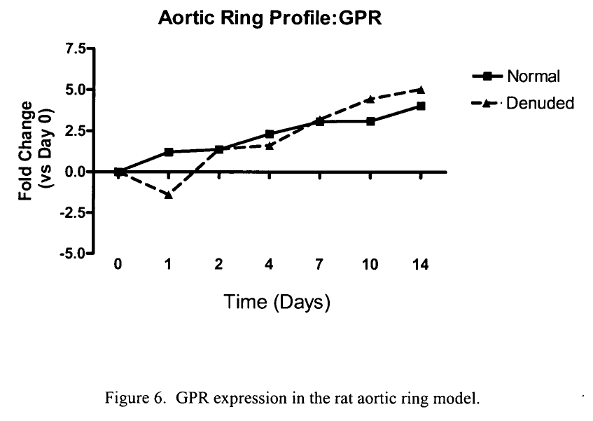

The gene expression profile of GPR indicates that this may be a gene of interest for angiogenesis. A novel GPCR, now designated GPR176, was cloned from rat and human cDNA libraries by using probes generated by PCR amplification with degenerate oligonucleotide primers (Ishizaka, N., Okazaki, H., Kurokawa, K., Kumada, M., and Takuwa, Y. (1994) Biochem. Biophys. Acta. 1218:173-180. and Hata, S., Emi, Y. Jyanagi, T. and Osumi, T. (1995) Biochem. Biophys. Acta. 1261:121-125). Data indicate that GPR is expressed in human endothelial cell lines and human vascular smooth muscle cell lines isolated from a variety of vascular beds (FIG. 4). In the rat cornea model, expression of GPR is increased 2-3 fold during vessel growth and maturation, and decreases towards normal levels after vessels remodel (FIG. 5). These data indicate that GPR expression may be regulated in both the limbal vessels and the neovascular sprouts. In the rat aortic ring model, increased expression of GPR is observed from day 4 onward (FIG. 6). Differential expression at earlier timepoints in normal and denuded rat aortic ring suggests that GPR may be playing a role in both the smooth muscle and endothelial cells.

### In Vivo Experiments

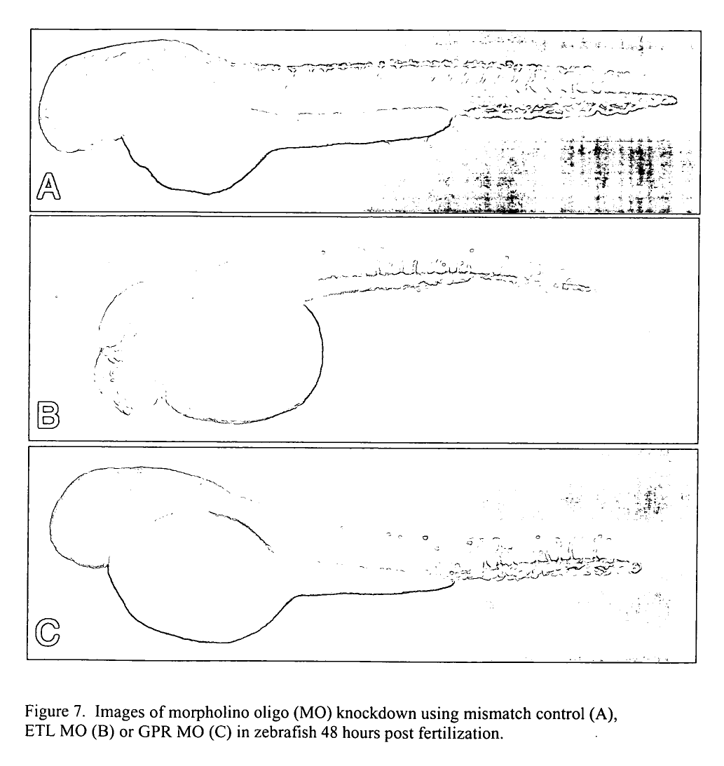

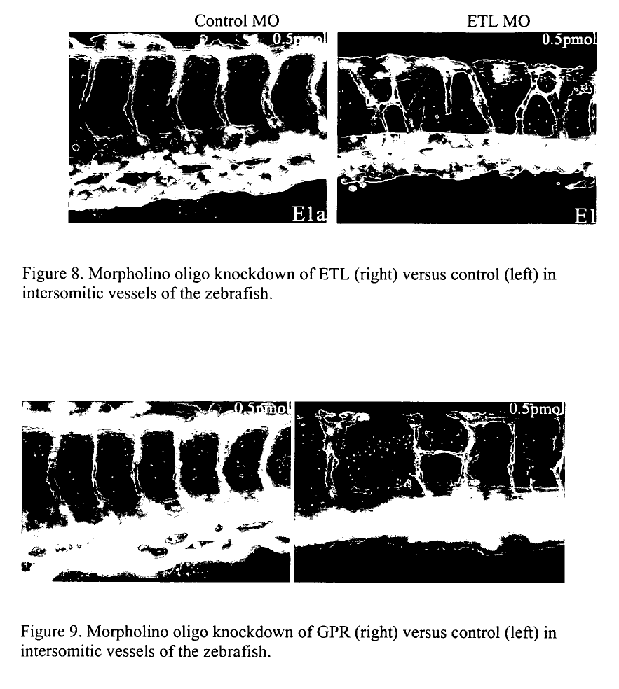

While transcriptional gene changes provide the basis for investigating ETL and GPR as regulators of vascular development, experiments are carried out to obtain functional evidence that ETL and GPR are involved in blood vessel growth and development. To accomplish this, a zebrafish model may be used with ETL or GPR morpholino oligos to reduce gene expression during vasculogenesis. Morpholino knockdown of ETL transcript levels results in developmental abnormalities (FIG. 7) and aberrant blood vessel formation (FIG. 8), suggesting an important role for this protein in blood vessel development. Experiments in zebrafish using 0.5 pmol ETL MO and their respective mismatch controls show that zebrafish injected with ETL MO have visible circulatory defects (slow circulation or incomplete circulation through the intersegmental vessels) and results in slightly curved body morphology in the live zebrafish at 48 hpf. This effect is not seen in control MO injections. Examination of ISVs by immunostaining at 48 hpf shows abnormal connections and incomplete vessel formation (FIG. 4) in 100% of the zebrafish in this experiment. Addition of 0.25 pmol dose of ETL MO shows the same phenotype as the 0.5 pmol dose, although the number of zebrafish affected is lower (70%) demonstrating that the phenotype may be specific and dose-dependent, but not due to toxic side effects.

Experiments in zebrafish using 0.5 pmol of GPR MO and its respective mismatch controls show that the 0.5 pmol dose of GPR MO causes visible circulatory defects (slow circulation or incomplete circulation through the ISVs) and results in slightly curved body morphology in the live zebrafish at 48 hpf (FIG. 7). This effect is not seen in control MO injections. Examination of ISVs by immunostaining at 48 hpf shows abnormal connections and incomplete vessel formation (FIG. 9) in 76% of the zebrafish assessed in experiment 1 and 94% in experiment 2. Addition of 0.25 pmol GPR MO shows a similar phenotype as the 0.5 pmol dose, although the number of zebrafish affected (37%) was lower, demonstrating that the phenotype may be specific and dose-dependent, but not due to toxic side effects

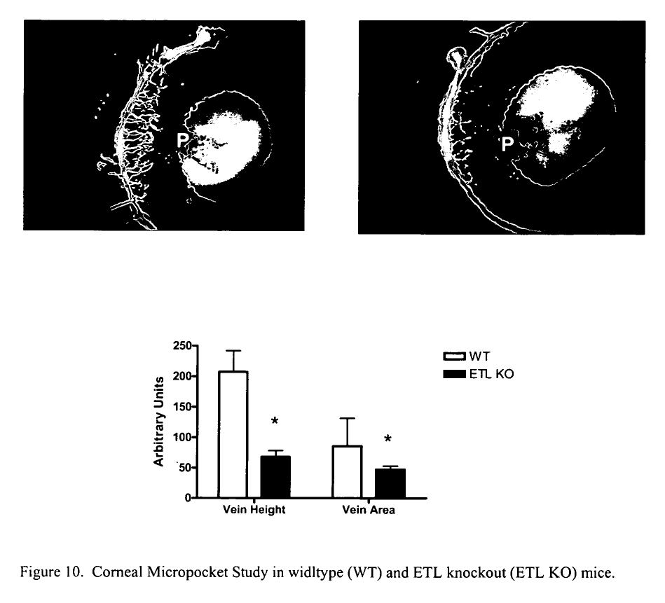

To further investigate the role of ETL in blood vessel growth, ETL knockout mice are obtained from Deltagene and cornea micropocket studies are performed as described (Kenyon, B. M et. al. Invest. Ophthal. Visula Sci. 37: 1625-1632). A 60 ng pellet of VEGF₁₆₅ is inserted and vessel growth is quantitated. ETL KO mice exhibit significantly reduced blood vessel growth towards the VEGF₁₆₅ pellet compared to WT mice, suggesting that ETL plays an important role in blood vessel growth and/or development (FIG. 10).

Except as otherwise noted, all amounts including quantities, percentages, portions, and proportions, are understood to be modified by the word “about”, and amounts are not intended to indicate significant digits.

Except as otherwise noted, the articles “a”, “an”, and “the” mean “one or more”.

All documents cited herein are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications may be made without departing from the spirit and scope of the invention.

It is therefore intended to cover in the appended claims all such changes and modifications that are within the scope of this invention.

TABLE 4

| | | Affymetrix | | | | |
|---|---|---|---|---|---|---|
| | Affymetrix | GenBank | | | | |
| | Probe Set | Accession | | Rat RefSeq | Human RefSeq | Mouse RefSeq |
| No. | Accession No. | No. | Rat RefSeq Description | Accession No. | Accession No. | Accession No. |
| 1 | 1373696_AT | AI171656 | LOC362536 (LOC362536), mRNA | XM_347061 | | |
| 2 | 1392471_AT | BI302750 | Guanine nucleotide binding protein 12 | XM_347046 | NM_018841 | NM_025278 |
| 3 | 1373695_AT | AI176675 | LOC360360 hypothetical gene supported by | XM_346651 | NM_002721 | NM_024209 |
| | | | NM_133589 | | | |
| 4 | 1374626_AT | BG371585 | Rattus norvegicus similar to leucine-rich | XM_346143 | NM_052972 | NM_029796 |
| | | | alpha-2-glycoprotein (LOC367455), mRNA | | | |
| 5 | 1389966_AT | AI176126 | Similar to alpha 3 type VI collagen isoform 1 | XM_346073 | NM_004369 | NM_009935 |
| | 1396055_AT | AW530165 | precursor; collagen VI, alpha-3 polypeptide | | NM_057164 | |
| | | | (LOC367313), mRNA | | NM_057165 | |
| | | | | | NM_057166 | |
| | | | | | NM_057167 | |
| 6 | 1388120_AT | BE328942 | programmed cell death 6 interacting protein | XM_346006 | NM_013374 | NM_011052 |
| | | | | AF192757 | | |
| 7 | 1391421_AT | AI599819 | Similar to Acyl-CoA dehydrogenase family | XM_345912 | NM_014384 | NM_025862 |
| | | | member 8, mitochondrial precursor (ACAD- | | | |
| | | | 8) (Isobutyryl-CoA dehydrogenase) | | | |
| | | | (LOC367046), mRNA | | | |
| 8 | 1373483_AT | AA963764 | Similar to NG28 (LOC366848), mRNA | XM_345803 | NM_198471 | NM_030697 |
| 9 | 1392490_AT | BM390676 | Similar to RIKEN cDNA 1110007C24 | XM_345621 | NM_018202 | NM_025382 |
| | | | (LOC366524), mRNA | | | |
| 10 | 1390846_AT | BG380309 | Similar to alpha 1 type XVI collagen | XM_345584 | NM_001856 | NM_028266 |
| | | | precursor; collagen XVI, alpha-1 polypeptide | | | |
| | | | (LOC366474), mRNA | | | |
| 11 | 1375277_AT | AA800680 | Similar to smooth muscle myosin | XM_345324 | | NM_025980 |
| | 1382138_AT | BG380221 | phosphatase regulatory subunit homolog | | | |
| | | | family member, Maternal Effect Lethal MEL- | | | |
| | | | 11 (110.6 kD) (mel-11) (LOC366002), | | | |
| | | | mRNA | | | |
| 12 | 1398348_AT | AA945604 | Similar to dimethylarginine | XM_345299 | NM_012137 | NM_026993 |
| | | | dimethylaminohydrolase 1; NG, NG | | | |
| | | | dimethylarginine dimethylaminohydrolase | | | |
| | | | (LOC365956), mRNA | | | |
| 13 | 1373151_AT | AW252169 | Similar to lipoma HMGIC fusion partner | XM_345210 | NM_005780 | NM_175386 |
| | | | (LOC365795), mRNA | | | |
| 14 | 1379335_AT | AI639045 | Similar to Polyadenylate binding protein- | XM_345160 | NM_006451 | NM_145457 |
| | | | interacting protein 1 (LOC365684), mRNA | | NM_182789 | |
| | | | | | NM_183323 | |
| 15 | 1374207_AT | BI275292 | Rattus norvegicus angiopoietin-2 (Agpt2), | XM_344544 | NM_001147 | NM_007426 |
| | | | mRNA | | | |
| 16 | 1399019_AT | AI407868 | Similar to RIKEN cDNA 1110035H23 | XM_344403 | NM_022060 | NM_134076 |
| | | | (LOC364380), mRNA | | | |
| 17 | 1371016_AT | BM386010 | Rattus norvegicus T cell receptor V alpha 23 | XM_344402 | | XM_356740 |
| | | | chain subunit mRNA, partial cds | | | |
| 18 | 1374104_AT | BI281680 | claudin 5 | XM_344058 | NM_003277 | NM_013805 |
| 19 | 1373557_AT | AI145081 | mini chromosome maintenance deficient 4 | XM_344048 | NM_005914 | NM_008565 |
| | | | homolog (S. cerevisiae) | | NM_182746 | |
| 20 | 1388492_AT | AW919111 | Similar to ABINl, A20-binding inhibitor of | XM_343894 | NM_006058 | NM_021327 |
| | | | NF-kappa B activation (large) (LOC363599), | | | |
| | | | mRNA | | | |
| 21 | 1373379_AT | BF396350 | Similar to Interleukin-1 receptor-associated | XM_343844 | NM_001569 | NM_008363 |
| | | | kinase 1 (IRAK-1) (IRAK) (Pelle-like protein | | | |
| | | | kinase) (mPLK) (LOC363520), mRNA | | | |
| 22 | 1372947_AT | BG672591 | Rattus norvegicus plastin 3 (T-isoform) | XM_343776 | NM_005032 | NM_145629 |
| 23 | 1371037_AT | U06230 | protein S | XM_343650 | NM_000313 | NM_011173 |
| 24 | 1388533_AT | BM390302 | Similar to golli-interacting protein | XM_343588 | NM_021198 | NM_153088 |
| | | | (LOC363249), mRNA | | NM_182642 | |
| 25 | 1388449_AT | AI175551 | Rattus norvegicus similar to eukaryotic | XM_343580 | NM_001959 | NM_018796 |
| | | | translation elongation factor 1 beta 2 | | NM_021121 | |
| | | | (LOC363241), mRNA | | | |
| 26 | 1373463_AT | AI172281 | Collagen, type V, alpha 2 | XM_343564 | NM_000393 | NM_007737 |
| 27 | 1389546_AT | BF418582 | Angiomotin like 2 | XM_343457 | NM_016201 | NM_019764 |
| 28 | 1374816_AT | AI103939 | Similar to hypothetical protein FLJ30973 | XM_343423 | NM_152451 | XM_194467 |
| | | | (LOC363091), mRNA | | | |
| 29 | 1388204_AT | M60616 | matrix metalloproteinase 13 | XM_343345 | NM_002427 | NM_008607 |
| 30 | 1374474_AT | BE099085 | Similar to copine family member | XM_343318 | NM_153634 | NM_025815 |
| | | | (LOC362988), | | | |
| 31 | 1388568_AT | AA891553 | Similar to eukaryotic translation initiation | XM_343283 | NM_003753 | NM_018749 |
| | | | factor 3 subunit 7; eukaryotic translation | | | |
| | | | initiation factor 3, subunit 7 (zeta, 66/67 kD); | | | |
| | | | translation initiation factor eIF3 p66 subunit | | | |
| | | | (LOC362952), mRNA | | | |
| 32 | 1372084_AT | AI104546 | Rattus norvegicus similar to protein tyrosine | XM_343259 | NM_032611 | NM_008975 |
| | | | phosphatase type IVA, member 3 isoform 1; | | NM_007079 | |
| | | | potentially prenylated protein tyrosine | | | |
| | | | phosphatase (LOC362930), mRNA | | | |
| 33 | 1370956_AT | BM390253 | decorin | XM_343201 | NM_001920 | NM_007833 |
| | | | | | NM_133503 | |
| | | | | | NM_133504 | |
| | | | | | NM_133505 | |
| | | | | | NM_133506 | |
| | | | | | NM_133507 | |
| 34 | 1393401_AT | BE109323 | Similar to 9530046H09Rik protein | XM_343181 | NM_016496 | NM_145486 |
| | | | (LOC362849), mRNA | | | |
| 35 | 1388602_AT | AI237358 | adipsin | XM_343169 | NM_001928 | NM_013459 |
| 36 | 1384900_AT | AW530806 | Similar to MOB-LAK (LOC362833), mRNA | XM_343161 | NM_024761 | NM_178061 |
| | | | | | NM_130807 | |
| 37 | 1373874_AT | AI169638 | LOC81536: Sphingosine-1-phosphate | XM_343081 | NM_030791 | NM_030750 |
| | | | phosphohydrolase | | | |
| 38 | 1376106_AT | AI010157 | Similar to hypothetical protein MGC33926 | XM_343007 | NM_152390 | NM_026516 |
| | | | (LOC362691), mRNA | | | |
| 39 | 1393067_AT | AI710931 | Tek: Endothelial-specific receptor tyrosine | XM_342863 | NM_000459 | NM_013690 |
| | | | kinase | | | |
| 40 | 1374176_AT | AI408727 | Similar to DNA segment, Chr 4, Brigham & | XM_342853 | NM_203403 | NM_026821 |
| | | | Womens Genetics 0951 expressed | | | |
| | | | (LOC362535), mRNA | | | |
| 41 | 1373881_AT | BF285771 | Similar to GDP-dissociation inhibitor | XM_342777 | NM_001175 | NM_007486 |
| | | | (LOC362456), mRNA | | | |
| 42 | 1367463_AT | AI235510 | B-cell receptor-associated protein 37 | XM_342755 | NM_007273 | NM_007531 |
| 43 | 1385057_AT | AA819868 | Similar to RIKEN cDNA 9030407H20 | XM_342740 | NM_018306 | NM_144805 |
| | | | (LOC362420), mRNA | | | |
| 44 | 1389153_AT | BI296087 | Hypothetical RNA binding protein RDA288 | XM_342705 | NM_015640 | NM_025814 |
| 45 | 1388493_AT | AI600030 | Similar to Expressed sequence AW146242 | XM_342698 | NM_030796 | NM_146168 |
| | | | (LOC362374), mRNA | | | |
| 46 | 1371999_AT | BI303641 | Rattus norvegicus similar to dJ862K6.2.2 | XM_342569 | NM_006275 | NM_026499 |
| | | | (splicing factor, arginine/serine-rich 6 | | | |
| | | | (SRP55-2)(isoform 2)) (LOC362264), mRNA | | | |
| 47 | 1372729_AT | AI137406 | Rattus norvegicus similar to endothelial cell | XM_342554 | NM_006404 | NM_011171 |
| | | | protein C/APC receptor (LOC362248), | | | |
| | | | mRNA | | | |
| 48 | 1371625_AT | AA800190 | brain glycogen phosphorylase | XM_342542 | NM_002862 | NM_153781 |
| 49 | 1370855_AT | BG666933 | cystatin C | XM_342538 | NM_000099 | NM_009976 |
| 50 | 1395663_AT | BM390763 | Similar to BENE protein (LOC362211), | XM_342514 | NM_005434 | NM_145532 |
| | | | mRNA | | | |
| 51 | 1389573_AT | AI170665 | Similar to RIKEN cDNA 1810008K03 | XM_342497 | NM_024111 | NM_026929 |
| | | | (LOC362196), mRNA | | | |
| 52 | 1388243_AT | BG663422 | putative G protein coupled receptor | XM_342493 | NM_007223 | NM_201367 |
| 53 | 1383863_AT | BF288303 | Similar to rhombotin homolog 2 - mouse | XM_342475 | NM_005574 | NM_008505 |
| | | | (LOC362176), mRNA | | | |
| 54 | 1376775_AT | AI059078 | Similar to RIKEN cDNA 2600010E01 | XM_342471 | NM_024841 | NM_175181 |
| | | | (LOC362171), mRNA | | | |
| 55 | 1374575_AT | BI304123 | Similar to old astrocyte specifically induced | XM_342466 | NM_052854 | NM_011957 |
| | | | substance (LOC362165), mRNA | | | |
| 56 | 1373374_AT | BM389691 | Similar to LIM domain only 4; LIM only 4; | XM_342353 | NM_006769 | NM_010723 |
| | | | ethanol induced 4 (LOC362051), mRNA | | | |
| 57 | 1388356_AT | AI406499 | Similar to RIKEN cDNA 2300002L21 | XM_342291 | NM_080388 | NM_026416 |
| | | | (LOC361991), mRNA | | | |
| 58 | 1373911_AT | BM389026 | Similar to osteoblast specific factor 2 | XM_342245 | NM_006475 | NM_015784 |
| | | | precursor (LOC361945), mRNA | | | |
| 59 | 1386080_AT | BE107815 | Hairy/enhancer-of-split related with YRPW | XM_342216 | NM_012258 | NM_010423 |
| | | | motif 1 | | | |
| 60 | 1374586_AT | AI103993 | Similar to RIKEN cDNA C230032K13 | XM_342197 | NM_019087 | NM_172595 |
| | | | (LOC361900), mRNA | | | |
| 61 | 1371369_AT | BI287851 | Rattus norvegicus similar to procollagen, type | XM_342115 | NM_001849 | NM_146007 |
| | | | VI, alpha 2 (LOC361821) | | NM_058174 | |
| | | | | | NM_058175 | |
| 62 | 1388521_AT | BG380693 | Similar to pyrroline-5-carboxylate synthetase | XM_342048 | NM_002860 | NM_019698 |
| | | | (LOC361755), mRNA | | | NM_153554 |
| 63 | 1373970_AT | AI716248 | Similar to RIKEN cDNA 9230117N10 | XM_342042 | NM_033439 | NM_133775 |
| | | | (LOC361749), mRNA | | | |
| 64 | 1373258_AT | AI169359 | Rattus norvegicus similar to cathepsin F | XM_341987 | NM_003793 | NM_019861 |
| | | | (LOC361704) | | | |
| 65 | 1373829_AT | AI412658 | Fibroblast growth factor receptor 2 | XM_341940 | NM_000141 | NM_010207 |
| | | | | | NM_022969 | NM_201601 |
| | | | | | NM_022970 | |
| | | | | | NM_022971 | |
| | | | | | NM_022972 | |
| | | | | | NM_022973 | |
| | | | | | NM_022974 | |
| | | | | | NM_022975 | |
| | | | | | NM_022976 | |
| | | | | | NM_023028 | |
| | | | | | NM_023029 | |
| | | | | | NM_023030 | |
| | | | | | NM_023031 | |
| 66 | 1388955_AT | BE110691 | Putative eps protein | XM_341794 | NM_014601 | NM_153068 |
| 67 | 1390141_AT | BF281848 | Similar to 2410004L15Rik protein | XM_341750 | NM_015440 | NM_172308 |
| | | | (LOC361472), mRNA | | | |
| 68 | 1390388_AT | BF283951 | Similar to ferrochelatase (LOC361338), | XM_341622 | NM_000140 | NM_007998 |
| | | | mRNA | | | |
| 69 | 1372818_AT | BI284441 | Collectin sub-family member 12 | XM_341574 | NM_030781 | NM_130449 |
| | | | | | NM_130386 | |
| 70 | 1373592_AT | AI407094 | Similar to SPI6 (LOC361241), mRNA | XM_341522 | | NM_009256 |
| | | | | | | NM_011452 |
| 71 | 1388945_AT | BM385779 | Similar to 1300014I06Rik protein | XM_341518 | NM_183373 | NM_025831 |
| | | | (LOC361238), mRNA | | | |
| 72 | 1375898_AT | AW252379 | Similar to RNA-binding protein with multiple | XM_341447 | NM_006867 | NM_019733 |
| | | | splicing (RBP-MS) (LOC361161), mRNA | | | |
| 73 | 1381190_AT | AI598833 | Similar to KIAA0858 protein (LOC361084), | XM_341368 | | XM_354836 |
| | | | mRNA | | | |
| 74 | 1371694_AT | AI317805 | Dihydropyrimidinase-like 2 | XM_341342 | NM_001386 | NM_009955 |
| | 1380728_AT | BE118404 | | | | |
| 75 | 1373515_AT | BI275737 | Rattus norvegicus similar to RIKEN cDNA | XM_341259 | NM_014181 | NM_173752 |
| | | | 1110067D22 (LOC360983) | | | |
| 76 | 1389464_AT | BE120953 | Rattus norvegicus similar to multi-PDZ- | XM_341204 | NM_032622 | NM_010727 |
| | | | domain-containing protein (LOC360926), | | | |
| | | | mRNA | | | |
| 77 | 1371250_AT | AI169104 | Similar to Platelet factor 4 precursor (PF-4) | XM_341192 | NM_002619 | NM_019932 |
| | | | (CXCL4) (LOC360918), | | | |
| 78 | 1371447_AT | BG378630 | Rattus norvegicus similar to onzin | XM_341188 | NM_016619 | NM_139198 |
| | | | (LOC360914), mRNA | | | |
| 79 | 1392171_AT | AA945643 | chitinase 3-like 1 (cartilage glycoprotein-39) | XM_341123 | NM_001276 | NM_007695 |
| 80 | 1383949_AT | AI703880 | LOC360817 similar to KIAA1025 protein | XM_341090 | NM_015335 | NM_172424 |
| 81 | 1388750_AT | BF417032 | Rattus norvegicus transferrin receptor (Tfrc) | XM_340999 | NM_003234 | NM_011638 |
| 82 | 1387897_AT | L16532 | cyclic nucleotide phosphodiesterase 1 | XM_340904 | NM_033133 | NM_009923 |
| | 1370693_A_AT | M18630 | | | | |
| 83 | 1388392_AT | BI274159 | Similar to Tax interaction protein 1 | XM_340841 | NM_014604 | NM_029564 |
| | | | (LOC360564), mRNA | | | |
| 84 | 1386833_AT | BF554138 | Rattus norvegicus similar to BAZF | XM_340830 | NM_181844 | NM_007528 |
| | 1394375_X_AT | | (LOC360551), mRNA | | | |
| 85 | 1372610_AT | BI274349 | Rattus norvegicus similar to Prolyl 4- | XM_340798 | NM_004199 | NM_011031 |
| | | | hydroxylase alpha IIa subunit (LOC360526), | | | |
| | | | mRNA | | | |
| 86 | 1370927_AT | BE108345 | Rattus norvegicus similar to Collagen alpha | XM_243912 | NM_004370 | NM_007730 |
| | | | 1(XII) chain precursor (LOC315838), mRNA | | NM_080645 | |
| 87 | 1372518_AT | AI176918 | Rattus norvegicus similar to Fbln1 protein | XM_243637 | NM_006487 | NM_010180 |
| | | | (LOC315191), mRNA | | NM_006485 | |
| | | | | | NM_001996 | |
| | | | | | NM_006486 | |
| 88 | 1388459_AT | AI101782 | collagen, type XVIII, alpha 1 | XM_241632 | NM_030582 | NM_009929 |
| | 1388143_AT | BI288582 | | | NM_130445 | |
| | | | | | NM_130444 | |
| 89 | 1390706_AT | BF392456 | spectrin beta 2 | XM_240072 | NM_003128 | NM_009260 |
| | | | | | NM_178313 | NM_175836 |
| 90 | 1388618_AT | BM389302 | Similar to Nidogen 2 (LOC302248), mRNA | XM_238622 | NM_007361 | NM_008695 |
| 91 | 1374477_AT | BE113700 | Paired related homeobox 2 | XM_238327 | NM_016307 | NM_009116 |
| 92 | 1385444_AT | AI071994 | Similar to Dickkopf related protein-2 | XM_238256 | NM_014421 | NM_020265 |
| | | | precursor (Dkk-2) (Dickkopf-2) (mDkk-2) | | | |
| | | | (LOC295445), mRNA | | | |
| 93 | 1393240_AT | AI112299 | Similar to mutant p53 binding protein 1 | XM_238154 | NM_016938 | NM_021474 |
| | | | (LOC293677), mRNA | | | |
| 94 | 1374678_AT | BE109578 | Similar to Semaphorin 4B (Semaphorin C) | XM_238119 | NM_020210 | NM_013659 |
| | | | (Sema C) (LOC293042), mRNA | | NM_198925 | |
| 95 | 1388131_AT | X03369 | Rat mRNA for beta-tubulin T beta15 | XM_238004 | NM_001069 | NM_009450 |
| 96 | 1388792_AT | AI599423 | Similar to GROWTH ARREST AND DNA- | XM_237999 | NM_006705 | NM_011817 |
| | | | DAMAGE-INDUCIBLE PROTEIN | | | |
| | | | GADD45 GAMMA (LOC291005), mRNA | | | |
| 97 | 1390403_AT | BE108405 | Similar to CG8312-PA (LOC288031), | XM_237838 | NM_153690 | NM_177632 |
| | | | mRNA | | | |
| 98 | 1371596_AT | AI008971 | Rattus norvegicus similar to Rnps1 protein | XM_237758 | NM_006711 | NM_009070 |
| | | | (LOC287113), mRNA | | NM_080594 | |
| 99 | 1367579_A_AT | BI285434 | Rattus norvegicus similar to tubulin, alpha 6; | XM_237718 | NM_032704 | NM_009448 |
| | | | tubulin alpha 6 (LOC300218) | | | |
| 100 | 1388926_AT | AI409048 | Similar to ectonucleotide | XM_236956 | NM_021572 | NM_032003 |
| | | | pyrophosphatase/phosphodiesterase 5 | | | |
| | | | (LOC316249), mRNA | | | |
| 101 | 1381311_AT | BE100625 | Rattus norvegicus similar to cell surface | XM_236797 | NM_001974 | NM_010130 |
| | | | glycoprotein (LOC316137), mRNA | | | |
| 102 | 1379461_AT | BE120147 | Rattus norvegicus similar to zinc finger | XM_236726 | NM_181489 | NM_173364 |
| | | | protein (LOC301076), mRNA | | | |
| 103 | 1371727_AT | AA893350 | Galactosidase, beta 1 | XM_236675 | NM_000404 | NM_009752 |
| 104 | 1383484_AT | AW920193 | Similar to KIAA1585 protein (LOC315959) | XM_236567 | NM_018133 | XM_150227 |
| 105 | 1374849_AT | BF285745 | Similar to a disintegrin and metalloprotease | XM_236471 | NM_014272 | XM_135041 |
| | | | with thrombospondin motifs-7 preproprotein | | | |
| | | | (LOC315879), mRNA | | | |
| 106 | 1374129_AT | AI104518 | similar to AI449441 protein (LOC300793), | XM_236355 | NM_002537 | NM_010952 |
| | | | Rattus norvegicus transcribed sequence with | | | |
| | | | strong similarity to protein sp: O95190 | | | |
| | | | (H. sapiens) OAZ2_HUMAN Ornithine | | | |
| | | | decarboxylase antizyme 2 (ODC-Az 2) (AZ2) | | | |
| 107 | 1374013_AT | BI278078 | Rattus norvegicus similar to complement-c1q | XM_236180 | NM_015645 | |
| | | | tumor necrosis factor-related protein | | | |
| | | | (LOC315598), mRNA | | | |
| 108 | 1388703_AT | BF285026 | Similar to endothelial cell-selective adhesion | XM_236058 | NM_138961 | NM_027102 |
| | | | molecule (LOC300519), mRNA | | | |
| 109 | 1390914_AT | AI071474 | Similar to retrovirus integration site | XM_235979 | NM_002017 | NM_008026 |
| | 1393249_AT | BF407924 | (LOC315532), mRNA | | | |
| 110 | 1389483_AT | AI408686 | Similar to KIAA0877 protein (LOC315496), | XM_235970 | XM_371891 | NM_172920 |
| | | | mRNA | | XM_374422 | |
| 111 | 1371356_AT | BI294836 | Rattus norvegicus similar to Tenc1 protein | XM_235710 | NM_015319 | |
| | | | (LOC315326), mRNA | | NM_170754 | |
| | | | | | NM_198316 | |
| 112 | 1388728_AT | BI282923 | Similar to lysosomal-associated | XM_235393 | NM_018407 | NM_033521 |
| | | | transmembrane protein 4 beta (LOC315047), | | | |
| | | | mRNA | | | |
| 113 | 1374134_AT | BM384057 | Similar to SERINE/THREONINE-PROTEIN | XM_235049 | NM_002595 | NM_146239 |
| | 1394473_AT | AI716206 | KINASE PCTAIRE-2 (LOC314743), mRNA | | | |
| 114 | 1372861_AT | BG380713 | Rattus norvegicus similar to R31449_3 | XM_234925 | NM_012398 | XM_137157 |
| | | | (LOC314641), mRNA | | | |
| 115 | 1373301_AT | BE113133 | Similar to protein phosphatase 1, regulatory | XM_234555 | NM_015316 | NM_011625 |
| | | | (inhibitor) subunit 13B; apoptosis-stimulating | | | |
| | | | protein of p53, 1 (LOC314465), mRNA | | | |
| 116 | 1390782_AT | BI295930 | Similar to RIKEN cDNA 1200003C23 | XM_234194 | NM_175060 | NM_025809 |
| | | | (LOC314148), mRNA | | | |
| 117 | 1372294_AT | BI278571 | Similar to mKIAA0230 protein | XM_234038 | XM_056455 | XM_283052 |
| | 1375862_AT | BM384701 | (LOC314016), mRNA | | | |
| 118 | 1390159_AT | AA819332 | Similar to RAS guanyl releasing protein 3 | XM_233873 | NM_170672 | NM_207246 |
| | | | (calcium and DAG-regulated); guanine | | | |
| | | | nucleotide exchange factor for Rap1 | | | |
| | | | (LOC313874), mRNA | | | |
| 119 | 1373554_AT | BE349698 | Similar to SPRY domain-containing SOCS | XM_233686 | NM_025106 | NM_029035 |
| | | | box protein SSB-1 | | | |
| 120 | 1372569_AT | AI171676 | Similar to four and half LIM domain protein | XM_233499 | NM_004468 | NM_010213 |
| | | | 3 (LOC313582), mRNA | | | |
| 121 | 1389324_AT | BI289129 | Tyrosine kinase receptor 1 | XM_233462 | NM_005424 | NM_011587 |
| 122 | 1397644_AT | BI278369 | Similar to 5-methylthioadenosine | XM_233168 | NM_002451 | NM_024433 |
| | | | phosphorylase (MTA phosphorylase) | | | |
| | | | (MTAPase) (LOC298227), mRNA | | | |
| 123 | 1372127_AT | BI275824 | Similar to ubiquitin-associated protein 2 | XM_232901 | NM_018449 | NM_026872 |
| | | | (LOC313169), mRNA | | NM_020867 | |
| | | | | | NM_148171 | |
| 124 | 1392990_AT | AI031004 | Similar to HMG-box transcription factor | XM_232640 | NM_022454 | NM_011441 |
| | | | (LOC312936), mRNA | | | |
| 125 | 1372088_AT | AI012434 | Similar to protein tyrosine phosphatase, | XM_232536 | NM_003622 | XM_132960 |
| | | | receptor-type, F interacting protein, binding | | NM_177444 | |
| | | | protein 2; liprin beta 2 (LOC312855), mRNA | | | |
| 126 | 1371602_AT | BI274110 | Rattus norvegicus similar to Tetraspan NET- | XM_232372 | NM_006675 | NM_175414 |
| | | | 5 (LOC312728), mRNA | | | |
| 127 | 1393917_AT | BF555488 | Similar to macrophage hemoglobin scavenger | XM_232342 | NM_004244 | NM_053094 |
| | | | receptor CD163 precursor (LOC312701), | | NM_203416 | |
| | | | mRNA | | | |
| 128 | 1389341_AT | AI102248 | Similar to plexin D1; KIAA0620 protein | XM_232283 | NM_015103 | XM_149784 |
| | | | (LOC312652), mRNA | | | |
| 129 | 1389533_AT | AA944398 | Fibulin 2 | XM_232197 | NM_001998 | NM_007992 |
| 130 | 1379847_AT | AW141858 | Similar to Dysferlin (Dystrophy associated | XM_232123 | NM_003494 | NM_021469 |
| | 1392786_AT | BG668812 | fer-1 like protein) (Fer-1 like protein 1) | | | |
| | | | (LOC312492), mRNA | | | |
| 131 | 1376265_AT | AI411542 | Similar to six transmembrane epithelial | XM_231400 | NM_152999 | XM_284053 |
| | | | antigen of prostate 2; six transmembrane | | | |
| | | | prostate protein; prostate cancer associated | | | |
| | | | gene 1; prostate cancer associated protien 1 | | | |
| | | | (LOC312052), mRNA | | | |
| 132 | 1382181_AT | AI555069 | Hypothetical LOC311984 (LOC311984), | XM_231302 | XM_373170 | |
| | | | mRNA | | | |
| 133 | 1381971_AT | AW528157 | Rattus norvegicus similar to Sry-related | XM_230968 | NM_018419 | NM_009236 |
| | | | HMG-box protein (LOC311723), mRNA | | | |
| 134 | 1377023_AT | AI408580 | Similar to dual specificity phosphatase 2 | XM_230575 | NM_004418 | NM_010090 |
| | | | (LOC311406), mRNA | | | |
| 135 | 1377625_AT | BF560079 | Similar to CGI-09 protein (LOC311441), | XM_230548 | NM_015939 | NM_175113 |
| | | | mRNA | | | |
| 136 | 1375420_AT | AI170535 | Similar to endoplasmic reticulum membrane | XM_230296 | NM_006034 | XM_203859 |
| | | | protein with at least 3 transmembrane | | | |
| | | | domains of bilaterial origin like (XB300) | | | |
| | | | (LOC311209), mRNA | | | |
| 137 | 1377595_AT | AI044347 | Similar to mKIAA1927 protein | XM_230036 | NM_006751 | NM_080558 |
| | | | (LOC311146), mRNA | | | |
| 138 | 1373000_AT | AA818334 | Rattus norvegicus similar to sushi-repeat | XM_228444 | NM_014467 | NM_026838 |
| | | | containing protein (LOC317181), mRNA | | | |
| 139 | 1382130_AT | AA997710 | Similar to bA99E24.1.1 (protocadherin 19 | XM_228429 | XM_033173 | XM_205287 |
| | | | (KIAA1313) protein) (LOC317183), mRNA | | | |
| 140 | 1371988_AT | AA892549 | Similar to Man9-mannosidase (LOC294567), | XM_228364 | NM_005907 | NM_008548 |
| | 1383574_AT | AI502956 | mRNA | | | |
| 141 | 1372266_AT | BI281607 | Similar to DNA polymerase zeta catalytic | XM_228273 | NM_002912 | NM_011264 |
| | | | subunit (LOC309812), mRNA | | | |
| 142 | 1389214_AT | BF406693 | Similar to Lama4 protein (LOC309816), | XM_228209 | NM_002290 | NM_010681 |
| | | | mRNA | | | |
| 143 | 1383131_AT | AI177292 | Similar to Integrin beta-2 precursor (Cell | XM_228072 | NM_000211 | NM_008404 |
| | | | surface adhesion glycoproteins LFA- | | | |
| | | | 1/CR3/P150,95 beta-subunit) (CD18) | | | |
| | | | (Complement receptor C3 beta-subunit) | | | |
| | | | (LOC309684), mRNA | | | |
| 144 | 1398397_AT | AI407483 | Similar to Parg1-pending protein | XM_227651 | NM_004815 | NM_172525 |
| | | | (LOC310833), mRNA | | | |
| 145 | 1371924_AT | BI274355 | Similar to RIKEN cDNA 2810002E22 | XM_227535 | NM_020190 | NM_133859 |
| | | | (LOC310743), mRNA | | | |
| 146 | 1377869_AT | BI284261 | Similar to carbon catabolite repression 4 | XM_227134 | NM_012118 | NM_009834 |
| | | | protein homolog (LOC310395), mRNA | | | |
| 147 | 1367538_AT | BF284303 | LOC310258 similar to polyhomeotic 3 | XM_226976 | NM_024947 | NM_153421 |
| 148 | 1384165_AT | BG671473 | Similar to Ras GTPase-activating-like protein | XM_226696 | | |
| | | | IQGAP2 (LOC310008), mRNA | | | |
| 149 | 1376583_AT | BE095847 | Similar to RIKEN cDNA 6430548M08 | XM_226529 | XM_375377 | NM_172286 |
| | | | (LOC307907), mRNA | | | |
| 150 | 1379322_AT | BI289174 | Similar to 26S proteasome non-ATPase | XM_226439 | NM_002811 | NM_010817 |
| | | | regulatory subunit 7 (26S proteasome | | | |
| | | | regulatory subunit S12) (Proteasome subunit | | | |
| | | | p40) (Mov34 protein) (LOC307821), mRNA | | | |
| 151 | 1378282_AT | BI290750 | Similar to casein kinase II, alpha prime | XM_226237 | NM_001896 | NM_009974 |
| | | | subunit (LOC307641), mRNA | | | |
| 152 | 1389255_AT | BE110616 | Similar to VE-cadherin (LOC307618), | XM_226213 | NM_001795 | NM_009868 |
| | | | mRNA | | | |
| 153 | 1391503_AT | BF281952 | Similar to FLJ00007 protein (LOC307482), | XM_225999 | NM_033449 | NM_175684 |
| | | | mRNA | | | |
| 154 | 1374266_AT | AA955773 | Rattus norvegicus similar to protocadherin 1 | XM_225997 | NM_002587 | NM_029357 |
| | | | isoform 2 precursor; protocadherin 42; | | NM_032420 | |
| | | | cadherin-like protein 1 (LOC307481), mRNA | | | |
| 155 | 1377950_AT | AA955213 | LOC307415: similar to interferon-inducible | XM_225909 | | NM_021792 |
| | | | GTPase | | | |
| 156 | 1398664_AT | C06752 | Similar to RIKEN cDNA 9130427A09 | XM_225748 | NM_023927 | NM_026240 |
| | | | (LOC307288), mRNA | | | |
| 157 | 1376640_AT | BF285466 | Similar to hypothetical protein (LOC307138), | XM_225599 | XM_167709 | XM_194000 |
| | | | mRNA | | | |
| 158 | 1392648_AT | BI294018 | Similar to macrophage mannose receptor | XM_225585 | NM_002438 | NM_008625 |
| | | | precursor (LOC291327), mRNA | | | |
| 159 | 1372028_AT | AA893215 | Similar to Protein CGI-117 (Protein | XM_225176 | NM_016391 | NM_178605 |
| | | | HSPC111) (LOC306768), mRNA | | | |
| 160 | 1376788_AT | AA818353 | Rattus norvegicus similar to Dapk1 protein | XM_225138 | NM_004938 | NM_029653 |
| 161 | 1388494_AT | BI281705 | Similar to Collagen alpha 2(IV) chain | XM_225043 | NM_001846 | NM_009932 |
| | | | precursor (LOC306628), mRNA | | | |
| 162 | 1375337_AT | AI011712 | Similar to | XM_224964 | NM_003816 | NM_007404 |
| | | | metalloprotease/disintegrin/cysteine rich | | | |
| | | | protein precursor (LOC290834), mRNA | | | |
| 163 | 1373599_AT | AI599525 | Similar to RNA polymerase III transcription | XM_224944 | NM_018310 | NM_025686 |
| | | | initiation factor BRF2 (LOC306542), mRNA | | | |
| 164 | 1386041_A_AT | BF288243 | Similar to Kruppel-like factor 2 (Lung | XM_224722 | NM_016270 | NM_008452 |
| | | | kruppel-like factor) (LOC306330), mRNA | | | |
| 165 | 1372136_AT | AA848776 | Similar to tetraspanin similar to TM4SF9 | XM_224699 | NM_030927 | NM_145928 |
| | | | (LOC306324), mRNA | | | |
| 166 | 1388401_AT | BI296155 | Similar to actin-binding protein homolog | XM_224561 | NM_001457 | XM_127565 |
| | 1391827_AT | AI013675 | ABP-278 (LOC306204), mRNA | | | |
| 167 | 1370905_AT | BI291229 | Thyroid regulating gene | XM_224538 | NM_015296 | XM_358315 |
| 168 | 1384907_AT | AI411835 | Rattus norvegicus similar to dachshund | XM_224440 | NM_004392 | NM_007826 |
| | | | variant 1 (LOC306096), mRNA | | NM_080759 | |
| | | | | | NM_080760 | |
| 169 | 1384415_AT | AI058292 | LOC290317 similar to mSox7 | XM_224283 | NM_031439 | NM_011446 |
| 170 | 1394597_AT | AI101356 | Similar to RIKEN cDNA 4921528E07 | XM_223930 | NM_030637 | NM_176845 |
| | | | (LOC305816), mRNA | | | |
| 171 | 1386721_AT | BF560938 | Similar to zinc-finger protein NOLZI | XM_223786 | NM_032772 | NM_145459 |
| | | | (LOC305687), mRNA | | | |
| 172 | 1390112_AT | BF284634 | Rattus norvegicus similar to EGF-containing | XM_223725 | NM_004105 | NM_146015 |
| | | | fibulin-like extracellular matrix protein 1 | | NM_018894 | |
| | | | precursor (Fibulin-3) (FIBL-3) (T16 protein) | | | |
| | | | (LOC305604), mRNA | | | |
| 173 | 1372776_AT | BM392017 | Similar to F-box and leucine-rich repeat | XM_223508 | NM_012161 | NM_178729 |
| | | | protein 5 isoform 1; F-box protein FBL5 | | NM_033535 | |
| | | | (LOC305424), mRNA | | | |
| 174 | 1376678_AT | BF283712 | Similar to Amyloid beta A4 precursor | XM_223399 | NM_173075 | NM_009686 |
| | | | protein-binding family B member 2 (Fe65- | | | |
| | | | like protein) (LOC305338), mRNA | | | |
| 175 | 1373751_AT | BF396319 | Similar to Kif21b (LOC289397), mRNA | XM_223090 | XM_371332 | NM_019962 |
| 176 | 1388335_AT | BI285865 | Similar to transgelin 2; SM22-alpha homolog | XM_222906 | NM_003564 | NM_178598 |
| | | | (LOC304983), mRNA | | | |
| 177 | 1374320_AT | AI717113 | coagulation factor 5 | XM_222831 | NM_000130 | NM_007976 |
| 178 | 1389787_AT | AI549469 | similar to Putative alpha-mannosidase | XM_222721 | NM_025191 | XM_193956 |
| | | | C1orf22 | | | |
| 179 | 1389059_AT | BI278651 | Similar to Lyl-1 protein (Lymphoblastic | XM_222472 | NM_005583 | NM_008535 |
| | | | leukemia derived sequence 1) (LOC304663), | | | |
| | | | mRNA | | | |
| 180 | 1371632_AT | AI231166 | Rattus norvegicus similar to slingshot 1 | XM_222275 | NM_014325 | NM_011779 |
| | | | (LOC304580), mRNA | | | |
| 181 | 1390031_AT | BG378798 | Similar to hypothetical protein FLJ14466 | XM_222178 | NM_032790 | NM_175423 |
| | | | (LOC304496), mRNA | | | |
| 182 | 1398335_AT | BI285665 | Similar to tweety homolog 2 (LOC304315), | XM_221962 | XM_166523 | NM_175274 |
| | | | mRNA | | XM_374388 | |
| 183 | 1393460_AT | BM388665 | Similar to GARP protein precursor (Garpin) | XM_221370 | NM_198565 | NM_146069 |
| | | | (Glycoprotein A repetitions predominant) | | | |
| | | | (LOC303875), mRNA | | | |
| 184 | 1389478_AT | BM392031 | Similar to hypothetical protein FLJ14360 | XM_221268 | NM_032775 | NM_145479 |
| | | | (LOC303792), mRNA | | | |
| 185 | 1376182_AT | BM383074 | Similar to fetal Alzheimer antigen isoform 2; | XM_221050 | NM_182641 | XM_126724 |
| | | | bromodomain and PHD domain transcription | | NM_004459 | |
| | | | factor; nucleosome remodeling factor, large | | | |
| | | | subunit; fetal Alz-50 reactive clone 1 | | | |
| | | | (LOC303617), mRNA | | | |
| 186 | 1372615_AT | AI070137 | Amine oxidase, copper containing 3 | XM_220990 | NM_003734 | NM_009675 |
| 187 | 1388151_AT | AI008369 | tumor specific antigen 70 kDa | XM_220167 | NM_024535 | NM_030205 |
| 188 | 1372164_AT | AI600255 | Similar to hypothetical protein FLJ20154 | XM_219958 | NM_017787 | NM_146099 |
| | 1375369_AT | Z83035 | (LOC309456), mRNA | | | |
| 189 | 1389227_AT | BG375902 | Rattus norvegicus similar to GTPase | XM_218977 | NM_001665 | NM_019566 |
| | | | (LOC308875), | | | |
| 190 | 1381305_AT | BM383011 | Similar to RIKEN cDNA 4931406P16 | XM_218503 | NM_014686 | NM_172741 |
| | | | (LOC308509), mRNA | | | |
| 191 | 1372769_AT | AI105290 | Rattus norvegicus similar to Lgtn protein | XM_218452 | NM_006893 | NM_010709 |
| | | | (LOC308432), mRNA | | | |
| 192 | 1376858_AT | BI281836 | Similar to Exocyst complex component Sec6 | XM_218421 | NM_138568 | XM_358663 |
| | | | (rSec6) (LOC308412), mRNA | | | |
| 193 | 1372593_AT | AI137283 | Rattus norvegicus transcribed sequence with | XM_218292 | NM_016325 | NM_022981 |
| | | | moderate similarity to protein | | NM_016324 | |
| | | | ref: NP_057409.1 (H. sapiens) zinc finger | | NM_133502 | |
| | | | protein 274, isoform a; KRAB zinc finger | | | |
| | | | protein HFB101; zinc finger protein zfp2 | | | |
| | | | [Homo sapiens] | | | |
| 194 | 1397272_AT | BI297130 | Similar to KIF13A (LOC308173), mRNA | XM_217893 | NM_022113 | NM_010617 |
| 195 | 1371525_AT | BI277550 | Rattus norvegicus similar to putative | XM_217744 | NM_006598 | NM_011390 |
| | | | potassium-chloride cotransporter-4; KCC4 | | | |
| | | | (LOC308069), mRNA | | | |
| 196 | 1390415_AT | BM392319 | Similar to thyroid hormone receptor | XM_217741 | NM_004237 | XM_127444 |
| | | | interactor 13; thyroid receptor interacting | | | |
| | | | protein 13 (LOC292206), mRNA | | | |
| 197 | 1383201_AT | BI289300 | LOC301443 similar to Bone morphogenetic | XM_217409 | NM_001204 | NM_007561 |
| | | | protein type II receptor | | NM_033346 | |
| 198 | 1372251_AT | BI282892 | Similar to RNA binding motif protein 5 | XM_217263 | NM_005778 | NM_148930 |
| | | | (LOC300996), mRNA | | | |
| 199 | 1373222_AT | AI230271 | Rattus norvegicus Hexose aminidase A | XM_217144 | NM_000520 | NM_010421 |
| | | | (alpha polypeptide) (Hexa), mRNA | | | |
| 200 | 1388495_AT | BI303583 | Similar to HPI alpha protein (LOC300266), | XM_217062 | NM_012117 | NM_007626 |
| | | | mRNA | | | |
| 201 | 1371360_AT | BM384099 | Rattus norvegicus similar to cytoplasmic | XM_216923 | NM_006096 | NM_010884 |
| | 1391458_AT | BE120446 | protein Ndr1 (LOC299923), mRNA | | | |
| 202 | 1388695_AT | BF285150 | Similar to serine hydroxymethyl transferase 2 | XM_216909 | NM_005412 | NM_028230 |
| | | | (mitochondrial) (LOC299857), mRNA | | | |
| 203 | 1371894_AT | AI409037 | Similar to N-acetylglucosamine-6-sulfatase | XM_216899 | NM_002076 | NM_029364 |
| | 1395265_AT | BF419323 | precursor (G6S) (Glucosamine-6-sulfatase) | | | |
| | | | (LOC299825), mRNA | | | |
| 204 | 1398962_AT | BM388453 | Similar to cDNA sequence BC005632 | XM_216853 | NM_031213 | NM_145421 |
| | | | (LOC299617), mRNA | | | |
| 205 | 1388773_AT | BI278479 | Similar to Tnfaip2 protein (LOC299340), | XM_216786 | NM_006291 | NM_009396 |
| | | | mRNA | | | |
| 206 | 1389617_AT | BM392135 | Similar to NimA-related protein kinase | XM_216755 | NM_033116 | NM_145138 |
| | | | (LOC299204), mRNA | | | |
| 207 | 1372835_AT | BM389644 | Similar to ras homolog gene family, member | XM_216737 | NM_020663 | NM_023275 |
| | | | J; TC10-like Rho GTPase (LOC299145), | | | |
| | | | mRNA | | | |
| 208 | 1383662_AT | AI043759 | Similar to angiopoietin-related protein 5 | XM_216613 | NM_031917 | NM_145154 |
| | | | (LOC298698), mRNA | | | |
| 209 | 1371487_AT | BG666075 | Similar to SH3 domain binding glutamic | XM_216547 | NM_031286 | NM_080559 |
| | | | acid-rich protein-like 3 (LOC298544), | | | |
| | | | mRNA | | | |
| 210 | 1388397_AT | BI281160 | EBNA1 binding protein 2 | XM_216516 | NM_006824 | NM_026932 |
| 211 | 1382680_AT | BG673602 | Adipose differentiation-related protein | XM_216438 | NM_001122 | NM_007408 |
| | 1390383_AT | BI285616 | | | | |
| 212 | 1379345_AT | BM386752 | Rattus norvegicus similar to type XV | XM_216399 | NM_001855 | NM_009928 |
| | | | collagen (LOC298069), mRNA | | | |
| 213 | 1393706_AT | BE109939 | Similar to six transmembrane epithelial | XM_216315 | NM_012449 | NM_027399 |
| | | | antigen of the prostate (LOC297738), mRNA | | | |
| 214 | 1371641_AT | BM388827 | Rattus norvegicus similar to CCTeta, eta | XM_216180 | NM_006429 | NM_007638 |
| | | | subunit of the chaperonin containing TCP-1 | | | |
| | | | (CCT) (LOC297406), mRNA | | | |
| 215 | 1388715_AT | AA818089 | Rattus norvegicus similar to Glycyl-tRNA | XM_216152 | NM_002047 | NM_180678 |
| | 1395730_AT | AW507352 | synthetase (LOC297113), mRNA | | | |
| 216 | 1371691_AT | BI282993 | Similar to CHO functionally unknown type II | XM_216142 | NM_002889 | NM_027852 |
| | | | transmembrane protein (LOC297073), | | | |
| | | | mRNA | | | |
| 217 | 1373590_AT | BI295949 | Similar to erthyrocyte band 7 integral | XM_216045 | NM_004099 | NM_013515 |
| | | | membrane protein, protein 7.2B, stomatin | | NM_198194 | |
| | | | (LOC296655), mRNA | | | |
| 218 | 1383240_AT | BE110753 | Integrin, alpha 6 | XM_215984 | NM_000210 | NM_008397 |
| 219 | 1391435_AT | BI278687 | Similar to phospholipid transfer protein | XM_215939 | NM_006227 | NM_011125 |
| | | | (LOC296371), mRNA | | NM_182676 | |
| 220 | 1371507_AT | BM384151 | Similar to 4921517L17Rik protein | XM_215902 | NM_080834 | XM_130609 |
| | | | (LOC296311), mRNA | | | |
| 221 | 1373615_AT | BM391538 | Rattus norvegicus similar to frezzled | XM_215757 | NM_001463 | NM_011356 |
| | | | (LOC295691), mRNA | | | |
| 222 | 1377369_AT | BF419070 | Rattus norvegicus similar to duodenal | XM_215749 | NM_024843 | XM_130253 |
| | | | cytochrome b (LOC295669), mRNA | | | |
| 223 | 1374061_AT | BF282054 | Similar to RIKEN cDNA 1110055L24 | XM_215741 | NM_014880 | NM_025422 |
| | | | (LOC295629), mRNA | | | |
| 224 | 1372587_AT | AI172271 | Rattus norvegicus similar to endomucin-1 | XM_215710 | NM_016242 | NM_016885 |
| | | | (LOC295490), mRNA | | | |
| 225 | 1372613_AT | AI232784 | Rattus norvegicus similar to RIKEN cDNA | XM_215705 | NM_020139 | NM_027208 |
| | | | 1810026B04 (LOC295458), mRNA | | | |
| 226 | 1373847_AT | AW435343 | Rattus norvegicus similar to L6 antigen | XM_215576 | NM_014220 | NM_008536 |
| | | | (LOC295061), mRNA | | | |
| 227 | 1378305_AT | AI578087 | Similar to L6 antigen (LOC295061), mRNA | XM_215576 | NM_014220 | NM_008536 |
| 228 | 1389476_AT | AI010238 | Similar to RIKEN cDNA D630029K19 | XM_215551 | XM_371706 | NM_172680 |
| | | | (LOC294978), mRNA | | | |
| 229 | 1388119_AT | BM392140 | Rattus norvegicus transcribed sequence with | XM_215530 | NM_015716 | NM_016713 |
| | | | moderate similarity to protein | | | |
| | | | ref: NP_056531.1 (H. sapiens) | | | |
| | | | Misshapen/NIK-related kinase [Homo | | | |
| | | | sapiens] | | | |
| 230 | 1398970_AT | BI303004 | Similar to Ab2-292 (LOC294912), mRNA | XM_215528 | NM_003262 | NM_027016 |
| 231 | 1376570_AT | BF417274 | Chaperonin containing TCP1, subunit 5 | XM_215516 | NM_012073 | NM_007637 |
| | | | (epsilon) | | | |
| 232 | 1395508_AT | BG665309 | Chaperonin containing TCP1, subunit 5 | XM_215516 | NM_012073 | NM_007637 |
| | | | (epsilon) | | | |
| 233 | 1388155_AT | BI286012 | similar to cytokeratin (LOC294853) | XM_215513 | NM_000224 | NM_010664 |
| | | | | | NM_199187 | |
| 234 | 1371926_AT | AI171807 | Interleukin 6 signal transducer | XM_215483 | NM_002184 | NM_010560 |
| | | | | | NM_175767 | |
| 235 | 1373363_AT | BI281702 | similar to Microtubule-associated protein 1b | XM_215469 | NM_005909 | NM_008634 |
| | 1395357_AT | BG672052 | | | NM_032010 | |
| 236 | 1388054_A_AT | AF072892 | chondroitin sulfate proteoglycan 2 (versican) | XM_215451 | NM_004385 | NM_019389 |
| | 1371232_A_AT | AF084544 | | | | |
| 237 | 1371349_AT | AI598402 | Rattus norvegicus similar to collagen alpha1 | XM_215375 | NM_001848 | NM_009933 |
| | | | type VI-precursor (LOC294337), mRNA | | | |
| 238 | 1382008_AT | AI044348 | Similar to hypothetical protein FLJ11218 | XM_215279 | NM_018363 | |
| | | | (LOC294098), mRNA | | | |
| 239 | 1390406_AT | AI232065 | Similar to Arhgap18 protein (LOC293947), | XM_215247 | NM_033515 | NM_176837 |
| | 1390420_AT | AI577849 | mRNA | | | |
| 240 | 1374034_AT | BG379410 | Similar to cysteine-tRNA ligase isoform b; | XM_215134 | NM_139273 | NM_013742 |
| | 1376754_AT | AI176610 | cysteine translase; cysteine-tRNA synthetase | | NM_001751 | |
| | | | (LOC293638), mRNA | | | |
| 241 | 1397674_AT | AI502930 | Similar to RIKEN cDNA 3230401O13 | XM_215080 | NM_003752 | NM_146200 |
| | | | (LOC293484), mRNA | | | |
| 242 | 1380688_AT | BI283107 | Hypothetical LOC293181 (LOC293181), | XM_215017 | NM_198516 | NM_173739 |
| | | | mRNA | | | |
| 243 | 1388970_AT | AA848597 | Similar to hypothetical protein FLJ20401 | XM_214916 | NM_017805 | NM_027253 |
| | | | (LOC292912), mRNA | | | |
| 244 | 1398873_AT | BI279608 | heterogeneous nuclear ribonucleoprotein L | XM_214878 | NM_001533 | NM_177301 |
| 245 | 1371341_AT | BG372885 | Rattus norvegicus similar to Small nuclear | XM_214847 | NM_004597 | XM_133225 |
| | | | ribonucleoprotein Sm D2 (snRNP core | | NM_177542 | |
| | | | protein D2) (Sm-D2) (LOC309893), mRNA | | | |
| 246 | 1375657_AT | BE107438 | similar to 4933407C03Rik protein | XM_214698 | NM_030629 | BC028834 |
| | | | (LOC292051) | | NM_198390 | |
| 247 | 1371246_AT | BE109988 | Rattus norvegicus similar to Nuclear | XM_214670 | NM_005796 | NM_026532 |
| | | | transport factor 2 (NTF-2) (Placental protein | | | |
| | | | 15) (PP15) (LOC291981), mRNA | | | |
| 248 | 1398356_AT | BF420718 | Rattus norvegicus similar to cleavage and | XM_214640 | NM_007006 | NM_026623 |
| | | | polyadenylation specific factor 5; cleavage | | | |
| | | | and polyadenylation specific factor 5, 25 kD | | | |
| | | | subunit (LOC291877), mRNA | | | |
| 249 | 1383073_AT | BG666028 | Similar to ubiquitin specific protease 14 | XM_214624 | NM_005151 | NM_021522 |
| | | | (LOC291796), mRNA | | | |
| 250 | 1388170_AT | BI285710 | vitamin A-deficient testicular protein 5 | XM_214617 | NM_198991 | |
| 251 | 1374479_AT | BM390240 | Similar to RIKEN cDNA D030070L09 | XM_214605 | NM_194281 | NM_172625 |
| | | | (LOC291737), | | | |
| 252 | 1374118_AT | AA944176 | Rattus norvegicus similar to leucyl-tRNA | XM_214566 | NM_020117 | |
| | | | synthetase (LOC291624), mRNA | | | |
| 253 | 1398893_AT | BG671512 | Similar to Nedd4 WW domain-binding | XM_214564 | NM_030571 | XM_128893 |
| | | | protein 5 (LOC291609), mRNA | | | |
| 254 | 1378134_AT | BI291629 | similar to ATPase, Class I, type 8B, member 1 | XM_214553 | NM_005603 | NM_001001488 |
| 255 | 1375267_AT | BI291292 | Rattus norvegicus similar to Chain A, | XM_214534 | NM_000943 | NM_008908 |
| | | | Cyclophilin C Complexed With Cyclosporin | | | |
| | | | A (LOC291463), mRNA | | | |
| 256 | 1393210_AT | AI070116 | Similar to Extracellular matrix protein 2 | XM_214443 | NM_001393 | |
| | | | precursor (Matrix glycoprotein SC1/ECM2) | | | |
| | | | (LOC291018), mRNA | | | |
| 257 | 1372439_AT | AI176393 | similar to collagen alpha 1(IV) chain | XM_214400 | NM_001845 | NM_009931 |
| | 1373245_AT | BE111752 | precursor | | | |
| 258 | 1389103_AT | BM391005 | Similar to RIKEN cDNA 2810037C03 | XM_214380 | NM_032336 | NM_024240 |
| | | | (LOC290842), mRNA | | | |
| 259 | 1372919_AT | AA851904 | Similar to Putative lysophosphatidic acid | XM_214379 | NM_178819 | NM_018743 |
| | | | acyltransferase (LOC290843), mRNA | | | |
| 260 | 1372050_AT | BI279577 | Similar to hypothetical protein MGC38524 | XM_214295 | NM_024656 | NM_146211 |
| | | | (LOC290637), mRNA | | | |
| 261 | 1374247_AT | BI279869 | Similar to stabilin-1 (LOC290559), mRNA | XM_214279 | NM_015136 | NM_138672 |
| 262 | 1371849_AT | BI285551 | Similar to hypothetical protein FLJ12442 | XM_214258 | NM_022908 | XM_354801 |
| | | | (LOC290558), mRNA | | | |
| 263 | 1371320_AT | BF282337 | Rattus norvegicus similar to E25B protein | XM_214228 | NM_021999 | NM_008410 |
| | | | (LOC290364), mRNA | | | |
| 264 | 1380474_AT | AI013470 | Similar to Lysyl oxidase homolog 2 precursor | XM_214225 | NM_002318 | NM_033325 |
| | 1391450_AT | AI044651 | (Lysyl oxidase-like protein 2) (Lysyl oxidase | | | |
| | | | related protein 2) (Lysyl oxidase-related | | | |
| | | | protein WS9-14) (LOC290350), mRNA | | | |
| 265 | 1373346_AT | AI009492 | Similar to hypothetical protein CL25084 | XM_214121 | NM_015701 | NM_025745 |
| | | | (LOC289874), mRNA | | | |
| 266 | 1375424_AT | BE107525 | Similar to actin-related protein 2 | XM_214109 | NM_005722 | NM_146243 |
| | 1398626_S_AT | AW920881 | (LOC289820) | | | |
| 267 | 1383824_AT | AI029445 | Similar to LIM domain binding 2; LIM | XM_214054 | NM_001290 | NM_010698 |
| | | | binding domain 2; LIM domain-binding | | | |
| | | | factor-2 (LOC289664), mRNA | | | |
| 268 | 1398994_AT | BI301193 | Similar to tyrosylprotein sulfotransferase-2; | XM_213800 | NM_003595 | NM_009419 |
| | | | TPST-2 (LOC288719), mRNA | | | |
| 269 | 1371498_AT | AI412685 | Rattus norvegicus similar to Jtv1-pending | XM_213710 | NM_006303 | NM_146165 |
| | | | protein (LOC288480), mRNA | | | |
| 270 | 1377390_AT | BG381587 | Similar to beta-site APP-cleaving enzyme 2 | XM_213642 | NM_012105 | NM_019517 |
| | | | isoform A preproprotein; beta secretase 2; | | NM_138992 | |
| | | | aspartyl protease 1; membrane-associated | | NM_138991 | |
| | | | aspartic protease 1; memapsin-1; Down | | | |
| | | | syndrome region aspartic protease; 56 kDa | | | |
| | | | aspartic-like protease; | | | |
| 271 | 1389040_AT | AI170825 | Similar to PEST-containing nuclear protein | XM_213636 | NM_020357 | XM_132579 |
| | | | (LOC288165), mRNA | | | XM_358858 |
| 272 | 1371541_AT | AI177055 | Rattus norvegicus similar to Mylk protein | XM_213611 | NM_005965 | NM_139300 |
| | | | (LOC288057), mRNA | | NM_053025 | |
| | | | | | NM_053026 | |
| | | | | | NM_053027 | |
| | | | | | NM_053028 | |
| | | | | | NM_053029 | |
| | | | | | NM_053030 | |
| | | | | | NM_053031 | |
| | | | | | NM_053032 | |
| 273 | 1389123_AT | BE095824 | Similar to Small inducible cytokine A6 | XM_213553 | NM_004166 | NM_009139 |
| | | | precursor (CCL6) (C10 protein) | | NM_032962 | |
| | | | (LOC287910), mRNA | | NM_032963 | |
| 274 | 1374897_AT | AI406842 | Similar to ALY (LOC287878), mRNA | XM_213542 | NM_005782 | NM_011568 |
| 275 | 1371790_AT | BI277658 | Similar to mitochondrial ribosomal protein | XM_213446 | NM_032351 | NM_025927 |
| | | | L45 (LOC287656), mRNA | | | |
| 276 | 1379935_AT | BF419899 | Similar to Small inducible cytokine A7 | XM_213419 | | NM_013654 |
| | | | precursor (CCL7) (Monocyte chemotactic | | | |
| | | | protein 3) (MCP-3) (Monocyte | | | |
| | | | chemoattractant protein 3) (LOC287561), | | | |
| | | | mRNA | | | |
| 277 | 1371329_AT | BI283681 | Rattus norvegicus similar to Eukaryotic | XM_213368 | NM_001970 | NM_181582 |
| | | | translation initiation factor 5A (eIF-5A) (eIF- | | | |
| | | | 4D) (Rev-binding factor) (LOC363547), | | | |
| | | | mRNA | | | |
| 278 | 1375368_AT | BF420654 | Similar to RIKEN cDNA 1700012G19 gene | XM_213235 | XM_208887 | NM_025954 |
| | | | (LOC287115), mRNA | | | |
| 279 | 1389470_AT | AI639117 | B-factor, properdin | NM_212466 | NM_001710 | NM_008198 |
| 280 | 1371392_AT | BI283882 | Rattus norvegicus glucose phosphate | NM_207592 | NM_000175 | NM_008155 |
| | | | isomerase | | | |
| 281 | 1372068_AT | BI289543 | BS69 protein | NM_203366 | NM_006624 | NM_144516 |
| | | | | NM_203367 | NM_212479 | |
| | | | | NM_203368 | | |
| | | | | NM_203369 | | |
| 282 | 1388699_AT | BE114558 | Similar to mannosidase 2, alpha B1 | NM_199404 | NM_000528 | NM_010764 |
| 283 | 1372254_AT | AW915763 | serine (or cysteine) proteinase inhibitor, clade | NM_199093 | NM_000062 | NM_009776 |
| | | | G (C1 inhibitor), member 1, (angioedema, | | | |
| | | | hereditary) | | | |
| 284 | 1388878_AT | BI279756 | LOC291996: Nin one binding protein | NM_199086 | NM_014062 | NM_026277 |
| 285 | 1392280_AT | AW526982 | toll-like receptor 2 variant 1 | NM_198769 | NM_003264 | NM_011905 |
| 286 | 1394483_AT | AW535310 | A disintegrin-like and metalloprotease | NM_198761 | NM_007038 | NM_011782 |
| | | | (reprolysin type) with thrombospondin type 1 | | | |
| | | | motif, 5 (aggrecanase-2) | | | |
| 287 | 1390738_AT | BM385476 | DAMP-1 protein | NM_198134 | NM_004335 | NM_198095 |
| 288 | 1388889_AT | AI598417 | Putative aminopeptidase Fxna | NM_184050 | NM_024896 | XM_358328 |
| | | | | | XM_291315 | |
| 289 | 1384183_AT | AA996869 | Myosin VIIA and Rab interacting protein | NM_182844 | NM_015460 | NM_144557 |
| 290 | 1384392_AT | BF397093 | cytochrome P450, family 26, subfamily b, | NM_181087 | NM_019885 | NM_175475 |
| | | | polypeptide 1 | | | |
| 291 | 1389207_AT | BI282122 | EGL nine homolog 1 (C. elegans) | NM_178334 | NM_022051 | NM_053207 |
| 292 | 1382431_AT | AI103530 | ATP-binding cassette, sub-family A (ABC1), | NM_178095 | NM_005502 | NM_013454 |
| | 1384381_AT | BF284523 | member 1 | | | |
| | 1394490_AT | AI502114 | | | | |
| 293 | 1388218_AT | X13722 | low density lipoprotein receptor | NM_175762 | NM_000527 | NM_010700 |
| 294 | 1371079_AT | X73371 | low affinity immunoglobulin gamma FC | NM_175756 | NM_004001 | NM_010187 |
| | | | region receptor II precursor | | | |
| 295 | 1398332_AT | BM386294 | Protein phosphatase 1F (PP2C domain | NM_175755 | NM_014634 | NM_176833 |
| | | | containing) | | | |
| 296 | 1370442_AT | U25684 | thymosin beta-like protein | NM_173313 | NM_194324 | NM_207267 |
| | | | | | NM_021992 | |
| 297 | 1370408_AT | AF313411 | putative small membrane protein NID67 | NM_173126 | NM_032947 | NM_134133 |
| 298 | 1398303_S_AT | BI295970 | tropomyosin isoform 6 | NM_173111 | NM_152263 | XM_355057 |
| | | | | | NM_153649 | NM_022314 |
| 299 | 1370290_AT | AB011679 | tubulin, beta 5 | NM_173102 | NM_178014 | NM_011655 |
| | 1387892_AT | BI285440 | | | | |
| 300 | 1398857_AT | AF182952 | Surfeit 1 | NM_172068 | NM_003172 | NM_013677 |
| 301 | 1370312_AT | M88469 | f-spondin | NM_172067 | NM_006108 | NM_145584 |
| 302 | 1370954_AT | BI274401 | prolyl 4-hydroxylase alpha subunit | NM_172062 | NM_000917 | NM_011030 |
| 303 | 1370845_AT | BI288690 | ectonucleoside triphosphate | NM_172030 | NM_203468 | NM_009849 |
| | | | diphosphohydrolase 2 | | NM_001246 | |
| 304 | 1375074_AT | BE113756 | ProSAPiP2 protein | NM_172021 | XM_375469 | NM_198100 |
| 305 | 1370838_S_AT | BM387423 | alpha-spectrin 2 | NM_171983 | NM_003127 | XM_355324 |
| 306 | 1392489_AT | BE108956 | Prrx1 paired related homeobox 1 | NM_153821 | NM_006902 | NM_011127 |
| | | | | | NM_022716 | |
| 307 | 1371600_AT | AA943815 | protein kinase inhibitor, gamma | NM_153469 | NM_181805 | NM_011106 |
| | | | | | NM_007066 | |
| | | | | | NM_181804 | |
| 308 | 1383469_AT | BG377269 | Aldehyde dehydrogenase family 1, subfamily | NM_153300 | NM_000693 | NM_053080 |
| | | | A3 | | | |
| 309 | 1388711_AT | BF282650 | Interleukin 13 receptor, alpha 1 | NM_145789 | NM_001560 | NM_133990 |
| 310 | 1370809_AT | AB015946 | tubulin, gamma 1 | NM_145778 | NM_001070 | NM_134024 |
| 311 | 1380617_AT | BI294743 | immune associated nucleotide 4 like 1 | NM_145680 | NM_018384 | NM_175035 |
| | | | (mouse) | | | |
| 312 | 1370570_AT | AF016296 | neuropilin | NM_145098 | NM_003873 | NM_008737 |
| 313 | 1370828_AT | AF228917 | zinc finger, DHHC domain containing 2 | NM_145096 | NM_016353 | NM_178395 |
| 314 | 1374399_AT | AA801107 | pincher | NM_139324 | NM_139265 | NM_133838 |
| | 1375739_AT | BF395171 | | | | |
| 315 | 1371618_S_AT | AI229029 | tubulin, beta 3 | NM_139254 | NM_006086 | NM_023279 |
| 316 | 1370854_AT | AA799423 | LOC246172: Nexilin | NM_139230 | NM_144573 | NM_199465 |
| | | | | NM_139231 | | |
| 317 | 1393427_S_AT | BF287675 | LOC245963: Estrogen-regulated protein | NM_139104 | NM_016215 | NM_178444 |
| | | | CBL20, 20.4 kD | | NM_201446 | NM_198724 |
| | | | | | | NM_198725 |
| 318 | 1370950_AT | AW253995 | ER transmembrane protein Dri 42 | NM_138905 | NM_003713 | NM_080555 |
| | 1372101_AT | AI177031 | | | NM_177414 | |
| 319 | 1387893_AT | D88250 | complement component 1, s subcomponent | NM_138900 | NM_001734 | NM_173864 |
| | | | | | NM_201442 | |
| 320 | 1391442_AT | AA957585 | EH-domain containing 3 | NM_138890 | NM_014600 | NM_020578 |
| 321 | 1369425_AT | AF494095 | cadherin 13 | NM_138889 | NM_001257 | NM_019707 |
| | 1375719_S_AT | BG381748 | | | | |
| 322 | 1369422_AT | AF493782 | fibroblast activation protein | NM_138850 | NM_004460 | NM_007986 |
| 323 | 1369895_S_AT | AF109393 | podocalyxin-like | NM_138848 | NM_005397 | NM_013723 |
| 324 | 1381996_AT | BG666712 | glycoprotein m6b | NM_138846 | NM_005278 | NM_023122 |
| 325 | 1370862_AT | J02582 | apolipoprotein E | NM_138828 | NM_000041 | NM_009696 |
| 326 | 1390311_AT | AW528602 | Tubulin tyrosine ligase | NM_138536 | NM_153712 | NM_027192 |
| 327 | 1389297_AT | AI146215 | ERO1-like (S. cerevisiae) (Ero11), | NM_138528 | NM_014584 | NM_015774 |
| 328 | 1370633_AT | D87927 | cytokine-induced neutrophil chemoattractant-2 | NM_138522 | ****** | NM_203320 |
| | 1370634_X_AT | | | ******* | NM_001511 | ****** |
| | 1388032_A_AT | | | NM_053647 | NM_002090 | NM_009140 |
| 329 | 1370328_AT | AF245040 | dickkopf homolog 3 (Xenopus laevis) | NM_138519 | NM_013253 | NM_015814 |
| | | | | | NM_015881 | |
| 330 | 1387896_AT | M62763 | sterol carrier protein 2 | NM_138508 | NM_002979 | NM_011327 |
| 331 | 1368200_AT | AF030358 | chemokine (C—X3—C motif) ligand 1 | NM_134455 | NM_002996 | NM_009142 |
| 332 | 1369955_AT | AF272662 | Collagen, type V, alpha 1 | NM_134452 | NM_000093 | NM_015734 |
| | 1376099_AT | AW254017 | | | | |
| 333 | 1387630_AT | AB071985 | fatty acid elongase 1 | NM_134382 | NM_021814 | NM_134255 |
| | 1388348_AT | BI278590 | | | | |
| 334 | 1368771_AT | AF230072 | sulfatase FP | NM_134378 | NM_015170 | NM_172294 |
| | 1373062_AT | BM388650 | | | | |
| 335 | 1368332_AT | M80367 | guanylate binding protein 2, interferon- | NM_133624 | XM_375746 | NM_010260 |
| | | | inducible | | | |
| 336 | 1388534_AT | AA851369 | Solute carrier family 31 (copper transporters), | NM_133600 | NM_001859 | NM_175090 |
| | | | member 1 | | | |
| 337 | 1389157_AT | BI275583 | Lectin, galactoside-binding, soluble, 2 | NM_133599 | NM_006498 | NM_025622 |
| | | | (galectin 2) | | | |
| 338 | 1367716_AT | AF480856 | hypothetical protein CDA08 | NM_133557 | NM_030790 | NM_028007 |
| 339 | 1377671_AT | BI279816 | Csf2rb1: colony stimulating factor 2 receptor, | NM_133555 | NM_000395 | NM_007780 |
| | | | beta 1, low-affinity (granulocyte- | | | |
| | | | macrophage) | | | |
| 340 | 1368052_AT | Y13275 | transmembrane 4 superfamily member 3 | NM_133526 | NM_004616 | NM_146010 |
| 341 | 1368657_AT | X02601 | matrix metalloproteinase 3 | NM_133523 | NM_002422 | NM_010809 |
| 342 | 1387789_AT | AB031088 | v-ets erythroblastosis virus E26 oncogene | NM_133397 | NM_182918 | NM_133659 |
| | | | like (avian) | | NM_004449 | |
| 343 | 1387870_AT | AB025017 | zinc finger protein 36 | NM_133290 | NM_003407 | NM_011756 |
| 344 | 1383785_AT | BE115061 | Lymphoid enhancer binding factor 1 | NM_130429 | NM_016269 | NM_010703 |
| 345 | 1370613_S_AT | AF461738 | UDP glycosyltransferase 1 family, | NM_130407 | NM_001072 | NM_145079 |
| | | | polypeptide A7 | | NM_205862 | |
| 346 | 1370062_AT | AY062253 | hypoxia induced gene 1 | NM_080902 | XM_374981 | NM_019814 |
| 347 | 1367786_AT | D10729 | proteosome (prosome, macropain) subunit, | NM_080767 | NM_004159 | NM_010724 |
| | | | beta type 8 | | NM_148919 | |
| 348 | 1373957_AT | BF281544 | reelin | NM_080394 | NM_005045 | NM_011261 |
| | | | | | NM_173054 | |
| 349 | 1372727_AT | BM384088 | Rattus norvegicus cytokine inducible SH2- | NM_058208 | NM_003877 | NM_007706 |
| | | | containing protein 2 | | | |
| 350 | 1387759_S_AT | J02612 | UDP glycosyltransferase 1 family, | NM_057105 | NM_001072 | NM_145079 |
| | | | polypeptide A6 | | NM_205862 | |
| 351 | 1369735_AT | D42148 | Growth arrest specific 6 | NM_057100 | NM_000820 | NM_019521 |
| | 1392784_AT | BF555051 | | | | |
| 352 | 1387294_AT | AB027562 | SH3-domain binding protein 5 (BTK- | NM_054011 | NM_004844 | NM_011894 |
| | | | associated) | | | |
| 353 | 1370048_AT | AF014418 | endothelial differentiation, lysophosphatidic | NM_053936 | NM_001401 | NM_010336 |
| | | | acid G-protein-coupled receptor, 2 | | NM_057159 | |
| 354 | 1368097_A_AT | U17604 | reticulon 1 | NM_053865 | NM_021136 | NM_153457 |
| | | | | | NM_206852 | |
| | | | | | NM_206857 | |
| 355 | 1370007_AT | M86870 | protein disulfide isomerase related protein | NM_053849 | NM_004911 | |
| | | | (calcium-binding protein, intestinal-related) | | | |
| 356 | 1367850_AT | M64370 | Fc receptor, IgG, low affinity III | NM_053843 | NM_021642 | NM_010188 |
| | 1398246_S_AT | | | | | |
| 357 | 1398765_AT | M23674 | adaptor-related protein complex 2, mu 1 | NM_053837 | NM_004068 | NM_009679 |
| | | | subunit | | | |
| 358 | 1367807_AT | L25331 | procollagen-lysine, 2-oxoglutarate 5- | NM_053827 | NM_000302 | NM_011122 |
| | | | dioxygenase (lysine hydroxylase, Ehlers- | | | |
| | | | Danlos syndrome type VI) | | | |
| 359 | 1368079_AT | L22294 | pyruvate dehydrogenase kinase 1 | NM_053826 | NM_002610 | NM_172665 |
| 360 | 1387001_AT | L19699 | v-ral simian leukemia viral oncogene | NM_053821 | NM_002881 | NM_022327 |
| | | | homolog B | | | |
| 361 | 1387976_AT | AF294257 | solute carrier family 9 (sodium/hydrogen | NM_053811 | NM_004785 | NM_023055 |
| | | | exchanger), isoform 3 regulator 2 | | | NM_023449 |
| 362 | 1387279_AT | AF276998 | junctional adhesion molecule 1 | NM_053796 | NM_016946 | NM_172647 |
| | | | | | NM_144501 | |
| | | | | | NM_144502 | |
| | | | | | NM_144503 | |
| | | | | | NM_144504 | |
| 363 | 1369956_AT | AF201901 | interferon gamma receptor | NM_053783 | NM_000416 | NM_010511 |
| 364 | 1387018_AT | AF026505 | Arg/Abl-interacting protein ArgBP2 | NM_053770 | NM_003603 | NM_172752 |
| | | | | | NM_021069 | |
| 365 | 1367453_AT | D26564 | cell division cycle 37 homolog (S. cerevisiae) | NM_053743 | NM_007065 | NM_016742 |
| 366 | 1372638_AT | BG371454 | PAK-interacting exchange factor beta | NM_053740 | NM_003899 | NM_017402 |
| | | | | | NM_145735 | |
| 367 | 1393883_AT | AI556852 | Rattus norvegicus fms-related tyrosine kinase | NM_053652 | NM_182925 | NM_008029 |
| | | | | | NM_002020 | |
| 368 | 1387306_A_AT | AB032420 | early growth response 2 | NM_053633 | NM_000399 | NM_010118 |
| 369 | 1387282_AT | AF314540 | crystallin, alpha C | NM_053612 | NM_014365 | NM_030704 |
| 370 | 1372844_AT | AW531877 | ephrin A1 | NM_053599 | NM_004428 | NM_010107 |
| | | | | | NM_182685 | |
| 371 | 1367801_AT | D29683 | endothelin converting enzyme 1 | NM_053596 | NM_001397 | NM_199307 |
| 372 | 1369950_AT | L11007 | cyclin-dependent kinase 4 | NM_053593 | NM_000075 | NM_009870 |
| 373 | 1368281_AT | L07316 | dipeptidase 1 | NM_053591 | NM_004413 | NM_007876 |
| 374 | 1387125_AT | L18948 | S100 calcium-binding protein A9 | NM_053587 | NM_002965 | NM_009114 |
| | | | (calgranulin B) | | | |
| 375 | 1398833_AT | AF094821 | membrane-bound transcription factor | NM_053569 | NM_003791 | NM_019709 |
| | | | protease, site 1 | | NM_201268 | |
| 376 | 1398840_AT | AF054826 | vesicle-associated membrane protein 5 | NM_053555 | NM_006634 | NM_016872 |
| 377 | 1368103_AT | AJ303374 | ATP-binding cassette, sub-family G | NM_053502 | NM_004915 | NM_009593 |
| | | | (WHITE), member 1 | | NM_016818 | |
| 378 | 1367661_AT | AF140232 | calcium binding protein A6 (calcyclin) | NM_053485 | NM_014624 | NM_011313 |
| 379 | 1370963_AT | AJ131902 | growth arrest specific 7 | NM_053484 | NM_003644 | NM_008088 |
| | | | | | NM_201432 | |
| | | | | | NM_201433 | |
| 380 | 1383516_AT | BG663284 | Fibrinogen-like 2 | NM_053455 | NM_006682 | NM_008013 |
| 381 | 1367590_AT | AF306457 | RAN, member RAS oncogene family | NM_053439 | NM_006325 | NM_009391 |
| 382 | 1387805_AT | AF243515 | BCL2/adenovirus E1B 19 kDa-interacting | NM_053420 | NM_004052 | NM_009760 |
| | | | protein 3, nuclear gene for mitochondrial | | | |
| | | | product | | | |
| 383 | 1368393_AT | AF160978 | Lymphocyte antigen 68 | NM_053383 | NM_012072 | NM_010740 |
| | 1376574_AT | BI282932 | | | | |
| 384 | 1367998_AT | AF151982 | secretory leukocyte protease inhibitor | NM_053372 | NM_003064 | NM_011414 |
| 385 | 1367940_AT | AF118816 | chemokine orphan receptor 1 | NM_053352 | NM_020311 | NM_007722 |
| 386 | 1370624_AT | AF310076 | coagulation factor II (thrombin) receptor-like 2 | NM_053313 | NM_004101 | NM_010170 |
| 387 | 1369621_S_AT | U69485 | FK506 binding protein 2 | NM_053308 | NM_004116 | NM_008019 |
| | | | | | NM_054033 | |
| 388 | 1369931_AT | X15800 | pyruvate kinase, muscle | NM_053297 | NM_002654 | NM_011099 |
| | | | | | NM_182470 | |
| | | | | | NM_182471 | |
| 389 | 1373466_AT | AA946474 | Calpastatin | NM_053295 | NM_001750 | NM_009817 |
| | | | | | NM_173060 | |
| | | | | | NM_173061 | |
| | | | | | NM_173062 | |
| 390 | 1388318_AT | BI279760 | Phosphoglycerate kinase 1 | NM_053291 | NM_000291 | NM_008828 |
| 391 | 1386864_AT | M76591 | phosphoglycerate mutase 1 | NM_053290 | NM_002629 | NM_023418 |
| 392 | 1368731_AT | J00696 | orosomucoid 1 | NM_053288 | NM_000607 | NM_011016 |
| 393 | 1367784_A_AT | AF314657 | clusterin | NM_053021 | NM_001831 | NM_013492 |
| | | | | | NM_203339 | |
| 394 | 1387885_AT | BE110597 | Fc receptor, IgG, alpha chain transporter | NM_033351 | NM_004107 | NM_010189 |
| 395 | 1367722_AT | AB048711 | dipeptidylpeptidase 7 | NM_031973 | NM_013379 | NM_031843 |
| 396 | 1368106_AT | AF136583 | serum-inducible kinase | NM_031821 | NM_006622 | NM_152804 |
| 397 | 1387197_AT | AF104362 | osteomodulin (osteoadherin) | NM_031817 | NM_005014 | NM_012050 |
| 398 | 1387975_AT | AF047707 | UDP-glucose:ceramide glycosyltransferase | NM_031795 | NM_003358 | NM_011673 |
| 399 | 1375951_AT | AA818521 | Thrombomodulin | NM_031771 | NM_000361 | NM_009378 |
| 400 | 1370221_AT | BF419320 | WNT1 inducible signaling pathway protein 1 | NM_031716 | NM_003882 | NM_018865 |
| | 1384051_AT | BF390066 | | | NM_080838 | |
| 401 | 1369313_AT | AB008571 | four and a half LIM domains 2 | NM_031677 | NM_001450 | NM_010212 |
| | 1371951_AT | AA800031 | | | NM_201555 | |
| | | | | | NM_201556 | |
| | | | | | NM_201557 | |
| 402 | 1383247_A_AT | BI291029 | MYB binding protein 1a | NM_031668 | NM_014520 | NM_016776 |
| | 1393282_AT | AA942816 | | | | |
| 403 | 1370051_AT | M57263 | transglutaminase 1 | NM_031659 | NM_000359 | NM_019984 |
| 404 | 1393987_S_AT | BI274742 | G protein-coupled receptor kinase 6 | NM_031657 | NM_002082 | NM_011938 |
| 405 | 1369960_AT | U72246 | FXYD domain-containing ion transport | NM_031648 | NM_005031 | NM_019503 |
| | | | regulator 1 | | NM_021902 | NM_052991 |
| | | | | | | NM_052992 |
| | | | | | | NM_194321 |
| 406 | 1367701_AT | AB042888 | receptor (calcitonin) activity modifying | NM_031646 | NM_005854 | NM_019444 |
| | | | protein 2 | | | |
| 407 | 1367760_AT | D13341 | mitogen activated protein kinase kinase 1 | NM_031643 | NM_002755 | NM_008927 |
| 408 | 1398851_AT | AA892351 | tyrosine 3-monooxygenase/tryptophan 5- | NM_031603 | NM_006761 | NM_009536 |
| | | | monooxygenase activation protein, epsilon | | | |
| | | | polypeptide | | | |
| 409 | 1369484_AT | AF259981 | WNT1 inducible signaling pathway protein 2 | NM_031590 | NM_003881 | NM_016873 |
| 410 | 1369947_AT | AF010306 | cathepsin K | NM_031560 | NM_000396 | NM_007802 |
| 411 | 1393281_AT | AW915173 | caveolin | NM_031556 | NM_001753 | NM_007616 |
| | 1372111_AT | BI285449 | | | | |
| 412 | 1367973_AT | AF058786 | small inducible cytokine A2 | NM_031530 | NM_005408 | NM_011333 |
| 413 | 1370642_S_AT | BM389426 | Rattus norvegicus transcribed sequences | NM_031525 | NM_002609 | NM_008809 |
| 414 | 1380110_AT | AI229643 | Janus kinase 2 | NM_031514 | NM_004972 | NM_008413 |
| 415 | 1398256_AT | M98820 | interleukin 1 beta | NM_031512 | NM_000576 | NM_008361 |
| 416 | 1367774_AT | AF111160 | glutathione S-transferase, alpha 1 | NM_031509 | NM_000846 | NM_008182 |
| | | | | | NM_153699 | NM_010356 |
| | | | | | NM_000847 | |
| 417 | 1370892_AT | BI285347 | complement component 4a | NM_031504 | NM_007293 | NM_009780 |
| | | | | | NM_000592 | |
| 418 | 1379772_AT | BG371624 | Angiotensin receptor-like 1 (Agtrl1) | NM_031349 | NM_005161 | NM_011784 |
| 419 | 1387808_AT | AF200684 | solute carrier family 7 (cationic amino acid | NM_031341 | NM_003982 | NM_011405 |
| | | | transporter, y + system), member 7 | | | |
| 420 | 1387259_AT | AF097593 | cadherin 2 | NM_031333 | NM_001792 | NM_007664 |
| 421 | 1370309_A_AT | AJ238854 | heterogeneous nuclear ribonucleoprotein A/B | NM_031330 | NM_031266 | NM_010448 |
| | | | | | NM_004499 | |
| 422 | 1367574_AT | X62952 | vimentin | NM_031140 | NM_003380 | NM_011701 |
| 423 | 1372466_AT | AI408571 | transforming growth factor, beta receptor II | NM_031132 | NM_003242 | NM_009371 |
| | | | | | | NM_029575 |
| 424 | 1388836_AT | AA799981 | Protein kinase C-eta | NM_031085 | NM_006255 | NM_008856 |
| 425 | 1368089_AT | U21101 | cyclic GMP stimulated phosphodiesterase | NM_031079 | NM_002599 | XM_133606 |
| 426 | 1387344_AT | AI407458 | methylmalonate semialdehyde dehydrogenase | NM_031057 | NM_005589 | NM_134042 |
| | | | gene | | | |
| 427 | 1369166_AT | U36476 | matrix metalloproteinase 9 (gelatinase B, 92-kDa | NM_031055 | NM_004994 | NM_013599 |
| | 1398275_AT | U24441 | type IV collagenase) | | | |
| 428 | 1370301_AT | U65656 | matrix metalloproteinase 2 (72 KDa type IV | NM_031054 | NM_004530 | NM_008610 |
| | | | collagenase) | | | |
| 429 | 1367749_AT | X84039 | lumican | NM_031050 | NM_002345 | NM_008524 |
| 430 | 1367627_AT | U07971 | glycine amidinotransferase (L- | NM_031031 | NM_001482 | NM_025961 |
| | | | arginine:glycine amidinotransferase) | | | |
| 431 | 1368404_AT | X59267 | drebrin 1 | NM_031024 | NM_004395 | NM_019813 |
| | | | | | NM_080881 | |
| 432 | 1386938_AT | M25073 | alanyl (membrane) aminopeptidase | NM_031012 | NM_001150 | NM_008486 |
| 433 | 1367732_AT | U34958 | guanine nucleotide binding protein, beta 1 | NM_030987 | NM_002074 | NM_008142 |
| 434 | 1371575_AT | BF281185 | moesin | NM_030863 | NM_002444 | NM_010833 |
| 435 | 1367682_AT | AF315950 | midkine | NM_030859 | NM_002391 | NM_010784 |
| 436 | 1369294_AT | D49955 | bone marrow stromal cell antigen 1 | NM_030848 | NM_004334 | NM_009763 |
| 437 | 1367914_AT | Y10889 | epithelial membrane protein 3 | NM_030847 | NM_001425 | NM_010129 |
| 438 | 1368223_AT | AF149118 | a disintegrin and metalloproteinase with | NM_024400 | NM_006988 | NM_009621 |
| | | | thrombospondin motifs 1 (ADAMTS-1) | | | |
| 439 | 1387270_AT | D86383 | hematopoietically expressed homeobox | NM_024385 | NM_002729 | NM_008245 |
| 440 | 1369269_AT | U35890 | Polypeptide GalNAc transferase T1 | NM_024373 | NM_020474 | NM_013814 |
| | 1399045_AT | AI236745 | | | | |
| 441 | 1387076_AT | AF057308 | hypoxia inducible factor 1, alpha subunit | NM_024359 | NM_001530 | NM_010431 |
| | | | | | NM_181054 | |
| 442 | 1372031_AT | AI407821 | Disabled homolog 2, mitogen-responsive | NM_024159 | NM_001343 | NM_023118 |
| | | | phosphoprotein (Drosophila) | | | |
| 443 | 1387817_AT | V01543 | brain specific mRNA b | NM_024128 | NM_014392 | NM_010942 |
| 444 | 1369703_AT | AJ277828 | Endothelial PAS domain protein 1 | NM_023090 | NM_001430 | NM_010137 |
| | 1398333_AT | AI598434 | | | | |
| 445 | 1368078_AT | U80818 | endothelial cell-specific molecule 1 | NM_022604 | NM_007036 | NM_023612 |
| 446 | 1367646_AT | X82396 | cathepsin B | NM_022597 | NM_001908 | NM_007798 |
| | | | | | NM_147780 | |
| 447 | 1368885_AT | AI009808 | ectonucleoside triphosphate | NM_022587 | NM_001776 | NM_009848 |
| | | | diphosphohydrolase 1 | | | |
| 448 | 1369958_AT | M74295 | rhoB gene | NM_022542 | NM_004040 | NM_007483 |
| 449 | 1369961_AT | U90556 | phosphatidate phosphohydrolase type 2a | NM_022538 | NM_003711 | NM_008247 |
| | | | | | NM_176895 | NM_008903 |
| 450 | 1367765_AT | AF054810 | transcobalamin II precursor | NM_022534 | NM_000355 | NM_015749 |
| 451 | 1369926_AT | D00680 | glutathione peroxidase 3 | NM_022525 | NM_002084 | NM_008161 |
| 452 | 1367902_AT | AF257110 | guanine nucleotide binding protein gamma | NM_022396 | NM_004126 | NM_025331 |
| | | | subunit 11 | | | |
| 453 | 1368464_AT | J05495 | macrophage galactose N-acetyl- | NM_022393 | | NM_010796 |
| | | | galactosamine specific lectin | | | |
| 454 | 1367671_AT | Y00047 | proliferating cell nuclear antigen | NM_022381 | NM_002592 | NM_011045 |
| | | | | | NM_182649 | |
| 455 | 1398350_AT | BG380454 | Brain acidic membrane protein | NM_022300 | NM_006317 | |
| 456 | 1387547_A_AT | AF192401 | ETL protein | NM_022294 | XM_371262 | NM_133222 |
| 457 | 1383328_X_AT | AI576297 | programmed cell death 4 | NM_022265 | NM_014456 | NM_011050 |
| | | | | | NM_145341 | |
| 458 | 1381470_AT | AI454052 | C-kit receptor tyrosine kinase | NM_022264 | NM_000222 | NM_021099 |
| | 1386540_AT | BG666051 | | | | |
| 459 | 1387160_AT | AJ271742 | mink-related peptide 2 | NM_022235 | NM_005472 | NM_020574 |
| 460 | 1370073_AT | AB017702 | protein kinase inhibitor p58 | NM_022232 | NM_006260 | NM_008929 |
| 461 | 1368430_AT | AF154349 | protease, cysteine, 1 (legumain) | NM_022226 | NM_005606 | NM_011175 |
| 462 | 1387648_AT | U90448 | CXC chemokine LIX | NM_022214 | NM_002993 | NM_009141 |
| | | | | | NM_002994 | |
| 463 | 1393799_AT | BI303989 | transmembrane receptor Unc5H2 | NM_022207 | NM_170744 | NM_029770 |
| 464 | 1370097_A_AT | U54791 | Chemokine receptor (LCR1) | NM_022205 | NM_003467 | NM_009911 |
| | 1373661_A_AT | AA945737 | | | | |
| | 1389244_X_AT | | | | | |
| 465 | 1369633_AT | AI171777 | chemokine (C—X—C motif) ligand 12 | NM_022177 | NM_199168 | NM_021704 |
| | 1388583_AT | BF283398 | | | NM_000609 | NM_013655 |
| 466 | 1370248_AT | AA851939 | FXYD domain-containing ion transport | NM_022005 | NM_022003 | NM_022004 |
| | | | regulator 6 | | | |
| 467 | 1375144_AT | BM388843 | tissue inhibitor of metalloproteinase 2 | NM_021989 | NM_003255 | NM_011594 |
| | 1386940_AT | S72594 | | | | |
| | 1367823_AT | BF523128 | | | | |
| | 1389836_A_AT | AI599265 | | | | |
| | 1388312_AT | BI274487 | | | | |
| 468 | 1371166_AT | AJ011116 | nitric oxide synthase 3, endothelial cell | NM_021838 | NM_000603 | NM_008713 |
| 469 | 1367619_AT | U63315 | progesterone receptor membrane component 1 | NM_021766 | NM_006667 | NM_016783 |
| 470 | 1392917_AT | BI302971 | Arfaptin 1 | NM_021763 | NM_014447 | XM_130985 |
| 471 | 1368347_AT | AF272661 | collagen, type V, alpha 3 | NM_021760 | NM_015719 | NM_016919 |
| 472 | 1368173_AT | AF194371 | Nopp140 associated protein | NM_021754 | NM_015934 | NM_018868 |
| 473 | 1398286_AT | M64755 | cysteine-sulfinate decarboxylase | NM_021750 | NM_015989 | NM_144942 |
| 474 | 1374779_AT | BM388525 | Coagulation factor XIIIa | NM_021698 | NM_000129 | NM_028784 |
| 475 | 1367658_AT | AF133301 | SH3/ankyrin domain gene 3 | NM_021676 | XM_037493 | NM_021423 |
| 476 | 1370023_AT | M76532 | gap junction membrane channel protein alpha 4 | NM_021654 | NM_002060 | NM_008120 |
| 477 | 1368448_AT | Y12760 | latent transforming growth factor beta | NM_021586 | NM_000428 | NM_013589 |
| | | | binding protein 2 | | | |
| 478 | 1370256_AT | AA944349 | Rattus norvegicus transcribed sequences | NM_021266 | NM_003505 | NM_021457 |
| 479 | 1387389_AT | AB030944 | receptor (calcitonin) activity modifying | NM_020100 | NM_005856 | NM_019511 |
| | | | protein 3 | | | |
| 480 | 1384227_AT | AI044031 | Rattus norvegicus transcribed sequence with | NM_020085 | NM_002844 | NM_008983 |
| | | | strong similarity to protein sp: Q15262 | | | |
| | | | (H. sapiens) PTPK_HUMAN Protein-tyrosine | | | |
| | | | phosphatase kappa precursor (R-PTP-kappa) | | | |
| 481 | 1368655_AT | K02934 | proteoglycan peptide core protein | NM_020074 | NM_002727 | NM_011157 |
| 482 | 1386866_AT | D17447 | tyrosine 3-monooxgenase/tryptophan 5- | NM_019376 | NM_012479 | NM_018871 |
| | | | monooxgenase activation protein, gamma | | | |
| | | | polypeptide | | | |
| 483 | 1367905_AT | U78787 | alkaline phosphodiesterase | NM_019370 | NM_005021 | XM_125594 |
| 484 | 1387856_AT | BI274457 | calponin 3, acidic | NM_019359 | NM_001839 | NM_028044 |
| 485 | 1386913_AT | U07797 | glycoprotein 38 | NM_019358 | NM_006474 | NM_010329 |
| | | | | | NM_198389 | |
| 486 | 1382108_AT | AA900536 | V-maf musculoaponeurotic fibrosarcoma | NM_019316 | NM_005461 | NM_010658 |
| | | | oncogene family, protein B (avian) (Mafb) | | | |
| 487 | 1369087_AT | D28498 | FMS-like tyrosine kinase 1 | NM_019306 | NM_002019 | NM_010228 |
| | 1375473_AT | BI296644 | | | | |
| | 1383019_AT | BF558478 | | | | |
| | 1393235_AT | AI059968 | | | | |
| 488 | 1367733_AT | X58294 | carbonic anhydrase 2 | NM_019291 | NM_000067 | NM_009801 |
| | 1386922_AT | AI408948 | | | | |
| 489 | 1371572_AT | BM986220 | amyloid beta (A4) precursor protein | NM_019288 | NM_000484 | NM_007471 |
| | | | | | NM_201413 | |
| | | | | | NM_201414 | |
| 490 | 1368370_AT | M80633 | adenylyl cyclase 4 | NM_019285 | NM_139247 | NM_080435 |
| 491 | 1386912_AT | U94710 | procollagen C-proteinase enhancer protein | NM_019237 | NM_002593 | NM_008788 |
| 492 | 1388089_A_AT | AY050655 | ring finger protein 4 | NM_019182 | NM_002938 | NM_011278 |
| 493 | 1367942_AT | M76110 | acid phosphatase 5 | NM_019144 | NM_001611 | NM_007388 |
| 494 | 1369897_S_AT | BI277035 | GNAS complex locus | NM_019132 | NM_000516 | NM_010309 |
| | | | | | NM_080426 | NM_010310 |
| | | | | | NM_080425 | NM_019690 |
| | | | | | NM_016592 | NM_022000 |
| 495 | 1387005_AT | L03201 | cathepsin S | NM_017320 | NM_004079 | NM_021281 |
| 496 | 1371840_AT | BI295971 | Rattus norvegicus endothelial differentiation | NM_017301 | NM_001400 | NM_007901 |
| | | | sphingolipid G-protein-coupled receptor 1 | | | |
| 497 | 1369065_A_AT | J04022 | ATPase, Ca++ transporting, cardiac muscle, | NM_017290 | NM_170665 | NM_009722 |
| | | | slow twitch 2 | | NM_001681 | |
| 498 | 1367959_A_AT | AF182949 | sodium channel, voltage-gated, type I, beta | NM_017288 | NM_001037 | NM_011322 |
| | | | polypeptide | | NM_199037 | |
| 499 | 1367663_AT | D45249 | protease (prosome, macropain) 28 subunit, | NM_017264 | NM_006263 | NM_011189 |
| | | | alpha | | NM_176783 | |
| 500 | 1367930_AT | X06338 | growth associated protein 43 | NM_017195 | NM_002045 | NM_008083 |
| 501 | 1387395_AT | M91466 | adenosine A2B receptor | NM_017161 | NM_000676 | NM_007413 |
| 502 | 1369973_AT | J05579 | xanthine dehydrogenase | NM_017154 | NM_000379 | NM_011723 |
| 503 | 1370057_AT | U09567 | cysteine and glycine-rich protein 1 | NM_017148 | NM_004078 | NM_007791 |
| 504 | 1370184_AT | BG666614 | cofilin 1 | NM_017147 | NM_005507 | NM_007687 |
| | 1387871_AT | BM389673 | | | | |
| 505 | 1371824_AT | AA891949 | Rattus norvegicus adenylate kinase 4 (Ak4), | NM_017135 | NM_013410 | NM_009647 |
| | | | mRNA | | | |
| 506 | 1372326_AT | AA901341 | solute carrier family 2, member 2 | NM_017102 | NM_006931 | NM_011401 |
| 507 | 1368280_AT | D90404 | cathepsin C | NM_017097 | NM_001814 | NM_009982 |
| | | | | | NM_148170 | |
| 508 | 1367594_AT | U17834 | biglycan | NM_017087 | NM_001711 | NM_007542 |
| 509 | 1387153_AT | X76454 | reversion induced LIM gene | NM_017062 | NM_003687 | NM_019417 |
| 510 | 1370172_AT | AA892254 | superoxide dismutase 2 | NM_017051 | NM_000636 | NM_013671 |
| 511 | 1368259_AT | S67721 | prostaglandin-endoperoxide synthase 1 | NM_017043 | NM_000962 | NM_008969 |
| | | | | | NM_080591 | |
| 512 | 1369044_A_AT | AF202733 | phosphodiesterase 4B | NM_017031 | NM_002600 | NM_019840 |
| 513 | 1368000_AT | X52477 | complement component 3 | NM_016994 | NM_000064 | NM_009778 |
| 514 | 1387402_AT | U31463 | myosin, heavy polypeptide 9 | NM_013194 | NM_002473 | NM_022410 |
| 515 | 1368183_AT | J03806 | phospholipase C, gamma 1 | NM_013187 | NM_002660 | NM_021280 |
| | | | | | NM_182811 | |
| 516 | 1368612_AT | U60096 | integrin beta 4 | NM_013180 | NM_000213 | XM_109756 |
| 517 | 1367859_AT | U03491 | transforming growth factor, beta 3 | NM_013174 | NM_003239 | NM_009368 |
| 518 | 1370244_AT | AI176595 | cathepsin L | NM_013156 | NM_001333 | NM_009984 |
| | | | | | (CSTL2) | |
| | | | | | NM_001912 | |
| | | | | | NM_145918 | |
| | | | | | (CSTL) | |
| 519 | 1389611_AT | AA849857 | very low density lipoprotein receptor | NM_013155 | NM_003383 | NM_013703 |
| 520 | 1387343_AT | M65149 | CCAAT/enhancer binding, protein (C/EBP) | NM_013154 | NM_005195 | NM_007679 |
| | 1368813_AT | BF419200 | delta | | | |
| 521 | 1387548_AT | AF008201 | hyaluronan synthase 2 | NM_013153 | NM_005328 | NM_008216 |
| 522 | 1367800_AT | M23697 | plasminogen activator, tissue | NM_013151 | NM_000930 | NM_008872 |
| | | | | | NM_000931 | |
| | | | | | NM_033011 | |
| 523 | 1372104_AT | BF289002 | Guanine nucleotide binding protein, alpha | NM_013145 | NM_002069 | XM_355574 |
| | | | inhibiting 1 | | | |
| 524 | 1389373_AT | AI029555 | MAD homolog 1 (Drosophila) | NM_013130 | NM_005900 | NM_008539 |
| 525 | 1391946_AT | BI296054 | selectin, platelet | NM_013114 | NM_003005 | NM_011347 |
| 526 | 1367814_AT | M14137 | ATPase Na+/K+ transporting beta 1 | NM_013113 | NM_001677 | NM_009721 |
| | 1386937_AT | AI232036 | polypeptide (not NME7) | | | |
| 527 | 1388201_AT | AW141680 | bone morphogenetic protein 6 | NM_013107 | NM_001718 | NM_007556 |
| 528 | 1398828_AT | U09386 | FK506-binding protein 1a | NM_013102 | NM_054014 | NM_008019 |
| | 1398829_AT | AI598372 | | NM_053308 | NM_000801 | |
| 529 | 1378925_AT | AW529408 | cAMP responsive element modulator | NM_013086 | NM_181571 | NM_013498 |
| | | | | NM_017334 | NM_182717 | |
| | | | | | NM_182718 | |
| | | | | | NM_182719 | |
| | | | | | NM_182720 | |
| | | | | | NM_182721 | |
| | | | | | NM_182722 | |
| | | | | | NM_182723 | |
| | | | | | NM_182724 | |
| | | | | | NM_182725 | |
| | | | | | NM_182769 | |
| | | | | | NM_182770 | |
| | | | | | NM_182771 | |
| | | | | | NM_182772 | |
| | | | | | NM_182850 | |
| | | | | | NM_182853 | |
| | | | | | NM_183011 | |
| | | | | | NM_183012 | |
| | | | | | NM_183013 | |
| | | | | | NM_183060 | |
| 530 | 1387675_AT | X63434 | plasminogen activator, urokinase | NM_013085 | NM_002658 | NM_008873 |
| | 1398287_AT | X65651 | | | | |
| 531 | 1367948_A_AT | U93307 | kinase insert domain protein receptor | NM_013062 | NM_002253 | NM_010612 |
| 532 | 1367693_AT | D17445 | tyrosine 3-monooxygenase/tryptophan 5- | NM_013052 | NM_003405 | NM_011738 |
| | | | monooxygenase activation protein, eta | | | |
| | | | polypeptide | | | |
| 533 | 1374171_AT | AI170507 | Abcc9: ATP-binding cassette, sub-family C | NM_013040 | NM_005691 | NM_011511 |
| | | | (CFTR/MRP), member 9 | | NM_020297 | NM_021041 |
| | | | | | NM_020298 | NM_021043 |
| | | | | | | NM_021042 |
| 534 | 1387273_AT | U04319 | interleukin 1 receptor-like 1 | NM_013037 | NM_016232 | NM_010743 |
| | | | | | NM_003856 | |
| | | | | | NM_173459 | |
| 535 | 1369815_AT | U22414 | chemokine (C-C motif) ligand 3 | NM_013025 | NM_002983 | NM_011337 |
| 536 | 1367881_AT | D85183 | protein tyrosine phosphatase, non-receptor | NM_013016 | NM_080792 | NM_007547 |
| | | | type substrate 1 | | | |
| 537 | 1368145_AT | M24852 | Purkinje cell protein 4 | NM_013002 | NM_006198 | NM_008791 |
| 538 | 1398757_AT | J03969 | nucleophosmin 1 | NM_012992 | NM_002520 | NM_008722 |
| | | | | | NM_199185 | |
| 539 | 1386948_AT | M34384 | nestin | NM_012987 | NM_006617 | NM_016701 |
| 540 | 1387202_AT | D00913 | intercellular adhesion molecule 1 | NM_012967 | NM_000201 | NM_010493 |
| 541 | 1387530_A_AT | U18913 | fos-like antigen 2 | NM_012954 | NM_005253 | NM_008037 |
| 542 | 1386899_AT | Y00708 | cathepsin H | NM_012939 | NM_004390 | NM_007801 |
| | | | | | NM_148979 | |
| 543 | 1368474_AT | M84488 | vascular cell adhesion molecule 1 | NM_012889 | NM_001078 | NM_011693 |
| | | | | | NM_080682 | |
| 544 | 1368989_AT | U27201 | tissue inhibitor of metalloproteinase 3 | NM_012886 | NM_000362 | NM_011595 |
| | 1375138_AT | AA893169 | | | | |
| 545 | 1367581_A_AT | AB001382 | secreted phosphoprotein 1 | NM_012881 | NM_000582 | NM_009263 |
| 546 | 1368322_AT | Z24721 | superoxide dismutase 3 | NM_012880 | NM_003102 | NM_011435 |
| 547 | 1392578_AT | AI070875 | Matrix Gla protein | NM_012862 | NM_000900 | NM_008597 |
| 548 | 1387137_AT | X72914 | cartilage oligomeric matrix protein | NM_012834 | NM_000095 | NM_016685 |
| 549 | 1367974_AT | M20559 | Annexin III (Lipocortin III) | NM_012823 | NM_005139 | NM_013470 |
| 550 | 1387348_AT | BE113270 | insulin-like growth factor-binding protein 5 | NM_012817 | NM_000599 | NM_010518 |
| 551 | 1368057_AT | D90038 | ATP-binding cassette, sub-family D (ALD), | NM_012804 | NM_002858 | NM_008991 |
| | | | member 3 | | | |
| 552 | 1368395_AT | M22400 | glypican 3 | NM_012774 | NM_004484 | NM_016697 |
| 553 | 1373683_AT | AI230396 | Rattus norvegicus fyn proto-oncogene (Fyn), | NM_012755 | NM_002037 | NM_008054 |
| | | | Mrna | | NM_153047 | |
| | | | | | NM_153048 | |
| 554 | 1369953_A_AT | BI285141 | CD24 antigen | NM_012752 | NM_013230 | NM_009846 |
| 555 | 1369006_AT | M68971 | hexokinase 2 | NM_012735 | NM_000189 | NM_013820 |
| 556 | 1387219_AT | U15419 | adrenomedullin | NM_012715 | NM_001124 | NM_009627 |
| 557 | 1369651_AT | X03150 | thymus cell antigen 1, theta | NM_012673 | NM_006288 | NM_009382 |
| | 1369652_AT | AI145313 | | | | |
| 558 | 1368049_AT | D90345 | t-complex protein 1 | NM_012670 | NM_030752 | NM_013686 |
| 559 | 1367721_AT | S61868 | syndecan 4 | NM_012649 | NM_002999 | NM_011521 |
| 560 | 1392264_S_AT | AI500951 | serine (or cysteine) proteinase inhibitor, | NM_012620 | NM_000602 | NM_008871 |
| | 1368519_AT | M24067 | member 1 | | | |
| 561 | 1367846_AT | J03628 | S100 calcium-binding protein A4 | NM_012618 | NM_002961 | NM_011311 |
| | | | | | NM_019554 | |
| 562 | 1387154_AT | M15880 | neuropeptide Y | NM_012614 | NM_000905 | NM_023456 |
| 563 | 1367652_AT | AI713966 | insulin-like growth factor binding protein 3 | NM_012588 | NM_000598 | NM_008343 |
| | 1386881_AT | M31837 | | | | |
| 564 | 1368221_AT | M14053 | nuclear receptor subfamily 3, group C, | NM_012576 | NM_000176 | NM_008173 |
| | | | member 1 | | | |
| 565 | 1387843_AT | AH005431 | follistatin | NM_012561 | NM_006350 | |
| | | | | | NM_013409 | |
| 566 | 1368851_AT | L20681 | v-ets erythroblastosis virus E26 oncogene | NM_012555 | NM_005238 | NM_011808 |
| | | | homolog 1 (avian) | | | |
| 567 | 1367575_AT | X02610 | enolase 1, alpha | NM_012554 | NM_001428 | NM_023119 |
| 568 | 1378342_AT | BF284819 | endothelin receptor type A | NM_012550 | NM_001957 | NM_010332 |
| | 1383641_AT | BF414702 | | | | |
| 569 | 1368419_AT | AF202115 | ceruloplasmin | NM_012532 | NM_000096 | NM_007752 |
| | 1368420_AT | L33869 | | | | |
| 570 | 1371440_AT | AW916647 | Beta-2 microglobulin | NM_012512 | NM_004048 | NM_009735 |
| 571 | 1388666_AT | AI179988 | Rattus norvegicus ectodermal-neural cortex 1 | NM_001003401 | NM_003633 | NM_007930 |
| | | | (Enc1), | | | |
| 572 | 1384302_AT | AI712791 | NTT4 (Ntt4) mRNA, complete cds | L06434 | | |
| 573 | 1372256_AT | BF550246 | Rattus norvegicus transcribed sequence with | BF550246 | NM_001311 | NM_007763 |
| | | | strong similarity to protein ref: NP_001302.1 | | | |
| | | | (H. sapiens) cysteine-rich protein 1 | | | |
| | | | (intestinal); cysteine-rich intestinal protein | | | |
| | | | [Homo sapiens] | | | |
| 574 | 1374953_AT | AI599484 | cDNA clone IMAGE: 7110106 | BC078951 | | XM_131302 |
| 575 | 1373666_AT | AI102560 | Guanine nucleotide exchange factor (Mrgef) | AY390379 | NM_012294 | NM_175930 |
| | | | mRNA, complete cds | | | |
| 576 | 1385397_AT | AA859085 | Ab1-219 mRNA, complete cds | AY325154 | NM_024636 | NM_054098 |
| 577 | 1381678_AT | AW525560 | Rattus norvegicus basophilic leukemia | AY268934 | NM_003088 | NM_007984 |
| | | | expressed sequence 06 (Bles06) mRNA, | | | |
| | | | partial cds | | | |
| 578 | 1370802_AT | AF336872 | integrin, beta 5 | AF336872 | NM_002213 | NM_010580 |
| 579 | 1392905_AT | BF552733 | Guanine nucleotide binding protein, gamma 2 | AF022087 | NM_012202 | NM_010316 |
| 580 | 1393252_AT | AA996804 | LOC315191 similar to Fbln1 protein | XM_243637 | NM_006486 | NM_010180 |
| 581 | 1393944_AT | AI764505 | similar to Ecotropic viral integration site 3 | XM_226153 | NM_015461 | NM_145492 |
| | | | | | | NM_181326 |
| 582 | 1371545_AT | AI010414 | | | | |
| 583 | 1371986_AT | AI576652 | | | | |
| 584 | 1372005_AT | BI294361 | | | | |
| 585 | 1372006_AT | BM391274 | | | | |
| 586 | 1372213_AT | BM390487 | | | | |
| 587 | 1372223_AT | BG374299 | | | | |
| 588 | 1372234_AT | AA800950 | | | | |
| 589 | 1372311_AT | BG380795 | Rattus norvegicus transcribed sequences | | NM_005471 | NM_011937 |
| 590 | 1372410_AT | AW251360 | | | | |
| 591 | 1372549_AT | AI409460 | Transcribed locus, highly similar to | | NM_203411 | NM_025915 |
| | | | NP_080191.2 RIKEN cDNA 2600017H02 | | | |
| | | | gene | | | |
| 592 | 1372579_AT | BF288089 | | | | |
| 593 | 1372585_AT | BM388445 | | | | |
| 594 | 1372761_AT | AI228076 | | | | |
| 595 | 1372836_AT | AI170399 | | | | |
| 596 | 1372838_AT | AI227902 | | | | |
| 597 | 1373079_AT | BI296427 | | | | |
| 598 | 1373102_AT | BI282750 | | | | |
| 599 | 1373140_AT | AA851740 | | | | |
| 600 | 1373180_AT | AI227919 | | | | |
| 601 | 1373181_AT | BE101285 | | | | |
| 602 | 1373217_AT | AI010234 | | | | |
| 603 | 1373286_AT | AA875261 | | | | |
| 604 | 1373310_AT | BF396512 | | | | |
| 605 | 1373401_AT | AI176034 | Highly hmologous to murine tenascin C | | NM_002160 | NM_011607 |
| | | | [Mus musculus] | | | |
| 606 | 1373415_AT | AI407050 | | | | |
| 607 | 1373487_AT | AI030552 | | | | |
| 608 | 1373488_AT | BF289154 | | | | |
| 609 | 1373533_AT | AI412460 | | | | |
| 610 | 1373628_AT | AA818342 | | | | |
| 611 | 1373740_AT | AA851385 | | | | |
| 612 | 1374057_AT | AI177143 | | | | |
| 613 | 1374172_AT | AI010883 | | | | |
| 614 | 1374273_AT | BG665433 | | | | |
| 615 | 1374276_AT | BE104102 | | | | |
| 616 | 1374284_AT | AI227769 | | | | |
| 617 | 1374285_AT | AW531275 | | | | |
| 618 | 1374306_AT | BF394933 | | | | |
| 619 | 1374529_AT | AI406660 | Highly hmologous to murine thrombospondin 1 | | NM_003246 | NM_011580 |
| 620 | 1374531_AT | AA926305 | Transcribed sequence with moderate | | | |
| | | | similarity to protein pir: T45059 (C. elegans) | | | |
| | | | T45059 hypothetical protein Y39B6B.gg | | | |
| | | | [imported] - Caenorhabditis elegans | | | |
| 621 | 1374635_AT | AW527151 | | | | |
| 622 | 1374742_AT | AA943983 | | | | |
| 623 | 1374771_AT | BM388859 | | | | |
| 624 | 1374868_AT | AW535434 | | | | |
| 625 | 1375028_AT | AI409635 | | | | |
| 626 | 1375051_AT | BE104792 | | | | |
| 627 | 1375270_AT | BM384026 | | | | |
| 628 | 1375377_AT | BE098713 | | | | |
| 629 | 1375463_AT | BG380847 | | | | |
| 630 | 1375721_AT | BF408438 | | | | |
| 631 | 1376004_AT | BF285297 | | | | |
| 632 | 1376045_AT | AW529960 | | | | |
| 633 | 1376055_AT | AA859768 | | | | |
| 634 | 1376128_AT | AI103937 | | | | |
| 635 | 1376624_AT | BF288208 | | | | |
| 636 | 1376631_AT | BF283675 | | | | |
| 637 | 1376655_AT | AA850435 | | | | |
| 638 | 1376786_A_AT | BF397709 | | | | |
| 639 | 1376848_AT | AI137988 | | | | |
| 640 | 1376919_AT | BG665267 | | | | |
| 641 | 1377630_AT | AI408602 | Transcribed sequence with moderate | | | |
| | | | similarity to protein pir: S12207 | | | |
| | | | (M. musculus) S12207 hypothetical protein | | | |
| 642 | 1377697_AT | BI275738 | | | | |
| 643 | 1377702_AT | BG380173 | Similar to Mus musculus purinergic receptor | | NM_005767 | NM_175116 |
| | | | (family A group 5) (P2y5) | | | |
| 644 | 1377723_AT | AI234810 | | | | |
| 645 | 1377862_AT | BI274618 | | | | |
| 646 | 1378321_AT | BG375029 | | | | |
| 647 | 1378440_AT | BM385137 | | | | |
| 648 | 1378507_AT | BE108914 | | | | |
| 649 | 1379294_AT | AA957424 | | | | |
| 650 | 1379331_AT | AA965084 | | | | |
| 651 | 1379497_AT | BI275261 | | | | |
| | 1391505_X_AT | | | | | |
| 652 | 1379604_AT | BF284937 | | | | |
| 653 | 1379790_AT | BI288729 | | | | |
| 654 | 1379799_AT | BF415056 | | | | |
| 655 | 1379882_A_AT | BE116018 | Locus highly similar to NP_072075.1 | | NM_006182 | NM_022563 |
| | | | discoidin domain receptor family, member 2 | | | |
| | | | [Mus musculus] | | | |
| 656 | 1380089_AT | AI407434 | | | | |
| 657 | 1380318_AT | BF401102 | | | | |
| 658 | 1380908_AT | AA924563 | | | | |
| 659 | 1381335_AT | BE349658 | | | | |
| 660 | 1381410_A_AT | AI555744 | | | | |
| 661 | 1381461_AT | AI058509 | | | | |
| 662 | 1381915_AT | AI111735 | | | | |
| 663 | 1382017_AT | AI012949 | | | | |
| 664 | 1382212_AT | AI385201 | | | | |
| 665 | 1382296_AT | BF291041 | | | | |
| 666 | 1382375_AT | AI639128 | | | | |
| 667 | 1382404_AT | BG669741 | | | | |
| 668 | 1382482_AT | AI178627 | | | | |
| 669 | 1382536_AT | BE109224 | | | | |
| 670 | 1382571_AT | AA925335 | | | | |
| 671 | 1382628_AT | BE111904 | | | | |
| 672 | 1382692_AT | AI045955 | | | | |
| 673 | 1382818_AT | AI177067 | | | | |
| 674 | 1382995_AT | AA859669 | | | | |
| 675 | 1383147_AT | AW526373 | | | | |
| 676 | 1383193_AT | BE112341 | | | | |
| 677 | 1383261_AT | BF560105 | | | | |
| 678 | 1383382_AT | BI294931 | Similar to Jumonji, AT rich interactive | | NM_004973 | NM_021878 |
| | | | domain 2 | | | |
| 679 | 1383489_AT | BG663025 | | | | |
| 680 | 1383498_AT | BG373354 | | | | |
| 681 | 1383533_AT | AI715743 | Similar to Homo sapiens REST corepressor 3 | | NM_018254 | NM_144814 |
| | | | (RCOR3) | | | |
| 682 | 1383776_AT | AW525120 | | | | |
| 683 | 1383844_AT | BI296898 | | | | |
| 684 | 1383914_AT | AI717613 | | | | |
| 685 | 1383935_AT | AW252428 | | | | |
| 686 | 1384188_AT | AW142766 | | | | |
| 687 | 1384192_AT | AA819913 | | | | |
| 688 | 1384232_AT | AI709768 | | | | |
| 689 | 1384335_AT | BE111885 | | | | |
| 690 | 1384724_AT | AA850766 | | | | |
| 691 | 1384899_AT | AA996921 | | | | |
| 692 | 1385359_AT | BF406890 | | | | |
| 693 | 1385506_AT | AW530507 | | | | |
| 694 | 1385925_AT | AA963975 | | | | |
| | 1385926_AT | BF550565 | | | | |
| 695 | 1388271_AT | BM383531 | | | | |
| 696 | 1388486_AT | BG671315 | | | | |
| 697 | 1388700_AT | AW253004 | highly similar to NP_899009.1 checkpoint | | NM_005197 | NM_183186 |
| | | | supressor 1 [Mus musculus] | | | |
| 698 | 1388705_AT | BI282694 | Rattus norvegicus transcribed sequence with | | NM_080430 | NM_053267 |
| | | | moderate similarity to protein | | | |
| | | | ref: NP_536355.1 (H. sapiens) selenoprotein | | | |
| | | | SelM [Homo sapiens] | | | |
| 699 | 1388742_AT | AA945877 | | | | |
| 700 | 1388870_AT | BF284305 | | | | |
| 701 | 1389018_AT | BF411239 | | | | |
| 702 | 1389039_AT | BM386520 | | | | |
| 703 | 1389151_AT | AI103440 | | | | |
| 704 | 1389186_AT | AA944175 | Similar to Mus musculus serologically | | NM_173485 | XM_130644 |
| | | | defined colon cancer antigen 33 like | | | |
| 705 | 1389213_AT | AI408955 | | | | |
| 706 | 1389590_AT | AA925513 | | | | |
| 707 | 1389651_AT | AI177057 | | | | |
| 708 | 1389789_AT | AI059662 | | | | |
| 709 | 1389883_AT | BF284719 | Transcribed locus, highly similar to | | NM_194291 | NM_175212 |
| | | | NP_919267.1 hypothetical protein BC017881 | | | |
| | | | [Homo sapiens] | | | |
| 710 | 1390173_AT | BG377886 | | | | |
| 711 | 1390300_AT | BM383635 | | | | |
| 712 | 1390306_AT | AW531909 | | | | |
| 713 | 1390380_AT | BE116633 | | | | |
| 714 | 1390536_AT | BF551311 | | | | |
| 715 | 1390555_AT | AW530876 | Transcribed locus, highly similar to | | NM_014011 | NM_019654 |
| | | | NP_054730.1 suppressor of cytokine | | | |
| | | | signaling 5 [Homo sapiens] | | | |
| 716 | 1390647_AT | AI410924 | | | | |
| 717 | 1390828_AT | BI395810 | | | | |
| 718 | 1390832_AT | BI294696 | | | | |
| 719 | 1390835_AT | AI013568 | | | | |
| 720 | 1391167_AT | AW532567 | | | | |
| 721 | 1391607_AT | BE109653 | | | NM_031459 | NM_144907 |
| 722 | 1391856_AT | BE108859 | | | | |
| 723 | 1391871_AT | AI763550 | | | | |
| 724 | 1392044_AT | BE117009 | | | | AK020182 |
| 725 | 1392220_AT | AW532634 | | | | |
| 726 | 1392497_AT | BE104938 | | | | |
| 727 | 1392597_AT | BG670559 | | | | |
| 728 | 1392736_AT | AW532939 | | | | |
| 729 | 1392934_AT | BM384509 | | | | |
| 730 | 1392948_AT | BI290737 | | | | |
| 731 | 1392953_AT | BI296250 | Transcribed locus, highly similar to | | NM_014241 | NM_013935 |
| | | | NP_038963.1 protein tyrosine phosphatase- | | | |
| | | | like (proline instead of catalytic arginine), | | | |
| | | | member a [Mus musculus] | | | |
| 732 | 1393003_AT | AA899900 | | | | |
| 733 | 1393316_AT | AI072658 | | | | |
| 734 | 1393730_AT | BI277836 | | | | |
| 735 | 1394109_AT | BF558056 | | | NM_003246 | NM_011580 |
| 736 | 1394315_AT | BF562800 | | | NM_003088 | NM_007984 |
| 737 | 1394459_AT | AI715738 | | | | |
| 738 | 1394612_AT | AW535206 | | | | |
| 739 | 1395079_AT | AI454784 | | | | |
| 740 | 1395197_AT | BI293027 | | | | |
| 741 | 1395635_AT | BE098317 | | | | |
| 742 | 1396208_AT | BM389807 | | | | |
| 743 | 1397317_AT | BI296984 | | | | |
| 744 | 1397808_AT | AW920064 | | | | |
| 745 | 1398131_AT | BE096035 | | | | |
| 746 | 1398380_AT | BF413643 | | | | |
| 747 | 1398390_AT | AA892854 | | | | |
| 748 | 1398597_AT | AI044699 | | | | |
| 749 | 1398656_AT | AI576497 | (human G protein-coupled receptor 133) | | NM_198827 | NM_177734 |
| 750 | 1398727_AT | BF398091 | | | | |
| 751 | 1399122_AT | BI278603 | | | | |
| 752 | 1377994_AT | AI501237 | | | | |
| 753 | 1378526_AT | AI012423 | | | | |
| 754 | 1383080_AT | BI292252 | | | | |
| 755 | 1383355_AT | AW918387 | Similar to ATP-binding cassette, sub-family | | NM_005502 | NM_013454 |
| | | | A (ABC1), member 1 | | | |
| 756 | 1383644_AT | BM385202 | | | | |

What is claimed is:

1. A method for identifying compounds for regulating angiogenesis, comprising:
a. contacting a compound with a protein identified in Table 4;
b. determining whether the compound binds or activates the protein; and
c. identifying those compounds that bind or activates the protein as compounds for regulating angiogenesis.

2. A method according to claim 1, further comprising:
a. selecting those compounds that bind or activate the protein, and further determining whether the compound regulates angiogenesis in an angiogenesis model system; and
b. identifying those compounds that regulates angiogenesis in an angiogenesis model system as compounds for regulating angiogenesis.

3. A method according to claim 1, further comprising: administering the compound identified in step (c) of claim 1 to a non-human animal, and determining whether the compound regulates angiogenesis in the animal, wherein compounds that regulate angiogenesis in the animal are identified as compounds for regulating angiogenesis in vivo.

4. A method according to claim 2, further comprising: administering the compound identified in step (b) of claim 2 to a non-human animal, and determining whether the compound regulates angiogenesis in the animal, wherein compounds that regulate angiogenesis in the animal are identified as compounds for regulating angiogenesis in vivo.

5. A method according to claim 1, wherein the protein is expressed on a cell.

6. A method for identifying compounds for regulating angiogenesis, comprising:
a. contacting a compound with a cell population expressing a gene identified in Table 4;
b. determining and comparing the level of expression of the gene in the cell population that is contacted with the compound to the level of expression of the gene in the cell population that is not contacted with the compound; and
c. identifying those compounds that modulate the expression of the gene in the cell population that is contacted with the compound compared to the expression of the gene in the cell population that is not contacted with the compound as compounds for regulating angiogenesis.

7. A method according to claim 6, further comprising:
a. determining whether the compound identified in step (c) of claim 6 regulates angiogenesis in an angiogenesis model system; and
b. identifying those compounds that regulate angiogenesis in an angiogenesis model system as compounds for regulating angiogenesis.

8. A method according to claim 6, further comprising: administering the compound identified in step (c) of claim 6 to a non-human animal, and determining whether the compound regulates angiogenesis in the animal, wherein compounds that regulate angiogenesis in the animal are identified as compounds for regulating angiogenesis.

9. A method according to claim 7, further comprising: administering the compound identified in step (b) of claim 7 to a non-human animal, and determining whether the compound regulates angiogenesis in the animal, wherein compounds that regulate angiogenesis in the animal are identified as compounds for regulating angiogenesis.

10. A method of monitoring the treatment or progression of a disorder in a patient with unregulated or improperly regulated angiogenesis, comprising:
a. preparing a gene expression profile for one or more gene involved in regulating angiogenesis identified in Table 4; or preparing a protein expression profile, or protein activity profile of one or more proteins involved in regulating angiogenesis identified in Table 4 from a subject;
b. administering a therapeutic regimen to the subject;
c. preparing a similar expression or activity profile as in step (a) from a cell or tissue sample from the subject after a suitable time after the therapeutic regimen;
d. comparing the profiles prior to the therapy with profiles after the therapy; and
e. repeating the steps (b), (c) and (d) during the course of the treatment or disorder and evaluating the data to monitor efficacy of the treatment or progression of the disorder.

11. A pharmaceutical composition comprising a safe and effective amount of a compound identified by the method according to claim 1.

12. A pharmaceutical composition comprising a safe and effective amount of a compound identified by the method according to claim 6.

13. A method according to claim 1, wherein the protein is ETL protein.

14. A method according to claim 6, wherein the gene is the gene coding for ETL protein.

15. A pharmaceutical composition according to claim 11, wherein the compound is an agonist or an antagonist of ETL protein.

16. A method according to claim 1, wherein the protein is GPR176 protein.

17. A method according to claim 6, wherein the gene is the gene coding for GPR176 protein.

18. A pharmaceutical composition according to claim 11, wherein the compound is an agonist or an antagonist of GPR176 protein.
